(19) United States
(12) Patent Application Publication
(10) Pub. No.: US 2026/0002139 A1
(43) Pub. Date: Jan. 1, 2026

(54) MICROHOMOLOGY MEDIATED REPAIR OF MICRODUPLICATION GENE MUTATIONS

(71) Applicant: UNIVERSITY OF MASSACHUSETTS, Westborough, MA (US)

(72) Inventors: Scot A. Wolfe, Winchester, MA (US); Charles P. Emerson, Lyndon, VT (US); Sukanya Iyer, Marlborough, MA (US); Sneha Suresh, Dracut, MA (US); Christian Mueller, Concord, MA (US); Jennifer Chen, Kingston (CA); Dongsheng Guo, Shrewsbury, MA (US); Oliver King, Somerville, MA (US)

(21) Appl. No.: 19/217,675

(22) Filed: May 23, 2025

Related U.S. Application Data
(62) Division of application No. 17/051,632, filed on Oct. 29, 2020, now Pat. No. 12,351,836.
(60) Provisional application No. 62/667,201, filed on May 4, 2018.
(60) Provisional application No. 62/823,173, filed on Mar. 25, 2019.

(51) Int. Cl. C12N 9/22; A61K 35/76; A61K 38/00

(52) U.S. Cl. CPC C12N 9/22; A61K 35/76; A61K 38/00; C12N 2310/20; C12N 2800/80

(57) ABSTRACT

The present invention is directed to the filed of gene therapy. In particular, compositions and methods are disclosed that repair gene microduplication mutations by reversion to a wild type sequence. For example, the creation of a double stranded break by a programmable nuclease protein within a microduplication induces the microhomology mediated end joining DNA repair pathway that in the process of DNA repair removes the microduplication mutation and restores the wild type sequence.

### CROSS-REFERENCE TO RELATED APPLICATIONS

This application is a U. S. Divisional Application of U.S. application Ser. No. 17/051,632, filed Oct. 10, 2020, now U.S. Pat. No. 12,351,836 issued Jul. 8, 2025; which is a U.S. national stage filing under 35 U.S.C. § 371 of and claims priority to, international application No. PCT/US19/30576, filed May 3, 2019, now expired, which claims priority under 35 U.S.C. § 119 (e) to U.S. Provisional Application Ser. No. 62/823,173, filed Mar. 25, 2019, now expired, and which claims priority under 35 U.S.C. § 119 (e) to U.S. Provisional Application Ser. No. 62/667,201, filed May 4, 2018, now expired, each of which is herein incorporated by reference in its entirety.

### REFERENCE TO AN ELECTRONIC SEQUENCE LISTING

The contents of the electronic sequence listing (20697.xml, created on Sep. 18, 2025, consisting of 1,148,302 bytes) is herein incorporated by reference in its entirety.

### FIELD OF THE INVENTION

The present invention is directed to the field of gene therapy. In particular, compositions and methods are disclosed that repair gene microduplication mutations by reversion to a wild type sequence. For example, the creation of a double stranded break within a microduplication by a programmable nuclease protein induces the microhomology mediated end joining DNA repair pathway that in the process of DNA repair removes the microduplication mutation and restores the wild type sequence.

### BACKGROUND

Genome editing by programmable nuclease systems has revolutionized biological research and is rapidly moving towards many clinical applications. In most instances, the successful repair of an aberrant gene to correct a disease entails precise correction of the genetic sequence typically via the Homology Directed Repair (HDR) pathway. This pathway requires not only the use of a programmable nuclease to generate a double-strand break (DSB) at the locus to initiate DNA repair, but also the delivery of exogenous donor DNA to precisely re-write the genomic sequence To date, HDR is inefficient in most cell types, particularly in post-mitotic differentiated cell types such as neurons and muscle [Suzuki, K. et al. In vivo genome editing via CRISPR/Cas9 mediated homology-independent targeted integration. Nature 540, 144-149 (2016).], which are the affected tissues in many devastating genetic disorders. This barrier significantly limits the clinical efficacy of the current generation of nuclease-based gene repair tools.

What is needed in the art are compositions and methods that can safely and efficiently target disease-causing microduplication mutations within a genome and cure the disease by reverting the microduplication mutation to a wild type sequence.

### SUMMARY

The present invention is directed to the filed of gene therapy. In particular, compositions and methods are disclosed that repair gene microduplication mutations by reversion to a wild type sequence. For example, the creation of a double stranded break within a microduplication by a programmable nuclease protein induces the microhomology mediated end joining DNA repair pathway that in the process of DNA repair removes the microduplication mutation and restores the wild type sequence.

In one embodiment, the present invention contemplates a programmable nuclease having sequence-specific DNA-binding affinity for a target gene or genomic locus, wherein said target gene or genomic locus comprises a microduplication mutation. In one embodiment, said nuclease further comprises a protospacer adjacent motif binding domain having said sequence-specific DNA-binding affinity for said target gene or genomic locus protospacer adjacent motif sequence. In one embodiment, the nuclease includes, but is not limited to, a Class II CRISPR single effector nuclease, a Cas9 nuclease, a Cas12 nuclease, a zinc finger nuclease and/or a transcription activator-like effector nuclease. In one embodiment, a duplicate sequence of the microduplication mutation has a length of between 1-40 nucleotides. In one embodiment, a duplicate sequence of the microduplication mutation has a length of greater than 40 nucleotides.

In one embodiment, the present invention contemplates a method, comprising; i) a subject comprising a target gene or genomic locus having a microduplication mutation; and ii) a pharmaceutical formulation comprising a programmable nuclease, the nuclease having sequence-specific DNA-binding affinity for a region that contains said microduplication mutation of the target gene or genomic locus; and b) administering said pharmaceutical formulation to the patient under conditions such that the microduplication mutation is replaced with a wild type sequence of the target gene or genomic locus. In one embodiment, said wild type sequence replacement comprises a correction through DNA repair. In one embodiment, the DNA repair correction is performed without assistance of an exogenously supplied donor DNA. In one embodiment, said nuclease further comprises a protospacer adjacent motif binding domain having said DNA-binding specificity for said target gene or genomic locus protospacer adjacent motif sequence. In one embodiment, the target gene includes, but is not limited to, TCAP, HPS1, HEXA, DOK7 and/or RAX2. In one embodiment, the subject further exhibits at least one symptom of a disease caused by the target gene microduplication mutation. In one embodiment, the disease includes, but is not limited to limb-girdle muscular dystrophy 2G, Hermanksy-Pudlak syndrome, Tay-Sachs Disease, familial limb-girdle myasthenia and/or cone-rod dystrophy 11. In one embodiment, administering further reduces the at least one symptom of the disease. In one embodiment, the nuclease includes, but is not limited to, a Class II CRISPR single effector nuclease, a Cas9 nuclease, a Cas12 nuclease, a zinc finger nuclease and/or a transcription activator-like effector nuclease. In one embodiment, the pharmaceutical formulation comprises an adeno-associated virus encoding said programmable nuclease.

### Definitions

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity but also plural entities and also includes the general class of which a specific example may be used for illustration. The terminology herein may be used to describe specific embodiments of the invention, but their usage does not delimit the invention, except as outlined in the claims.

The term “about” as used herein, in the context of any of any assay measurements refers to +/−5% of a given measurement.

As used herein, the term “CRISPRs” or “Clustered Regularly Interspaced Short Palindromic Repeats” refers to an acronym for DNA loci that contain multiple, short, direct repetitions of base sequences. Each repetition contains a series of bases followed by the same series in reverse and then by 30 or so base pairs known as “spacer DNA”. The spacers are short segments of DNA from a virus and may serve as a ‘memory’ of past exposures to facilitate an adaptive defense against future invasions (PMID 25430774).

As used herein, the term “Cas” or “CRISPR-associated (cas)” refers to genes often associated with CRISPR repeat-spacer arrays (PMID 25430774).

As used herein, the term “Cas9” refers to a nuclease from Type II CRISPR systems, an enzyme specialized for generating double-strand breaks in DNA, with two active cutting sites (the HNH and RuvC domains), one for each strand of the double helix. Jinek combined tracrRNA and spacer RNA into a “single-guide RNA” (sgRNA) molecule that, mixed with Cas9, could find and cleave DNA targets through Watson-Crick pairing between the guide sequence within the sgRNA and the target DNA sequence (PMID 22745249).

As used herein, the term “catalytically active Cas9” refers to an unmodified Cas9 nuclease comprising full nuclease activity.

The term “nickase” as used herein, refers to a nuclease that cleaves only a single DNA strand, either due to its natural function or because it has been engineered to cleave only a single DNA strand. Cas9 nickase variants that have either the RuvC or the HNH domain mutated provide control over which DNA strand is cleaved and which remains intact (Jinek, et al. 2012 (PMID 22745249) and Cong, et al. 2013 (PMID 23287718)).

As used herein, the term “Cas12” (or Cpf1) refers to a nuclease from Type V CRISPR systems, an enzyme specialized for generating double-strand breaks in DNA, with one active cutting sites (the RuvC domain), that cuts both DNA strands. Zetsche demonstrated that when programmed with its crRNA Cas12 (Cpf1), could find and cleave DNA targets through Watson-Crick pairing between the guide sequence within the crRNA and the target DNA sequence (PMID 26422227).

The term, “trans-activating crRNA”, “tracrRNA” as used herein, refers to a small trans-encoded RNA. For example, CRISPR/Cas (clustered, regularly interspaced short palindromic repeats/CRISPR-associated proteins) constitutes an RNA-mediated defense system, which protects against viruses and plasmids. This defensive pathway has three steps. First a copy of the invading nucleic acid is integrated into the CRISPR locus. Next, CRISPR RNAs (crRNAs) are transcribed from this CRISPR locus. The crRNAs are then incorporated into effector complexes, where the crRNA guides the complex to the invading nucleic acid and the Cas proteins degrade this nucleic acid. There are several pathways of CRISPR activation, one of which requires a tracrRNA, which plays a role in the maturation of crRNA. TracrRNA is complementary to base pairs with a pre-crRNA forming an RNA duplex. This is cleaved by RNase III, an RNA-specific ribonuclease, to form a crRNA/tracrRNA hybrid. This hybrid acts as a guide for the endonuclease Cas9, which cleaves the invading nucleic acid.

The term “nuclease” as used herein, refers to any protein comprising a pre-determined sequence of amino acids that bind to a specific nucleotide sequence and create a double stranded break. Such nucleases can include, but are not limited to, a Class II CRISPR single effector nuclease, a Cas9 nuclease, a Cas12 nuclease (also known as Cpf1), a zinc finger nuclease (ZFN) protein and/or a transcription activator-like effector nuclease (TALEN). For example, a Class II CRISPR single effector nuclease and/or a Cas9 nuclease may be assembled into a CRISPR complex.

The term “protospacer adjacent motif” (or PAM) as used herein, refers to a DNA sequence that may be required for a Cas9/sgRNA to form an R-loop to interrogate a specific DNA sequence through Watson-Crick pairing of its guide RNA with the genome.

The term “protospacer adjacent motif recognition domain” as used herein, refers to a nuclease C-terminus amino acid sequence having specific DNA-binding specificity to a target gene PAM sequence.

The term “target gene” as used herein, refers to a specific genomic region, usually comprising at least one allele, whose dysfunction is associated with a disease. For example, a target gene may have a microduplication mutation that is a causative factor for a disease. A microduplication can be composed of a tandem repeat. Tandem repeats in DNA are a pattern of one or more nucleotides that are repeated and the repetitions are directly adjacent to each other.

As used herein, the term “sgRNA” refers to single guide RNA used in conjunction with CRISPR associated systems (Cas). sgRNAs are a fusion of crRNA and tracrRNA and contain nucleotides of sequence complementary to the desired target site (Jinek, et al. 2012 (PMID 22745249)). Watson-Crick pairing of the sgRNA with the target site permits R-loop formation, which in conjunction with a functional PAM permits DNA cleavage or in the case of nuclease-deficient Cas9 allows binds to the DNA at that locus.

The term “patient” or “subject”, as used herein, is a human or animal and need not be hospitalized. For example, out-patients, persons in nursing homes are “patients.” A patient may comprise any age of a human or non-human animal and therefore includes both adult and juveniles (i.e., children). It is not intended that the term “patient” connote a need for medical treatment, therefore, a patient may voluntarily or involuntarily be part of experimentation whether clinical or in support of basic science studies.

The term “affinity” as used herein, refers to any attractive force between substances or particles that causes them to enter into and remain in chemical combination. For example, an inhibitor compound that has a high affinity for a receptor will provide greater efficacy in preventing the receptor from interacting with its natural ligands, than an inhibitor with a low affinity.

As used herein, the term “orthogonal” refers to targets that are non-overlapping, uncorrelated, or independent. For example, if two orthogonal Cas9 isoforms were utilized, they would employ orthogonal sgRNAs that only program one of the Cas9 isoforms for DNA recognition and cleavage (Esvelt, et al. 2013 (PMID 24076762)). For example, this would allow one Cas9 isoform (e.g. S. pyogenes Cas9 or spCas9) to function as a nuclease programmed by a sgRNA that may be specific to it, and another Cas9 isoform (e.g. N. meningitidis Cas9 or nmCas9) to operate as a nuclease dead Cas9 that provides DNA targeting to a binding site through its PAM specificity and orthogonal sgRNA. Other Cas9s include S. aureus Cas9 or SaCas9 and A. naeslundii Cas9 or AnCas9.

The term “truncated” as used herein, when used in reference to either a polynucleotide sequence or an amino acid sequence means that at least a portion of the wild type sequence may be absent. In some cases truncated guide sequences within the sgRNA or crRNA may improve the editing precision of Cas9 (Fu, et al. 2014 (PMID 24463574)).

The term “base pairs” as used herein, refer to specific nucleobases (also termed nitrogenous bases), that are the building blocks of nucleotide sequences that form a primary structure of both DNA and RNA. Double stranded DNA may be characterized by specific hydrogen bonding patterns, base pairs may include, but are not limited to, guanine-cytosine and adenine-thymine) base pairs.

The term “genomic locus” or “target gene” as used herein, refers to any pre-determined nucleotide sequence capable of binding to a Cas9 protein contemplated herein. The target may include, but may be not limited to, a nucleotide sequence complementary to a programmable DNA binding domain or an orthogonal Cas9 protein programmed with its own guide RNA, a nucleotide sequence complementary to a single guide RNA, a protospacer adjacent motif recognition sequence, an on-target binding sequence and an off-target binding sequence.

The term “on-target binding sequence” as used herein, refers to a subsequence of a specific genomic target that may be completely complementary to a programmable DNA binding domain and/or a single guide RNA sequence.

The term “off-target binding sequence” as used herein, refers to a subsequence of a specific genomic target that may be partially complementary to a programmable DNA binding domain and/or a single guide RNA sequence.

The term “cleavage” or “break” as used herein, may be defined as the generation of a break in the DNA. This could be either a single-stranded break or a double-stranded break depending on the type of nuclease that may be employed.

As used herein, the term “edit”, “editing” or “edited” refers to a method of altering a nucleic acid sequence of a polynucleotide (e.g., for example, a wild type naturally occurring nucleic acid sequence or a mutated naturally occurring sequence) by selective deletion of a specific genomic target or the specific inclusion of new sequence through the use of an exogenously supplied DNA template. Such a specific genomic target includes, but may be not limited to, a chromosomal region, mitochondrial DNA, a gene, a promoter, an open reading frame or any nucleic acid sequence.

The term “delete”, “deleted”, “deleting” or “deletion” as used herein, may be defined as a change in either nucleotide or amino acid sequence in which one or more nucleotides or amino acid residues, respectively, are, or become, absent.

As used herein, the terms “complementary” or “complementarity” are used in reference to “polynucleotides” and “oligonucleotides” (which are interchangeable terms that refer to a sequence of nucleotides) related by the base-pairing rules. For example, the sequence “C-A-G-T,” may be complementary to the sequence “A-C-T-G.” Complementarity can be “partial” or “total.” “Partial” complementarity may be where one or more nucleic acid bases may be not matched according to the base pairing rules. “Total” or “complete” complementarity between nucleic acids may be where each and every nucleic acid base may be matched with another base under the base pairing rules. The degree of complementarity between nucleic acid strands has significant effects on the efficiency and strength of hybridization between nucleic acid strands. This may be of particular importance in amplification reactions, as well as detection methods which depend upon binding between nucleic acids.

The terms “homology” and “homologous” as used herein in reference to nucleotide sequences refer to a degree of complementarity with other nucleotide sequences. There may be partial homology or complete homology (i.e., identity). A nucleotide sequence which may be partially complementary, i.e., “substantially homologous,” to a nucleic acid sequence may be one that at least partially inhibits a completely complementary sequence from hybridizing to a target nucleic acid sequence. The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous sequence to a target sequence under conditions of low stringency. This may be not to say that conditions of low stringency are such that non-specific binding may be permitted; low stringency conditions require that the binding of two sequences to one another be a specific (i.e., selective) interaction. The absence of non-specific binding may be tested by the use of a second target sequence which lacks even a partial degree of complementarity (e.g., less than about 30% identity); in the absence of non-specific binding the probe will not hybridize to the second non-complementary target.

The terms “homology” and “homologous” as used herein in reference to amino acid sequences refer to the degree of identity of the primary structure between two amino acid sequences. Such a degree of identity may be detected in a portion of each amino acid sequence, or along the entire length of the amino acid sequence. Two or more amino acid sequences that are “substantially homologous” may have at least 50% identity, preferably at least 75% identity, more preferably at least 85% identity, most preferably at least 95%, or 100% identity.

An oligonucleotide sequence which may be a “homolog” may be defined herein as an oligonucleotide sequence which exhibits greater than or equal to 50% identity to a sequence, when sequences having a length of 100 bp or larger are compared.

As used herein, the term “gene” means the deoxyribonucleotide sequences comprising the coding region of a structural gene and including sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into heterogeneous nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

The term “gene of interest” as used herein, refers to any pre-determined gene for which deletion may be desired.

The term “allele” as used herein, refers to any one of a number of alternative forms of the same gene or same genetic locus.

The term “protein” as used herein, refers to any of numerous naturally occurring extremely complex substances (as an enzyme or antibody) that consist of amino acid residues joined by peptide bonds, contain the elements carbon, hydrogen, nitrogen, oxygen, usually sulfur. In general, a protein comprises amino acids having an order of magnitude within the hundreds.

The term “peptide” as used herein, refers to any of various amides that are derived from two or more amino acids by combination of the amino group of one acid with the carboxyl group of another and are usually obtained by partial hydrolysis of proteins. In general, a peptide comprises amino acids having an order of magnitude within the tens.

The term “polypeptide”, refers to any of various amides that are derived from two or more amino acids by combination of the amino group of one acid with the carboxyl group of another and are usually obtained by partial hydrolysis of proteins. In general, a polypeptide comprises amino acids having an order of magnitude within the tens or larger.

“Nucleic acid sequence” and “nucleotide sequence” as used herein refer to an oligonucleotide or polynucleotide, and fragments or portions thereof, and to DNA or RNA of genomic or synthetic origin which may be single- or double-stranded, and represent the sense or antisense strand.

The term “an isolated nucleic acid”, as used herein, refers to any nucleic acid molecule that has been removed from its natural state (e.g., removed from a cell and may be, in a preferred embodiment, free of other genomic nucleic acid).

The terms “amino acid sequence” and “polypeptide sequence” as used herein, are interchangeable and to refer to a sequence of amino acids.

As used herein the term “portion” when in reference to a protein (as in “a portion of a given protein”) refers to fragments of that protein. The fragments may range in size from four amino acid residues to the entire amino acid sequence minus one amino acid.

The term “portion” when used in reference to a nucleotide sequence refers to fragments of that nucleotide sequence. The fragments may range in size from 5 nucleotide residues to the entire nucleotide sequence minus one nucleic acid residue.

As used herein, the term “hybridization” may be used in reference to the pairing of complementary nucleic acids using any process by which a strand of nucleic acid joins with a complementary strand through base pairing to form a hybridization complex. Hybridization and the strength of hybridization (i.e., the strength of the association between the nucleic acids) may be impacted by such factors as the degree of complementarity between the nucleic acids, stringency of the conditions involved, the Tm of the formed hybrid, and the G: C ratio within the nucleic acids.

As used herein the term “hybridization complex” refers to a complex formed between two nucleic acid sequences by virtue of the formation of hydrogen bounds between complementary G and C bases and between complementary A and T bases; these hydrogen bonds may be further stabilized by base stacking interactions. The two complementary nucleic acid sequences hydrogen bond in an antiparallel configuration. A hybridization complex may be formed in solution (e.g., C₀ tor R₀ t analysis) or between one nucleic acid sequence present in solution and another nucleic acid sequence immobilized to a solid support (e.g., a nylon membrane or a nitrocellulose filter as employed in Southern and Northern blotting, dot blotting or a glass slide as employed in in situ hybridization, including FISH (fluorescent in situ hybridization)).

As used herein, the term “Tₘ” may be used in reference to the “melting temperature.” The melting temperature may be the temperature at which a population of double-stranded nucleic acid molecules becomes half dissociated into single strands. As indicated by standard references, a simple estimate of the Tₘ value may be calculated by the equation: Tₘ=81.5+0.41 (% G+C), when a nucleic acid may be in aqueous solution at 1M NaCl. Anderson et al., “Quantitative Filter Hybridization” In: Nucleic Acid Hybridization (1985). More sophisticated computations take structural, as well as sequence characteristics, into account for the calculation of Tₘ.

As used herein the term “stringency” may be used in reference to the conditions of temperature, ionic strength, and the presence of other compounds such as organic solvents, under which nucleic acid hybridizations are conducted. “Stringency” typically occurs in a range from about Tₘ to about 20° C. to 25° C. below Tₘ. A “stringent hybridization” can be used to identify or detect identical polynucleotide sequences or to identify or detect similar or related polynucleotide sequences. For example, when fragments are employed in hybridization reactions under stringent conditions the hybridization of fragments which contain unique sequences (i.e., regions which are either non-homologous to or which contain less than about 50% homology or complementarity) are favored. Alternatively, when conditions of “weak” or “low” stringency are used hybridization may occur with nucleic acids that are derived from organisms that are genetically diverse (i.e., for example, the frequency of complementary sequences may be usually low between such organisms).

As used herein, the terms “restriction endonucleases” and “restriction enzymes” refer to bacterial enzymes, each of which cut double-stranded DNA at or near a specific nucleotide sequence.

DNA molecules are said to have “5′ ends” and “3′ ends” because mononucleotides are reacted to make oligonucleotides in a manner such that the 5′ phosphate of one mononucleotide pentose ring may be attached to the 3′ oxygen of its neighbor in one direction via a phosphodiester linkage. Therefore, an end of an oligonucleotide may be referred to as the “5′ end” if its 5′ phosphate may be not linked to the 3′ oxygen of a mononucleotide pentose ring. An end of an oligonucleotide may be referred to as the “3′ end” if its 3′ oxygen may be not linked to a 5′ phosphate of another mononucleotide pentose ring. As used herein, a nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements which direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

As used herein, the term “an oligonucleotide having a nucleotide sequence encoding a gene” means a nucleic acid sequence comprising the coding region of a gene, i.e. the nucleic acid sequence which encodes a gene product. The coding region may be present in a cDNA, genomic DNA or RNA form. When present in a DNA form, the oligonucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

As used herein, the terms “nucleic acid molecule encoding”, “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

The term “bind”, “binding”, or “bound” as used herein, includes any physical attachment or close association, which may be permanent or temporary. Generally, an interaction of hydrogen bonding, hydrophobic forces, van der Waals forces, covalent and ionic bonding etc., facilitates physical attachment between the molecule of interest and the analyte being measuring.

The “binding” interaction may be brief as in the situation where binding causes a chemical reaction to occur. That may be typical when the binding component may be an enzyme and the analyte may be a substrate for the enzyme. Reactions resulting from contact between the binding agent and the analyte are also within the definition of binding for the purposes of the present invention.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 shows an overview of several naturally occurring mutagenic DNA repair pathways. Microhomology Mediated End Joining (MMEJ) mediated repair is the center pathway involving a 5′ end resection and annealing of homologous sequences.

FIG. 2 presents exemplary sequences of microduplication targets within a TCAP gene and a HSP1 gene. The microduplicated sequences in each exon are highlighted with and without boxes. The target sites for SpCas9 are shown below each sequence with the GG PAM element in bold and the spacer (guide) sequence underlined. The position of the DSB in the sequence is indicated by a “·”.

FIG. 3 presents exemplary data of a TIDE analysis of sanger sequence chromatogram from SpCas9 treated LGMD2G iPSCs. The estimated mutagenesis rate is 87% with 53% of the alleles containing an 8 bp deletion (oval), which would be consistent with a reversion to the WT sequence.

FIG. 4 presents exemplary data of an ICE analysis of sanger sequence chromatogram from SpCas9 treated HPS1 B-EBV cells. The estimated mutagenesis rate is 41% with 30% of the alleles containing a 16 bp deletion (oval), which would be consistent with a reversion to the WT sequence.

FIG. 5 presents exemplary data of a deep sequencing analysis of the editing rates and outcomes at TCAP in iPSCs or iPSC derived myoblasts that have an 8 bp duplication in both alleles. More than 50% of the alleles are either the precise 8 bp deletion or are mutations that produce an in frame sequence.

FIG. 6 presents exemplary data of TIDE analyses of sanger sequencing of individual iPSC clones from the 8 bp duplication TCAP line following treatment with a TCAP targeting SpCas9 RNP. In these four instances at least one allele was converted to the wild-type sequence.

FIG. 7 presents exemplary data for a phenotype prediction iPSC clones based on the genotypes that were observed from the sequencing of the Cas9 modified alleles. 75% of the clones that could be conclusively characterized contained at least one wild-type TCAP sequence.

FIG. 8 presents an overview of duplicated repeat collapse by MMEJ-mediated DNA repair pathways. A nuclease targeted near the center of the duplicated segment can lead to the collapse of the duplication. As disclosed herein, the present method targets this collapse and restores the wild-type sequence.

FIG. 9 presents exemplary data showing that a mutant HPS1 allele contains a 16 bp duplication (annotation as in FIG. 2).

FIG. 10A-E shows that MMEJ-based repair efficiently and precisely corrects TCAP allele containing an 8-bp duplication.

FIG. 10A: Schematic of MMEJ-based pathway for repair of a microduplication. A DSB at the centre of a microduplication (reptition) is expected to initiate 5′ end resection to expose the microhomologies on each side. These repeats anneal with each other and are repaired via the MMEJ pathway to yield the wild-type (WT) sequence.

FIG. 10B: The pathogenic 8-bp microduplication within TCAP (bold grey text) with the SpyCas9 protospacer-adjacent motif (PAM) sequence in the box and the protospacer sequence underlined. A SpyCas9-induced DSB (carets) is expected to drive MMEJ repair to revert the mutant allele to the wild-type sequence (grey text).

FIG. 10C: Percentage of 8-bp deletion (white bars) and total indels (grey bars) resulting from SpyCas9 RNP treatment of LGMD2G iPSCs homozygous for the 8-bp microduplication or wild-type iPSCs. Bars denote mean and dots indicate individual data points. n=3 biological replicates.

FIG. 10D: Genotype analysis of 22 LGMD2G iPSC clones after treatment with SpyCas9 RNPs. Hypo, hypomorphic allele.

FIG. 10E: Percentage of 8-bp deletion (white bars) and total indels (grey bars) resulting from SpyCas9 treatment of myoblasts derived from patient-derived LGMD2G iPSCs. Bars denote mean and dots indicate individual data points. n=3 biological replicates.

FIG. 11A-C shows that MMEJ-based repair efficiently and precisely corrects HPS1 allele containing 16-bp microduplication.

FIG. 11A: The 16-bp microduplication repeats are shown in bold grey and grey text. For six SpyCas9 guides targeting the microduplication, the PAM sequence is demarcated in the box and the protospacer sequence is underlined. A DSB (carets with distance from the repeat centre indicated) is expected to drive reversion to the wild-type sequence (grey text). Sequence underlined with grey bold lines in target site 6 indicates an alternate 16-bp microhomology within this repeat.

FIG. 11B: Percentage of 16-bp deletions (white) and total indels (grey) for guides shown in a based on UMI-based Illumina sequencing. Bars denote mean and dots indicate individual data points. n=3 biological replicates.

FIG. 11C: Percentage of wild-type reverted alleles (16-bp deletion) among all alleles with insertions or deletions (indels) from b. Mean±s.e.m., dots indicate individual data points. n=3 biological replicates.

FIG. 12A-D presents exemplary data showing that PARP-1 inhibition decreases efficiency of MMEJ-based repair.

FIG. 12A: Experimental design. HPS1 B-LCL cells were treated with rucaparib 24 h before and after electroporation with SpyCas9 RNPs targeting the HPS1 locus and collected for subsequent UMI-based Illumina sequencing¹⁵.

FIG. 12B: Percentage of microhomology (MH)-mediated deletion (white) and total indels (grey) in cells treated with SpyCas9 in the presence of 0, 10 or 20 μM rucaparib, measured by UMI-based Illumina deep sequencing. Bars denote mean and dots indicate individual data points. n=3 biological replicates.

FIG. 12C: Percentage of microhomology-mediated deletion alleles among all other alleles with indels from FIG. 12B. Mean±s.e.m., dots indicate individual data points. n=3 biological replicates. ****P=0.00003, unpaired two tailed t-test.

FIG. 12D: Left, alignment of resulting sequences observed by Illumina sequencing upon SpyCas9 RNP treatment of HPS1 B-LCL cells. Right, heatmap showing percentage of alleles generated by SpyCas9 for cells exposed to 0, 10 or 20 μM rucaparib. Gradient scale indicates the percentage occurrence of that sequence.

FIG. 13A-C presents exemplary data showing that MMEJ-based approach efficiently achieves precise collapse of endogenous microduplications across various repeat lengths.

FIG. 13A: Non-pathogenic endogenous microduplications ranging in size from 4 bp to 36 bp. Microduplication repeats are shown as bold text. The SpyCas9 PAM sequence is shown in the box on the right and the LbaCas12a PAM sequence is shown in the off center light grey box. Anticipated DSBs produced by SpyCas9 and LbaCas12a are denoted by grey and white carets, respectively.

FIG. 13B: Percentage of microhomology-mediated deletion (white) and total indels (grey) produced at each endogenous site following SpyCas9 treatment, calculated using UMI-based Illumina sequencing. Bars denote mean and dots indicate individual data points. n=3 biological replicates.

FIG. 13C: Percentage of microhomology-mediated deletions (white) and total indels (grey) produced at three endogenous sites when treated with SpyCas9 or LbaCas12a. Bars denote mean and dots indicate individual data points. n=3 biological replicates.

FIG. 14 show the disease-causing GATA microduplication (grey underlined-grey tandem segment) in the Tay-Sachs HEXA gene.

FIG. 15 presents exemplary data showing indel/deletion ratios subsequent to HEXA gene editing with Cas12a RNPs in a Tay-Sachs patient-derived B-EBV cells with a Cas9: sgRNA concentration ratio of 60 pmol protein to 120 pmol guide RNA.

FIG. 16 presents exemplary data showing representative sequence chromatograms of the edited HEXA genes, where sequencing is on the complementary strand.

FIG. 17 presents exemplary data showing indel/deletion ratios subsequent to HEXA gene editing with Cas12a RNPs in a Tay-Sachs patient-derived B-EBV cells with a Cas9: sgRNA concentration ratio of 90 pmol protein to 180 pmol guide RNA.

FIG. 18A-E presents exemplary data showing the effect of rucaparib on the profile of microhomology-mediated deletion products at AAVS1 locus in patient-derived HPS1 B-LCL cells.

FIG. 18A: Schematic of two prominent DNA double-strand break repair pathways. A DSB can be repaired through various pathways that produce different DNA sequence end-products. The NHEJ pathway is the dominant DSB repair pathway in most cells. The MMEJ pathway uses end-resection to discover small homologies on each side of the break that can be used to template the fusion of the broken ends. PARP-1 regulates DSB flux through the MMEJ pathway. Treatment of cells with rucaparib—an inhibitor of PARP-1—attenuates DSB flux down the MMEJ repair pathway.

FIG. 18B: Percentage of microhomology-mediated deletions (white) and total indels (grey) resulting from SpyCas9 treatment of cells in the presence of 0, 10 and 20 μM rucaparib. Bars show mean and dots show individual data points from three biological replicates based on UMI-based Illumina deep sequencing.

FIG. 18C: Percentage of 1-bp insertions (black), microhomology mediated deletions (white) and other deletions (grey) produced by SpyCas9 RNP with a sgRNA targeting the AAVS1 locus with the addition of increasing amounts of rucaparib. Mean±s.e.m. from three biological replicates based on UMI-based Illumina deep sequencing.

FIG. 18D: Percentage of microhomology-mediated deletions out of total indels in cells treated with SpyCas9 in the presence of rucaparib. Mean±s.e.m., dots represent individual data points from three biological replicates. P values determined using two-tailed unpaired t-test. ***P=0.0004, ****P=6.5×10⁻⁷.

FIG. 18E: Left, alignment of allele sequences obtained from deep sequencing analysis from samples treated with SpyCas9 RNP in the presence of different rucaparib concentrations. Microhomologies present at the AAVS1 locus are shown in by grey. Microhomology-mediated deletion is indicated by two-toned text. The carets indicate site of DSB created by SpyCas9. Inserted bases (ins) are shown in lower case, deleted bases (del) are shown as black dashes. Right, heatmap depicting the percentage of alleles generated after SpyCas9 treatment of cells in the presence of different concentrations of rucaparib (0, 10 or 20 μM). The grey colour gradient scale indicates the percentage of occurrence of that sequence. Heatmap represents mean values from a total of three independent biological replicates.

FIG. 19A-G presents exemplary data showing gene editing with SpyCas9 and LbCas12a at endogenous microduplications.

FIG. 19A: Percentage of microhomology-mediated deletions out of total indels at endogenous sites in cells treated with SpyCas9 and LbaCas12a. Mean±s.e.m., dots represent individual data points from three biological replicates.

FIG. 19B: Schematic of endogenous site containing a 24-bp microduplication for SpyCas9 target sites 1-3. The 24-bp microduplication repeats are shown. The PAM sequence is outlined and the protospacer sequence is underlined. The carets indicate the site of DSB.

FIG. 19C: Percentage of alleles with 24-bp deletion (white) and total indels (grey) for all three guides from TIDE analysis. Guide 3 produces primarily 23-bp deletions, but not 24-bp deletions, probably because it recuts the collapsed DNA sequence. Bars shows the mean from n=3 biological repeats, individual data points are represented by dots.

FIG. 19D: Proportion of the 24-bp deletion out of total indels as individual data points (dots), with mean±s.e.m. n=3 biological repeats.

FIG. 19E: Schematic of endogenous site containing a 27-bp microduplication for SpyCas9 target sites 1 and 2.

FIG. 19F: Percentage of alleles with 27-bp deletion (white) and total indels (grey) for both guides from UMI-based Illumina deep sequencing. Bars show the mean from n=3 biological repeats, individual data points are represented by dots.

FIG. 19G: Proportion of the 27-bp deletion out of total indels as individual data points (dots) with mean±s.e.m. n=3 biological replicates.

FIG. 20A-D presents exemplary data showing indel populations resulting from SpyCas9 editing at the TCAP locus.

FIG. 20A: Indel percentages resulting from SpyCas9 RNP treatment in patient-derived iPSCs homozygous for the 8-bp microduplication or in wild-type iPSCs. Mean±s.e.m. from three biological replicates.

FIG. 20B: Breakdown of indel classes resulting from SpyCas9 treatment of myoblasts derived from patient-derived LGMD2G iPSCs. Mean±s.e.m. from three biological replicates.

FIG. 20C: Sequence alignment of the edited alleles resulting from SpyCas9 RNP treatment of LGMD2G iPSCs. Grey text indicates DNA repeats that constitute the microduplication, and collapse is indicated by underlined text. Dashes indicate deleted bases and lower case text indicates inserted bases. Data are from one biological replicate out of three independent biological replicates.

FIG. 20D: Sequence alignment of the edited alleles resulting from SpyCas9 RNP treatment of myoblasts derived from patient-derived LGMD2G iPSCs. Data are from one biological replicate out of three independent biological replicates.

FIG. 21A-B presents exemplary data showing PacBio long-read sequencing analysis for SpyCas9-edited LGMD2G iPSCs at the TCAP locus.

FIG. 21A: Percentage of gene modification observed from PacBio sequencing (one replicate from FIG. 10C out of three biological replicates). black, alleles containing the 8-bp deletion; grey, other small indels (≤100 bp); light grey, large insertions (0.14%, not visible on the graph); white, large deletions (>100 bp).

FIG. 21B: IGV graphs depicting representative reads obtained for unedited (top) and edited (bottom) LGMD2G iPSCs, spanning a genomic region of about 2,035 bp surrounding the TCAP target site. The carat indicates the 8-bp deletion site. Data represent one replicate out of three independent biological replicates.

FIG. 22 presents exemplary data showing PacBio long-read sequencing analysis of SpyCas9-edited LGMD2G iPSCs clones and a complex colony at the TCAP locus. IGV graphs depicting representative reads obtained for clonal isolates of edited LGMD2G iPSCs (FIG. 10D), spanning a genomic region of about 2,035 bp surrounding the TCAP target site. The genotype of the clones (deduced by Illumina deep sequencing) is indicated beside an enlargement of the TCAP target region within the PacBio data. The sequences of the two alleles (listed above the IGV plot) obtained from sequencing are shown with repeats. Alleles that reverted to wild-type as a result of collapse of microduplication are underlined. Bottom, IGV plot for one complex iPSC colony that appears to have been nucleated by more than one cell, with large deletions present in the genome (sizes indicated).

FIG. 23A-E presents exemplary data showing Detection of telethonin expression by flow cytometry in patient-derived cells treated with SpyCas9.

FIG. 23A: Contour plots from a representative flow cytometry assay to detect telethonin expression in healthy control cells (TCAP^{+/+}), patient cells (TCAP/-), and SpyCas9-treated homozygous and heterozygous iPS clone-derived myoblasts differentiated for 10 days in culture. Plots are representative of three independent replicates.

FIG. 23B: Histograms from a representative flow cytometry assay to detect telethonin expression. Left, overlay of anti-telethonin antibody staining for four representative samples for different TCAP genotypes. Right, comparison between patient cells and healthy control cells, and SpyCas9-treated homozygous and heterozygous iPS clone-derived myoblasts differentiated for 10 days in culture. Histograms are representative of three independent replicates.

FIG. 23C: Cells were selected by removing cell debris first as shown by gate P1, and then single cells were selected from P1 by removing clustered cells as shown by gate P2. The cells in gate P2 were used for flow analysis. Plots are representative of one biological replicate.

FIG. 23D: Average percentage of telethonin-expressing cells from two technical replicates of three biological replicates. Error bars indicate s.e.m (n=6) and circles represent individual data points. P values (0.33 for patient versus heterozygous and 0.04 for patient versus homozygous clones) were calculated by two-sided Student's t-test.

FIG. 23E: Western blot showing validation of anti-telethonin antibody (Santa Cruz Biotechnology). Human muscle lysate and lysate from HEK293T cells transfected with haemagglutinin-tagged-telethonin expression construct were separated on an SDS 4-12% acrylamide gradient gel and the resulting blot was probed with anti-telethonin antibody.

FIG. 24 presents exemplary data showing a standard curve generated with genomic DNA of wild-type and HPS1 mutant B-LCLs from UMI-based Illumina deep sequencing. Genomic DNA from wild-type cells and HPS1 cells homozygous for the 16-bp microduplication were mixed at different ratios (x-axis). These mixed DNAs were used for the construction of a UMI-based Illumina library to determine the ratio of the alleles through deep sequencing (y-axis). These data are fitted to a regression line with the R2 value reported. n=1 biological replicate.

FIG. 25A-F presents exemplary data showing an Indel spectrum generated by SpyCas9 editing at the HPS1 locus in HPS1 B-LCL cells. Indel spectra of SpyCas9 nuclease cells treated with different sgRNAs determined by UMI-based Illumina deep sequencing. White bar indicates 16-bp deletion that corresponds to the deletion of one of the microduplication repeats. Data show indel spectra from one representative biological replicate out of three independent biological replicates.

FIG. 25A: Target site 1.

FIG. 25B: Target site 2.

FIG. 25C: Target site 3.

FIG. 25D: Target site 4.

FIG. 25E: Target site 5.

FIG. 25F: Target site 6.

FIG. 26A-B presents exemplary data showing pathogenic microduplications and their prevalence in human populations.

FIG. 26A: Number of insertion variants of length >1 bp that are annotated as Pathogenic or Pathogenic/likely pathogenic in ClinVar. Variants are binned by length, with all those of length 40 bp or greater combined. The insertions (grey) are stratified into progressively finer categories: duplications (red); ‘simple’ duplications (described in text, orange); and the subset of these observed at least once in gnomAD exome/genome databases (green).

FIG. 26B: Number of insertion variants of length >1 bp that are observed at least once in the ‘coding’ regions of the gnomAD exome/genome databases. As above, insertions (grey) are stratified into progressively finer categories: duplications (red); ‘simple’ duplications (orange); the subset of these listed in ClinVar (cyan); and the subset annotated as Pathogenic or Pathogenic/likely pathogenic in ClinVar (green). Cyan and green bars are not visible at this resolution.

FIG. 27 presents an exemplary bioinformatics pipeline for identification of disease alleles. Schematic shows the bioinformatics pipeline used to identify all microduplications amendable to efficient MMEJ-mediated collapse from the ‘coding’ regions (exome_calling_regions.v1; mainly exons plus 50 flanking bases) in the gnomAD genome and exome databases (version 2.0.2). Insertion variants observed in both databases were used for analysis (variants occurring in both databases were counted once). Insertions that do not add a repeat-unit to an existing tandem repeat and are not themselves a perfect repeat were filtered to constrain only duplications that spanned 2-40 bp in length and are amendable to CRISPR-Cas9 targeting. This data set was then cross-referenced against the Clin Var database (clinvar_20180225.vcf) to apply further filters for variants reported as pathogenic, which ultimately yielded 143 likely disease-causing microduplications.

### DETAILED DESCRIPTION OF THE INVENTION

The present invention is directed to the field of gene therapy. In particular, compositions and methods are disclosed that repair gene microduplication mutations by reversion to a wild type sequence. For example, the creation of double stranded breaks by a nuclease protein induces the microhomology mediated end joining DNA repair pathway that corrects the microduplication back to the wild type sequence without the assistance of an exogenously supplied donor DNA.

In one embodiment, the present invention contempates a subset of disease-causing alleles within the human population that are the product of small duplications (microduplications of 1 to 40 base pairs) within a gene sequence. These alleles occur in human subpopulations with substantial frequencies and result in rare diseases such as Limb Girdle Muscular Dystrophy 2G (LGMD2G) [Nigro, V. & Savarese, M. Genetic basis of limb-girdle muscular dystrophies: the 2014 update. Acta Myol 33, 1-12 (2014)], Tay-Sachs Disease [Fernandes Filho, J. A. & Shapiro, B. E. Tay-Sachs disease. Arch. Neurol. 61, 1466-1468 (2004).], and Hermansky-Pudlak syndrome (HPS) [El-Chemaly, S. & Young, L. R. Hermansky-Pudlak Syndrome. Clin. Chest Med. 37, 505-511 (2016).] among others (Table 1).

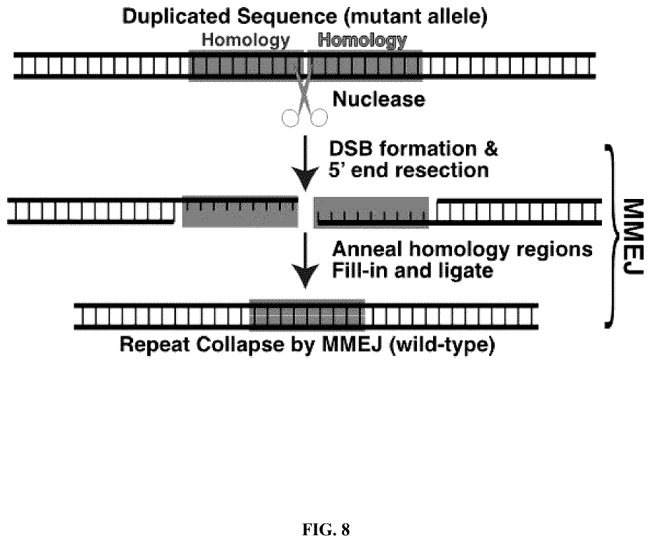

In one embodiment, the present invention contemplates a method demonstrating that disease-causing microduplications can be reverted to the wild-type sequence simply through the generation of a DSB near the center of the duplication, enabling development of simplified Cas9-based therapeutic interventions tailored to each disorder. Our discovery was based initially on the theoretical idea that a nuclease-generated DSB would harness a common cellular DNA repair pathway—microhomology mediated end joining (MMEJ). Sfeir et al., “Microhomology-Mediated End Joining: A Back-up Survival Mechanism or Dedicated Pathway?” Trends Biochem Sci 40:701-714 (2015). MMEJ utilizes small regions of sequence homology on each side of the break to collapse the DNA sequence. See, FIG. 8. The idea of microhomology collapse of sequences has been observed in programmable nuclease editing data for some time. Bae et al., “Microhomology-based choice of Cas9 nuclease target sites” Nature Methods 11:705-706 (2014). However, the realization that it could be applied to disease correction to achieve highly efficient reversion to the wild-type sequence has not been described.

The data presented herein demonstrates a successful, efficient correction of disease-causing alleles in patient-derived cell lines harboring microduplications including, but not limited to, TCAP (LGMD2G) and HPS1 (HPS). The data shows that this correction can be successfully performed in iPSC, stem cell progenitor cells and adult somatic cells, opening up multiple route for the delivery of a nuclease-based therapy. Based on a computational analysis of human allele variants described herein, more than 100 diseases have been identified that should be amenable to this type of genetic correction. As the introduced nuclease is programmed to target a mutant DNA sequence, the reverted wild-type sequence is not a substrate, and thus should be stable even in the presence of the nuclease. Furthermore, microhomolgy-mediated correction does not require a DNA cassette to regenerate the wild-type sequence, only the transient delivery of the nuclease (e.g. Cas9 and its sgRNA) to target the locus. The demonstrated high rate of correction for these two distinct genetic disorders suggests that our correction approach will have broad application to a wide variety of important genetic disorders associated with microduplications for which there are no therapeutics currently available, providing patients with a definitive cure.

Although it is not necessary to understand the mechanism of an invention, it is believed that targeting a double strand break to a microduplication can cause the collapse of the microduplication back to the wild-type sequence with high efficiency and that this might be used to correct disease alleles without the need for a DNA repair template.

Current programmable nuclease-based methods (for example, CRISPR-Cas9) for precise correction of a disease-causing genetic mutation harness the homology-directed repair pathway. However, this repair process requires co-delivery of an exogenous DNA donor to recode the sequence and can be inefficient in many cell types. In some embodiments, the present invention contemplates disease-causing frameshift mutations resulting from microduplications which can be efficiently reverted to the wild-type sequence simply by generating a double-stranded break near the centre of the duplication. It has been demonstrated herein using patient-derived cell lines: for example, limb-girdle muscular dystrophy 2G (LGMD2G)¹, Hermansky-Pudlak syndrome type 1 (HPS1)² and Tay-Sachs Disease. Clonal analysis of inducible pluripotent stem cells (iPSCs) from the LGMD2G cell line, which contain a mutation in TCAP, treated with the Streptococcus pyogenes Cas9 (SpyCas9) nuclease revealed that about 80% contained at least one wild-type TCAP allele; this correction also restored TCAP expression in LGMD2G iPSC-derived myotubes. SpyCas9 also efficiently corrected the genotype of an HPS1 patient-derived B-lymphoblastoid cell line. Inhibition of polyADP-ribose polymerase 1 (PARP-1) suppressed the nuclease-mediated collapse of the microduplication to the wild-type sequence, confirming that precise correction is mediated by the microhomology-mediated end joining (MMEJ) pathway. Analysis of editing by SpyCas9 and Lachnospiraceae bacterium ND2006 Cas12a (LbaCas12a) at non-pathogenic 4-36-base pair microduplications within the genome indicates that the correction strategy is broadly applicable to a wide range of microduplication lengths and can be initiated by a variety of nucleases. Finally, LbaCas12a was employed to achieve precise correction of the four base pair duplication in HEXA Tay-Sachs patient-derived B-lymphoblastoid cell line. The simplicity, reliability and efficacy of this MMEJ-based therapeutic strategy should permit the development of nuclease-based gene correction therapies for a variety of diseases that are associated with microduplications.

### I. Double Stranded Deoxyribonucleotide Break Repair Mechanisms

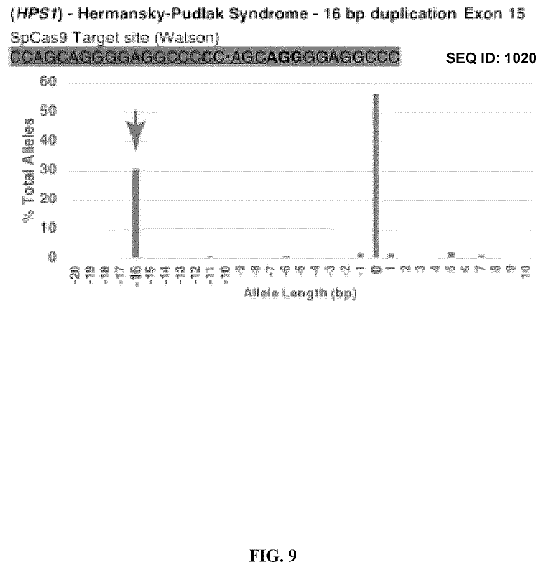

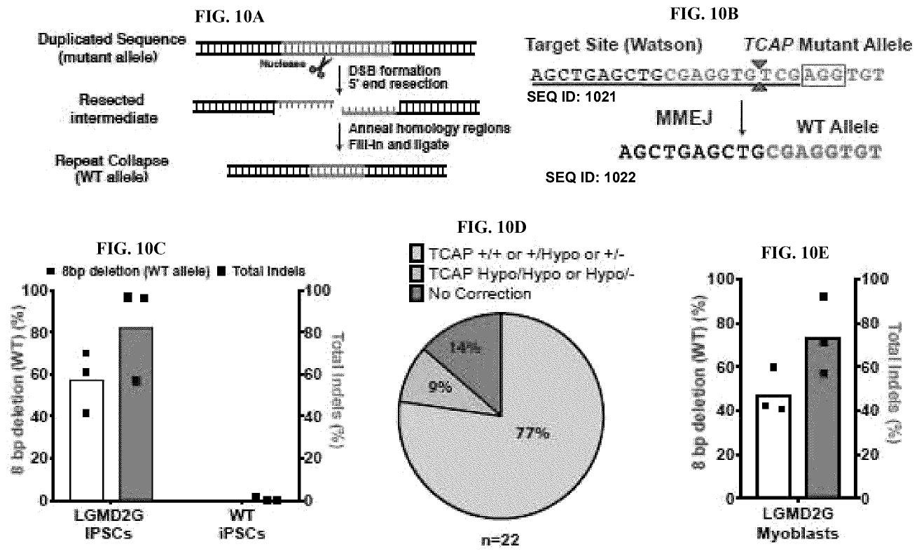

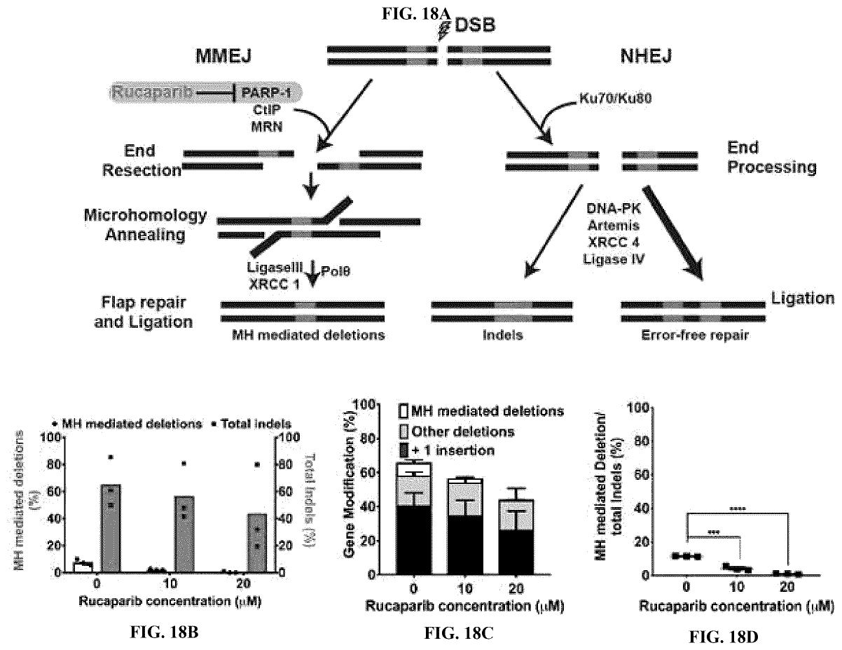

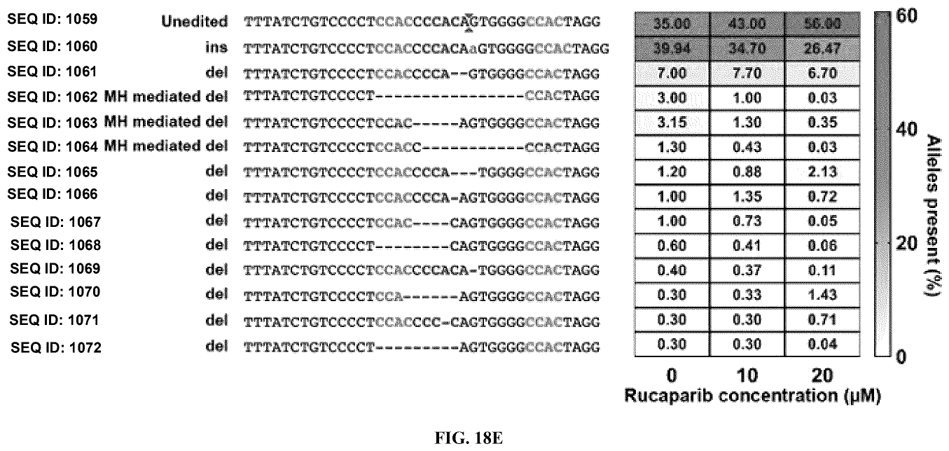

MMEJ is an error-prone double-stranded break (DSB) DNA repair pathway that uses regions of microhomology (2-25 bp) on each side of a DSB to define the boundaries at which DNA segments are rejoined³. This mutagenic process generates deletions that result in the loss of one of the repeat sequences and the intervening region. See FIG. 10A and FIG. 18. Hallmarks of MMEJ repair on DNA products generated through editing of programmable nucleases have been observed in a variety of cell types and their effect on gene inactivation rates has been appreciated^{4,5}. The MMEJ pathway has also been harnessed for the targeted insertion of exogenous donor DNAs in mammalian cells and zebrafish and frog embryos^{6,7}. Herein, a nuclease-based therapeutic approach is described that harnesses the MMEJ pathway to precisely correct frameshift mutations resulting from microduplications (e.g., tandem duplications). It was reasoned that MMEJ-based repair of a programmable nuclease-induced DSB near the centre of a disease-causing microduplication would achieve precise reversion to the wild-type genomic sequence. This strategy might be an effective alternative to homology-directed repair-based gene correction approaches and would not require co-delivery of a donor DNA. Furthermore, the reverted wild-type sequence would no longer be complementary to the single-guide RNA (sgRNA) targeting the microduplication, leading to stable correction even in the presence of Cas9 nuclease.

To evaluate the efficacy of the presently contemplated MMEJ-based correction strategy, LGMD2G and HPS1 were selected as exemplary diseases that affect different human tissues and whose causes include pathogenic microduplications of different lengths. Both of these diseases are autosomal recessive disorders that are represented at modest frequencies in different human subpopulations and currently have no treatments. One of the disease alleles identified in LGMD2G patients features an 8-bp duplication in exon 1 of TCAP, a mutation that is found in the East Asian population at a frequency of approximately 1 in 1,000 alleles. TCAP encodes the telethonin protein, a 19-kDa cardiac and striated muscle-specific structural protein located in the Z-disc of sarcomeres that links titin proteins to stabilize the contractile apparatus for muscle contraction⁸. Homozygous or compound heterozygous inactivating mutations in TCAP manifest as severe muscle atrophy and cardiomyopathy that typically develop during late adolescence into early adulthood^{1,9}.

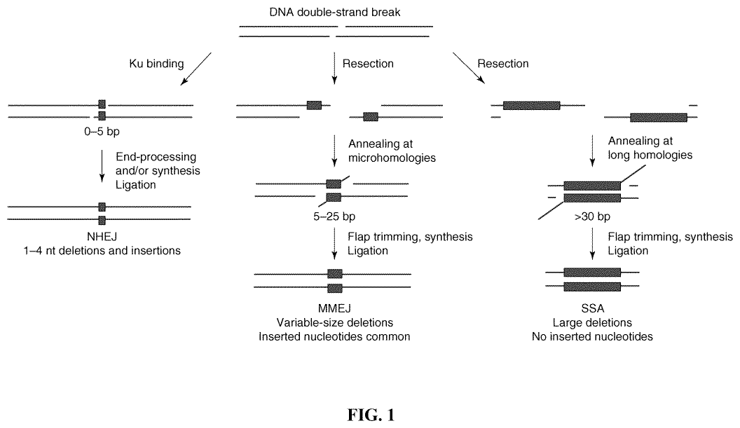

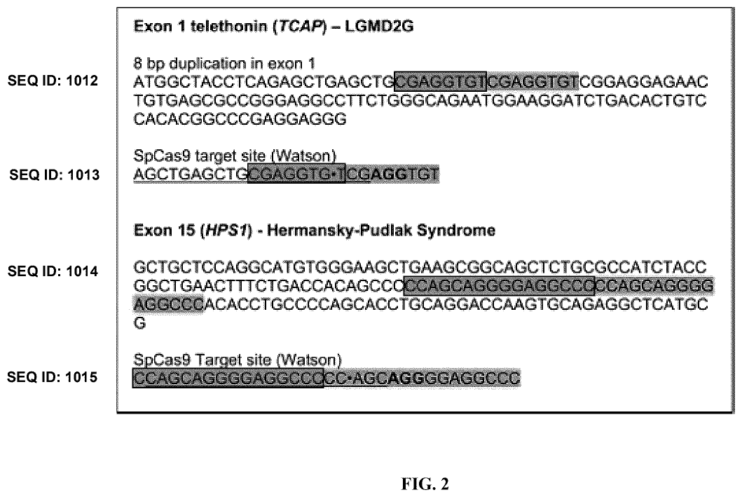

The double strand breaks (DSBs) that are generated within the genomes of eukaryotic systems are potentially repaired by a number of different DNA-damage response pathways such as canonical non-homologous end joining (cNHEJ), homologous recombination (HR), and alternate non-homologous end joining (aNHEJ). McVey et al., “MMEJ repair of double-strand breaks (director's cut): deleted sequences and alternative endings” Trends in Genetics 24:529-538 (2008). cNHEJ is a precise repair pathway where ends are rejoined and typically reconstitute the original DNA sequence. HR uses a DNA template with homology to sequences flanking the DSB to copy a homologous sequence to repair the broken site. aNHEJ is a mutation prone process that utilizes resection of 5′ ends of the DSB to complete the repair. The Microhomology Mediated End Joining (MMEJ) pathway involves rejoining the DNA ends using short regions of homology on each side of the break (e.g., usually >2 bases) where the intervening sequence is deleted. See, FIG. 1.

When artificial nucleases are introduced into the cell to target the genome, the DSBs that are generated are likely to proceed down the cNHEJ pathway where they are precisely repaired, which restores the existing nuclease target sequence, whether wild type or mutated. Eventually, however, mutations are inevitably generated that disrupt the target site. Sequencing information on these deletions suggests that in many instances the resulting deletion mutations are generated by MMEJ, due to the sequence scars that contain microhomologies that are are both sides of the break. Analysis of Cas9 nuclease DNA target sequences suggests that there is a correlation between the efficiency of collapse and the length of the microhomology on each side of the break. Bae et al., “Microhomology-based choice of Cas9 nuclease target sites” Nature Methods 11:705-706 (2014).

DSBs at most genomic sites are repaired primarily through the NHEJ pathway, which can produce small insertions or deletions during imprecise repair (for example, AAVS1). 28 See, FIG. 18. The data presented herein, which span DSBs in twelve sequences, indicate that microduplications are preferentially repaired via the MMEJ pathway, which yields predictable and efficient collapse. For this class of pathogenic mutations, precise repair via the MMEJ pathway provides a favourable alternative to homology-directed repair, which is inefficient in many cell types²⁹. Consistent with the present findings, MMEJ-mediated repair was recently used to efficiently correct the pathogenic microduplication associated with HPS¹³⁰. Although the use of allele frequencies from gnomAD can help to prioritize potential targets for MMEJ-based repair, this underestimates the extent of genetic diseases—particularly dominant ones—caused by microduplications.

### II. Microhomology Mediated End Joining Disease Mutation Repair

To test the generality of the presently contemplated MMEJ-based repair approach and the range of sequence lengths over which duplication collapse is efficient, editing products generated by SpyCas9 targeting endogenous microduplications within the human genome were evaluated.

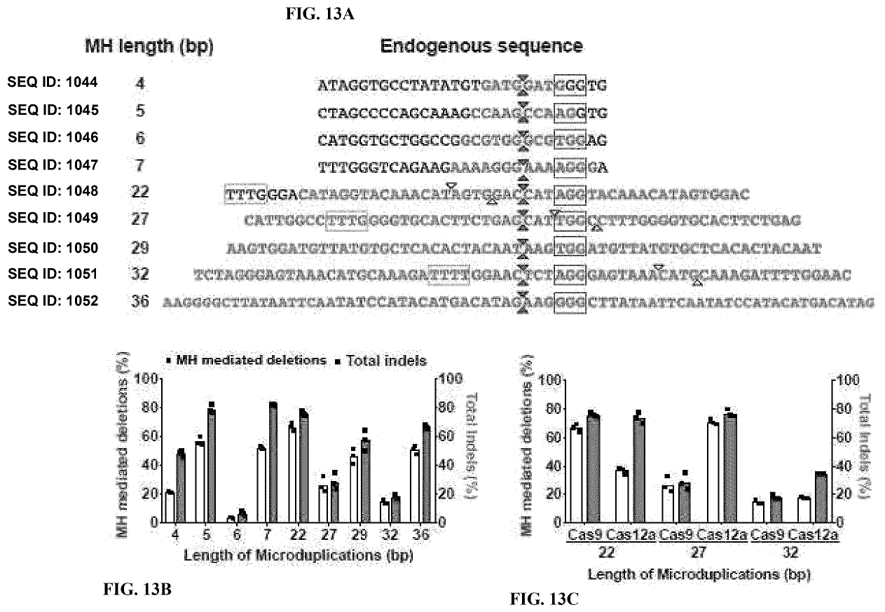

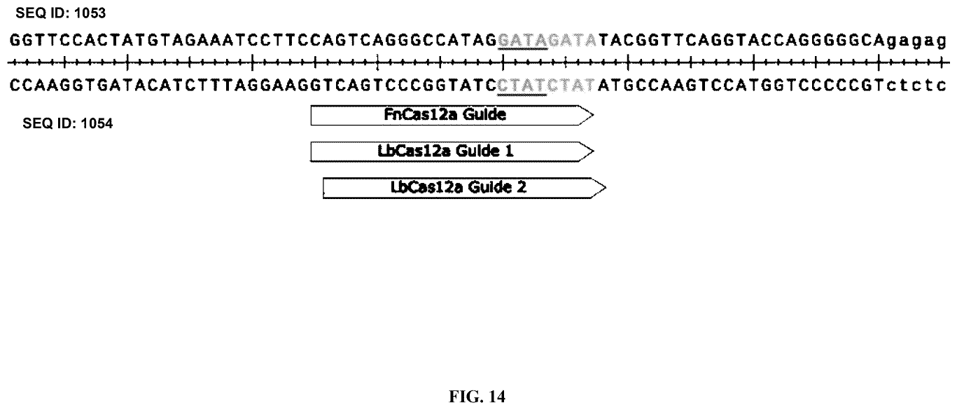

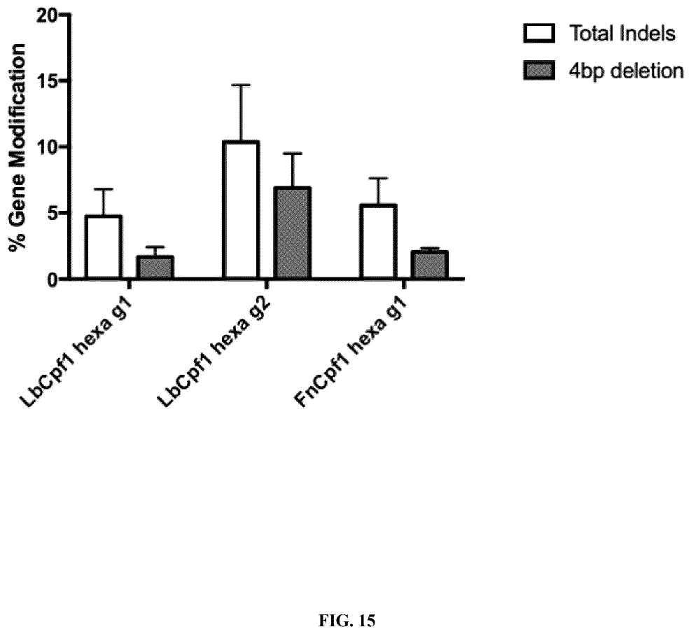

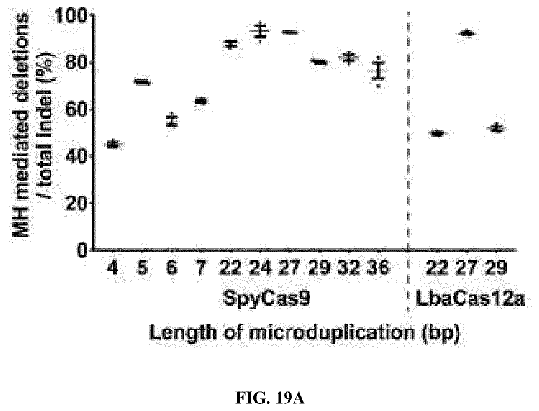

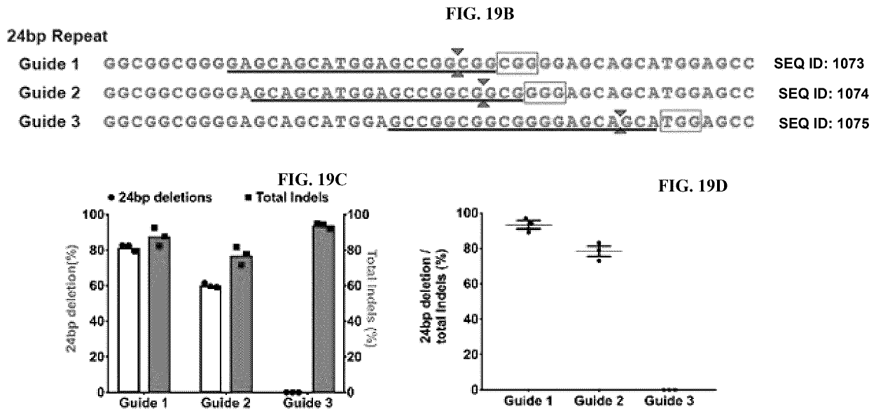

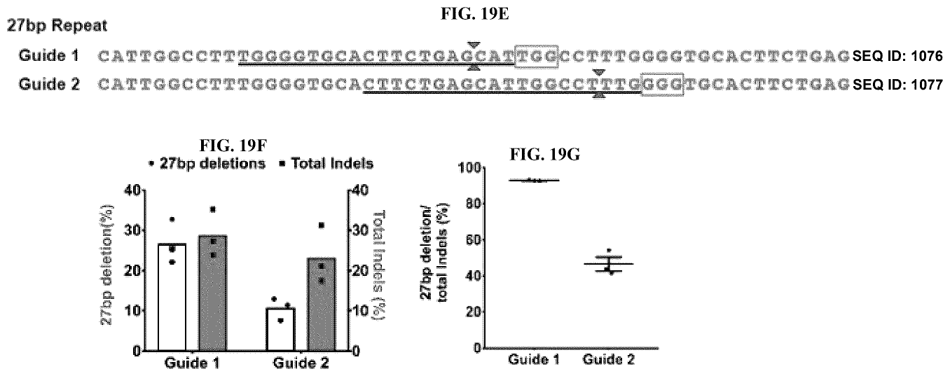

A bioinformatic analysis was performed to identify non-pathogenic, unique endogenous microduplications ranging from 4 bp to 36 bp in length in the human genome. See, FIG. 13A. The efficiency of microduplication collapse resulting from a SpyCas9 produced DSB at the centre of the microduplications in HEK 293T cells at these sites was examined. Although the bulk editing rate varied across these target sites, it was consistently found that duplication collapse was the major end-product within the edited alleles (ranging from 45% to 93%), regardless of the microduplication length. See, FIG. 13B and FIG. 19A. Consistent with the analysis at an HPS1 locus, a decrease in the duplication collapse efficiency was observed for 24- and 27-bp-long microduplications as cut sites were moved away from the centre. See, FIG. 19B-19G.

Whereas SpyCas9 generates blunt DSBs, the type V CRISPR-Cas nuclease Cas12a generates DSBs with 5′ overhangs¹⁹. It was then investigated whether LbaCas12a-generated breaks might be preferentially repaired by a resection-dependent pathway such as MMEJ by comparing the efficiency of microduplication collapse engendered by SpyCas9 and LbaCas12a nucleases at three endogenous sites. Efficient repeat collapse (50-90% of edited alleles) could be achieved with LbaCas12a at all three of these sites, with efficiencies similar to those of SpyCas9. See, FIG. 13C and FIG. 19A. In addition, LbaCas12a could drive repeat collapse for the four base pair duplication in HEXA that is associated with Tay-Sachs Disease. See, FIGS. 14-17. Overall, these data demonstrate that the MMEJ-based editing approach can be used to efficiently collapse microduplications up to lengths of at least 36 bp using either Cas9 or Cas12a programmable nucleases.

Recently, an algorithm has been developed to more reliably predict target loci that would be predisposed to generate a more homogeneous mutant allele population through MMEJ. Ata et al., “Toward Precision Molecular Surgery: Robust, Selective Induction of Microhomology-mediated End Joining in vivo” BioRxiv, (posted online Mar. 28, 2018). Thus, the goal of this algorithm was to identify sites in genes where the generation of a double strand break (DSB) will be repaired through the use of microhomologies on each side of the break to collapse the DNA sequence such that it is out-of-frame with regards to its translation and thus will not produce a functional protein. Termed the “MENTHU” algorithm, it appears primarily to be a way of post-processing predictions generated from an earlier reported algorithm (Bae et al., nature.com/articles/nmeth.3015 (2014)) to improve the prediction for when a DSB will be repaired by MMEJ in a fairly homogeneous way. This is useful if one wants to do precision genome editing, whereas Bae et al were considering the blunter application of making (any) out-of-frame deletions for gene knock-out.

The Ata et al. paper invites users to access this algorithm to facilitate the scanning of reference wild-type genes using Genbank IDs or RefSeq IDs to identify sites that will collapse primarily through a single MMEJ event down to a specific sequence. genesculpt.org/menthu/.

The Ata et al. algorithm is designed for the primary application of making knockouts in model organisms, e.g. the source-code repository for MENTHU has the subtitle “MENTHU knockout site recommender”. Consequently, Ata et al. discloses making mutants in zebrafish embryos through the injection of a programmable nuclease (TALENs or SpCas9), and then analyzing the resulting genetic products and phenotypes of these mutant animals.

Although the MENTHU algorithm appears to be set up to analyze genes, in principle any DNA sequence can be evaluated, e.g. with variant alleles and flanking sequence, but this is user dependent—not a function of the algorithm. In addition, most of the known pathogenic variant alleles that are duplications cause frame-shifts, and the algorithm is not set up to define going from an out-of-frame sequence to an in-frame sequence, let alone restoring the wild-type sequence.

In some embodiments, the present invention contemplates an alternative method that is focused on capturing abutting duplications within the ExAC database or gnomAD databases—a database of variants identified in whole-genome and whole-exome sequencing data aggregated from many large-scale projects (and subsuming the earlier ExAC exome-only database)—that may be suitable for MMEJ repair. Importantly, the basic representation of variants in gnomAD lists the genomic position, reference (REF) sequence starting at that position, and alternate (ALT) sequence starting at that position; it is not typically readily apparent if a variant is a duplication, as typically only the base immediately preceding an insertion is used as the reference allele, whereas to establish whether the inserted sequence is a duplication requires examining more of the flanking regions (e.g. the HEXA duplication has REF=G, ALT=GGATA, where only a single copy of the duplication is present). See, Tables 5 and 6 Furthermore, the gnomAD webpages and downloadable vcf files are not compatible with the MENTHU program in their raw form: the webpages for variants show surrounding genomic reference sequence only as a PNG graphic (not in text form) or via links to the UCSC genome browser for the reference genome; the vcf files sometime indicate when variants are duplications in the HGVSc fields added by Ensembl VEP, but again these files do not directly provide sequence of the duplication (both reference-copy and extra inserted copy) together with enough genomic flanking sequence to use for identifying cleavage sites that would be suitable for MMEJ. This alternative technology rebuilt the surrounding genomic sequence and identified common positions for nuclease cleavage around these duplications that could be tested to achieve collapse of the duplication and restore the wild-type sequence. Never is this concept mentioned in the MENTHU manuscript or algorithm.

In addition, the present invention—unlike the Ata et al. algorithm captures allele frequencies that allow the prioritization of potential targets based on the associated diseases, where the information on pathogenicity is extracted from the Clin Var database and combined with gnomAD and 1000 Genome Project phase 3 databases to determine how common the variants are overall and in specific human subpopulations.

Thus, the embodiments contemplated herein represent a completely novel analysis of a human genome variant database to extract information of disease alleles that may be amenable to gene correction by replacing microduplication mutations sequences with their requisite wild type sequences via an MMEJ strategy.

### III. Gene Microduplication Diseases

There are a number of diseases that have causative alleles within the human population that are associated with microduplications within the genome. See, Table 1.

TABLE 1
Exemplary Disease alleles associated with microduplications

| | | | | | | gnomAD |
|---|---|---|---|---|---|---|
| SEQ | | | | | clin Var | allele |
| ID | Disease | locus | duplication | dbSNP ID | ID | frequency |
| 144 | LGMD2G | TCAP | CGAGGTGT | rs778568339 | ND | 8.126e−5 |
| 145 | HPS | HPS1 | CCAGCAGGGGAGGCCC | rs281865163 | 5277 | 2.845e−5 |
| 146 | Tay-Sachs | HEXA | GATA | rs387906309 | 3889 | 0.0008041 |
| 147 | familial | DOK7 | GCCT | rs764365793 | 1273 | 0.0006367 |
| | limb-girdle | | | | | |
| | myasthenia | | | | | |
| 148 | Cone-rod | RAX2 | CCCGGG | rs549932754 | 1242 | 0.0007684 |
| | dystrophy | | | | | |
| | 11 | | | | | |

There are likely to be many more microduplications that are associated with diseases. But most disease phenotypes have not been linked to a specific microduplication. For example, ˜90% GWAS disease-associated SNPs are found in non-coding sequences, though which variants are themselves causal, as opposed to being in linkage disequilibrium with causal variants, is in many cases not yet known. Hindorff et al., “Potential etiologic and functional implications of genome-wide association loci for human diseases and traits” Proceedings of the National Academy of Sciences 106:9362-9367 (2009). In addition, repeat expansion diseases (e.g., Huntington's disease, ALS [C9ORF72], etc.) could be thought of as extended microduplications as well.

In one embodiment, the present invention contemplates a method for reverting a gene comprising a nucleotide microduplication mutation to a wild-type sequence. In one embodiment, the method comprises generating a DSB near the center of the nucleotide microduplication. In one embodiment, the nucleotide microduplication causes a disease. In one embodiment, the DSB is created by targeting a nuclease to the nucleotide microduplication center. In one embodiment, the nuclease includes, but is not limited to Cas9, CRISPR, Cas12 (Cpf1), zinc finger nucleases and/or TALEN. Although it is not necessary to understand the mechanism of an invention, it is believed that since the nuclease is targeting a mutated sequence, once the mutation reversion to a wild type sequence has occurred, the repaired target sequence would no longer be recognized by the nuclease, and thus remains a wild type sequence in the presence of the repairing nuclease. It is further believed that a correction DNA cassette is not needed for an MMEJ repair back to the wild-type sequence, only the nuclease (e.g. Cas9) and a targeting moiety having affinity for the mutant locus (e.g. sgRNA).

The data presented herein describes successful correction of disease causing alleles in patient-derived cell lines harboring microduplications in TCAP and HPS1. A high rate of correction is achieved in patient cells lines through the delivery of a nuclease suggesting that nuclease-induced MMEJ repair of microduplications within a genome can be programmed for other gene microduplication targets causing other diseases (e.g. HEXA-Tay-Sachs syndrome, other diseases in Table 1) leading to cures for these diseases.

### A. TCAP and HSP1 Microduplication Repair

The site-specific nuclease, S. pyogenes Cas9 (SpCas9) was used in the following therapeutic gene editing method. Jinek et al., “A programmable dual-RNA-guided DNA endonuclease in adaptive bacterial immunity” Science 337:816-821 (2012). Nonetheless, it is contemplated herein that similar results can be obtained using any Cas9 (or CRISPR), a Cpf1 nuclease or any other programmable nuclease system including, but not limited to, zinc finger nucleotide (ZFN), TALEN, mega-TAL or meganuclease all of which can be targeted to a gene microduplication sequence. The data presented herein show two different proof-of-principle targets (TCAP and HSP1) that may have therapeutic value. Both of these diseases are associated with substantial morbidity, and no curative therapies are currently available.

The mutant TCAP allele contains an 8 base duplication that leads to an out of frame coding sequence. UCSC: genome.ucsc.edu/cgi-bin/hgTracks?db=hg19&highlight=hg19. chr17% 3A37821635-37821635&position=chr17% 3A37821610-37821660. This TCAP allele has a frequency of ˜1 in 1000 in the east Asian population. gnomad.broadinstitute.org/variant/17-37821635-G-GCGAGGTGT. Individuals with homozygous inactivating mutations in TCAP have Limb Girdle Muscular Dystrophy 2G (LGMD2G).

The mutant HPS1 allele contains a 16 bp duplication that leads to an out of frame coding sequence. gnomad.broadinstitute.org/variant/10-100183554-T-TGGGCCTCCCCTGCTGG (SEQ ID NO: 149). This allele has a frequency of ˜1 in 21 in populations within Puerto Rico. MPH, S. E.-C. M. & MD, L. R. Y. Hermansky-Pudlak Syndrome 1-7 (2017). Individuals with homozygous inactivating mutations in HPS1 have Hermanksy-Pudlak syndrome (HPS1).

B-EBV cells were obtained from a patient that contains a homozygous 16 bp microduplication in the HPS1 gene with SpCas9 by nucleofection. ICE (similar to TIDE¹⁶) analysis of sanger sequence chromatogram from SpCas9 treated HPS1 B-EBV cells. The estimated mutagenesis rate is 41% with 30% of the alleles containing a 16 bp deletion (red arrow), which is a reversion to the wild-type sequence. Zero (x-axis) is no change in the length or composition of the 16 bp microduplication. See, FIG. 9.

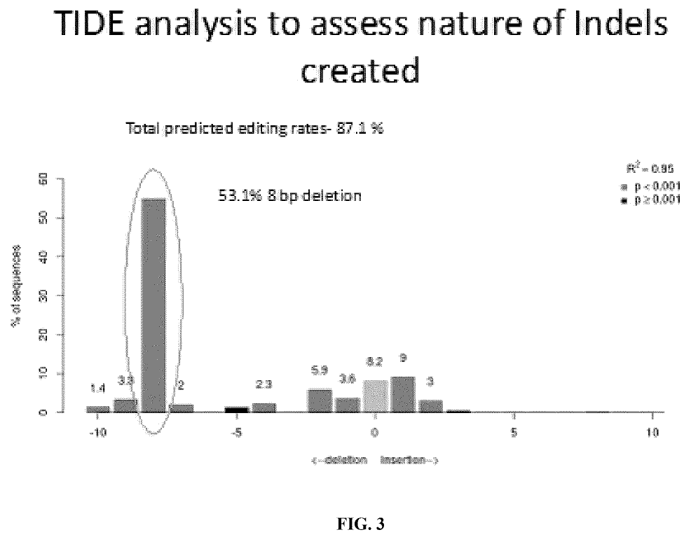

Patient-derived cells were obtained to test the potential for a nuclease targeting these deletions to revert the duplicated mutant allele. For TCAP, iPSCs were derived from fibroblasts from an individual that is a homozygous carrier of the 8 bp duplication. See, FIG. 3. For HPS1, B-EBV cells were purchased from Coriell generated from a patient that is homozygous for the 16 bp duplication in HPS1 [GM14606]. For the nuclease system, a 3×NLS-SpCas9 protein was used complemented with sgRNAs (Synthego) for the target sequences. See, Table 2.

TABLE 2
Guide Sequences in sgRNAs targeting duplications

| SEQ ID | Microduplication | |
|---|---|---|
| NO: | locus | SpCas9 Guide sequence |
| 150 | TCAP | AGCTGAGCTGCGAGGTGTCG |
| 151 | HPS1 | CAGCAGGGGAGGCCCCCAGC |

These Cas9-sgRNA complexes (e.g., Cas9 RNPs) were delivered by nucleofection to LGMD2G iPSCs, myoblast derived from LGMD2G iPSCs, or to the HPS1 B-EBV line. Following recovery and expansion of the nuclease-treated cells in culture the target genomic region was amplified by PCR from the population of treated cells and the mutagenic products were characterized by TIDE or ICE analysis of the sequence chromatograms. Brinkman et al., “Easy quantitative assessment of genome editing by sequence trace decomposition” Nucleic Acids Research (2014). TIDE analysis of Cas9 RNP treated LGMD2G iPSCs revealed that ˜53% of the alleles were converted back to a wild-type length. See, FIG. 3. ICE (Synthego) analysis of SpCas9 treated HPS1 B-EBV cells revealed that ˜30% of the alleles were converted back to a wild-type length in the context of a ˜41% total editing rate. See, FIG. 4. In summary, approximately 75% of the edited HPS1 alleles were converted to wild-type length.

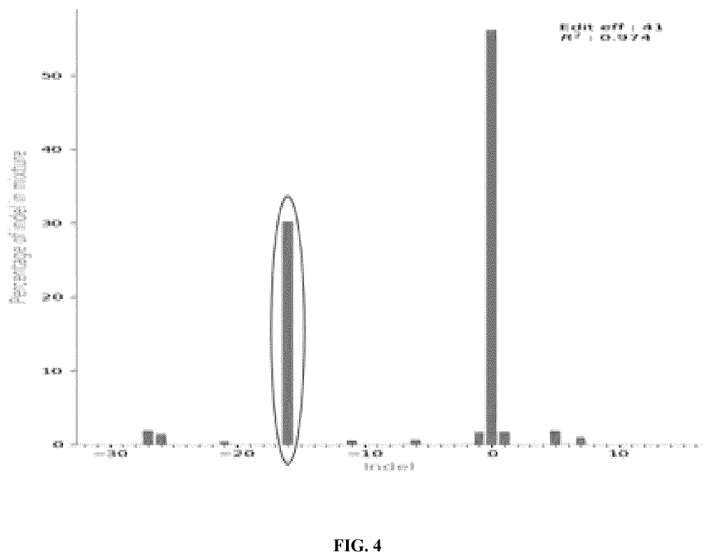

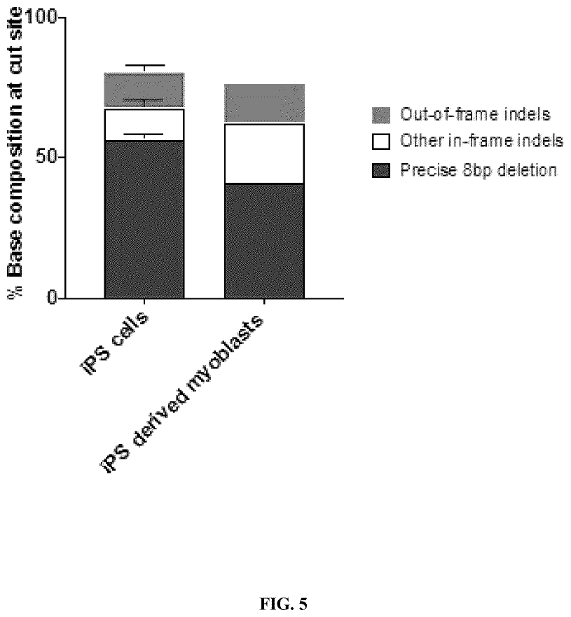

To confirm the TIDE analysis of TCAP alleles a deep sequencing analysis was performed on SpCas9 RNP treated iPSCs and iPSC derived myoblasts. See, FIG. 5. The iPSC data is from two biological replicates and the myoblast data is from a single experiment. The data confirm that the majority of the editing products are micro-homology mediated deletion of one of the duplicate sequences. There are also a large number of additional sequence that have been shifted back in frame through mutagenesis—although they are not the wild-type coding sequence. These mutant in-frame sequences may also be functional.

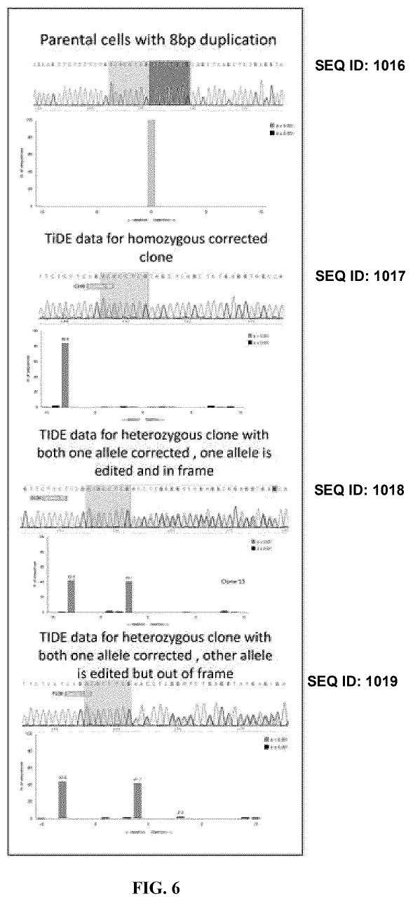

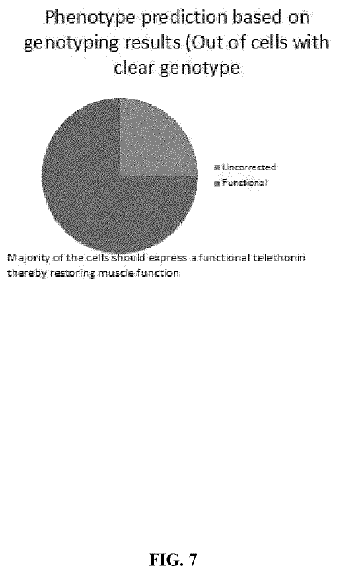

Individual iPSC clones were taken from a SpCas9 RNP treated TCAP microduplication cell population and expanded to determine the genotype. A variety of different genotypes were observed within the clones that were analyzed. See, FIG. 6. Two of the 24 clones that were analyzed were homozygous for the wild type (WT) allele. Six “clones” could not be defined conclusively—likely because the colonies were initiated from 2 cells generating more than 2 allele sequences. Overall the analysis of 18 clones demonstrate that the majority of iPSC clones (˜75%) contain at least one corrected allele of wild type sequence. See, FIG. 7.

In one embodiment, the present invention contemplates a method of differentiating SpCas9 treated iPSC clones into myoblasts to determine the number that display expression of the TCAP encoded protein telethonin.

The methods disclosed herein show an ability to precisely revert a microduplication back to its parental (e.g., wild type) sequence that can correct genetic microduplication mutations underlying of a number of diseases. Aside from those listed in Table 1 (supra)—there may be a number of diseases that stem from microduplications given the limited depth of genomic data that is associated with rare diseases.

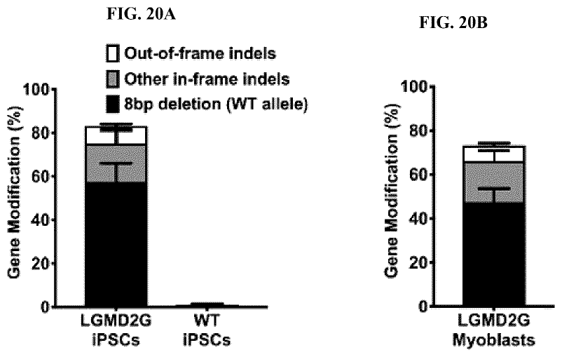

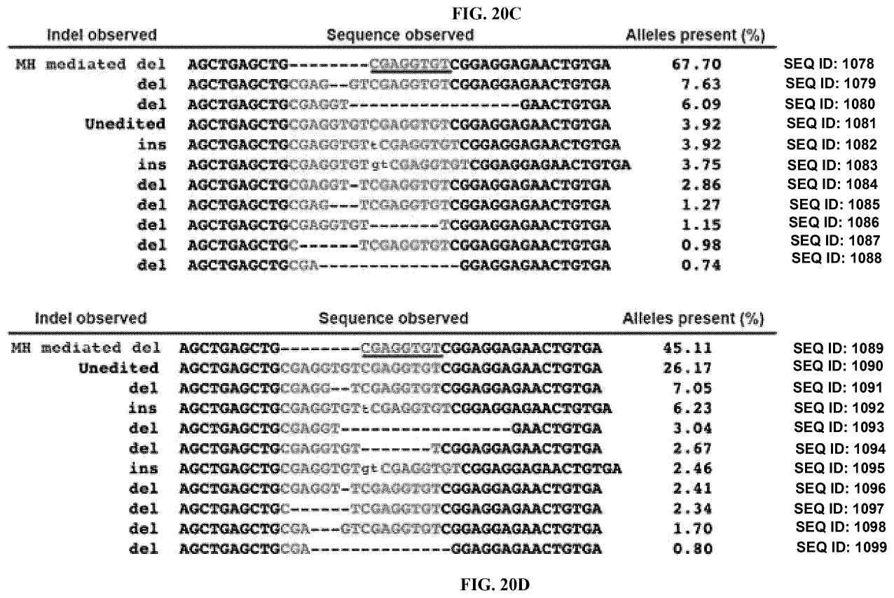

An sgRNA was designed and tested for SpyCas9 to generate a DSB one base pair away from the middle of the TCAP 8-bp microduplication. See, FIG. 10B. Purified SpyCas9 protein was complexed with a synthetic sgRNA (RNP) and electroporated into iPSCs homozygous for the TCAP microduplication that were derived from patients with LGMD2G. After four days, deep sequencing analysis was used to analyse the genomic region of interest for insertions and deletions (indels). Robust gene editing (about 80% indel rate) was observed, indicating that the SpyCas9 RNP can efficiently generate DSBs at this site. Closer examination of the sequence variants revealed that on average about 57% of the alleles contained a precise 8-bp deletion corresponding to the wild-type allele. See, FIG. 10C and FIG. 20A.

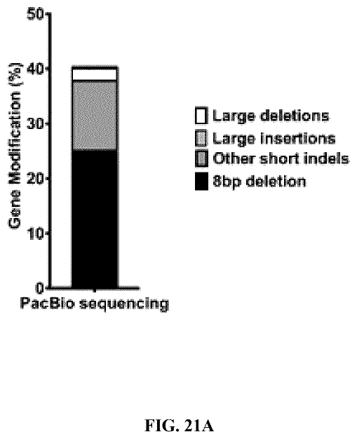

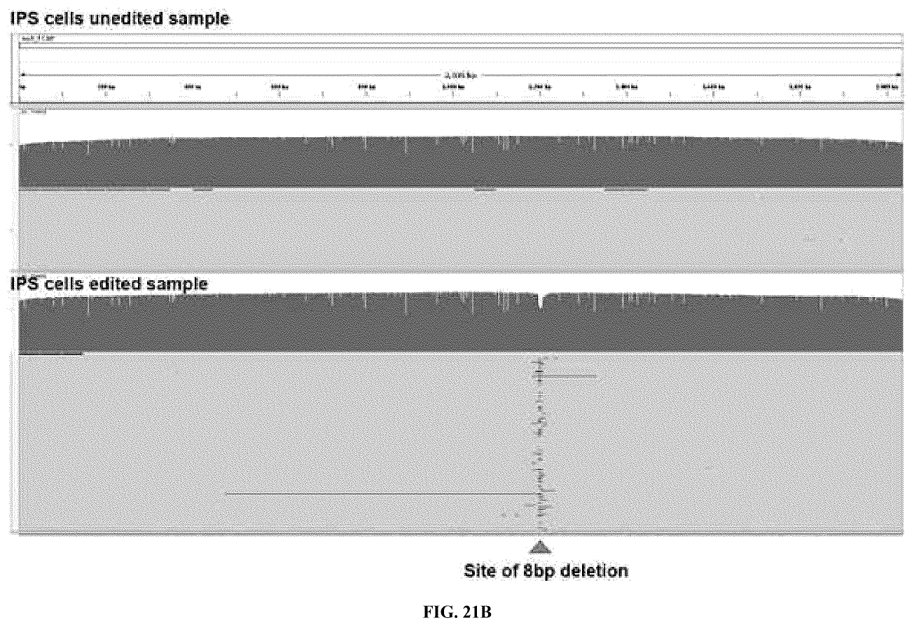

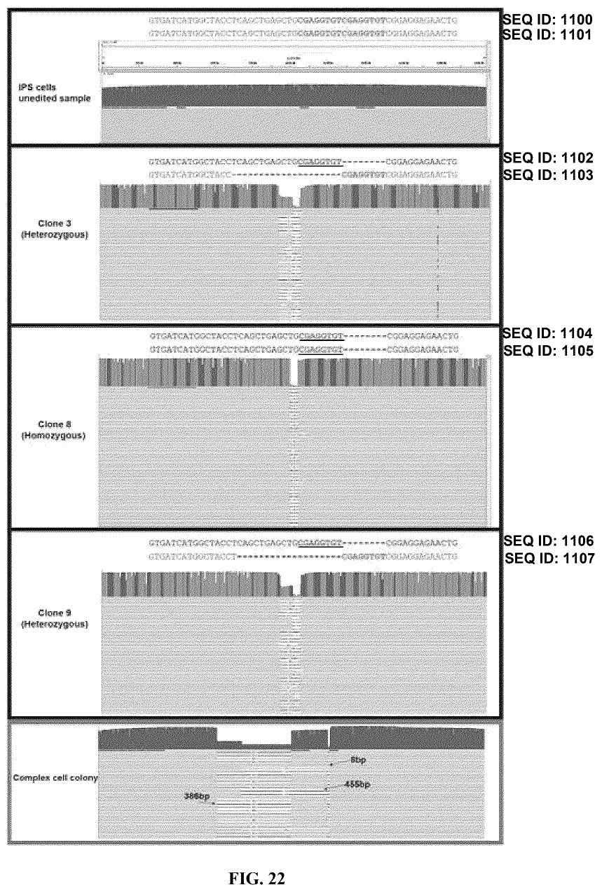

Notably, when introduced into wild-type cells containing functional TCAP, the SpyCas9 RNPs did not cause measurable editing at the TCAP allele, indicating that the corrected allele in the mutant cells is not subject to unintended damage following MMEJ-mediated reversion. See, FIG. 10C. In addition to the precise 8-bp deletion, it was also observed that an additional approximately 17% of the alleles contained in-frame mutations, and therefore may encode hypomorphic alleles with some restoration of function. See, FIG. 20A and FIG. 20C. Genotyping of 22 clones generated from a nuclease-treated LGMD2G iPSC population revealed that 77% contained at least one wild-type allele, indicating that the majority of nuclease-treated cells would be phenotypically corrected. See, FIG. 10D. To independently verify the duplication collapse rates observed in edited iPSCs by Illumina short-read sequencing, a 2-kb amplicon was sequenced spanning the TCAP locus from a population of SpyCas9-edited iPSCs using the Pacific Biosciences long-read sequencing platform (PacBio). Analysis of these reads revealed that 67% of the edited alleles with insertions or deletions below 100 bp in length corresponded to the 8-bp collapse, which is similar to the 73% rate of 8-bp collapse determined by Illumina sequencing for this sample. See FIG. 21. Treatment of cells with Cas9 nuclease can produce large deletions (>100 bp) at the target locus at a modest frequency¹⁰. Consistent with these findings, the PacBio analysis revealed the presence of large deletions (100-1,000 bp) that would not have been detected by Illumina sequencing at a frequency of about 2% in bulk edited iPSCs. A genotypically complex iPS cell colony was also isolated that harboured two large deletions at the TCAP locus. See, FIG. 22.

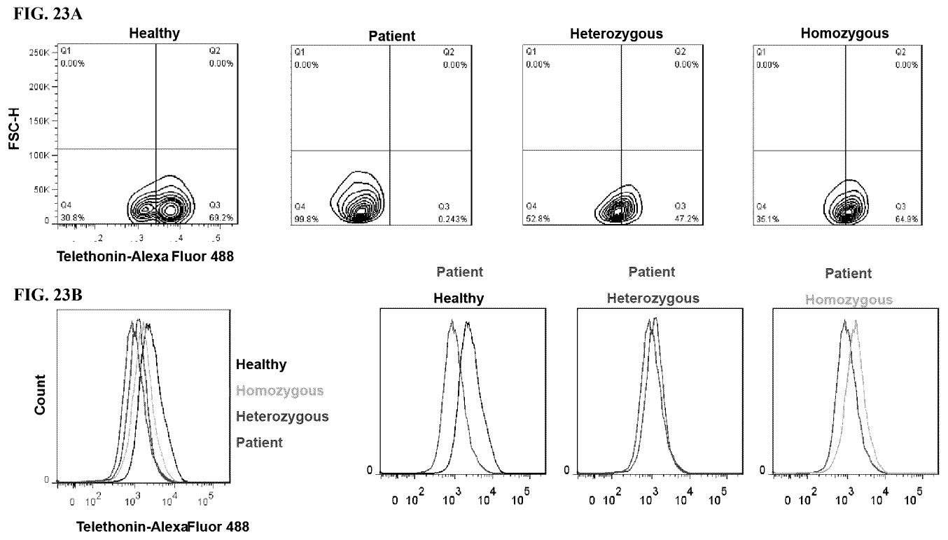

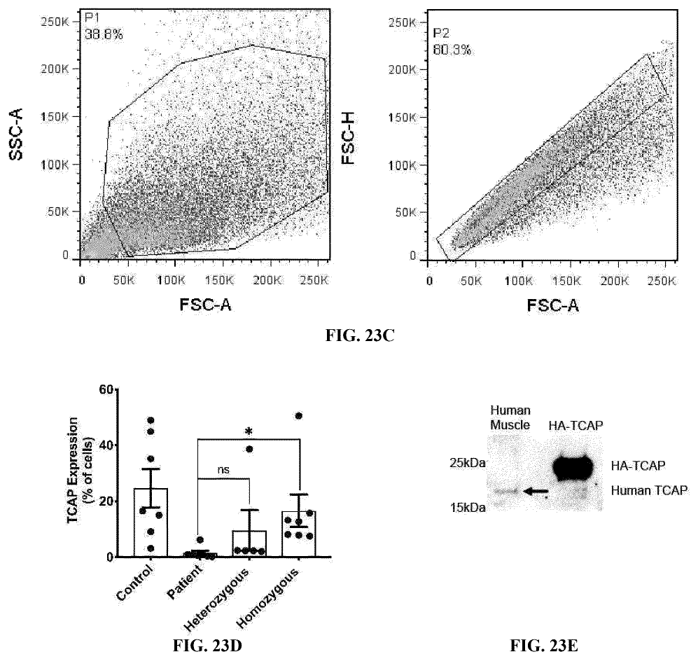

To demonstrate the translatability of the present approach to muscle cell types, LGMD2G iPSCs were differentiated into proliferative skeletal myoblasts that can be induced to terminally differentiate into myotubes¹¹. iPSC-derived myoblasts can repair damaged muscle in a similar way to myogenic satellite cells (one of the primary targets of gene therapy for myopathies). Myoblasts were electroporated with SpyCas9 RNPs programmed to target the 8-bp microduplication. Following editing, about 45% of the alleles were precisely repaired back to the wild-type sequence. See, FIG. 10E, FIG. 20B, and FIG. 20D. Immunostaining of myotubes derived from corrected LGMD2G iPSC clones with an anti-telethonin antibody showed that genetic correction restored telethonin expression. See, FIG. 23. Collectively, these data show that introducing a DSB close to the centre of microduplication can efficiently achieve precise in vitro correction of the 8-bp microduplication associated with LGMD2G in iPSCs and in myoblasts that mimic cell populations that would be therapeutically targeted in vivo.

The present approach was further tested on a 16-bp pathogenic microduplication in exon 15 of HPS1, which is associated with HPS1 and leads to the production of a truncated protein responsible for this autosomal recessive disease¹². HPS1 has a high prevalence in the Puerto Rican population, with a carrier rate of approximately 1 in 21 in the northwest region². HPS proteins are involved in the biogenesis of lysosome-related organelle complexes (BLOCs), which are necessary for the proper trafficking of cargo to melanosomes, dense granules and lysosomes¹³. HPS1 patients suffer from albinism, bleeding disorders, vision loss and progressive pulmonary fibrosis, which leads to premature death¹⁴.

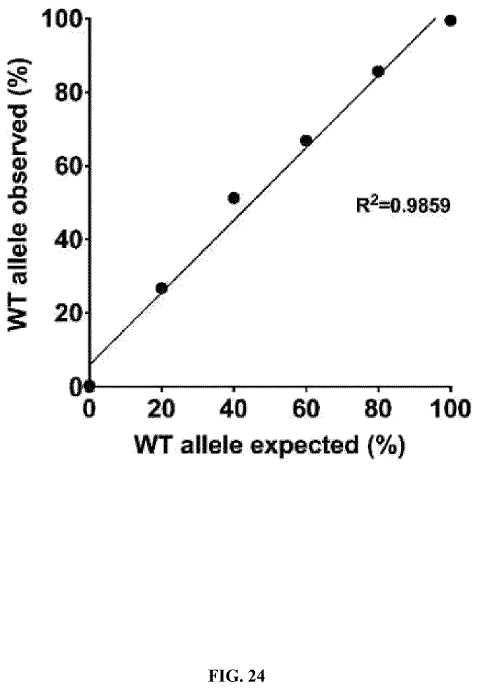

Gene correction efficacy was determined in a patient-derived B lymphocyte cell line (B-LCL) homozygous for the 16-bp microduplication by electroporating these cells with SpyCas9 RNPs programmed to cleave two base pairs away from the centre of the microduplication. See, FIG. 11A (target site 1). To accurately assess the observed editing rates, unique molecular identifiers (UMIs) were added to the PCR amplicons during Illumina library construction to allow the removal of any amplification bias¹⁵. It was confirmed that this approach accurately captured the relative percentage of HPS1 microduplication and wild-type alleles present in a series of test populations. See, FIG. 24. At HPS1 target site 1, editing was observed at about 46% of the alleles with around 35% restored to the wild-type sequence. See, FIG. 11B & FIG. 11C.

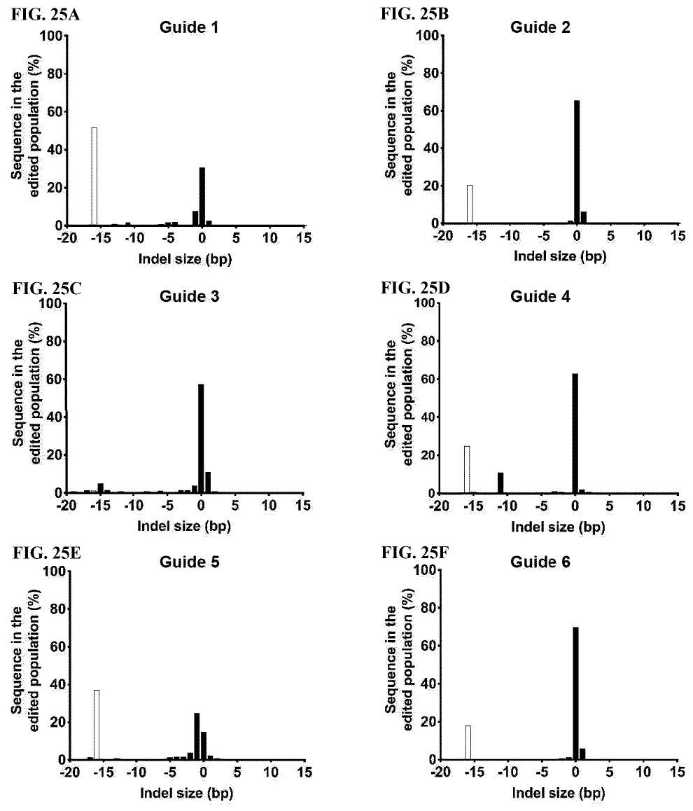

The effect of the position of the DSB within the microduplication was further examined on the efficiency of MMEJ-mediated repair by designing five additional sgRNAs that targeted the DSB to different positions relative to the centre of the microduplication See, FIG. 11A (target sites 2-6). As the break site was shifted away from the centre, there was a decrease in the efficiency of achieving the precise 16-bp deletion. See, FIG. 11B & FIG. 11C. However, target sites 3 and 6 were notable exceptions to this trend. Target site 3 was observed to be quite efficient at generating indels to the exclusion of the 16-bp deletion, probably because the wild-type sequence, once regenerated, can also be targeted by this sgRNA for further mutagenesis. See, FIG. 25. On the other hand, target site 6 achieved efficient deletion of the 16-bp microduplication (more than 50% of the modified alleles), despite being the most distal of the cleavage sites (10 bp from the centre of the microduplication). Its efficiency may be due to the extended regions of homology that surround the cleavage site at this end of the microduplication. See, FIG. 11A (target site 6). Overall, these results demonstrate that the cleavage position within the microduplication and the presence of alternate regions of microhomology can influence the production of the desired wild-type end product. See, FIG. 11C.

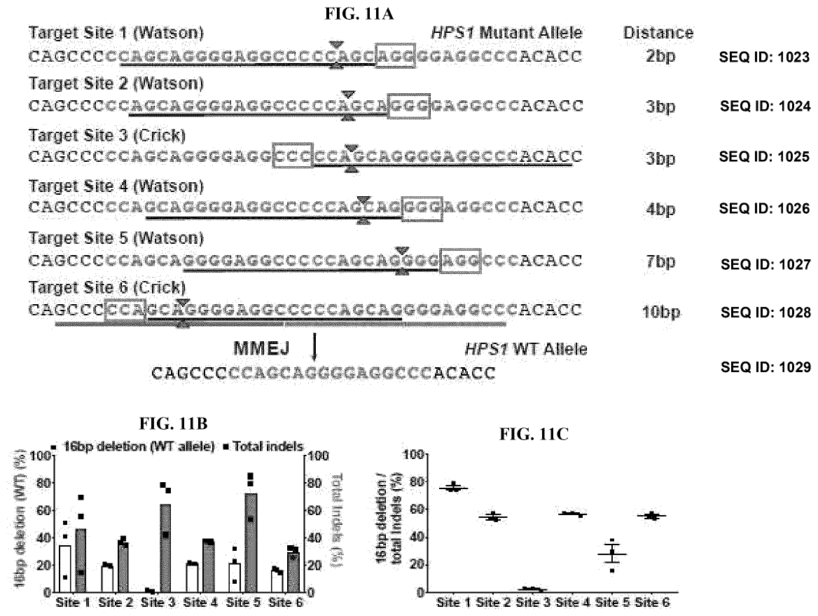

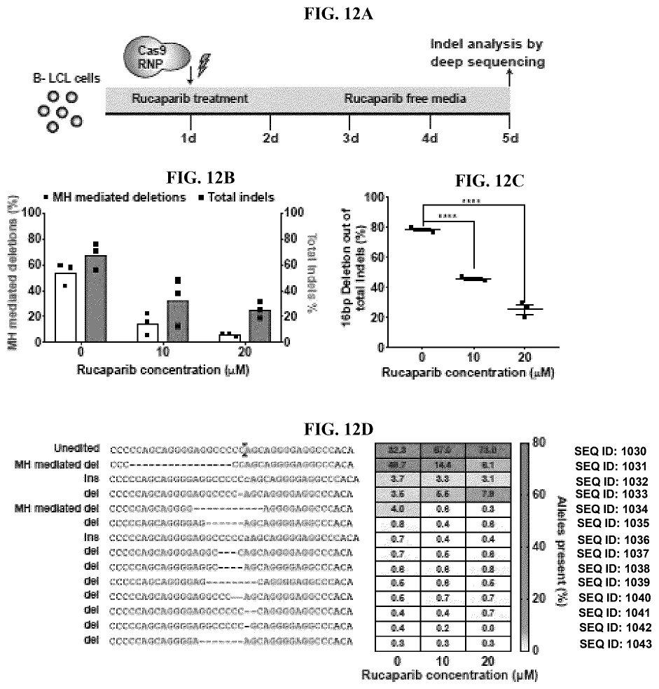

To investigate whether nuclease-mediated collapse of a microduplication occurs via the MMEJ pathway, a DNA repair factor (PARP-1) that regulates DSB flux through this pathway was inhibited. PARP-1 influences the repair of a DSB through resection-dependent DNA repair pathways, such as MMEJ^{3,16}, which are in competition with the non-homologous end joining pathway (NHEJ) for DSB repair¹⁷. See FIG. 18A. Inhibition of the catalytic activity of PARP-1 by rucaparib reduces DSB flux through the MMEJ pathway, resulting in a decrease in microhomology-based deletion products in the resulting repair events¹⁸. See, FIG. 18A. Patient-derived HPS1 B-LCL cells were treated with 10 μM or 20 μM rucaparib before and after treatment with SpyCas9 RNP to suppress MMEJ-mediated repair of DSBs. See, FIG. 12A. An overall reduction in editing rates was observed at the HPS1 locus upon rucaparib treatment. See, FIG. 12B. These lower editing rates were primarily the result of a reduction in the 16-bp deletion product, which decreased from about 50% in untreated cells to around 15% and 6% in cells treated with 10 μM and 20 μM rucaparib, respectively. See, FIG. 12B-12D. A similar reduction in microhomology-based deletions was observed with SpyCas9 RNP targeting the AAVS1 locus in patient-derived HPS1 B-LCL cells. See, FIG. 18. Thus, the MMEJ pathway underlies the robust correction of the microduplications for LGMD2G and HPS1 in the presence of a targeted DSB.

### B. Tay-Sachs Disease (HEXA Gene)

In one embodiment, the present invention contemplates a method for HEXA editing by Cas12a to correct a mutated sequence of the Tay-Sachs locus.

Two different Cas12a (also known as Cpf1) orthologs (LbCas12a and FnCas12a) were tested for their ability to drive microhomology-mediated end joining (MMEJ) to collapse the common GATA microduplication in HEXA that is associated with Tay-Sachs disease. The GATA·GATA duplication (repeated segments) results in a frameshift within the gene that inactivates it and leads to Tay-Sachs if both HEXA alleles are disrupted). See, FIG. 14. This allele occurs with a frequency of ˜1 in 100 individuals in some Jewish populations.

crRNAs were designed to target Cas 12a cleavage to the region spanning the microduplication to revert it to the wild-type sequence through MMEJ repair. One crRNA was designed for FnCas12a to utilize a TTC PAM (FnCas12a Guide). See, FIG. 14. Two crRNAs were designed for LbCas12a to utilize either a CTTC PAM (LbCas12a Guide 1) or a TTCC PAM (LbCas12a Guide 2). See, FIG. 14. For LbCas12a, these PAMs are not the optimal TTTV sequence, which may result in lower activity.

crRNAs (120 μmol) were complexed with 60 pmol of purified FnCas12a-2×NLS or LbCas12a-2×NLS protein and then electroporated into a B-EBV cell line that is homozygous for the GATA microduplication in HEXA (Coriell GM11852). See, Table 3.

TABLE 3
crRNA Sequences for crRNAs targeting HEXA
duplication

| SEQ | crRNA/ | |
|---|---|---|
| ID | Cas12a | Cas12a crRNA sequence |
| 152 | LbCas12a | UAAUUUCUACUAAGUGUAGAUCAGUCAGGGCCAUA |
| | Hexa | GGAUAGAUA |
| | guide 1 | |
| 153 | LbCas12a | UAAUUUCUACUAAGUGUAGAUAGUCAGGGCCAUAG |
| | Hexa | GAUAGAUAU |
| | guide 2 | |
| 154 | FnCas12a | UAAUUUCUACUGUUGUAGAUCAGUCAGGGCCAUAG |
| | Hexa | GAUAGAUA |
| | guide | |

In the crRNA sequences the constant region is in bold. Double underline indicates the base pairing regions of the hairpin stem. Single underlined sequence is the guide sequence (23 nt)

After 72 hours the genomic DNA from treated cells were harvested and the genomic region of interest within HEXA was PCR amplified and submitted for Sanger sequencing. Mutation rates were determined by TIDE analysis (tide.deskgen.com) in comparison to an unedited sequence chromatogram from the same genomic region. Total indels were modest (˜5 to 10%). See, FIG. (grey bars). A reversion to the wild-type sequence was observed in all of the samples (brown bars), where for LbCas12a guide 2 the majority of the alleles that were edited restored the desired wild-type sequence. The experiment was performed in biological triplicate, where the error bars represent the standard error of the mean.

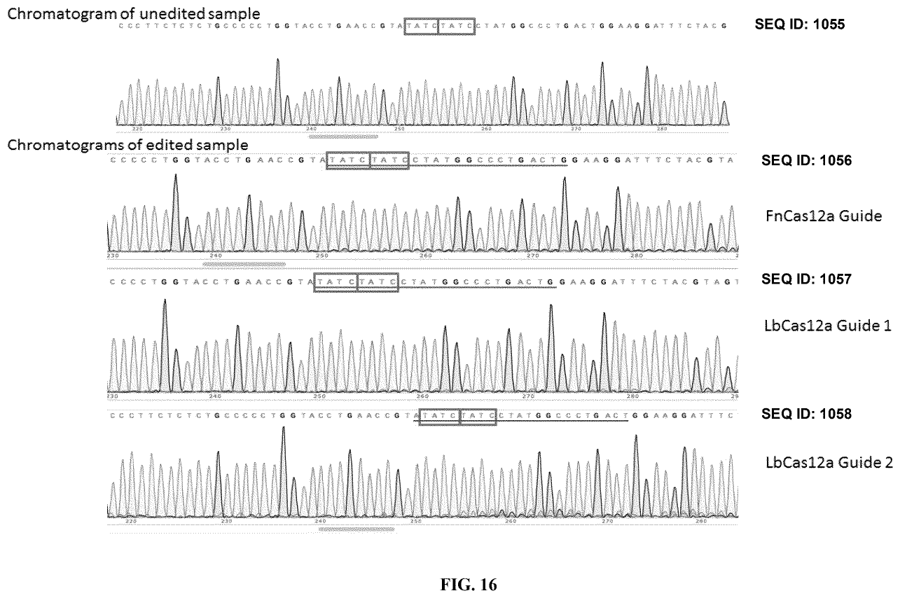

Representative sequence chromatograms show sequencing on the complementary strand. See, FIG. 16. The TATC duplication (complement of GATA) is boxed in and the respective guide target sequences for each nuclease are underlined.

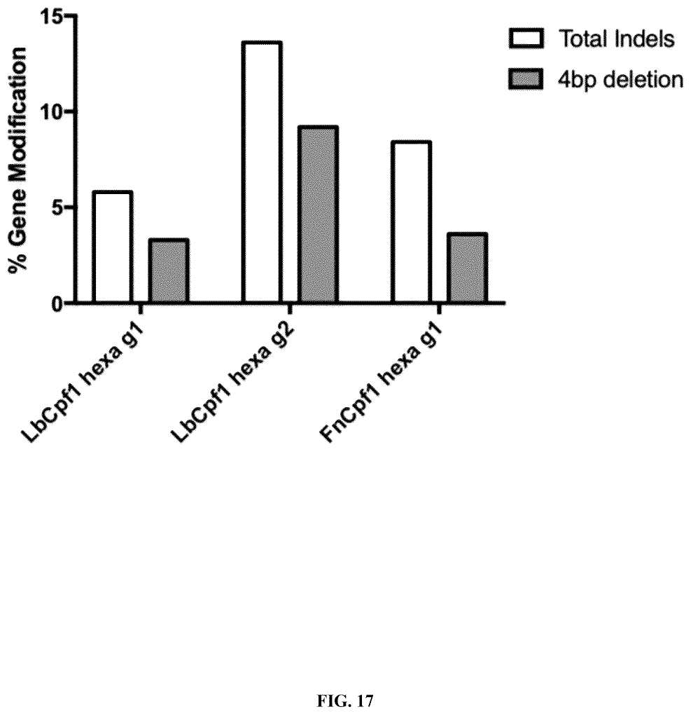

The concentration of the delivered Cas12a:crRNA was then increased for each nuclease: guide combination from 90 pmol to 180 pmol. The editing rates after electroporation of the HEXA GATA duplication in the B-EBV line in a single experiment were improved (white bars) and the rate of wild-type sequence reversion was also increased, reaching nearly 10% for the LbCas12a guide 2 treated cells (grey bars). See, FIG. 17. These data demonstrate the feasibility of reverting the mutant allele to the wild-type DNA sequence through the introduction of a targeted double-strand break without the need of a donor DNA sequence (or homology directed repair) for this restoration.

Those in the art would appreciate that the current data showing the correction disease-causing alleles for two different diseases provides an expectation that the technology has widespread applicability. Furthermore, as more diverse programmable nuclease systems are defined (e.g., CRISPR systems) that have broader targeting range and better delivery properties, this type of approach will become easier to perform in vivo. Although it is not necessary to understand the mechanism of an invention, it is believed that this approach may also work efficiently for genetic diseases based upon repeat expansion mutations if DSBs can be targeted just inside the edges of the repeat elements to allow the induction of long-range microhomology mediated repair.

### IV. Genomic Microduplication Variants

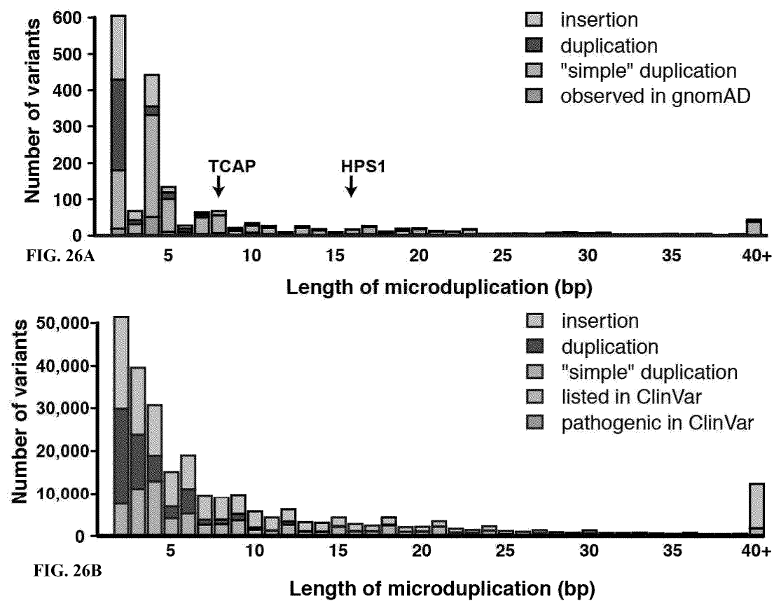

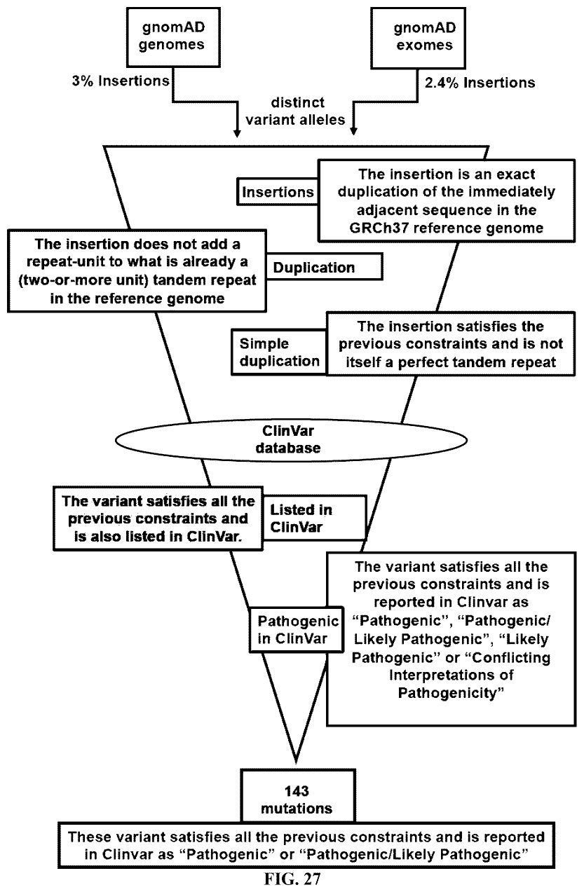

To investigate whether this MMEJ-based therapeutic strategy can be applied more broadly for correcting human genetic disorders, a bioinformatic analysis was performed to gauge the prevalence of disease-causing microduplications in human populations. The Clin Var database²⁰ includes about 4,700 duplications that are annotated as ‘pathogenic’ or ‘pathogenic/likely pathogenic’. See, FIG. 26A. Duplications of lengths ranging from 2 to 40 bp were of particular interest because the data presented herein indicate that microhomologies within this range can be precisely repaired via the MMEJ pathway. See, FIG. 13. ‘Simple’ duplications—those for which the duplicated sequence is not part of a more complex repeat structure—were also evaluated as to whether they improve the odds that the primary homology-based collapse would result in the desired wild-type sequence. Finally, all duplications in ‘coding’ regions (mainly exons plus 50 flanking bases) were examined from the gnomAD exome and genome sequencing databases²¹ to prioritize pathogenic duplications according to their frequencies in human populations. See, FIG. 26A and FIG. 27. The present analysis yielded 143 likely disease-causing microduplications of lengths 2-40 bp that were observed at least once in gnomAD, some of which occur in specific subpopulations at substantial frequencies (for example, Tay-Sachs disease), See, FIG. 26B.

To facilitate the utilization of a bioinformatics analysis, the present invention was accompanied by the creation of an interactive, searchable webtool (rambutan.umassmed.edu/duplications/). This bioinformatices analysis also included the identification of potential Cas9 and Cas12a cleavage sites within these microduplications²². As shown within the tool, ‘tiling’ data across HPS1 microduplications and endogenous microduplication sites, the position of the DSB break within the duplication, and the use of a guide design that avoids cleavage of the wild-type allele, facilitate an efficient, stable collapse of microduplications. Rapid advances are being made in characterizing nucleases with alternate specificities^{23,24} and in engineering nucleases with alternate or expanded recognition preferences²⁵⁻²⁷, which will make correction of disease-causing microduplications using the MMEJ-based approach even more effective.

The results below for the most part are based on the files of “coding” variants from gnomAD genomes and exomes, version 2.0.2. gnomad.broadinstitute.org/downloads. This database comprises variants in the intervals used for the ExAC database. Most of these intervals correspond to exons plus 50 flanking bases on each side, and they collectively cover 60 million bases, about 2% of the genome. Note that there are no variant calls for the Y chromosome, and these are not strictly all coding variants, as some are in introns, UTRs, miRNA, ncRNA.

The 1000 Genome Project data was taken from ftp.1000genomes.ebi.ac.uk/voll/ftp/release/20130502/. The vcf files there include precomputed allele-frequencies for five broad super-populations and allele-frequencies for 26 more-specific populations computed from the per-individual genotypes in the vcf files aggregated using the population assignments from the file integrated_call_samples_v3.20130502.ALL.panel.

The Clin Var annotations were taken from the file ftp.ncbi.nlm.nih.gov/pub/clinvar/vcf_GRCh37/clinvar_20180225.vcf.gz. Here, the variants have been normalized (trimmed and left-aligned) for the purpose of matching them up, but the HGNC notation used at Clin Var may follow the right-aligned (3′-most position) convention, in which duplications are taken to occur immediately after the repeated sequence rather than immediately before the repeated sequence.

The gnomAD genome files contain a total of 4851138 distinct variant alleles, of which 145892 (˜3%) are insertions. The gnomAD exome files above contain a total of 17009588 distinct variant alleles, of which 414576 (˜2.4%) are insertions. Note that many of these variants are common to both the exomes and genomes, but in the tables below variants that occur in both are counted only once.

Table 4 below focuses on the insertions, and in particular the duplications. The second column (insertions) gives the counts of all the distinct insertion variant alleles, binned by the length of the insertion (length), with all variants of length at least 40 combined into one bin. Subsequent columns give the number of variants that satisfy additional criteria, as follows:

dup: the insertion is an exact duplication of the immediately adjacent sequence in the GRCh37 reference genome (immediately 3′ with this normalization). Note that there may be polymorphism in this adjacent sequence that affect whether an insertion is indeed a perfect duplication for any given individual.
dup2: the insertion does not add a repeat-unit to what is already a (two-or-more unit) tandem repeat in the reference genome. This eliminates e.g. the duplication of CCCGGG (SEQ ID NO: 155) in RAX2, as the reference genome already has two immediately adjacent (3′) tandem copies of this: ncbi.nlm.nih.gov/projects/SNP/snp_ref.cgi?rs=rs549932754
dup2i: the insertion satisfies the previous constraints and is not itself a perfect tandem repeat (e.g., for a duplicated six-mer, it is not of the form XXXXXX, XYXYXY or XYZXYZ). Note that even if a duplicated sequence is not in itself a perfect tandem repeat it may contain internal tandem repeats—e.g. the AGGAGG (SEQ ID NO: 156) in the duplicated AAGGAGGATC (SEQ ID NO: 157) in NCF4—so depending on where the Cas9 cleavage site is this may need to be considered, to prevent a shorter internal microduplication from being collapsed instead of the full duplication.
dup2iC: the variant satisfies the previous constraints and is also listed in Clin Var.
dup2iL: the variant satisfies the previous constraints and is reported in Clinvar as “Pathogenic”, “Pathogenic/Likely_pathogenic”, “Likely_pathogenic” or “Conflicting_interpretations_of_pathogenicity”
dup2iP: the variant satisfies the previous constraints and is reported in Clinvar as “Pathogenic” or “Pathogenic/Likely_pathogenic”

TABLE 4
Microduplication Variant Characteristics
In Clin Var And gnomAD Databases

| length | insertions | dup | dup2 | dup2i | dup2iC | dup2iL | dup2iP |
|---|---|---|---|---|---|---|---|
| 1 | 210230 | 179654 | 59169 | 59169 | 399 | 242 | 182 |
| 2 | 51418 | 29880 | 11919 | 7562 | 53 | 25 | 19 |
| 3 | 39579 | 23795 | 12892 | 11141 | 77 | 11 | 4 |
| 4 | 30704 | 18835 | 14615 | 13010 | 112 | 70 | 52 |
| 5 | 15142 | 6890 | 4754 | 4189 | 28 | 16 | 10 |
| 6 | 18971 | 11102 | 6125 | 5251 | 46 | 7 | 3 |
| 7 | 9634 | 3793 | 2976 | 2623 | 10 | 5 | 4 |
| 8 | 9123 | 3819 | 3038 | 2739 | 12 | 9 | 7 |
| 9 | 9818 | 5155 | 3979 | 3686 | 17 | 3 | 2 |
| 10 | 5756 | 1997 | 1683 | 1502 | 12 | 9 | 8 |
| 11 | 4326 | 1311 | 1236 | 1195 | 10 | 6 | 4 |
| 12 | 6249 | 3384 | 2957 | 2649 | 18 | 3 | 0 |
| 13 | 3207 | 1099 | 1068 | 1042 | 7 | 4 | 2 |
| 14 | 3068 | 1031 | 993 | 942 | 5 | 2 | 1 |
| 15 | 4307 | 2311 | 2190 | 2110 | 19 | 7 | 3 |
| 16 | 2813 | 1173 | 1128 | 1086 | 8 | 4 | 4 |
| 17 | 2438 | 1099 | 1069 | 1067 | 9 | 7 | 6 |
| 18 | 4316 | 2646 | 2552 | 2459 | 14 | 4 | 4 |
| 19 | 2065 | 1012 | 997 | 997 | 5 | 3 | 1 |
| 20 | 2148 | 1082 | 1045 | 1001 | 6 | 5 | 3 |
| 21 | 3463 | 2218 | 2141 | 2127 | 11 | 2 | 1 |
| 22 | 1687 | 818 | 806 | 799 | 1 | 0 | 0 |
| 23 | 1395 | 690 | 670 | 670 | 3 | 3 | 3 |
| 24 | 2272 | 1283 | 1244 | 1221 | 7 | 2 | 1 |
| 25 | 1149 | 485 | 477 | 471 | 1 | 1 | 1 |
| 26 | 1006 | 356 | 353 | 350 | 3 | 0 | 0 |
| 27 | 1373 | 653 | 635 | 631 | 6 | 1 | 0 |
| 28 | 878 | 314 | 308 | 304 | 3 | 1 | 1 |
| 29 | 751 | 239 | 233 | 233 | 1 | 1 | 0 |
| 30 | 1321 | 579 | 549 | 536 | 1 | 0 | 0 |
| 31 | 693 | 194 | 189 | 189 | 1 | 1 | 1 |
| 32 | 695 | 193 | 187 | 182 | 0 | 0 | 0 |
| 33 | 772 | 272 | 263 | 262 | 3 | 0 | 0 |
| 34 | 590 | 169 | 164 | 157 | 0 | 0 | 0 |
| 35 | 528 | 121 | 117 | 116 | 0 | 0 | 0 |
| 36 | 743 | 244 | 236 | 225 | 1 | 0 | 0 |
| 37 | 457 | 106 | 102 | 102 | 1 | 0 | 0 |
| 38 | 474 | 122 | 115 | 113 | 0 | 0 | 0 |
| 39 | 524 | 149 | 140 | 140 | 2 | 0 | 0 |
| 40+ | 12413 | 1818 | 1800 | 1756 | 7 | 1 | 1 |

Table 4 Totals:

| | | | | | | | |
|---|---|---|---|---|---|---|---|
| 1-40+ | 468496 | 312091 | 147114 | 136004 | 919 | 455 | 328 |

Note that the filters used in columns dup2 and dup2i are included to increase the chances of MMEJ restoring the duplication to exactly its wild-type form, via removal of exactly one complete copy of the duplicated sequence, but these filters may not be strictly necessary when suitable positions in the duplication can be specifically targeted for cleavage.

A lot of duplications, do not appear annotated as “Pathogenic” in ClinVar. Certainly there are many variants listed in Clin Var that are not observed in either the gnomAD genomes or exomes, so are not accounted for in the table above, and this includes 2189 duplications that satisfy all the additional conditions for being in column dup2iP above. But 2183 of these are in these “coding” intervals, so if the variants had been observed at all in gnomAD they would have been reported in these vcfs. It also wouldn't be surprising to miss variants that are extremely rare in general, or even not-terribly-rare variants that are concentrated in populations without many samples: with only ˜100 subjects per population in the TGP data one would expect to miss out on ˜13% of alleles with frequency 0.01 in these populations. And a few other possibilities:
Subjects known to have severe pediatric disease were not included in the gnomAD dataset, so variants that cause these diseases may be under-represented, in particular those with dominant inheritance.
About 8% of the genome was masked during the gnomAD variant calling (e.g. some repetitive sequence), so any Clin Var variants the fall in these regions will not be reported in gnomAD. But it appears that only one of the ˜13000 insertions from Clin Var falls in one of these masked region—a benign variant in SHOX1 which is masked since it's in a PAR on the Y chromosome.
gnomAD doesn't report variants on the Y chromosome at all, whether in masked regions or not. But the only “Pathogenic” duplication on the Y chromosome in ClinVar is a single-base insertion, Y:2655380:C/CT in the gene SRY: ncbi.nlm.nih.gov/clinvar/variation/470195/
The longest insertion reported in the gnomAD coding regions has length 621, and there are 1431 insertions of length at least 100, but it's possible that the detection sensitivity may decline for longer insertions.

The above described variants are listed below; they are mainly variants in UTRs or in intronic regions >50 bases from the nearest exons (and hence not in the “coding” intervals list). gnomAD and TGP vcfs are mainly variants in UTRs or in intronic regions >50 bases from the nearest exons. These sequences are expected to represent “Pathogenic” duplications with a length of at least 100 from Clin Var that satisfy the conditions of the column dup2iP above, none of which are observed in gnomAD. See, Table 5. The First base in the reference allele, the subsequent bases are the inserted sequence

TABLE 5
Duplications Not Contained In “Coding “ Inverval List Or Having Length At Least 100
That Are In Clin Var But Not Present In Either Clin Var Or gnomAD Databases

GTGAGCCACTGCGCCCAGCAGATTCAAGCTTTTTAAATGGAATTTTGAGCTGATTTAGTTG
AGACTTACGTGCTTAGTTGATAAATTTTAATTTTATACTAAAATATTTTACATTAATTCAAG
TTAATTTATTTCAGATTGAATTTAGTGGAAGCTTTTGTAGAAGATGCAGAATTGAGGCAGA
CTTTACAAGAAGATTTACTTCGTCGATTCCCAGATCTTAACCGACTTGCCAAGAAGTTTCA
AAGACAAGCAGCAAACTTACAAGATTGTTACCGACTCTATCAGGGTATAAATCAACTACC
TAATGTTATACAGGCTCTGGAAAAACATGAAGGTAACAAGTGATTTTGTTTTTTTGTTTTC
CTTCAACTCATACAATATATACTTGGCAATGTGCTGTCCTCATAAAGTTGGTGGTGGTGAC
TCACTCTTAGGACACATTCAGATTTCTT (SEQ ID NO: 158)
AG
GT
GAGCTTATCAGGTTCTCCATTGGCAGGCAGGGCTCTAAGTGCAGTAACTTGATTTGCTGTT
GTATTTGCTTAGGAAGAGCAGCACTTCAGAAAAGAGTGATGGCACTGCTGAGGCGCATTG
AGCATCCCACTGCAGGAAACACTGAGGTATGCCCTTAGCAACAGAAACACCCCTCCCAGG
CGCCCACCCTCAATTTGGAAGCCTCTTGTTACATATGTGTGATCAGGAATAGCTTTTGAAG
TAAATCCAAGATACGTGCATATTACAAGTATAATATCTGAGTATTTAATATACATCAAGTT
TGAAACTTGGCTGTAGCTGATTGATGTTTAGCTCT (SEQ ID NO: 159)
TGGGTACGAGTGTCTGCGTATATCTGTATGCTTATTTGGCTCTATGCCTGTGGGTGCACTTA
CTCTGTGTGTTTAGATCAGTCAGTTTCATCTCTCTAGGGGGTCTGTCTTCTGGGCATTGATG
GCAAATCATTAATGTATTTGTTCTTTCTTTAGGTTTTATTGACTGATACCAATACTCAATTT
GTAGAACAAACCATAGCTATAATGAAGAACTTGCTAGATAATCATACTGAAGGCAGCTCT
GAACATCTAGGGCAAGCTAGCATTGAAACAATGATGTTAAATCTGGTCAGGTAAGCATTC
TACTGAAATGTAGCAGAAACATTTTAAGAGATAAGAAAAACCTCTTACACACTGATACTG
GTAGTAATTGATAAAATAACTGGCCATTCTTTACTGCACACAAACTA (SEQ ID NO: 160)
ACCGGTTCCGGCGGCCGGGGCTG (SEQ ID NO: 161)
GTGAGCCACTGCGCCCAGCAGATTCAAGCTTTTTAAATGGAATTTTGAGCTGATTT
AGTTGAGACTTACGTGCTTAGTTGATAAATTTTAATTTTATACTAAAATATTTTAC
ATTAATTCAAGTTAATTTATTTCAGATTGAATTTAGTGGAAGCTTTTGTAGAAGAT
GCAGAATTGAGGCAGACTTTACAAGAAGATTTACTTCGTCGATTCCCAGATCTTAA
CCGACTTGCCAAGAAGTTTCAAAGACAAGCAGCAAACTTACAAGATTGTTACCGA
CTCTATCAGGGTATAAATCAACTACCTAATGTTATACAGGCTCTGGAAAAACATG
AAGGTAACAAGTGATTTTGTTTTTTTGTTTTCCTTCAACTCATACAATATATACTTG
GCAATGTGCTGTCCTCATAAAGTTGGTGGTGGTGACTCACTCTTAGGACACATTCA
GATTTCTT (SEQ ID NO: 162)
TTGGGAGCTAACGGCTTGGAGCTTCTTTCCAGGGATGGGGACCTGGAATTTGAGT
ACTGGTAGACTTTTCGTTGTTCAAACCATTCCTTCACAAATTCCTGAGGAAGGCCC
ACAGC (SEQ ID NO: 163)
TACCTTGGGCCTGGGCCGCAGAGCTGTGAGAATACCCCAGGGCCAGGAGCGCAGT
CTCCACCAGCTGGCTAAAAAGCACATCTTTCCGCACCAGGACAAACTCGGCGTGT
TCTTCTCTGTTGTCATATTCAAGAGAGCCGTCCAACTGCTCCACGACACAAAAGAC
AGGAATCATCAA (SEQ ID NO: 164)
GTGCGCGGGCGGCGGCCGGAAGGGCCTCTTCATGCGGCGGCGGCGCCGGTAGTTG
CCCTTCTCGAACATGTCTTCGCAGGCCGGGTCCAGCGTCCAGTAGTTGCCCTTGCG
CTCGCCGCCGCCCT (SEQ ID NO: 165)
CCGGGCTGGAGAGGGGGATGTTGAGGAGGCTGGGGGTGGGGGCGGGGCATCGAG
GGAGCTCCTGGTACTGGCGGCCCCGACTGTCCCCCCAGAAGCTGAAAATGTTGGA
CACTCCTGAGAAGGCGCCTGCAGCCAGAGAGCAGAGCTGGGTGAGCGGGGTAGA
CGCACCACCGCTGCCACGCCCGGTCCTCCCTCGCCCGCCCGTCGCCCGGGATACCT
GACAGGGGGTTGCAAGTGTCGCTGCTCTTCTCGCAGTCCTCCATCAGGGGCTCCCC
A (SEQ ID NO: 166)
GAGCTTATCAGGTTCTCCATTGGCAGGCAGGGCTCTAAGTGCAGTAACTTGATTTG
CTGTTGTATTTGCTTAGGAAGAGCAGCACTTCAGAAAAGAGTGATGGCACTGCTG
AGGCGCATTGAGCATCCCACTGCAGGAAACACTGAGGTATGCCCTTAGCAACAGA
AACACCCCTCCCAGGCGCCCACCCTCAATTTGGAAGCCTCTTGTTACATATGTGTG
ATCAGGAATAGCTTTTGAAGTAAATCCAAGATACGTGCATATTACAAGTATAATA
TCTGAGTATTTAATATACATCAAGTTTGAAACTTGGCTGTAGCTGATTGATGTTTA
GCTCT (SEQ ID NO: 167)
TGGGTACGAGTGTCTGCGTATATCTGTATGCTTATTTGGCTCTATGCCTGTGGGTG
CACTTACTCTGTGTGTTTAGATCAGTCAGTTTCATCTCTCTAGGGGGTCTGTCTTCT
GGGCATTGATGGCAAATCATTAATGTATTTGTTCTTTCTTTAGGTTTTATTGACTGA
TACCAATACTCAATTTGTAGAACAAACCATAGCTATAATGAAGAACTTGCTAGAT
AATCATACTGAAGGCAGCTCTGAACATCTAGGGCAAGCTAGCATTGAAACAATGA
TGTTAAATCTGGTCAGGTAAGCATTCTACTGAAATGTAGCAGAAACATTTTAAGA
GATAAGAAAAACCTCTTACACACTGATACTGGTAGTAATTGATAAAATAACTGGC
CATTCTTTACTGCACACAAACTA (SEQ ID NO: 168)
CCCAATTCAATGTAGACAGACGTCTTTTGAGGTTGTATCCGCTGCTTTGTCCTCAG
AGTTCTCACAGTTCCAAGGTTAGAGAGTTGGACACTGAGACTGGTTTCCTGCTAAA
CAGTATGGTAAAGAACAGTCAAGCAATTGTTGGCCAGTTCTGTGCTTTTCCTCCTG
AAGAGAAACTTGACACCATGGACAAAATAAATTGACCATCATCAGTCAGCTAACA
TGTATGATGCCTGGAAAAAATGCCCAGGAATTTACACACTAAAATGTCTGGGGCT
GGGAGCGGTAGCTCATGCCTATAATCCCAGCACTTTGGGAGGCTGGAGCAGGACT
GCTTGAGGCCAGGAGTTCAAGACCAGCATAAGCAACAGAGTGAGACCCAGTCTCT
ACAAAATAATAGTAGTAGTAATAATAAAATGTGTGGGATATGTGTGATTTGAATT
TTTTTTTCTGTTGTCTTAAATTTTTCAAACCTGATTATGTATTATTTGTGTAATTTTT
GAAGTATTAATATAGCATATTTTGAAGCTGATACTTGATATACATTCCAATCACAT
CTGATAACTTTTTTTTTTGTTTTGGGGGGTGTACAGAGTCCTGCTCTGTCACCCAGG
CTGGAGTGCAGTGGCGCAATCTCAGCTCACTGCAACCTCCGCCTCCTAAGTTCAAG
AGATTCTCCTGCCTCAGCCTCCTGAGTAGCTGGGTCTACAAGCGTGTGCAACTATG
CCTGGCTAATTTGTGTGTGTGTGTGTATATATATATACATATATATGTGTGTGTGTG
TGTATATATATATATAACATATATATAACATATATATATTATATATATATAACATA
TATATAACATATATATATGTTATATATATATAACATATATATAACATATATATATA
TATATATATAATATATATATATATATATATATATGTAATCCCAGCACTTTGGGATA
TATGTGTATATATGTTTTTTTTTTTTGAGACAGAATCTTGCTCTGTTGCCAGGCTAG
AGTGCAGTGGCGTGATCTCGGCACACTGCAACCTCCACCTCCCTGGTTCAG (SEQ
ID NO: 169)
GGGGGCCATTGTGGAAAAGAGCCTGCAGGGAGAGCAAACAGCGCGGTCATGGCC
TCGGGAGCTGTGCGCGGCGCCTCGGGCAGCGTCTCCCGCCGCTTGTCGCC (SEQ ID
NO: 170)

Duplication variants were identified as annotated as “Pathogenic” or “Pathogenic/Likely_pathogenic” in the Clin Var database (Table 4, dup2iP column), and observed in the gnomAD exome database. See, Table 6.

Table 6 has the following headings:
SEQ_DUP shows the duplicated sequence in upper-case (including the extra copy on the variant allele), and flanking sequence in lower case.
DUP_NUM labels the copies of the duplicated segment (1 for first copy, 2 for second). these positions are also color-coded yellow and cyan in the html table, but the colors are lost when exporting the table as a CSV or Excel file.
Cas9_Wa shows cleavage sites for Cas9 and xCas9 enzymes on the Watson strand, 3 bases left of PAM starts from PAM_FW_* columns.
Cas9_Cr shows cleavage sites for Cas9 enzymes and xCas9 on the Crick strand, 4 bases right of PAM starts from PAM_RC_* columns.
Cpf1_Wa shows approximate cleavage sites for Cpf1 enzymes on the Watson strand, 19 bases right of PAM ends (from the PAM starts in PAM_FW_* columns and adjusted for motif widths).
Cpf1_Cr shows approximate cleavage site for Cpf1 enzymes on the Crick strand, 18 bases left of PAM ends (from the PAM starts in PAM_FW_* and adjusted for motif widths).

TABLE 6
Selected Genes Having Microduplication Variants

| | | | SEQ |
|---|---|---|---|
| | | | ID |
| SYMBOL | MICRODUPLICATION INFORMATION | | NO: |
| PEX10 | ctgccgctgcctgaaaccgtacagcTTgcagccccatggacagcaccaggtg | SEQ_DUP; | 171 |
| | .........................12......................... | DUP_NUM; | |
| | .A..A...aB.J..A....A...A..A......aaJ..A.....aa.A..A. | Cas9_Wa; | |
| | .A.a.a..Aa.a..Aa.....Aa...A..a...A..Aaaa.....A..A.aa | Cas9_Cr; | |
| | .....L......NL..................................N... | Cpf1_Wa; | |
| | ................L................................... | Cpf1_Cr; | |
| CLCNKB | cgtggctggagggatcaccaatcccAAtcatgccaggggggtatgctctggc | SEQ_DUP; | 172 |
| | .........................12......................... | DUP_NUM; | |
| | A..aa.aaaC.................A...aaaaaA...A....aA..aaH | Cas9_Wa; | |
| | ..A.Ca....A........Ca.aa..CaaaI.Ca..IAa...........AJ | Cas9_Cr; | |
| | ....k.M..NLL.................................L...... | Cpf1_Wa; | |
| | ....N............................................... | Cpf1_Cr; | |
| ZMPSTE24 | tgtgggagatgccggccgagaagcgTTatcttcggggccgtgctgctctttt | SEQ_DUP; | 173 |
| | .........................12......................... | DUP_NUM; | |
| | aa.aC.A..aA..a.aB.a.A........aaaA..a.A..A..........a | Cas9_Wa; | |
| | A.a.............Aa..Aa......A....Ca.Ba....Aa...A..a. | Cas9_Cr; | |
| | ......................k.........................N.M. | Cpf1_Wa; | |
| | ..................................L...............L. | Cpf1_Cr; | |
| MMACHC | tgtggcctaccatctgggccgtgttAAgagaggtgaggaaggctcagttttc | SEQ_DUP; | 174 |
| | .........................12......................... | DUP_NUM; | |
| | A..........aaA..a.A....a.a.aa.a.aaB.aA....A......... | Cas9_Wa; | |
| | a.......J.aa..Aa.Ca....Aa......J...............A.a.. | Cas9_Cr; | |
| | ..............................................N..... | Cpf1_Wa; | |
| | ....N...........LN........................k......... | Cpf1_Cr; | |
| ACADM | tccagtccccctaattagaagagccTTgggaacttggtttaatgaacacaca | SEQ_DUP; | 175 |
| | .........................12......................... | DUP_NUM; | |
| | A............aB.a.A....aaaB....aA.....Jab.J......... | Cas9_Wa; | |
| | .....Caah..aaaaa............Aa.......A.............A | Cas9_Cr; | |
| | ......N.....k..N......M.L....L.......N..........N... | Cpf1_Wa; | |
| | .......LLN.........N..LN.................N........N. | Cpf1_Cr; | |
| ACADM | aaaactaatgagggatgccaaaatcTTatcaggtaaggttaaagatgatttt | SEQ_DUP; | 176 |
| | .........................12......................... | DUP_NUM; | |
| | .....a.aaaC.A..............aA...aA.....aC.aC.......a | Cas9_Wa; | |
| | .........A............Aa....Ca..ICa................. | Cas9_Cr; | |
| | .k...........M..................................N.M. | Cpf1_Wa; | |
| | .............N.....k.....................N.........N | Cpf1_Cr; | |
| ABCA4 | ttacgatgtcccagaggaagttggtCCacccagtaggtggtggggctcactc | SEQ_DUP; | 177 |
| | .........................12......................... | DUP_NUM; | |
| | aC.A.....a.aaB.A..aA........A..aa.aa.aaaA........... | Cas9_Wa; | |
| | ..A.a...A....aaa..............aa.aaaH.............A. | Cas9_Cr; | |
| | .......................N........L..........N....L... | Cpf1_Wa; | |
| | ....................................M.k....L........ | Cpf1_Cr; | |
| AGL | gaactgaccaatgagaatgcccagtAActgtcctttcagctgtgaaacacaa | SEQ_DUP; | 178 |
| | .........................12......................... | DUP_NUM; | |
| | .a......aHaB..A....A.....A........A..a.aB.......A... | Cas9_Wa; | |
| | .Baa....A...Aa..........Aaa.....A...aa..Ba..A....... | Cas9_Cr; | |
| | ...L...N..........NLL............................... | Cpf1_Wa; | |
| | ....N.................Lk.....N.........N............ | Cpf1_Cr; | |
| FLG | tgataatgataagaactagaactgtGGaggactgccacgtgactgtattcct | SEQ_DUP; | 179 |
| | .........................12......................... | DUP_NUM; | |
| | ...aC...aB....aB...a.aa.aa...A....a.a...A......Ha.a. | Cas9_Wa; | |
| | a...................A.....A.........A..Aa.a....A.... | Cas9_Cr; | |
| | .k.....kL.............N............................. | Cpf1_Wa; | |
| | ....L...............................M.......N....... | Cpf1_Cr; | |
| GBA | cccacgacactgcctgaagtagaagCCaatcctgtgaggctgccagccatga | SEQ_DUP; | 180 |
| | .........................12......................... | DUP_NUM; | |
| | .a.....A...aB.A..aB.A........a.a.aA..A...A....aC.A.. | Cas9_Wa; | |
| | .....Aaa.a..A.a..Aa...........Aa..CaaHI.....A..Aa..A | Cas9_Cr; | |
| | ....N............................................... | Cpf1_Wa; | |
| | .N....N......................................k...... | Cpf1_Cr; | |
| ASPM | ggtgcatattttgaatatcctttcgTTactttaaagcctctgtaataagact | SEQ_DUP; | 181 |
| | .........................12......................... | DUP_NUM; | |
| | .......HaB..........A..........A.....A......a..JA... | Cas9_Wa; | |
| | ..Aaa....A..J.........CaaH.Ba....A.......Aa.a....... | Cas9_Cr; | |
| | ........N........................k.....M....kL..N... | Cpf1_Wa; | |
| | ...........k..........N..N.......M...k...N.......... | Cpf1_Cr; | |
| ADCK3 | gatgagccttttgattttggcactcAAgagcaccaccgagaagatccacaac | SEQ_DUP; | 182 |
| | .........................12......................... | DUP_NUM; | |
| | .A...I..aC..J.aA......Ja.A.......a.aB.aC.......I..aC | Cas9_Wa; | |
| | aa.a.......Aa............A.a.aH....A.aa.aa.......Caa | Cas9_Cr; | |
| | ..............NL.................k.....k............ | Cpf1_Wa; | |
| | ....N..............N........N....................... | Cpf1_Cr; | |
| HAAO | aagggcagttcccagcggcctcaccAAgggatgggctttcctgttctgtact | SEQ_DUP; | 183 |
| | .........................12......................... | DUP_NUM; | |
| | A..A......a.aA.........aaaC.aaA.......A.J..A....A... | Cas9_Wa; | |
| | ..........A...Baaa..A..Aa.a.aa..........A..Baa...Ba. | Cas9_Cr; | |
| | ...............................NLL.................. | Cpf1_Wa; | |
| | ....N..L.................................LN..N...... | Cpf1_Cr; | |
| MSH6 | aggtaggcacaacttacgtaacagaTTaagagtgaagaagataatgaaattg | SEQ_DUP; | 184 |
| | .........................12......................... | DUP_NUM; | |
| | .aA..........A..I..aC...Ha.a.aB.aB.aC....aB....aHa.a | Cas9_Wa; | |
| | a...A.......A.a..A...A....A......................... | Cas9_Cr; | |
| | ..........kL..N.....................N...........N... | Cpf1_Wa; | |
| | ......N.....LN..N..M.N..Lk..........LN....LN........ | Cpf1_Cr; | |
| MSH6 | tgaaaaggctcgaaagactggacttAAttactcccaaagcaggctttgactc | SEQ_DUP; | 185 |
| | .........................12......................... | DUP_NUM; | |
| | ..aA...aB..a...aa.................A..aA....a..I..aC. | Cas9_Wa; | |
| | ..Aa.........A.a......A....A.......A.aaa....A...A... | Cas9_Cr; | |
| | ................k.............................N...N. | Cpf1_Wa; | |
| | ....N.........k...........................N......... | Cpf1_Cr; | |
| MSH6 | agtgtgcaggctcacaccaattgatAAgagtgtttactagacttggtgcctc | SEQ_DUP; | 186 |
| | .........................12......................... | DUP_NUM; | |
| | .A..aAJ...........aC..Ha.a.A.......a....aa.A.....a.. | Cas9_Wa; | |
| | .A.........AJ.JA.a.a.aa.................JA....A..... | Cas9_Cr; | |
| | .....N..N.....L............................N........ | Cpf1_Wa; | |
| | ..M.N................................N..N........... | Cpf1_Cr; | |
| DYSF | gcgaaaaaatgaggattcgtatcatAAgactggtgagttctgagtcttggag | SEQ_DUP; | 187 |
| | .........................12......................... | DUP_NUM; | |
| | ......aHaaC...A........a...aaHa.A...Ha.A....aa.A.... | Cas9_Wa; | |
| | a.....A..J...........BaH..Ca.J....A........Ba.....a. | Cas9_Cr; | |
| | ..NL.......kLL...L....................NL...M........ | Cpf1_Wa; | |
| | ..M.N......................L........................ | Cpf1_Cr; | |
| DYSF | ctcccagaagacccagccatccccaTTgcccccaagacagttccaccagctg | SEQ_DUP; | 188 |
| | .........................12......................... | DUP_NUM; | |
| | ..aB.a.....A...........A.......a...A........A..aA..A | Cas9_Wa; | |
| | .Caaaa.aaa......AaaH.Aa.Caaaa....Aaaaa....A...Baa.aa | Cas9_Cr; | |
| | ........NL......k.M.L....L.................L....N... | Cpf1_Wa; | |
| | ............N...........................L........... | Cpf1_Cr; | |
| DYSF | tggaaatgaccttggagattgtagcAAgagagtgagcatgaggagcggcctg | SEQ_DUP; | 189 |
| | .........................12......................... | DUP_NUM; | |
| | ...a.....aa.aC..A..A...aHa.a.a.A...a.aa.a.aA...A..aA | Cas9_Wa; | |
| | A...A.........Aa.............A...........A........A. | Cas9_Cr; | |
| | ........L.........................N......N.......... | Cpf1_Wa; | |
| | ...N........................................L....... | Cpf1_Cr; | |
| ALMS1 | agtaccttcaggttccttctcacatAAgagagaagcccagtattttctatca | SEQ_DUP; | 190 |
| | .........................12......................... | DUP_NUM; | |
| | ......aA...............a.a.aB.A....A...............a | Cas9_Wa; | |
| | aa..A....Aa.ba....Baa.Ba.a.a............Aaa.......ba | Cas9_Cr; | |
| | ...........L.................NL....NL..N............ | Cpf1_Wa; | |
| | ..M.N......N..................N..............M...... | Cpf1_Cr; | |
| ALMS1 | aacctctacttcttactcacaacatAAcagagaagccgagtattttctacca | SEQ_DUP; | 191 |
| | .........................12......................... | DUP_NUM; | |
| | .........................a.aB.A.Ha.A...............A | Cas9_Wa; | |
| | .A.....Aa.a..A.Ba...A.aHa..A....A.......Aa........ba | Cas9_Cr; | |
| | ................................N..N................ | Cpf1_Wa; | |
| | ..M.N......N..................N..................... | Cpf1_Cr; | |
| ERCC3 | aaaatttaaatccacaattttgtcaTTcttcgcggacgagggtcgcagtcaa | SEQ_DUP; | 192 |
| | .........................12......................... | DUP_NUM; | |
| | .................A.........a.aa..aHaaA..A..A........ | Cas9_Wa; | |
| | .Ca..I.........CaaHa........a..BaHBa.a...A......a.a. | Cas9_Cr; | |
| | L........N..................k.....L.......k.....N..N | Cpf1_Wa; | |
| | ............L................k...................... | Cpf1_Cr; | |
| MMADHC | acactctactctggcactttcaaagTTaagtttctgcactgttaatttcttg | SEQ_DUP; | 193 |
| | .........................12......................... | DUP_NUM; | |
| | ......J.aA..........A....A..J..A....A..........A.... | Cas9_Wa; | |
| | ....a.a.a.aH.A.aH..A.a..Ba...........Ba..A.a........ | Cas9_Cr; | |
| | ..N................N.....................kL.....N... | Cpf1_Wa; | |
| | k.....N...............N...........N................. | Cpf1_Cr; | |
| NEB | ctgcatctcaggagtgacaggggttGGcggtggctttccccacattttcttt | SEQ_DUP; | 194 |
| | .........................12......................... | DUP_NUM; | |
| | ......aa.a.a...aaaA..aa.aa.aA...............J..A.... | Cas9_Wa; | |
| | .A.aHa..A.Ca.a........A.........A.....A..Baaaa.a.... | Cas9_Cr; | |
| | ..k..N...N.....k..............................N..... | Cpf1_Wa; | |
| | ...................................k.........LLN.... | Cpf1_Cr; | |
| GALNT3 | taaaaatgtgagcgtttcagctgttGGcgactgttgctcctagcaaccgagc | SEQ_DUP; | 195 |
| | .........................12......................... | DUP_NUM; | |
| | ...a.a.a.A.....A..A..aa.a...A..A......A.....a.A..a.a | Cas9_Wa; | |
| | .A.Ca............A...Ba..A......A..A.....A.aa...A..A | Cas9_Cr; | |
| | ...M.M...N............................kL......N..... | Cpf1_Wa; | |
| | .......................N............................ | Cpf1_Cr; | |
| TTN | ccatgacagacacggccttggtcccGGctggcatttttcactgttaaagtgt | SEQ_DUP; | 196 |
| | .........................12......................... | DUP_NUM; | |
| | a.J.a....aA....aA....aA..aA...........A.....a.A..... | Cas9_Wa; | |
| | .....Aa...A...A.a..Aa......aaa..A...A.....Ba.a...... | Cas9_Cr; | |
| | .......N..........k.....................N....L...... | Cpf1_Wa; | |
| | ........................k........................... | Cpf1_Cr; | |
| TTN | gatcagtgactgtatatcttgttttCCacacatgcctctgcattcaccttgt | SEQ_DUP; | 197 |
| | .........................12......................... | DUP_NUM; | |
| | .a.a...A........A...J........A.....A..........a.A... | Cas9_Wa; | |
| | .......Ca.....A......Ca......Baa.a.a...Aa.a..A..BaHa | Cas9_Cr; | |
| | .k....N..NL.......N.................M.M..N....kLL... | Cpf1_Wa; | |
| | ..........................................N......... | Cpf1_Cr; | |
| OBSL1 | ccacgggccagctgacctttgactgTTgacgttggccacggtgcgcacccgg | SEQ_DUP; | 198 |
| | .........................12......................... | DUP_NUM; | |
| | aaA...A..a......a...A..a..A..aA....aaJa.A.....aA.... | Cas9_Wa; | |
| | a.Baaaa.a...Aa..A...Aa.....A......A.....Aa.a....A.aJ | Cas9_Cr; | |
| | ......L............NLL...................k......N... | Cpf1_Wa; | |
| | .................................................... | Cpf1_Cr; | |
| CHRNG | tcttccgttccgcctgctctatctcAAgtcacctacttccccttcgactggc | SEQ_DUP; | 199 |
| | .........................12......................... | DUP_NUM; | |
| | ..A....A...A...........A.................a...aA..aB. | Cas9_Wa; | |
| | ..Aa.Ca.Baa..Baa.aa..A.a..Ca.a....a.aa..A.Baaaa.Ba.. | Cas9_Cr; | |
| | .M.......................NLL..NLL..........M........ | Cpf1_Wa; | |
| | ....N............................N.................. | Cpf1_Cr; | |
| GLB1 | ccccgtacccgggtcccgcagacttAAcgcgcaagccgcgcgtagggcccag | SEQ_DUP; | 200 |
| | .........................12......................... | DUP_NUM; | |
| | A....HaaA....A..a.......a.A...A..a.a.A..aaA....aB.A. | Cas9_Wa; | |
| | ..Aa.aaaa...Aaah...aaa.a...A....A.a.a...Aa.a.a...... | Cas9_Cr; | |
| | ........L..............L.............L........N..... | Cpf1_Wa; | |
| | ....N......N...................N........N......N.... | Cpf1_Cr; | |
| COL7A1 | tcccacagctccagtaggtccagtcAAggccctggaggaagagaaagttcag | SEQ_DUP; | 201 |
| | .........................12......................... | DUP_NUM; | |
| | ...A.....A..aA....A....aA....aa.aaB.a.aB..A....aaA.. | Cas9_Wa; | |
| | ......aaa.a..A.aa.......aa...a....Aaa............... | Cas9_Cr; | |
| | ...............L........L........L........L......... | Cpf1_Wa; | |
| | ....N.......L...LN....k.................N........... | Cpf1_Cr; | |
| COL7A1 | acccacagcaaatagcttgacccccTTgccccttcagcctttgggcagctgt | SEQ_DUP; | 202 |
| | .........................12......................... | DUP_NUM; | |
| | ...A......A...a........A........A.....aaA..A..a.A... | Cas9_Wa; | |
| | .aa...AaaHa..A......A....Aaaaa...Aaaa.Ba..Aa......A. | Cas9_Cr; | |
| | .....................N.................N........N... | Cpf1_Wa; | |
| | ..............................................L..... | Cpf1_Cr; | |
| DNAH1 | ggaacaccgtcaccccgcggctgatGGcgtcacttcaactacctgtctttcg | SEQ_DUP; | 203 |
| | .........................12......................... | DUP_NUM; | |
| | ....A.......a.aA..aC.aa.A...............A......A..a. | Cas9_Wa; | |
| | ...A.....A.aa..a.aaaa.a..A......A..a.a.Ba..A..Aa...a | Cas9_Cr; | |
| | .................................................... | Cpf1_Wa; | |
| | ...............N......................L............N | Cpf1_Cr; | |
| AGTR1 | gtcatgattcctactttatacagtaTTcatctttgtggtgggaatatttgga | SEQ_DUP; | 204 |
| | .........................12......................... | DUP_NUM; | |
| | .aC.......J.......A...........a.aa.aaaB......aaB.... | Cas9_Wa; | |
| | .......a.....Baa..A......A.....BahCa................ | Cas9_Cr; | |
| | .............N.......k........NL......k.M.......NL.. | Cpf1_Wa; | |
| | ...................LLN......LLk...............M..... | Cpf1_Cr; | |
| PCYT1A | ggagggggagcgctcgcgagtagggCCtgctgctggggctctgcttcgggct | SEQ_DUP; | 205 |
| | .........................12......................... | DUP_NUM; | |
| | aaaa.a.A...aHa.A..aaA...A..A..aaaA....A....aaA..aC.a | Cas9_Wa; | |
| | ...............A.a.a.a........Aa..A..A.....A.a..A.Ba | Cas9_Cr; | |
| | .................................................... | Cpf1_Wa; | |
| | .................................................... | Cpf1_Cr; | |
| WDR19 | acattttgtggtcatttctactcatAActggagagcttggtcaagagatatt | SEQ_DUP; | 206 |
| | .........................12......................... | DUP_NUM; | |
| | ...a.aA..................aa.a.A...aA....a.aC.......a | Cas9_Wa; | |
| | ......A..........a...Ba..A.aH...A.......A.....a..... | Cas9_Cr; | |
| | .......N......kL............k.........k............. | Cpf1_Wa; | |
| | ..M.N...L............N....M..............N...M.k...M | Cpf1_Cr; | |
| SCARB2 | ccctccatagaaagaagcaaaacttAAcacaaagtcatctaattttttgaca | SEQ_DUP; | 207 |
| | .........................12......................... | DUP_NUM; | |
| | .....aB..aB.A......J.........A..............a....... | Cas9_Wa; | |
| | A...aaaa.aa..........A.....A....A.a.....a.Ca........ | Cas9_Cr; | |
| | .....................L.....L..................N..... | Cpf1_Wa; | |
| | ....N....k.........N..........M.k...........LLN..... | Cpf1_Cr; | |
| FAM175A | actactaccagtatctgctttagatCCgtttgtcttgtgtatctaacaaccg | SEQ_DUP; | 208 |
| | .........................12......................... | DUP_NUM; | |
| | ......A.....A.....aC...A...A....a.A............a.aC. | Cas9_Wa; | |
| | ......A..A..Aa....Ca.JA......CaaH.....a.......Ca.J.A | Cas9_Cr; | |
| | ...k........k.M..N...N.............M......k.....L... | Cpf1_Wa; | |
| | .......................N..N.............LN.......... | Cpf1_Cr; | |
| MFSD8 | gcaataacccagcccaaaaaacttgTTatcagctgtcggatcaatctgcaga | SEQ_DUP; | 209 |
| | .........................12......................... | DUP_NUM; | |
| | .......A............A......A..A.HaaC.......A..aB..J. | Cas9_Wa; | |
| | ......A.....AaaH.Aaa......A......Ca..A...a...Ca..Ca. | Cas9_Cr; | |
| | ....k...........M.M..........................N..N.M. | Cpf1_Wa; | |
| | ................L....N........k.................N..N | Cpf1_Cr; | |
| ETFDH | tttgtgcagcatatcagtgctttcaTTgccttaaaaattaagaaaaattatc | SEQ_DUP; | 210 |
| | .........................12......................... | DUP_NUM; | |
| | .A..A.......a.A........A.............aB............. | Cas9_Wa; | |
| | ...........A..a...Ca....A..ba....Aa................. | Cas9_Cr; | |
| | ..............kL........k..........M........kL..N... | Cpf1_Wa; | |
| | ...........k......N..k.........................N.... | Cpf1_Cr; | |
| AGA | acaaactaagaagtcataccttggcAAggaagcgcatcaatatatcaccatt | SEQ_DUP; | 211 |
| | .........................12......................... | DUP_NUM; | |
| | .....aB.a.........aA...aaB.a.A...................... | Cas9_Wa; | |
| | ...BaHa...A........a...Aa....A.......A.a.Ca......Ca. | Cas9_Cr; | |
| | N...L...............NL.....................N........ | Cpf1_Wa; | |
| | ....N..LN........N..M............................... | Cpf1_Cr; | |
| DNAH5 | agttcttggtttgcacgggaatgggTTaaatttacagatgctatctccaagg | SEQ_DUP; | 212 |
| | .........................12......................... | DUP_NUM; | |
| | ...aAJ..A...aaaB.HaaA...........aC.A..........aA.... | Cas9_Wa; | |
| | ........Ba........A.a..................A.....A..Ca.a | Cas9_Cr; | |
| | ...kL........N..N.M......N..N....k..............N... | Cpf1_Wa; | |
| | ......k....................N........k.....k.......N. | Cpf1_Cr; | |
| SLC25A46 | agatatccccggcagccgcaacctgCCactggggcgagaagagcccgcccta | SEQ_DUP; | 213 |
| | .........................12......................... | DUP_NUM; | |
| | ......aA..A..A......A.....aaaa.a.aB.a.A...A......aa. | Cas9_Wa; | |
| | aaaaa.....Caaaa..A..Aa.a..Aa..Aa.a.....A........Aaa. | Cas9_Cr; | |
| | ..................L........M.L...................... | Cpf1_Wa; | |
| | .................N.................................. | Cpf1_Cr; | |
| SLC22A5 | ctgctgcaatatttgccccggcgctAAttccatggccactgccctcttcctg | SEQ_DUP; | 214 |
| | .........................12......................... | DUP_NUM; | |
| | .A........A....aa.A..........aA.....A.........Haaa.a | Cas9_Wa; | |
| | ..A..A..A..A........Aaaa..A.a....Baa....Aa.a..Aaa.a. | Cas9_Cr; | |
| | ......M....N.......................k..............NL | Cpf1_Wa; | |
| | ....N............................................... | Cpf1_Cr; | |
| RAD50 | aattatcactttcttcagcccccttAAcaattttggttggacccaatggggc | SEQ_DUP; | 215 |
| | .........................12......................... | DUP_NUM; | |
| | .............A................aA..aa.......aaaa.aaaB | Cas9_Wa; | |
| | ...A......Ca.a..Ba.Ba..Aaaaa....A.............AaaH.. | Cas9_Cr; | |
| | .........................N.M.....k..NL........N..... | Cpf1_Wa; | |
| | ....N..N.........L.....N........LLk........N........ | Cpf1_Cr; | |
| RAD50 | agaaacaagagaaacagcacaagttAAgacacaggtaatacagtctgtgtcc | SEQ_DUP; | 216 |
| | .........................12......................... | DUP_NUM; | |
| | ....a.aB.J..A.....A..J.a.....aa.......A...a.A....J.. | Cas9_Wa; | |
| | Aa........A........A..A.a.........A.a........A...a.. | Cas9_Cr; | |
| | ......L.......................................N..... | Cpf1_Wa; | |
| | ....N..........N.........................M.......... | Cpf1_Cr; | |
| RAD50 | gagactcatgagacaagatattgatAAcacagaaggtaggtctgttttgctt | SEQ_DUP; | 217 |
| | .........................12......................... | DUP_NUM; | |
| | .....a.a....aC....ac.......aB.aA..aA...A....A.....aC | Cas9_Wa; | |
| | .........A.aH.....A.............A.a...........a..... | Cas9_Cr; | |
| | .....................M.....................N........ | Cpf1_Wa; | |
| | ..M.N......N......................M................. | Cpf1_Cr; | |
| GRXCR2 | tgcaaatctggcaaggctgtaggccAAttctcattgcaggcagggcacctca | SEQ_DUP; | 218 |
| | .........................12......................... | DUP_NUM; | |
| | .....aA...aA..A..aA............A..aA.JaaA.......aaA. | Cas9_Wa; | |
| | .......A...Ca..IA....A......Aa...BaHa....A...A....A. | Cas9_Cr; | |
| | ..................N...N...........................N. | Cpf1_Wa; | |
| | ....N.................................M...........k. | Cpf1_Cr; | |
| DSP | agagaattgaagagaggtgcaggcgTTaagctggaggattctaccagggaga | SEQ_DUP; | 219 |
| | .........................12......................... | DUP_NUM; | |
| | B...aB.a.a.aa.A..aa.A....A..aaHaaC........aaa.a..... | Cas9_Wa; | |
| | ..Ca....................A..JA......A........BaH.Aa.. | Cas9_Cr; | |
| | .........L...................N..................N... | Cpf1_Wa; | |
| | ......N....L..............L...................k..... | Cpf1_Cr; | |
| GCM2 | cacaaattgtgttctcacctgacctAAcctgaaaaaagatcgcgttgccatc | SEQ_DUP; | 220 |
| | .........................12......................... | DUP_NUM; | |
| | ....a.A.........a.........aB.....aC..a.A..A.......A. | Cas9_Wa; | |
| | CaahCa.a.........BaJa.aa...Aa...Aa..........Ca.a.... | Cas9_Cr; | |
| | .................M.L.........N....N................. | Cpf1_Wa; | |
| | ....N....Lk....................N.......k............ | Cpf1_Cr; | |
| AARS2 | ctgacatcccagggaagatgccatcAAgagatgcagacactgagtgtgcgga | SEQ_DUP; | 221 |
| | .........................12......................... | DUP_NUM; | |
| | .......aaaB.aC.A.......a.aC.AJ.a....Ha.a.a.aHaaC.a.a | Cas9_Wa; | |
| | ....Aa...A.Caaa..........Aa.Ca........A...A.a....... | Cas9_Cr; | |
| | ....NLL.......................L..................... | Cpf1_Wa; | |
| | ....N.......................L....................... | Cpf1_Cr; | |
| MUT | catggatggttctggaggaaaaatgTTatgtatttcgaaccataaattcctt | SEQ_DUP; | 222 |
| | .........................12......................... | DUP_NUM; | |
| | aC.aA....aa.aaB.....A....A......aB................A. | Cas9_Wa; | |
| | ....Ba.........Ba......................Ba...Aa...... | Cas9_Cr; | |
| | L.....M..............kL.........N...............N.M. | Cpf1_Wa; | |
| | ...............LN...M.k............M.........L...... | Cpf1_Cr; | |
| PKHD1 | aacttcacacacctttaatgtgcagTTaagttgaggatgcttgtgttagtgt | SEQ_DUP; | 223 |
| | .........................12......................... | D?P_NUM; | |
| | .J.............a.A..A....A..aHaaC.A...a.A...a.A....A | Cas9_Wa; | |
| | a..A...A.Ba.a.a.aa.........A..J.............A....... | Cas9_Cr; | |
| | ..........N...............NL.........k..........N... | Cpf1_Wa; | |
| | ......N...............................N...N......... | Cpf1_Cr; | |
| PKHD1 | agtgaatgctgaccccattgatagaGGacggaaattctgtggagaccagctg | SEQ_DUP; | 224 |
| | .........................12......................... | DUP_NUM; | |
| | B..A..a........aC..a.aa..aaB......a.aa.a....A..aA..a | Cas9_Wa; | |
| | H............A...Aaaa............A......BaH.......Aa | Cas9_Cr; | |
| | ..................N.....................N........... | Cpf1_Wa; | |
| | .......LLk.........L................................ | Cpf1_Cr; | |
| WISP3 | cttctgcttgctggcctggcacaggTTaagtcctctcccccgactctttccc | SEQ_DUP; | 225 |
| | .........................12......................... | DUP_NUM; | |
| | .A...A..aA.J.aA....aA....A...........a.............. | Cas9_Wa; | |
| | aa.a.aHBa..A...A...Aa...A.a.........aa.a.aaaaa..A.aH | Cas9_Cr; | |
| | ..............N...L.....N.....N.................N... | Cpf1_Wa; | |
| | ......N............................L................ | Cpf1_Cr; | |
| BRAT1 | ctggctccccaaagagccctggtagTTaactccccctgctgggaagcaaaaa | SEQ_DUP; | 226 |
| | .........................12......................... | DUP_NUM; | |
| | ..........a.A....aA..A...........A..aaaB.A........aB | Cas9_Wa; | |
| | ....aa...A.aaaa.......Aaa.........A.aaaaa..A.......A | Cas9_Cr; | |
| | .....N.......................L..................N... | Cpf1_Wa; | |
| | ......N..............LLN...k......N................. | Cpf1_Cr; | |
| ISPD | gtggtccgcgccgcgctgaccactcAAggcaaggacccggctccgccggcct | SEQ_DUP; | 227 |
| | .........................12......................... | DUP_NUM; | |
| | ...a.A..a.A..a.........aA...aa....aA....A..aA...aa.A | Cas9_Wa; | |
| | .Aa......Jaa.a.aa.a.a...Aa.a.aH...A.....AaaH.A.aa.aa | Cas9_Cr; | |
| | ............................L....................... | Cpf1_Wa; | |
| | ....N....N.......................................... | Cpf1_Cr; | |
| FAM126A | actttgtggctcctggataactttaTTagagagatgaaactaaagaactctt | SEQ_DUP; | 228 |
| | .........................12......................... | DUP_NUM; | |
| | .a.aA....HaaC...........a.a.aC.aB.......aB......A... | Cas9_Wa; | |
| | JAa.a.a.......A.aa.......A..................A....... | Cas9_Cr; | |
| | ................N.........k..................k..N... | Cpf1_Wa; | |
| | ..............Lk....k...N.............k..........M.N | Cpf1_Cr; | |
| SLC25A13 | gtagaaccatcgctgtagcaattcgTTaagtcagcaaagttacaccaaactg | SEQ_DUP; | 229 |
| | .........................12......................... | DUP_NUM; | |
| | B......A..A..A......A....A...A....a............aaaa. | Cas9_Wa; | |
| | Ca..I......Aa.Ca.a.....A...BaH......a..A.......A.aa. | Cas9_Cr; | |
| | ..N.........L...............................NL..N... | Cpf1_Wa; | |
| | ......N.......k..........k................Lk........ | Cpf1_Cr; | |
| PEX2 | attcctgatttcagtggctgcagacTTgtgtacttctgtgccacacttagga | SEQ_DUP; | 230 |
| | .........................12......................... | DUP_NUM; | |
| | ..aC.....a.aA..A..a...Ja.A.......a.a.........aa....A | Cas9_Wa; | |
| | Ca.a...Baa....Ba......A..A...A.......AJBa....Aa.a.a. | Cas9_Cr; | |
| | ......k...N.............NL......kL..............N... | Cpf1_Wa; | |
| | ................................N...k...........k... | Cpf1_Cr; | |
| NBN | gaactttcacatcaatttctaactcTTggttttgtgtccttgaataactgtt | SEQ_DUP; | 231 |
| | .........................12......................... | DUP_NUM; | |
| | .......................aA....a.A....HaB......A..J... | Cas9_Wa; | |
| | ........A..Ba.a.Ca....Ba...A.aH...........aaJ....... | Cas9_Cr; | |
| | .N.......NL..........k......kL.........k........N... | Cpf1_Wa; | |
| | ....................LN..N........N.................. | Cpf1_Cr; | |
| NDUFAF6 | cctgtggccattgaactatggaaggTTaaaaaaaaaaaaataccacttttaa | SEQ_DUP; | 232 |
| | .........................12......................... | DUP_NUM; | |
| | .aA....aB......aaB.aA...........J..................A | Cas9_Wa; | |
| | .Ca..Aa.....Aa......A..........................Aa.a. | Cas9_Cr; | |
| | ...........L.....................N......M.......N... | Cpf1_Wa; | |
| | N.....k......................N........LN............ | Cpf1_Cr; | |
| VPS13B | ctctagagcgccagagaagattgttAAcatttaaaatgttcatcactcagtt | SEQ_DUP; | 233 |
| | .........................12......................... | DUP_NUM; | |
| | .a.a.A...a.aB.aC..A..............A...........A....A. | Cas9_Wa; | |
| | .Ba.aa.a.....A.aa...............A...........Ba.Ca.a. | Cas9_Cr; | |
| | .......N..N...L...k........................N..N..... | Cpf1_Wa; | |
| | ....N......k...................N.................... | Cpf1_Cr; | |
| SPAG1 | aaaagaaggaaactgcagtggctgcAAttcaagattgtaacaggtaaactgc | SEQ_DUP; | 234 |
| | .........................12......................... | DUP_NUM; | |
| | aB.aaB....A..a.aA..A.....I..aC..A.....aA...J..A...A. | Cas9_Wa; | |
| | ..Aa.............A..A.....A..A...BaH.........A...... | Cas9_Cr; | |
| | L................N................................NL | Cpf1_Wa; | |
| | ....N....N.......N......k.......................N... | Cpf1_Cr; | |
| SLC52A2 | cctgcaggttggagcctcccctcttAAcgtctctgtgcttgtggctctgggg | SEQ_DUP; | 235 |
| | .........................12......................... | DUP_NUM; | |
| | ..aA..aa.A..............A.....a.A...a.aA....aaaaB... | Cas9_Wa; | |
| | aa.Baaa..A.........Aa.aaaa.a...A..a.a.....A..J...A.a | Cas9_Cr; | |
| | .................L..NLL.......N.........L.....N..... | Cpf1_Wa; | |
| | ....N.......................LLN..................... | Cpf1_Cr; | |
| CDKN2A | acccacctggatcggcctccgaccgTTaactattcggtgcgttgggcagcgc | SEQ_DUP; | 236 |
| | .........................12......................... | DUP_NUM; | |
| | ...HaaC..aA.....a...A..........aa.a.A..aaA..a.A..... | Cas9_Wa; | |
| | aaHa..AaaHaa....Ca..Aa.aa..Aa.....A...BaH...A..J...A | Cas9_Cr; | |
| | ......L..................................L......N... | Cpf1_Wa; | |
| | ......N............................................. | Cpf1_Cr; | |
| APTX | aaaatctacaatcaccttttcccccAAcagtgtgcatatgcttaaggagttc | SEQ_DUP; | 237 |
| | .........................12......................... | DUP_NUM; | |
| | .........................a.a.A.....A.....aa.A.....aa | Cas9_Wa; | |
| | aaH.A....Ca..a..Ca.aa...Baaaaa..A......AJ.J..A...... | Cas9_Cr; | |
| | .........k...............................kLL........ | Cpf1_Wa; | |
| | ....N.........M.......N........N.................... | Cpf1_Cr; | |
| DNAI1 | ccccataaacagcctcataagcaggTTaacgtacgcacaccttccttctgat | SEQ_DUP; | 238 |
| | .........................12......................... | DUP_NUM; | |
| | .......A........A..aA..J..AJ.JA..............aC.a... | Cas9_Wa; | |
| | ...A.aaaa.....A..Aa.a.....A.......A...A.aJa.aa.Baa.B | Cas9_Cr; | |
| | ...NL.....N............L........................N... | Cpf1_Wa; | |
| | ......N............................................. | Cpf1_Cr; | |
| FANCC | tagtctgtgctctctgctgcctcccAAtcacgggggccgtagtagaaggcca | SEQ_DUP; | 239 |
| | .........................12......................... | DUP_NUM; | |
| | ..a.A......A..A............aaaaA..A..A..aB.aA....a.A | Cas9_Wa; | |
| | aHaa.....a....A.aJa..A..Aa.aaa..Ca.aI....Aa......... | Cas9_Cr; | |
| | .............................................L...... | Cpf1_Wa; | |
| | ....N...................N.....N..................... | Cpf1_Cr; | |
| MUSK | caacattccactggtacatattcttAActctggttgccttcagcggaactga | SEQ_DUP; | 240 |
| | .........................12......................... | DUP_NUM; | |
| | ......J.aA.................aA..A......a.aaB...a.aB.. | Cas9_Wa; | |
| | A.a..a..A..Baa.a.....A..J.BaH...A.aH.....Aa.Ba..A... | Cas9_Cr; | |
| | ...N........................NLL............N..N..... | Cpf1_Wa; | |
| | ....N..................LLN......k.......k........... | Cpf1_Cr; | |
| TSC1 | tgctgtgcgcgtctgctccctgctgTTatcagtctgtccagcacttccattg | SEQ_DUP; | 241 |
| | .........................12......................... | DUP_NUM; | |
| | a.a.a.A...A......A..A......A...A.J..A..........aaaa. | Cas9_Wa; | |
| | A.aa...A....A.aJ.a..A.aaa..A.....Ca...a...aa..A.a.Ba | Cas9_Cr; | |
| | ...L..N...N..NL.......N.................L.......N.M. | Cpf1_Wa; | |
| | ................................L................... | Cpf1_Cr; | |
| ADAMTS13 | ggcagcaggtgctctactgggagtcAAgagagcagccaggctgagatggagt | SEQ_DUP; | 242 |
| | .........................12......................... | DUP_NUM; | |
| | A..aa.A.......aaa.A....a.a.A..A...aA..a.aC.aa.A....a | Cas9_Wa; | |
| | aa.....A..A.....A.aJ.A.......a.......A..Aa...A...... | Cas9_Cr; | |
| | .N..............NLL................................. | Cpf1_Wa; | |
| | ....N.....................L....................LN... | Cpf1_Cr; | |
| AGPAT2 | gtacatgatgaggcccacgggccccAAggaagagcagctcccgcttggcgat | SEQ_DUP; | 243 |
| | .........................12......................... | DUP_NUM; | |
| | ..aC.a.aA.....aaA......aaB.a.A..A.....A...aa.aCJ..A. | Cas9_Wa; | |
| | aa......A..J......Aaa.a...Aaaa.........A..A.aaa.a... | Cas9_Cr; | |
| | N................................................... | Cpf1_Wa; | |
| | .....N..LN.....................................LLN.. | Cpf1_Cr; | |
| C10orf11 | attgcaggtcactggaaggactgagCCgcattcaggagcctggaggaactca | SEQ_DUP; | 244 |
| | .........................12......................... | DUP_NUM; | |
| | ..aA.....aaB.aa...a.A..A......aa.A...aa.aaB......... | Cas9_Wa; | |
| | ....a....A....a.a........A....Aa.a..BaH....Aa....... | Cas9_Cr; | |
| | ......kL....N.....k.....N........................... | Cpf1_Wa; | |
| | ....................L...LN.........L...N..N......... | Cpf1_Cr; | |
| PTEN | gaaagggacgaactggtgtaatgatAAtgtgcatatttattacatcggggca | SEQ_DUP; | 245 |
| | .........................12......................... | DUP_NUM; | |
| | aaa..aB...aa.A....aC....a.A...............aaaA...... | Cas9_Wa; | |
| | ..A..........A...A........J.........A..J.......A.Ca. | Cas9_Cr; | |
| | ........NL.......................................... | Cpf1_Wa; | |
| | ..M.N......M..................k.......k.......N..... | Cpf1_Cr; | |
| PTEN | tgtactttgagttccctcagccgttAAcctgtgtgtggtgatatcaaagtag | SEQ_DUP; | 246 |
| | .........................12......................... | DUP_NUM; | |
| | ...Ha.A........A..A.......a.a.a.aa.aC.......A.Ha.A.. | Cas9_Wa; | |
| | ..Ba.....A..J....Baaa.a..Aa.....Aa.......J.J....Ca.. | Cas9_Cr; | |
| | ...................NL........k....NLL.........N..... | Cpf1_Wa; | |
| | ....N..............M....k.................k.....N..N | Cpf1_Cr; | |
| PNPLA2 | ggacagctccaccaacatccacgagCCtgcgggtcaccaacaccagcatcca | SEQ_DUP; | 247 |
| | .........................12......................... | DUP_NUM; | |
| | .A................a.A...aHaaA...J........A......A... | Cas9_Wa; | |
| | aa.a....A..A.aa.aa..A.CaaHa...Aa..A....a.aa..A.aa..A | Cas9_Cr; | |
| | ..................L............L.........L.......... | Cpf1_Wa; | |
| | .................N.................N........N....... | Cpf1_Cr; | |
| KCNQ1 | cctcgagcgtcccaccggctggaaaTTgcttcgtttaccacttcgccgtgtg | SEQ_DUP; | 248 |
| | .........................12......................... | DUP_NUM; | |
| | a.a.A.......aA..aaB....A....A...........A..a.aHa.A.. | Cas9_Wa; | |
| | ..A.Baa.a...A..aaa.aa..A.........A.Ba.....Aa.a.Ba.aa | Cas9_Cr; | |
| | ..L..................NL..........L..............N... | Cpf1_Wa; | |
| | Lk.................................................. | Cpf1_Cr; | |
| HBB | ttgtccaggtgagccaggccatcacTTaaaggcaccgagcactttcttgcca | SEQ_DUP; | 249 |
| | .........................12......................... | DUP_NUM; | |
| | ...aa.a.A...aA.....J.aA........Ja.A.........A....a.A | Cas9_Wa; | |
| | .........aa.......Aa...Aa.Ca.a.......A.aa...A.a..Ba. | Cas9_Cr; | |
| | .................N.....N...L....................N... | Cpf1_Wa; | |
| | ......k............................................. | Cpf1_Cr; | |
| HBB | cttcatccacgttcaccttgccccaCCagggcagtaacggcagacttctcct | SEQ_DUP; | 250 |
| | .........................12......................... | DUP_NUM; | |
| | ......A........A........aaA..A....aA..a...........aa | Cas9_Wa; | |
| | .aa..A.Ba.CaaHa..Ba.aa...Aaaa.aa....A.....A..A...A.B | Cas9_Cr; | |
| | ........................NL...L....NL....N........... | Cpf1_Wa; | |
| | ..............N..................................... | Cpf1_Cr; | |
| HBB | tgccccacagggcagtaacggcagaCCttctcctcaggagtcagatgcacca | SEQ_DUP; | 251 |
| | .........................12......................... | DUP_NUM; | |
| | .....aaA..A....aA..a............aa.A...aC.A......aa. | Cas9_Wa; | |
| | a.aa...Aaaa.a....A.....A..A...Aa.Ba.aa.a......a..... | Cas9_Cr; | |
| | ......NL...L....NL....N...........................N. | Cpf1_Wa; | |
| | .................................................... | Cpf1_Cr; | |
| ANO5 | gagaactgtagcttctaaagctcatAAgcataggtgtttggcaagacattct | SEQ_DUP; | 252 |
| | .........................12......................... | DUP_NUM; | |
| | ...A..A........A.......A....aa.A...aA...a........... | Cas9_Wa; | |
| | ..A.......A.....A.Ba.....A.a.....A..........J.A....A | Cas9_Cr; | |
| | .......N...........................N................ | Cpf1_Wa; | |
| | ..M.N...M............N.............LLN.............. | Cpf1_Cr; | |
| MYBPC3 | cctcgatcatgcgccgcgcttcatgAActcagctcctgaatcaggtcgaagt | SEQ_DUP; | 253 |
| | .........................12......................... | DUP_NUM; | |
| | aC....a.A..a.A......aB.....A....HaB....aA..aB.A....A | Cas9_Wa; | |
| | a.a.aaa.a..Ca...A.aa.a.a.Ba.....A.aH.A.aa....Ca..I.a | Cas9_Cr; | |
| | ..........................................NL........ | Cpf1_Wa; | |
| | ...LN...........LN........LN........................ | Cpf1_Cr; | |
| MYBPC3 | cgccatcgtaggcaggcggctcccaCCtgtactgtgcaggagtcctctccca | SEQ_DUP; | 254 |
| | .........................12......................... | DUP_NUM; | |
| | ...A..aA..aa.aA......J..A....a.A..aa.A...........A.. | Cas9_Wa; | |
| | A..Aaa.aa.Ca.....A...A..A.aaa.aa....A..J.A..J...aa.a | Cas9_Cr; | |
| | ............................................L....... | Cpf1_Wa; | |
| | .................................................... | Cpf1_Cr; | |
| MYBPC3 | aacacccactcatcgctgtcacctgTTgtcctctggggcatctggggctggc | SEQ_DUP; | 255 |
| | .........................12......................... | DUP_NUM; | |
| | ..........A..A......A..A......aaaA.....aaaA..aA...aA | Cas9_Wa; | |
| | ...a...A.aaaHa.aHCa.a...a.aa......aa.a.....A.Ca..... | Cas9_Cr; | |
| | ..............N..N..............................N... | Cpf1_Wa; | |
| | .................................................... | Cpf1_Cr; | |
| MYBPC3 | atggggtcatcgggggctccaggggTTaggaccattgagagctgctgagctt | SEQ_DUP; | 256 |
| | .........................12......................... | DUP_NUM; | |
| | aA.....aaaaA.....aaaA...aa......a.a.A..A..a.A...a... | Cas9_Wa; | |
| | ..Aa........a.Ca.....A.aa...........Aa........A..A.. | Cas9_Cr; | |
| | .........................................L......N... | Cpf1_Wa; | |
| | ...........................................k........ | Cpf1_Cr; | |
| BSCL2 | gactctggcagctcaagctctaaggTTaacacgatacggctgtccatacatc | SEQ_DUP; | 257 |
| | .........................12......................... | DUP_NUM; | |
| | ..aA..A.....A......aAJ....J.aC...aA..AJ.......J..A.. | Cas9_Wa; | |
| | a......A.aH..A..A.a...A.a.........A.a....A..A...aa.. | Cas9_Cr; | |
| | ..........N...NL................................N... | Cpf1_Wa; | |
| | N.....N.....M...........M................k.......... | Cpf1_Cr; | |
| BBS1 | cctgcgaagatggccgctgcgtcctCCatcggattccgacgcctgcggagct | SEQ_DUP; | 258 |
| | .........................12......................... | DUP_NUM; | |
| | .aB.aC.aA..A..a.A.......IHaaC....a..A...a.aa.A..a.a. | Cas9_Wa; | |
| | A..A.aa..A........Aa.a..A..aa.aa.Ca....Baa..A.aa..A. | Cas9_Cr; | |
| | .........N......................................L... | Cpf1_Wa; | |
| | .........L...............L............LN............ | Cpf1_Cr; | |
| LRP5 | tgactgtatgcacaacaacgggcagTTgtgggcagctgtgccttgccatccc | SEQ_DUP; | 259 |
| | .........................12......................... | DUP_NUM; | |
| | .AJ..A.........aaA..A..a.aaA..A..a.A....A........aa. | Cas9_Wa; | |
| | a.a.....A......AJa..A..A...A.........A..A....Aa.J.Aa | Cas9_Cr; | |
| | ...L..........................M.................N... | Cpf1_Wa; | |
| | .................................................... | Cpf1_Cr; | |
| TYR | cggcgatggtaggggccgtcctcacTTgccctgctggcagggcttgtgagct | SEQ_DUP; | 260 |
| | .........................12......................... | DUP_NUM; | |
| | aC.aA..aaaA..A.........A....A..aA..aaA...a.a.A...A.. | Cas9_Wa; | |
| | .....A..A...........Aa..aa.a.a...Aaa..A...A....A.... | Cas9_Cr; | |
| | .................N..............................N... | Cpf1_Wa; | |
| | .............................................N....k. | Cpf1_Cr;. | |
| TYR | ggcagggcttgtgagcttgctgtgtCCgtcacaagagaaagcagcttcctga | SEQ_DUP; | 261 |
| | .........................12......................... | DUP_NUM; | |
| | aaA...a.a.A...A..a.A...A......a.aB..A..A......aB.aB. | Cas9_Wa; | |
| | a..A...A....A.......A...A.....aaJ.a.a.........A..A.B | Cas9_Cr; | |
| | ...............................N.......N........L... | Cpf1_Wa; | |
| | ...........N....k............LN..k.................. | Cpf1_Cr; | |
| MRE11A | tccacaaatttctggctaaagcgaaGGaacactgaaaggttcaaaacctcca | SEQ_DUP; | 262 |
| | .........................12......................... | DUP_NUM; | |
| | .........aA.....a.aB.aaB.....aB..aA................. | Cas9_Wa; | |
| | AaaH.CaaHa.....Ba...A.....A.......A.a........Ba....A | Cas9_Cr; | |
| | ......k....N.............L......k................... | Cpf1_Wa; | |
| | .LN..LN.....Lk.......k...........M..........Lk...... | Cpf1_Cr; | |
| ATM | acagaacaatcccagcctaaaacttAAcatacacagaatgtctgagggtttg | SEQ_DUP; | 263 |
| | .........................12......................... | DUP_NUM; | |
| | B.........A..........J.J......HaB..A...aHaaA...a.aA. | Cas9_Wa; | |
| | a.....A....A..CaaaI.Aa.....A....A...A.a.......a..... | Cas9_Cr; | |
| | ..k.......k.....k..N.M...........L............N..... | Cpf1_Wa; | |
| | ....N..M.......N...................N............k... | Cpf1_Cr; | |
| ATM | aggagtggaagaaggcactgtgctcAAgtgttggtggacaagtgaatttgct | SEQ_DUP; | 264 |
| | .........................12......................... | DUP_NUM; | |
| | a.aaB.ab.aA....a.A.....a.A..aa.aa....aHaB....AJ..... | Cas9_Wa; | |
| | A...................A.a....A.aJ.......J....A........ | Cas9_Cr; | |
| | .................................................... | Cpf1_Wa; | |
| | ....N.........L...N...LN........M...........M....... | Cpf1_Cr; | |
| DPAGT1 | tgtggtagagcaatcccaaagtggtGGaaaaaaaagggtatcatgaagtaga | SEQ_DUP; | 265 |
| | .........................12......................... | DUP_NUM; | |
| | A..a.A..........a.aa.aaB......HaaA......aB.A..a.aaaB | Cas9_Wa; | |
| | Aa.............A..CaaaI......................Ca.J... | Cas9_Cr; | |
| | .M.L...........N......N..............L.............. | Cpf1_Wa; | |
| | ....LLk................LN.......LLk................. | Cpf1_Cr; | |
| PKP2 | aggagaggttatgaagaatgcacacAAcaattctccgtggcctgagaaaaca | SEQ_DUP; | 266 |
| | .........................12......................... | DUP_NUM; | |
| | a.aA....aBHab.JA................a.aA...a.aB.....aa.. | Cas9_Wa; | |
| | A........................A.a.a..A...BaHaa....Aa..... | Cas9_Cr; | |
| | ..............L................N.M.................. | Cpf1_Wa; | |
| | ....N..N.................k.........N..N...........M. | Cpf1_Cr; | |
| PKP2 | ggcccgcctgctttcttggtggtgcAAgggtgtgcccagcctggcttctctg | SEQ_DUP; | 267 |
| | .........................12......................... | DUP_NUM; | |
| | .A...A.......aa.aa.A..Haaa.a.A....A...aA.......aa..A | Cas9_Wa; | |
| | ....A..Aaa.aa..A..Ba.........A..J......AaaJ.Aa...A.B | Cas9_Cr; | |
| | .M.................................k..N............. | Cpf1_Wa; | |
| | ....N.....................................N......... | Cpf1_Cr; | |
| AAAS | ggtcccagaccatggagtgagcctcTTcccccaagcctgtgggtaaggacag | SEQ_DUP; | 268 |
| | .........................12......................... | DUP_NUM; | |
| | ...a.....aa.a.a.A.............A...aHaaA...aa...aA... | Cas9_Wa; | |
| | .a......aaa...Aa..........Aa.a.Baaaaa...Aa.......... | Cas9_Cr; | |
| | ............NLL...........L.....................NLL. | Cpf1_Wa; | |
| | ...........N...........N............................ | Cpf1_Cr; | |
| MVK | ttcatggagaacatgccgtggtacaTTggcaaggtacaaagccgttagagcc | SEQ_DUP; | 269 |
| | .........................12......................... | DUP_NUM; | |
| | .aa.aB....A..a.aA......aA.J.aA......A..A...a.A.....I | Cas9_Wa; | |
| | a.CaaHBa........A...Aa......A..J..A......A..J.Aa.... | Cas9_Cr; | |
| | .......L...............NL.......................N... | Cpf1_Wa; | |
| | .........N......k.................N................. | Cpf1_Cr; | |
| TCTN2 | tccggccctgcggtcagcgcgtcccTTggtcggagacaccgagggtgtgacc | SEQ_DUP; | 270 |
| | .........................12......................... | DUP_NUM; | |
| | A....a.aA...a.a.A......aA..aa.a.....aHaaa.a.a...a.A. | Cas9_Wa; | |
| | H.....aa..Aaa..A...a..A.a..aaa.....a.....A.aa....... | Cas9_Cr; | |
| | L...L.......k.M.L.......L....................L..N... | Cpf1_Wa; | |
| | ..........L......................................... | Cpf1_Cr; | |
| ATP6V0A2 | agtcgggcacggcctacgagtgcctCCagcgccctgggcgagaaaggcctgg | SEQ_DUP; | 271 |
| | .........................12......................... | DUP_NUM; | |
| | aaA...aA....Ha.a.A......a.A....aaa.a.aB..aA...aA.... | Cas9_Wa; | |
| | aa..A...a...A.a..Aa..A.....Aa.aa..A.aaa....A........ | Cas9_Cr; | |
| | ................NL..............................L... | Cpf1_Wa; | |
| | .....................k.............................. | Cpf1_Cr; | |
| GJB2 | cttcctcttcttctcatgtctccggTTaggccacgtgcatggccactaggag | SEQ_DUP; | 272 |
| | .........................12......................... | DUP_NUM; | |
| | .............A.....aA...aA....a.A...aA......aa.a.A.. | Cas9_Wa; | |
| | .....A.Baa.a.Ba.Ba.a....a.aa.......Aa.a...A..J.Aa.a. | Cas9_Cr; | |
| | ....k.M...L....N........NL....N..N..........L...N... | Cpf1_Wa; | |
| | ........................................L.....LN.... | Cpf1_Cr; | |
| GJB2 | ccggtaggccacgtgcatggccactAAggagcgctggcgtggacacgaagat | SEQ_DUP; | 273 |
| | .........................12......................... | DUP_NUM; | |
| | ..aA....a.A...aA.......aa.a.A..aa.aJaa....aB.aC...A. | Cas9_Wa; | |
| | ...a.aa......Aa.a...A..J.Aa.a.......A.a...A.....A.a. | Cas9_Cr; | |
| | ...NL....N..N..........L............................ | Cpf1_Wa; | |
| | ...N...............L.....LN..................M.....L | Cpf1_Cr; | |
| CENPJ | ccctttttaatgcaaggaaaggctgTTatgggtttcagatttatctgactgt | SEQ_DUP; | 274 |
| | .........................12......................... | DUP_NUM; | |
| | .......A...aaB..aA..A...HaaA..I..aC.......a...a.aA.. | Cas9_Wa; | |
| | .....aaa.........A.........A...........Ba.......Ca.. | Cas9_Cr; | |
| | .......................L.....k..................N.M. | Cpf1_Wa; | |
| | ......................................N............. | Cpf1_Cr; | |
| BRCA2 | cttattttaactcctacttccaaggAAtgttctgtcaaacctagtcatgatt | SEQ_DUP; | 275 |
| | .........................12......................... | DUP_NUM; | |
| | ...................aaB..A....A.........A.I..aC...... | Cas9_Wa; | |
| | .A.a.aH........A.aa..A.Baa.........Ba...a...Aa....a. | Cas9_Cr; | |
| | M.......................N....k..........NLL......... | Cpf1_Wa; | |
| | N..LN..........k........................k...N...M... | Cpf1_Cr; | |
| BRCA2 | attgtaaaaatagtcatataaccccTTcagatgttattttccaagcaggatt | SEQ_DUP; | 276 |
| | .........................12......................... | DUP_NUM; | |
| | ........A................aC.A...........AIHaaC...... | Cas9_Wa; | |
| | .Ba................a......Aaaa.Ba...........Baa...A. | Cas9_Cr; | |
| | ..............N...k.....N...............M.......NL.. | Cpf1_Wa; | |
| | .....................N...........N....k....M.N....N. | Cpf1_Cr; | |
| BRCA2 | atcaccagttttagccatcaatgggCCaaagaccctaaagtacagagaggcc | SEQ_DUP; | 277 |
| | .........................12......................... | DUP_NUM; | |
| | ...A.....A........aaA.....a.....J..A....a.a.aA...A.. | Cas9_Wa; | |
| | J.....Ca.aa........Aa.Ca......Aa.....AaaH......A..J. | Cas9_Cr; | |
| | .................................k.................. | Cpf1_Wa; | |
| | ......k........k..................k........LN....... | Cpf1_Cr; | |
| BRCA2 | caaacgaaaattatggcaggttgttAAcgaggcattggatgattcagaggat | SEQ_DUP; | 278 |
| | .........................12......................... | DUP_NUM; | |
| | .aB.......aA..aA..A.....a.aA...HaaC.aC....aHaaC..... | Cas9_Wa; | |
| | ....Aa...A...........A..........A....A..........BaH. | Cas9_Cr; | |
| | ...................k.............N.M.......N..N..... | Cpf1_Wa; | |
| | ....N..........L.............M........M.N.........M. | Cpf1_Cr; | |
| BRCA2 | tttctgaaatagaagatagtaccaaGGcaagtcttttccaaagtattgttta | SEQ_DUP; | 279 |
| | .........................12......................... | DUP_NUM; | |
| | .aB....aB.ac..A......aA...A...........A....A.......A | Cas9_Wa; | |
| | .......ba.................Aa.J..A....a...Baa........ | Cas9_Cr; | |
| | .N......................k........................... | Cpf1_Wa; | |
| | ..N....N..........k...........k.....N..LN........... | Cpf1_Cr; | |
| BRCA2 | gtttctttagagccgattacctgtgTTaccctttcggtaagacatgtttaaa | SEQ_DUP; | 280 |
| | .........................12......................... | DUP_NUM; | |
| | .....a.a..aC......a.A..........aA...a....A.......... | Cas9_Wa; | |
| | .Ca.....Ba.......Aa.....Aa.......aaaH.Ba.......A.... | Cas9_Cr; | |
| | ........N...k.M..........k...k.........N........N... | Cpf1_Wa; | |
| | .................N..........k.........k......N...... | Cpf1_Cr; | |
| BRCA2 | tgcccctttcgtctatttgtcagacGGaatgttacaatttactggcaataaa | SEQ_DUP; | 281 |
| | .........................12......................... | DUP_NUM; | |
| | .....A........A...a..aaB..A............aA.......A... | Cas9_Wa; | |
| | ...A...Aaaa..Ba..a.......a...A.........A......A...A. | Cas9_Cr; | |
| | ........kLL..N........N.......kL.......k............ | Cpf1_Wa; | |
| | ....LLN.......N..........N..k.......L.M........N.... | Cpf1_Cr; | |
| BRCA2 | agatgtcttctcctaattgtgagatAAtattatcaaagtcctttatcacttt | SEQ_DUP; | 282 |
| | .........................12......................... | DUP_NUM; | |
| | A.............a.a.aC.............A..............A... | Cas9_Wa; | |
| | ...Ca......a.Ba.aa...................Ca.....aa....Ca | Cas9_Cr; | |
| | .........kL.......k.M.........N........N............ | Cpf1_Wa; | |
| | ..M.N........k.........................k....LN...... | Cpf1_Cr; | |
| ATP7B | caggagcacccgcatgaccctggggAAcgccatgggtaatggtgccagtctt | SEQ_DUP; | 283 |
| | .........................12......................... | DUP_NUM; | |
| | .A.....A...a.....aaaaB..A...HaaA....aa.A...A....A... | Cas9_Wa; | |
| | aaa..A.....A.aaaHa....AaaH......A.aa.............Aa. | Cas9_Cr; | |
| | ................L................................... | Cpf1_Wa; | |
| | ..LLN...........N.................k................. | Cpf1_Cr; | |
| ATP7B | gtccatgttggctgacctgtgtctcAAgagatttgtaggcctgaacgtagaa | SEQ_DUP; | 284 |
| | .........................12......................... | DUP_NUM; | |
| | ..A..aA..a....a.A.....Ia.aC...A..aA...aB..A..aB.A... | Cas9_Wa; | |
| | ..A.a..aa.......A...Aa.....a.a..............Aa....A. | Cas9_Cr; | |
| | .N.......................L....N..................... | Cpf1_Wa; | |
| | ....N................LN......N.....................L | Cpf1_Cr; | |
| NRL | cccagctgctgctgcagggtagccaGGccagtacagctcctccaggcctggc | SEQ_DUP; | 285 |
| | .........................12......................... | DUP_NUM; | |
| | A..A..A..A.HaaA..A...aAJ..A....A........aA...aA.Haaa | Cas9_Wa; | |
| | a..Aaaaa..A..A..A..A.......Aa...Aa....A..a.aa.aa...A | Cas9_Cr; | |
| | ...............L.................................... | Cpf1_Wa; | |
| | .................................................... | Cpf1_Cr; | |
| RDH12 | caggagcccgagtctatattgcctgCCagagatgtactgaagggggagtctg | SEQ_DUP; | 286 |
| | .........................12......................... | DUP_NUM; | |
| | .A..Ha.A........A...A...a.aC.A....aB.aaaaa.A...A..A. | Cas9_Wa; | |
| | ...AaaH....Aaa....a.......Aa..Aa.........A..J....... | Cas9_Cr; | |
| | ...........k....N.....................M..N.......... | Cpf1_Wa; | |
| | .................LN....L...............Lk.........L. | Cpf1_Cr; | |
| SPATA7 | atgccaaagaaaaaatagctcctttAAcctttagaagggcatgactcaacat | SEQ_DUP; | 287 |
| | .........................12......................... | DUP_NUM; | |
| | ....aB.......A...............aB.aaA...a.........aaaC | Cas9_Wa; | |
| | ........Aa.............A.aa.....Aa..........A....A.a | Cas9_Cr; | |
| | .........N..........M.........................k..... | Cpf1_Wa; | |
| | ....N........N............N.....L.........N......... | Cpf1_Cr; | |
| OCA2 | cttctcggaggaggcagatgcagacAAgaccagacacctccctgcttagcag | SEQ_DUP; | 288 |
| | .........................12......................... | DUP_NUM; | |
| | ..aa.aa.aA..aC.A..a....a..J.a..........A....A..aA... | Cas9_Wa; | |
| | .a.aaa.Ba.a........A.....A...A....Aa...A.aa.aaa..A.. | Cas9_Cr; | |
| | ..........L..........L..N........................... | Cpf1_Wa; | |
| | ....N............................................... | Cpf1_Cr; | |
| EIF2AK4 | ataagctcttgtgacctcctggttgTTaagtgttggccagatgtctatgtcc | SEQ_DUP; | 289 |
| | .........................12......................... | DUP_NUM; | |
| | A.....a.a.......aA..A....a.A..aA...aC.A.....A....aaA | Cas9_Wa; | |
| | ...A......A.a......Aa.aa................JAa......a.. | Cas9_Cr; | |
| | ......k......L....N............N.............N..N... | Cpf1_Wa; | |
| | ......N.................................N....N...... | Cpf1_Cr; | |
| CAPN3 | ctgagttcccaccggatgagacctcTTctcttttatagccagaagttcccca | SEQ_DUP; | 290 |
| | .........................12......................... | DUP_NUM; | |
| | A.......HaaC.a.a................A...aB.A............ | Cas9_Wa; | |
| | ...AaaH....Baaa.aa........Aa.a.Ba.a........Aa......B | Cas9_Cr; | |
| | ........N...k.M.............NLL.................N... | Cpf1_Wa; | |
| | .............M.......N.....................LLN...... | Cpf1_Cr; | |
| TUBGCP4 | aacagctctcagcctggatgctccaTTggactcctcttggaccagcatgaag | SEQ_DUP; | 291 |
| | .........................12......................... | DUP_NUM; | |
| | A......A..HaaC.A.......aa.........aa....A...aBHaB... | Cas9_Wa; | |
| | ......JA..A.a.a..Aa......A.aa......A.aa.a.....Aa..A. | Cas9_Cr; | |
| | ...k................M........................L..N... | Cpf1_Wa; | |
| | ......L..........L.........LN..N..........k......... | Cpf1_Cr; | |
| SPG11 | cataccttggcaagatcatacagacAAgaggacctgtcgacagtagttcttc | SEQ_DUP; | 292 |
| | .........................12......................... | DUP_NUM; | |
| | ....aA...ac.......a....a.aa....A..a...A..A.......... | Cas9_Wa; | |
| | ...A.a...Aa....A....Ca...A...A.......Aa...a..A...... | Cas9_Cr; | |
| | .................N...........N...................... | Cpf1_Wa; | |
| | ....N..............................L................ | Cpf1_Cr; | |
| SPG11 | tgggtctccaaatcccagagggtaaTTggtatagcccatcctttccacttcc | SEQ_DUP; | 293 |
| | .........................12......................... | DUP_NUM; | |
| | .............aHaaA.....aA....A.....................A | Cas9_Wa; | |
| | ..Aaa.....a.aa...CaaaI.................aaa.CaaH.Baa. | Cas9_Cr; | |
| | ..............................L.....L...........N... | Cpf1_Wa; | |
| | ..N......M......................N...k...........LN.. | Cpf1_Cr; | |
| DUOXA2 | ttcacctggcgtctgaaagagaattAAcgccgcggagtacgcgaacgcactg | SEQ_DUP; | 294 |
| | .........................12......................... | DUP_NUM; | |
| | ...aa.A...aB..aHaB......A..a.aa.A...a.ab..A....aa.aB | Cas9_Wa; | |
| | ..A...Ba.aa...A..a..............A.aa.a......A.aJ..A. | Cas9_Cr; | |
| | ......N................NL.....................N..... | Cpf1_Wa; | |
| | N...N.......L........LN.......L...N..........L...... | Cpf1_Cr; | |
| FBN1 | aagcccaaagccttcaaagacacttAAccttggcaccttcttccactggagg | SEQ_DUP; | 295 |
| | .........................12......................... | DUP_NUM; | |
| | .....A......J............J.aA..............aa.aa.... | Cas9_Wa; | |
| | ........Aaa....Aa.Ba.....A.a....Aa....A.aa.Ba.Baa.a. | Cas9_Cr; | |
| | ...................N...............NL.........N..... | Cpf1_Wa; | |
| | ....N.....................L......N..Lk..........L... | Cpf1_Cr; | |
| KIF7 | gtgccgcgccgcgttgcccatctccAAggaggctcagcacctcatccaggcc | SEQ_DUP; | 296 |
| | .........................12......................... | DUP_NUM; | |
| | .a.A..a.A..A...........aa.aA.J..A...........aA...... | Cas9_Wa; | |
| | aaa.....Aa.a.aa.a....Aaa.Ca.aa.......A.a..A.aa.a.Caa | Cas9_Cr; | |
| | ...N..............L.................N.........L..... | Cpf1_Wa; | |
| | ....N............................................... | Cpf1_Cr; | |
| BLM | aagcagtatttttttttccaactagTTggggacatgattttcgtcaagatta | SEQ_DUP; | 297 |
| | .........................12......................... | DUP_NUM; | |
| | .A..................A..aaaa.I..aC.....A.I..aC....... | Cas9_Wa; | |
| | a.......A.......J....Baa..A..........A.......Ba..a.. | Cas9_Cr; | |
| | .........L............................kLL.......N... | Cpf1_Wa; | |
| | ........L...............N.......k....N..LN.......... | Cpf1_Cr; | |
| BLM | tccttctgttccggtgatggctcttAAcggccacagctaatcccagggtaca | SEQ_DUP; | 298 |
| | .........................12......................... | DUP_NUM; | |
| | ...A....aa.aC.aA........aA.....A........haaA....aB.a | Cas9_Wa; | |
| | .....Baa.Ba...Baa........A.a....A..Aa.a..A...CaaaI.. | Cas9_Cr; | |
| | ......M...NL..........kL..N....NLL............N..... | Cpf1_Wa; | |
| | ....N............N..............N................... | Cpf1_Cr; | |
| BLM | tgtccattacttcaatatttttaatAAccgtcactctcaagaagcttgcagg | SEQ_DUP; | 299 |
| | .........................12......................... | DUP_NUM; | |
| | .........................A..........aB.A...A..aahaaA | Cas9_Wa; | |
| | ........aaJ...A.Ba..............Aa..a.a.aHa......A.. | Cas9_Cr; | |
| | ...................k......L..N...NL........k........ | Cpf1_Wa; | |
| | .N..N............N..N............................... | Cpf1_Cr; | |
| ERCC4 | gtactacatgaagtggagccaagatAAcgtggttctttatgacgcagagcta | SEQ_DUP; | 300 |
| | .........................12......................... | DUP_NUM; | |
| | .....aB.a.aa.A....aC....a.aA........a..A..a.A....... | Cas9_Wa; | |
| | A.......A..a...........Aa.......A.....Ba.......A.a.. | Cas9_Cr; | |
| | .........M.......................................... | Cpf1_Wa; | |
| | ..M.N.........................N..................Lk. | Cpf1_Cr; | |
| PALB2 | cagaaagggtcccactgctactaacTTagcctcctctttgtcaggccaagca | SEQ_DUP; | 301 |
| | .........................12......................... | DUP_NUM; | |
| | .HaaA.......A...........A..........A...aA.J..A....A. | Cas9_Wa; | |
| | ....Ca.........aaa.a..A..A...A....Aa.aa.a.....a...Aa | Cas9_Cr; | |
| | .........N.....N..N..............L..............N... | Cpf1_Wa; | |
| | .N........................N............M............ | Cpf1_Cr; | |
| PALB2 | gtgctgactactaccgctatctgatAAgagtctgtaaaggaactgtagtcgc | SEQ_DUP; | 302 |
| | .........................12......................... | DUP_NUM; | |
| | .a.........A......aC..Ha.A...A....aaB...A..A..A....a | Cas9_Wa; | |
| | BaJ.....A..JA..A..Aa.a..Ca..........a..........A.... | Cas9_Cr; | |
| | ...k.............NL......................M.......... | Cpf1_Wa; | |
| | ..M.N.........k...LN.................Lk............. | Cpf1_Cr; | |
| CLN3 | ctcttaccagcggtattgctgagcgTTgactcagggaagtgttccagaaaaa | SEQ_DUP; | 303 |
| | .........................12......................... | DUP_NUM; | |
| | .....a.aA....A..a.a.A..a.....aaaB.a.A.....aB....a.aa | Cas9_Wa; | |
| | a.a.aa.a...Aa..A.......a....A.....A.aH.........Baa.. | Cas9_Cr; | |
| | ..L.......................N...........N.........N... | Cpf1_Wa; | |
| | .............LLN..........k.............k......N..k. | Cpf1_Cr; | |
| CLN3 | atggacagcagggtctgctgaggggAAgaggccggcctgggtgaggcccagg | SEQ_DUP; | 304 |
| | .........................12......................... | DUP_NUM; | |
| | ...A.HaaA...A..a.aaaaB.a.aA..aA..Haaa.a.aA....aA..aa | Cas9_Wa; | |
| | aaH.A.....A..A.....a..A.............Aa..Aa.........A | Cas9_Cr; | |
| | .................................................... | Cpf1_Wa; | |
| | ..LLN............................................... | Cpf1_Cr; | |
| TK2 | ggcggcccagccccgcagcggccacAAgcagcatagccgggcgagcggatcc | SEQ_DUP; | 305 |
| | .........................12......................... | DUP_NUM; | |
| | A....A....A..a.aA......A..A....A..aaa.a.aHaaC....a.a | Cas9_Wa; | |
| | .aaa...A..Aaa..Aaaa.a..A..Aa.a...A..A....Aa...A...A. | Cas9_Cr; | |
| | .................................................... | Cpf1_Wa; | |
| | ...N.......M.............L.......................... | Cpf1_Cr; | |
| APRT | agcagttggctgcggggagacccttAAccaccagtggccagcagatcatcca | SEQ_DUP; | 306 |
| | .........................12......................... | DUP_NUM; | |
| | A..aA..a.aaaa.a..............a.aA...A..aC........a.. | Cas9_Wa; | |
| | .Aa.a..A......A..A.......AaaH...Aa.aa.....Aa..A...Ca | Cas9_Cr; | |
| | .L...........N..............N.................N..... | Cpf1_Wa; | |
| | ....N............................................... | Cpf1_Cr; | |
| SPG7 | ttcagccaaggcctgcagcagatgaTTggaccatgtgagtcggctctggcca | SEQ_DUP; | 307 |
| | .........................12......................... | DUP_NUM; | |
| | A....aA...A..A..aC.aC..aa.....aHa.A..aA....aa....... | Cas9_Wa; | |
| | .aaaa.Ba..Aa....Aa..A..A...........Aa........a..A.a. | Cas9_Cr; | |
| | .......................NL.......................N... | Cpf1_Wa; | |
| | ......L............................................. | Cpf1_Cr; | |
| CTNS | cttcagcctgcacgcggttgtcctcAAcgctgatcatcatcgtgcagtgctg | SEQ_DUP; | 308 |
| | .........................12......................... | DUP_NUM; | |
| | .AJ..A...a.aA..A........A..aC........a.A..a.A..A...A | Cas9_Wa; | |
| | ..a.Ba.Ba..Aa..A.a.a......aa.a..A.a...Ca.Ca.Ca...A.. | Cas9_Cr; | |
| | .....................N..NL..............N........... | Cpf1_Wa; | |
| | ....N............................................... | Cpf1_Cr; | |
| CTNS | ctcgcccccagcgcggtggccagcgCCgtgtcctggcctgccatcggcttcc | SEQ_DUP; | 309 |
| | .........................12......................... | DUP_NUM; | |
| | ......a.a.aa.aA...a.A..a.A....aA...A.....aA......aa. | Cas9_Wa; | |
| | a.aa.a.a.aaaaa..A.a.....Aa..A.aa....aaJ..Aa..Aa.Ca.. | Cas9_Cr; | |
| | .....L.............................................. | Cpf1_Wa; | |
| | .................................................... | Cpf1_Cr; | |
| FLCN | cgtactggctgctgtatgggatgatGGcggacgcagcccacgggaagcatgg | SEQ_DUP; | 310 |
| | .........................12......................... | DUP_NUM; | |
| | ..aA..A..A...aaaC.aC.aa.aa..A..A.....aaaB.A...aA...a | Cas9_Wa; | |
| | a.aa.a...A..JA..A.......J.......A...A.a..Aaa.a...... | Cas9_Cr; | |
| | ....N.................................M............. | Cpf1_Wa; | |
| | .......L.............LLN............................ | Cpf1_Cr; | |
| FLCN | ggcctggcggacaatgctgaagagcTTgggggtggctggggtgctggtggct | SEQ_DUP; | 311 |
| | .........................12......................... | DUP_NUM; | |
| | .aa.aa.....A..aB.a.A...aaaaa.aA..aaaa.A..aa.aA..a..A | Cas9_Wa; | |
| | .A.a...Aa...A...A....A.......A..........A.......A..J | Cas9_Cr; | |
| | ..............L.................................N... | Cpf1_Wa; | |
| | ........................................N..L........ | Cpf1_Cr; | |
| RAD51D | gcacagtccgaccctgagcacgcccAAtgttccccgcaggccggaacagccc | SEQ_DUP; | 312 |
| | .........................12......................... | DUP_NUM; | |
| | .A...a....Ja.A...A......A......A..aA..aaB...A.....aa | Cas9_Wa; | |
| | Aa....A.a...aa..AaaH...A.a.aaa.....Baaaa.a...Aa....A | Cas9_Cr; | |
| | ..............................L..................... | Cpf1_Wa;. | |
| | ....N................LLN............L............... | Cpf1_Cr; | |
| BRCA1 | cactctcgggtcaccacaggtgcctCCacacatctgcccaattgctggagac | SEQ_DUP; | 313 |
| | .........................12......................... | DUP_NUM; | |
| | ..HaaA........aa.A..J..........A.......A..aa.a...aJa | Cas9_Wa; | |
| | .A.aaaHa.aHa....a.aa.a.....Aa.aa.a.a.Ca..Aaa.....A.. | Cas9_Cr; | |
| | ...............L................................L... | Cpf1_Wa; | |
| | ..................N......L.........N....N........... | Cpf1_Cr; | |
| BRCA1 | agggaagctcttcatcctcactagaTTaagttctcttctgaggactctaatt | SEQ_DUP; | 314 |
| | .........................12......................... | DUP_NUM; | |
| | B.A.............I..aC....A.........a.aa............. | Cas9_Wa; | |
| | J...A.......A.a.Ba.CaaHa.a..........Ba.a.Ba......A.a | Cas9_Cr; | |
| | ...............N.................NL.............N... | Cpf1_Wa; | |
| | ......N.....................N....................N.. | Cpf1_Cr; | |
| BRCA1 | tagataagttctcttctgaggactcTTaatttcttggcccctcttcggtaac | SEQ_DUP; | 315 |
| | .........................12......................... | DUP_NUM; | |
| | ...A.........a.aa..............aA.........aA.......a | Cas9_Wa; | |
| | aHa.a.........Ba.a.Ba......A.aH.....Ba....Aaaa.a.Ba. | Cas9_Cr; | |
| | ............NL.................N....N...........N... | Cpf1_Wa; | |
| | ......N.....................N..........k............ | Cpf1_Cr; | |
| BRCA1 | cttttgttttattctcatgaccactAAttagtaatattcatcacttgaccat | SEQ_DUP; | 316 |
| | .........................12......................... | DUP_NUM; | |
| | .A............a...........A...............a........A | Cas9_Wa; | |
| | .Ca.aa...........BaHa....Aa.a.............BaHCa.a... | Cas9_Cr; | |
| | .k.....N..................k....k..N...............N. | Cpf1_Wa; | |
| | ....N......N........................................ | Cpf1_Cr; | |
| RAD51C | gaatgtctcacaaataaaccaagatAAtgctggtacatctgagtcacacaag | SEQ_DUP; | 317 |
| | .........................12......................... | DUP_NUM; | |
| | A.................aC....AJ.aA......Ha.a........aBJa. | Cas9_Wa; | |
| | ...........a.a.a.......Aa.........A.....A.Ca.....a.a | Cas9_Cr; | |
| | ............N.M..................................... | Cpf1_Wa; | |
| | ..M.N.......................N..N.............LLN.... | Cpf1_Cr; | |
| BRIP1 | tgaggtactgtactttaaagaggtcAActtcaagtgtagactcattgtcctg | SEQ_DUP; | 318 |
| | .........................12......................... | DUP_NUM; | |
| | A.J..A.........a.aA..........a.A..a.......A....A.... | Cas9_Wa; | |
| | ............A..J.A..J........a..A.Ba........JA.aH... | Cas9_Cr; | |
| | .N......NLL..........................k.............N | Cpf1_Wa; | |
| | ....N.....N.....................M................... | Cpf1_Cr; | |
| BRIP1 | actcactttaccacgacaaactgctAAccaggagagctccatcttaaacaac | SEQ_DUP; | 319 |
| | .........................12......................... | DUP_NUM; | |
| | ..........a.......A.......aa.a.A.................aB. | Cas9_Wa; | |
| | AaaHa.a.aHa....Aa.a..A...A..A...Aa.......A.aa.Ca.... | Cas9_Cr; | |
| | .............L................k..................... | Cpf1_Wa; | |
| | ....N...................k...N....k.......M.....k.... | Cpf1_Cr; | |
| BRIP1 | ttagcctccagctggatagtaaatgTTaacaccaagttctgacgaaaaggat | SEQ_DUP; | 320 |
| | .........................12......................... | DUP_NUM; | |
| | ......A.HaaC..A.....AJ.........A....a..aB.IHaaC..... | Cas9_Wa; | |
| | .........Aa.aa..A.................A.aa....Ba...A.... | Cas9_Cr; | |
| | ...........k.......M...N......L.................N... | Cpf1_Wa; | |
| | ......N.....N.........Lk.................M.N........ | Cpf1_Cr; | |
| CANT1 | tggcgggcgcctggccgagcctccaGGttgtgtgcattgtgggtggggggcc | SEQ_DUP; | 321 |
| | .........................12......................... | DUP_NUM; | |
| | aaa.A...aA..a.A......aA..a.a.A....aHaaa.aaaaaA...A.. | Cas9_Wa; | |
| | Aa.J....A...A.aa...Aa...Aa.aa..........AJ.J......... | Cas9_Cr; | |
| | .............N........N......................L....N. | Cpf1_Wa; | |
| | ..................................................L. | Cpf1_Cr; | |
| PYCR1 | cccacctgggggcctacctgctgctGGtgaagcccttggccagggcaaaagc | SEQ_DUP; | 322 |
| | .........................12......................... | DUP_NUM; | |
| | ...aaaaA.......A..A..aa.aB.A.....aA...aaA.....A...A. | Cas9_Wa; | |
| | a..aaaaa.aa......Aa..Aa..A..A........Aaa....Aa....A. | Cas9_Cr; | |
| | ...L.................L.............................. | Cpf1_Wa; | |
| | .......LN................k.......................LN. | Cpf1_Cr; | |
| NPC1 | aaaaggatgacaggaacatactgggAAgccacttctcctaggaccctgccca | SEQ_DUP; | 323 |
| | .........................12......................... | DUP_NUM; | |
| | aaC.a...aab.......aaaB.A............aa.....A....A..A | Cas9_Wa; | |
| | a.a............A.....A...A.......Aa.a.Ba.aa.....AaaH | Cas9_Cr; | |
| | .................................................... | Cpf1_Wa; | |
| | ..LLN............................................... | Cpf1_Cr; | |
| LAMA3 | tcaaagtcaactgcaagcgagttatGGtggagtttagacccagccaggtaac | SEQ_DUP; | 324 |
| | .........................12......................... | DUP_NUM; | |
| | .A......A...aHa.A....aa.aa.A....a.....A...aA....A... | Cas9_Wa; | |
| | aaa...a.....a..A..A...A....................AaaH.Aa.. | Cas9_Cr; | |
| | .......k..NL......L.........................N.M..... | Cpf1_Wa; | |
| | .......L....................N..........N............ | Cpf1_Cr; | |
| SERPINB7 | ctgctgtaatggtgctggtgaatgcTTgtgtacttcaaaggcaagtggcaat | SEQ_DUP; | 325 |
| | .........................12......................... | DUP_NUM; | |
| | .A....aa.A..aaHaB..A..Ja.A.........aA...a.aA......A. | Cas9_Wa; | |
| | A.a.Ca..A..........A..J......A.......AJBa.....A..... | Cas9_Cr; | |
| | ................................................N... | Cpf1_Wa; | |
| | ...............k.....N......N.............N....Lk... | Cpf1_Cr; | |
| LDLR | acggctcagacgagcaaggctgtcgTTaagtgtggccctgcctttgctattg | SEQ_DUP; | 326 |
| | .........................12......................... | DUP_NUM; | |
| | ....a..a.A...aA..A..A....a.a.aA....A.....A.....a.A.. | Cas9_Wa; | |
| | A..A..A..A.a...A...A....A...a...........aaa..Aa....A | Cas9_Cr; | |
| | ................................................N... | Cpf1_Wa; | |
| | ......N..........................................L.. | Cpf1_Cr; | |
| LDLR | agcccccaagacgtgctcccaggacGGagtttcgctgccacgatgggaagtg | SEQ_DUP; | 327 |
| | .........................12......................... | DUP_NUM; | |
| | .....a..a.A......aa..aa.A....A..A....aC.aaaB.a.A.... | Cas9_Wa; | |
| | aa..A..Aaaaa....A...A.aaa....A......Ba.a..Aa.a...... | Cas9_Cr; | |
| | .............L...L......................L........... | Cpf1_Wa; | |
| | ....L...................LLN......................... | Cpf1_Cr; | |
| LDLR | gagtgcctgtgccccgacggcttccAAgctggtggcccagcgaagatgcgaa | SEQ_DUP; | 328 |
| | .........................12......................... | DUP_NUM; | |
| | A...a.A....a..aA.......A..aa.aA....a.aB.aC.a.aB.aa.a | Cas9_Wa; | |
| | .A..A.....Aa.J..Aaaa..A..A.Baa...A......Aaa..A...... | Cas9_Cr; | |
| | .........N..................................NLL..... | Cpf1_Wa; | |
| | ....N...............LN......LN................L..... | Cpf1_Cr; | |
| LDLR | atctgtgcctccctgccccgcagatCCaacccccactcgcccaagtttacct | SEQ_DUP; | 329 |
| | .........................12......................... | DUP_NUM; | |
| | a.A.......A....A..aC..............A.....A.......a.A. | Cas9_Wa; | |
| | Aa....Ca.J..Aa.aaa..Aaaa.a...CaaH.Aaaaa.a.aHaaa..... | Cas9_Cr; | |
| | .......................M.........L..............L... | Cpf1_Wa; | |
| | ......N..............N...................L.......... | Cpf1_Cr; | |
| LDLR | aggctaaaggtcagctccacagccgTTaaggacacagcacacaaccacccga | SEQ_DUP; | 330 |
| | .........................12......................... | DUP_NUM; | |
| | ....aA...A.......A..A...Jaa..J.JA.............a....A | Cas9_Wa; | |
| | aa..a...A.......a..A.aa.a..Aa........A.a..A.a.a..Aa. | Cas9_Cr; | |
| | ...............L.......................L........N... | Cpf1_Wa; | |
| | ......N..............N.............................. | Cpf1_Cr; | |
| ACP5 | agcgcagatagccgttggggaccttGGcgctggtgccgctttgaggggtcca | SEQ_DUP; | 331 |
| | .........................12......................... | DUP_NUM; | |
| | ..aC..A..A..aaaa.....aa.A..aa.A..A.......a.aaaA..aB. | Cas9_Wa; | |
| | .......A.a......Aa........Aa....A.a........Aa.a..... | Cas9_Cr; | |
| | ......NL....L........................N........N..... | Cpf1_Wa; | |
| | ................................Lk.................. | Cpf1_Cr; | |
| CYP4F22 | ctggggctggagaagacggcgttccGGcatatacgcggtgtccacccttctc | SEQ_DUP; | 332 |
| | .........................12......................... | DUP_NUM; | |
| | aA..aa.aB.a..aa.A....aAJ......a.aa.A................ | Cas9_Wa; | |
| | .aa.aa.....A.........A..A..Baa..A.....A.a.....aaJaaa | Cas9_Cr; | |
| | ............................................NLL..... | Cpf1_Wa; | |
| | .......M.M.......................................... | Cpf1_Cr; | |
| JAK3 | ggaggaaggggctcacctttgggtcTTggggatacagcaggaagtgagggtc | SEQ_DUP; | 333 |
| | .........................12......................... | DUP_NUM; | |
| | aB.aaaA........HaaA....aaaaC....A..aaB.a.aHaaA.....A | Cas9_Wa; | |
| | aa..............A.a.aa.......a.........A..A......... | Cas9_Cr; | |
| | ................NLL......................k......N... | Cpf1_Wa; | |
| | .......L.M.........LN............................... | Cpf1_Cr; | |
| ADCK4 | ctgggggagactcacctcgatgtaaTTggtctgtgaactctgtcccaaactc | SEQ_DUP; | 334 |
| | .........................12......................... | DUP_NUM; | |
| | aaa.a.........aC.A.....aA...a.aB.....A............aA | Cas9_Wa; | |
| | .....a.........A.aHaa.a............a......A.aH..aaa. | Cas9_Cr; | |
| | ......kL........................................N... | Cpf1_Wa; | |
| | ..N..........LN..........k.................k........ | Cpf1_Cr; | |
| ZNF575 | agtttggtccccggactgaccagcaTTagagcagccgcagccccagctcctt | SEQ_DUP; | 335 |
| | .........................12......................... | DUP_NUM; | |
| | .aA.....aa...a....A.....a.A..A..A..A.....A......aC.. | Cas9_Wa; | |
| | aaH..........aaaa...A...Aa..A.......A..Aa.a..Aaaa..A | Cas9_Cr; | |
| | ..................L.......k....L................N... | Cpf1_Wa; | |
| | .................................................... | Cpf1_Cr; | |
| PNKP | cctcggcttccagctctcggagcttAAcggggaatctctgggtacaagatcc | SEQ_DUP; | 336 |
| | .........................12......................... | DUP_NUM; | |
| | aA......A.....aa.A......aaaaB.....haaA.....aC......a | Cas9_Wa; | |
| | ....Aaa.a..A.Baa..A.a.a....A....A......Ca.aI.....A.. | Cas9_Cr; | |
| | ................N.............NLL.............N..... | Cpf1_Wa; | |
| | ....N....LLN............N..................LN....... | Cpf1_Cr; | |
| NLRP12 | cccaggggataccccagggatacttAAcagctgcaccaacagcgtgtgcgct | SEQ_DUP; | 337 |
| | .........................12......................... | DUP_NUM; | |
| | aaaaC.......aaaC.........a..A........a.a.a.a.A.....a | Cas9_Wa; | |
| | .A..Aaaa........Aaaa.......A....A..A..A.aa..A..A.... | Cas9_Cr; | |
| | ..............................................N..... | Cpf1_Wa; | |
| | ....N...........N...................L............... | Cpf1_Cr; | |
| DNAAF3 | tcatgcgcaggtcccagtcgctgacAAccgcgccgggcgtcgtagcgggagc | SEQ_DUP; | 338 |
| | .........................12......................... | DUP_NUM; | |
| | a.A..aA.....A..A..a......a.A..aaa.A..A..a.aaa.A....a | Cas9_Wa; | |
| | A..A.Ba...A.a....aaa...a.a...A..Aa.a.aa...A..a....A. | Cas9_Cr; | |
| | ......................NL...........L................ | Cpf1_Wa; | |
| | ....N.....................L......................... | Cpf1_Cr; | |
| DNAAF3 | caggtgccgtccatccacagagcccAAgaagcagcacatctagctcggggtt | SEQ_DUP; | 339 |
| | .........................12......................... | DUP_NUM; | |
| | .A..A..........a.A.....aB.a..A........A...aaaA..A.Ja | Cas9_Wa; | |
| | a.a..A.....Aa.Jaa.CaaHa....Aaa......A..A.a.Ca...A.a. | Cas9_Cr; | |
| | NL...............L...............L...L.............. | Cpf1_Wa; | |
| | ....N..N............................................ | Cpf1_Cr; | |
| KCNE1 | gaaagggcgtcaccgctgtggtgttAAgacaggatcatcctgggcattaagg | SEQ_DUP; | 340 |
| | .........................12......................... | DUP_NUM; | |
| | aaa.A.....A..a.aa.A....a..HaaC.......aaA......aA.... | Cas9_Wa; | |
| | ...a........A..a.aa.a..........J..A....Ca.CaaH...A.. | Cas9_Cr; | |
| | ................N.............................N..... | Cpf1_Wa; | |
| | ...N.......................N....................k... | Cpf1_Cr; | |
| HLCS | tcgggaatggactccctggtagcaaTTgaccaacagcagacagttgtccgtc | SEQ_DUP; | 341 |
| | .........................12......................... | DUP_NUM; | |
| | aB..aa.......aA..A.....a.......A..a...A..A...A....Ha | Cas9_Wa; | |
| | .J.a.Ba.........A.aaa......A......Aa..A..A...A...... | Cas9_Cr; | |
| | ........N.............NL............L...........N... | Cpf1_Wa; | |
| | ..N.......N......................................... | Cpf1_Cr; | |
| TMPRSS3 | aggtaaccacgtggccagaggcacaTTccctccctaaagcggagaaaaagta | SEQ_DUP; | 342 |
| | .........................12......................... | DUP_NUM; | |
| | ......a.aA...a.aA.................a.aa.aB....A..aA.. | Cas9_Wa; | |
| | aJ.A.......Aa.a....Aa.....A.a..Baaa.aaa.....A....... | Cas9_Cr; | |
| | .......N........................................NLL. | Cpf1_Wa; | |
| | ..............k....L...k...............Lk........... | Cpf1_Cr; | |
| PCNT | ctggcagccgcctccacctaggttcTTgcccgcagggctgccggctcggatg | SEQ_DUP; | 343 |
| | .........................12......................... | DUP_NUM; | |
| | ..A..A..........aA.....A...A..aaA..A..aA..HaaC.a.aa. | Cas9_Wa; | |
| | ....Aa.J.A..Aa.aa.aa.aa.....Ba...Aaa.a....A..Aa..A.a | Cas9_Cr; | |
| | ....................................L........N..N... | Cpf1_Wa; | |
| | ..........................L.....L...........LLN..... | Cpf1_Cr; | |
| PEX26 | agctcattggaggtgaagtgctcccTTgtgtgttgtggggatccaggccctg | SEQ_DUP; | 344 |
| | .........................12......................... | DUP_NUM; | |
| | ...aa.aa.aB.a.A........a.a.A..a.aaaaC....aA....aA..a | Cas9_Wa; | |
| | A...A..A.a...............A.aaa........J.J.....CaaH.. | Cas9_Cr; | |
| | ......N.....k................N...............L..N... | Cpf1_Wa; | |
| | ................L..................k...L..........N. | Cpf1_Cr; | |
| SNAP29 | agaacaggaagcaaagtaccaggccAAgccacccaaaccttagaaagctgga | SEQ_DUP; | 345 |
| | .........................12......................... | DUP_NUM; | |
| | ..aaB.A.J..A.....aA....A..............aB..A.Haac.aC. | Cas9_Wa; | |
| | .J.......A......A......Aa.J.Aa...Aa.aaaH..Aa........ | Cas9_Cr; | |
| | .........M.......................................... | Cpf1_Wa; | |
| | ....N........k........k.....L....M.......N...L.M.... | Cpf1_Cr; | |
| CHEK2 | accctttcatattcatacctttctcTTgagacttctgcccagacttcaggaa | SEQ_DUP; | 346 |
| | .........................12......................... | DUP_NUM; | |
| | .......J...............a.a......A....a......aaB..aB. | Cas9_Wa; | |
| | BaH...AaaH.Ba....BaH..Aa..Ba.a......A.Ba..Aaa...A.Ba | Cas9_Cr; | |
| | ...k.............N..N.......kL....NL.......k....N... | Cpf1_Wa; | |
| | ............................LN..LN.................. | Cpf1_Cr; | |
| CHEK2 | gaacaggcactgctgccatgagactGGctgagcctcaacatccgactcccga | SEQ_DUP; | 347 |
| | .........................12......................... | DUP_NUM; | |
| | .aA....A..A....a.a...aA..a.A...........a......a.a... | Cas9_Wa; | |
| | .A......A...A.a..A..Aa......A...A....Aa.a..A.CaaH.A. | Cas9_Cr; | |
| | .................................................... | Cpf1_Wa; | |
| | ...............N.............................k.....k | Cpf1_Cr; | |
| CYBB | tgagtaaacaaagcatctccaactcTTgagtctggccctcggggagtgcatt | SEQ_DUP; | 348 |
| | .........................12......................... | DUP_NUM; | |
| | ........A.............Ha.A...aA.....aaaa.a.A........ | Cas9_Wa; | |
| | ..AaaH.......A....A.Ca.aa..A.aH.....a...Aaa.a....... | Cas9_Cr; | |
| | ..........N..............................L......N... | Cpf1_Wa; | |
| | .....................L.................N..N..Lk..... | Cpf1_Cr; | |
| CHM | tcatgagttatcaattatttttcttAAcctatctccatttcagtatatggaa | SEQ_DUP; | 349 |
| | .........................12......................... | DUP_NUM; | |
| | a.A...................................A.....aaB..... | Cas9_Wa; | |
| | .Baa.Ca........Ca.........Ba....Aa..Ca.aa...Ba...... | Cas9_Cr; | |
| | M......N....N.....kLL.........N.M....N.....k..N..... | Cpf1_Wa; | |
| | ....N..................M...LLN..k...N..........N.... | Cpf1_Cr; | |
| CHM | gaacagcaaaagttcctggttcctcTTgctggcactgtcaaaatggaaatct | SEQ_DUP; | 350 |
| | .........................12......................... | DUP_NUM; | |
| | .A.....A.....aA........AJ.aA....A.......aaB......... | Cas9_Wa; | |
| | ..AaaH..A..A......Baa....Baa.a...A...A.a...a........ | Cas9_Cr; | |
| | ...................................NL.....NL....N... | Cpf1_Wa; | |
| | ..................k....LLk.....k......k.....k....... | Cpf1_Cr; | |
| ABCA4 | tctcccacaaaggcaatggcaaccgACACagctttctctgcatgccacctggag | SEQ_DUP; | 351 |
| | .........................1122......................... | DUP_NUM; | |
| | .......aA....aA...J.a.....A........A...A......aa.aA... | Cas9_Wa; | |
| | ...A.Ca.aaa.a.....A.....A..Aa..A.a..A..Ba.a..A...Aa.aa | Cas9_Cr; | |
| | ...L.............N........L........................... | Cpf1_Wa; | |
| | .............................L.......N..N......L...... | Cpf1_Cr; | |
| FLAD1 | cactcatgatgatgtgacctttgagGCGCagtggcacaggcctttggagatgag | SEQ_DUP; | 352 |
| | .........................1122......................... | DUP_NUM; | |
| | ...aC.aC.a.a......a.aa.A..a.aA....aA.....aa.aC.a.A..aB | Cas9_Wa; | |
| | ..Aaaa.a.aH...........Aa.......A.a.....A.a...Aa....... | Cas9_Cr; | |
| | ...........................................k.......... | Cpf1_Wa; | |
| | ........................L..........LN...........N....L | Cpf1_Cr; | |
| MSH6 | atttggccgttattcagattccctgGTGTgcagaagggctataaagtagcacga | SEQ_DUP; | 353 |
| | .........................1122......................... | DUP_NUM; | |
| | aA..A....I..aC......aa.a.A..aB.aaA.......a..A..Ha.a.aa | Cas9_Wa; | |
| | ....A......Aa.....BaH...Baaa.......AJ.J....A.......... | Cas9_Cr; | |
| | ..N.......kL.......N.....k......N..NL....NLL.......... | Cpf1_Wa; | |
| | ............N......M.k.............LLN................ | Cpf1_Cr; | |
| SLC19A3 | ataaggaatggttctgagggtctcaTCTCatggagaaaaaaccaaataagcaga | SEQ_DUP; | 354 |
| | .........................1122......................... | DUP_NUM; | |
| | aaB..aA....aHaaA...........aa.aB.............A..aHaaC. | Cas9_Wa; | |
| | .................Ba.......a.a.Ca.a............Aa...... | Cas9_Cr; | |
| | .M......L.........M...............N................... | Cpf1_Wa; | |
| | ..........L...k.......k...N.....................k..... | Cpf1_Cr; | |
| AGXT | tccaacctgcctcctcgcatcatggCACAgccggggggctgcagatgatcgggt | SEQ_DUP; | 355 |
| | .........................1122......................... | DUP_NUM; | |
| | ....A.......A....J.aA....A..aaaaaA..A..aC.aC.HaaA..... | Cas9_Wa; | |
| | ..aa.Baa..Aa..Aa.aa.a.a.Ca...A.a..Aa.......A..A......C | Cas9_Cr; | |
| | ......................NLL............................. | Cpf1_Wa; | |
| | .........................................N...M........ | Cpf1_Cr; | |
| DNAH5 | cattttgttccatcagctgtcgcacTATAtctcattcgtaacctacaaaagaca | SEQ_DUP; | 356 |
| | .........................1122......................... | DUP_NUM; | |
| | ..A........A..a..A...............A............a....... | Cas9_Wa; | |
| | ...CaaHI.....Baa.Ca..A...a.a.a....Ca.a..BaH...Aa..A... | Cas9_Cr; | |
| | ....N........N.M......L....k..NLL................M.M.. | Cpf1_Wa; | |
| | .....M............N......k......N........N...k........ | Cpf1_Cr; | |
| DNAH8 | acaatattagaacaaattttttgatAGAGacaccattgcaaaacaacataaagt | SEQ_DUP; | 357 |
| | .........................1122......................... | DUP_NUM; | |
| | .....aB...........aC..a.a........A..............A...A. | Cas9_Wa; | |
| | ......A..........A.................A.aa....A....A..A.. | Cas9_Cr; | |
| | .......k...NLL......N........N.............k.......... | Cpf1_Wa; | |
| | ..M...............k....N..M.k....N.....N....M......... | Cpf1_Cr; | |
| WISP3 | atttgtcttttctggatgctcaagtACACtcagagttacaaacccactttttgt | SEQ_DUP; | 358 |
| | .........................1122......................... | DUP_NUM; | |
| | A.......HaaC.A..J.JA.......Ha.A.................a.aaB. | Cas9_Wa; | |
| | ...........a...Ba......A.a.....A.aJaH......A...AaaHa.. | Cas9_Cr; | |
| | kLL.......L..............k......k..................... | Cpf1_Wa; | |
| | N.................k..............LLN........L.M.N..... | Cpf1_Cr; | |
| AHI1 | agctggatggaatttagccgtgtaaACACaaaagaaggatgaggtaaaactctg | SEQ_DUP; | 359 |
| | .........................1122......................... | DUP_NUM; | |
| | aaC.aaB.....A..a.AJ..........aBHaaC.a.aA.........aB..A | Cas9_Wa; | |
| | a...A..A..............Aa......JA.a.................... | Cas9_Cr; | |
| | ...................N................k................. | Cpf1_Wa; | |
| | ..k.....k....N..........k.......Lk.................... | Cpf1_Cr; | |
| CFTR | tattatgtgttttacatttacgtggGAGAgtagccgacactttgcttgctatgg | SEQ_DUP; | 360 |
| | .........................1122......................... | DUP_NUM; | |
| | ..a.A............a.aaaHa.A..AJ.a.......A...A...IaaaC.. | Cas9_Wa; | |
| | ..Ba......J......J.A.....A............Aa..A.a....A...A | Cas9_Cr; | |
| | ...N.M.............NL....N.M......k.....k............. | Cpf1_Wa; | |
| | ...L............................L..................... | Cpf1_Cr; | |
| TMEM70 | gtccccgggcctctgtctcccgggcGTGTcctccagcagcgggccttcggggcc | SEQ_DUP; | 361 |
| | .........................1122......................... | DUP_NUM; | |
| | ..aaA.....A......aaa.a.A.......A..a.aaA.....aaaA..aA.. | Cas9_Wa; | |
| | aa.....aaaa...Aa.a...a.aaa...A....aaJaa..A..A...Aa.Ba. | Cas9_Cr; | |
| | ..................L......L...............L............ | Cpf1_Wa; | |
| | ............................................L......... | Cpf1_Cr; | |
| BRCA2 | actctgaagaacttttctcagacaaTGTGagaataattttgtcttccaagtagc | SEQ_DUP; | 362 |
| | .........................1122......................... | DUP_NUM; | |
| | .aB.aB..........a.....a.aHaB........A........A..A..... | Cas9_Wa; | |
| | CaaHI.A.aH......A...Ba.a...A...................a.Baa.. | Cas9_Cr; | |
| | kL.................L.................k................ | Cpf1_Wa; | |
| | ..N.......N..N............N.......N..Lk...LN..N....... | Cpf1_Cr; | |
| POMT2 | gttgttgtagtccttgtgcaaatagGTGTggtgacctgggtggggggtgggggc | SEQ_DUP; | 363 |
| | .........................1122......................... | DUP_NUM; | |
| | ..A..A....a.A.......aa.a.aa.a...Haaa.aaaaaa.aaaaa.aa.a | Cas9_Wa; | |
| | aHa.............aa.....A..J.........J..Aa............. | .Cas9_Cr; | |
| | N.M...k..N.......L......N..N........N................. | Cpf1_Wa; | |
| | .................................L...LLN....LN........ | Cpf1_Cr; | |
| PALB2 | atagagtctgtaaaggaactgtagtCGCGccctggtgaaattaggtcttcttag | SEQ_DUP; | 364 |
| | .........................1122......................... | DUP_NUM; | |
| | .A...A....aaB...A..A..a.A....aa.aB.....aA........aaB.. | Cas9_Wa; | |
| | .Ca.........a..........A......a.a.aaa..............a.B | Cas9_Cr; | |
| | ..................M................................... | Cpf1_Wa; | |
| | ...........Lk....................LN.......N........... | Cpf1_Cr; | |
| RAD51D | gtatagagaccagcatcaagcagttTATAtcaagactgatggcagaagagaaga | SEQ_DUP; | 365 |
| | .........................1122......................... | DUP_NUM; | |
| | .a.a....A......A..A..........a...aC.aA..aB.a.aB.aB...J | Cas9_Wa; | |
| | a.aa.......J..Aa..A.Ca...A........Ca....A......A...... | Cas9_Cr; | |
| | .......kL.......N...L....M.....................k.M.M.. | Cpf1_Wa; | |
| | .....M....N.............N....N..k.....N....N.......... | Cpf1_Cr; | |
| BRCA1 | gcacacacacacacgctttttacctGAGAgtggttaaaatgtcactctgagagg | SEQ_DUP; | 366 |
| | .........................1122......................... | DUP_NUM; | |
| | .J.J......A..........aHa.a.aA.......A.......a.aHaaC..A | Cas9_Wa; | |
| | a.....A.aJa.a.a.a.a.a......Aa..................a.a.aH. | Cas9_Cr; | |
| | ..........................................k........... | Cpf1_Wa; | |
| | ..............k..................M.................... | Cpf1_Cr; | |
| BRCA1 | ccttgattttcttccttttgttcacATATtcaaaagtgacttttggactttgtt | SEQ_DUP; | 367 |
| | .........................1122......................... | DUP_NUM; | |
| | aC.............A...............a.a......aa.....A...... | Cas9_Wa; | |
| | BaH.Baa.......Ba.Baa......Ba.a....BaH.......A.......A. | Cas9_Cr; | |
| | ..N.....N........N...kL..N.....k..NL....k..NL......NL. | Cpf1_Wa; | |
| | ....M.....k............L..............N............... | Cpf1_Cr; | |
| BRCA1 | agcgcatgaatatgcctggtagaagACACttcctcctcagcctattctttttag | SEQ_DUP; | 368 |
| | .........................1122......................... | DUP_NUM; | |
| | ..HaB....A...aA..ab.a..............A.............aa.A. | Cas9_Wa; | |
| | ..Ba...A.a.........Aa..........A.a.Baa.aa.a..Aa...BaH. | Cas9_Cr; | |
| | ...k...............NL.............M.................NL | Cpf1_Wa; | |
| | .N.......................................LN.....L.M... | Cpf1_Cr; | |
| KPTN | ggcctctttcggggcctctcctaccTATActggtcaggttcgtcagctctggga | SEQ_DUP; | 369 |
| | .........................1122......................... | DUP_NUM; | |
| | ......aaaA.........J.......aA...aA...A...A....aaaB.a.a | Cas9_Wa; | |
| | aa.....Aa.a..Ba....Aa.a.aa..Aa....A....a....Ba..a..A.a | Cas9_Cr; | |
| | ................L.............kL.................M.... | Cpf1_Wa; | |
| | .....M........................LLN..................... | Cpf1_Cr; | |
| SPTA1 | actgctggaacttcagggcccgcagCAACAActggtcacccttctccagggtcagc | SEQ_DUP; | 370 |
| | .........................111222......................... | DUP_NUM; | |
| | ..aaB......aaA...A..A........aA............HaaA...A..... | Cas9_Wa; | |
| | ..A..JA..A.....A.Ba....Aaa.a..A..A..A....a.aaaHBa.aa.... | Cas9_Cr; | |
| | ..................................NL.................... | Cpf1_Wa; | |
| | .....N..N.............................N................. | Cpf1_Cr; | |
| TREX1 | ctccagactcgcacacggctgagggTGATGAtgtcctggccctgctcagcatctgt | SEQ_DUP; | 371 |
| | .........................111222......................... | DUP_NUM; | |
| | .a.J.JA.....aA..aHaaa.aC.aC.A....aA....A....A.....A...a. | Cas9_Wa; | |
| | ..aaaa.aa...A.aHa.a.a..A...............aa...Aaa..A.a..A. | Cas9_Cr; | |
| | ...........M........L....L.............................. | Cpf1_Wa; | |
| | .......................................L................ | Cpf1_Cr; | |
| SLC26A3 | acgatatacacatctaccttgatccTGATGAtaaattcttgcaaaatctgttaaaa | SEQ_DUP; | 372 |
| | .........................111222......................... | DUP_NUM; | |
| | J...............aC....aC.aC.........A........A.......aB. | Cas9_Wa; | |
| | .Baa..A......A.a.Ca..Aa....CaaH..........BaH..A....Ca..I | Cas9_Cr; | |
| | ........N.........NLL.......M............N.............. | Cpf1_Wa; | |
| | .........M.k.........k.........k.....k.....M............ | Cpf1_Cr; | |
| CDKN2A | agctcctcagccaggtccacgggcaGACGACggccccaggcatcgcgcacgtccag | SEQ_DUP; | 373 |
| | .........................111222......................... | DUP_NUM; | |
| | .....A...aA.....aaA..a..a..aA.....aA...Ja.A...A....A..a. | Cas9_Wa; | |
| | ..Aaa..A.aa.a..Aa....aa.a...A...A..A..Aaaa...A.Ca.a.a.a. | Cas9_Cr; | |
| | .......................................L................ | Cpf1_Wa; | |
| | ........................................................ | Cpf1_Cr; | |
| USH2A | ctgtgccaaagggtggacccgcgggTGGCTGGCtgccagggcaacggcaatgtgattg | SEQ_DUP; | 374 |
| | .........................11112222......................... | DUP_NUM; | |
| | A....Haaa.aa....aHaaa.aA..aA..A...aaA....aA...Ia.aC..aaA.. | Cas9_Wa; | |
| | ..Aa.a....Aa.J........AaaHa......A...A..Aa....A..A..A..... | Cas9_Cr; | |
| | ......N................................................... | Cpf1_Wa; | |
| | .....................N.....N................N.......N..... | Cpf1_Cr; | |
| TPO | gcgggccctgcttcctggccggagaCGGCCGGCcgcgccagcgaggtcccctccctga | SEQ_DUP; | 375 |
| | .........................11112222......................... | DUP_NUM; | |
| | A....A......aA..aa.a..aA..aA..a.A...a.a.aA..........aJ.aA. | Cas9_Wa; | |
| | ..AaaHa...Aaa..A.Baa...Aa.....A..Aa..Aa.a.aa..A.....aaaa.a | Cas9_Cr; | |
| | .........L........................NL...................... | Cpf1_Wa; | |
| | .......................................................... | Cpf1_Cr; | |
| HADHA | caaatccttcctcttcacaccctccTGATTGATagatgtaaaagcccttcccagattt | SEQ_DUP; | 376 |
| | .........................11112222......................... | DUP_NUM; | |
| | .........J.........I..aC..aC..aC.A.....A......I..aC.....a. | Cas9_Wa; | |
| | ....BaH..CaaHbaa.a.Ba.a.aaaHaa...................Aaa.Baaa. | Cas9_Cr; | |
| | ........M.L..........NL.......NL....NL.............N...... | Cpf1_Wa; | |
| | ..........M.......k........................k.........N..k. | Cpf1_Cr; | |
| HADHB | tgggccactctgcagaccgactggcCGCTCGCTgcctttgctgtttctcggctggaac | SEQ_DUP; | 377 |
| | .........................11112222......................... | DUP_NUM; | |
| | .......A..a...a...aA..A...A..A.....A..A......aA..aaB..HaaC | Cas9_Wa; | |
| | ..Aa.....Aa.a.aH.A...Aa..A...Aa.a.a.a..Aa....A....Ba.a..A. | Cas9_Cr; | |
| | ....N...L................................................. | Cpf1_Wa; | |
| | ................................LLN.......LN.............. | Cpf1_Cr; | |
| MSH6 | tttttttggagatgattttattcctAATGAATGacattctaataggctgtgaggaaga | SEQ_DUP; | 378 |
| | .........................11112222......................... | DUP_NUM; | |
| | ...aa.aC.aC............HaB..a...........aA..a.a.aaB.a.aa.A | Cas9_Wa; | |
| | ....A.....................Baa..........A..BaH......A...... | Cas9_Cr; | |
| | ........N....N..............k...........k..NL............. | Cpf1_Wa; | |
| | ....N..LN..........N............LN..........Lk.......k.... | Cpf1_Cr; | |
| MSH6 | tttaaggtgaaagtacattttttgtTGAATGAAttaagtgaaactgccagcatactca | SEQ_DUP; | 379 |
| | .........................11112222......................... | DUP_NUM; | |
| | .aa.ab..A..........A.HaB.HaB.....a.aB....A..JA.........A.. | Cas9_Wa; | |
| | ....................A..J........................A..Aa..A.. | Cas9_Cr; | |
| | ..NL...k................k...................k..N........N. | Cpf1_Wa; | |
| | .....LN..LN...N...Lk..........M.............N............. | Cpf1_Cr; | |
| MSH6 | ttactttaacaggaagaggtactgcAACAAACAtttgatgggacggcaatagcaaatg | SEQ_DUP; | 380 |
| | .........................11112222......................... | DUP_NUM; | |
| | .......aaB.a.aA....A............aC.aaa..aA.....A.....A..A. | Cas9_Wa; | |
| | ........A.....A...........A..a..A...A...........A..A.....A | Cas9_Cr; | |
| | ....N...NL..N.M........k....k............................k | Cpf1_Wa; | |
| | ....N..k...........L......N.....k...........k...N......... | Cpf1_Cr; | |
| MSH6 | cattattttcaactcactaccattcATTAATTAgtagaagattattctcaaaatgttg | SEQ_DUP; | 381 |
| | .........................11112222......................... | DUP_NUM; | |
| | .............................A..aB.aC.............A..A..a. | Cas9_Wa; | |
| | .a...A..J....Ba..A.aHa..Aa..BaH...................BaHa.... | Cas9_Cr; | |
| | ..........M..............N....kL.............NL..N...N.... | Cpf1_Wa; | |
| | .......N........N...........k.......................M..... | Cpf1_Cr; | |
| MSH6 | aaaagcaagagaatttgagaagatgAATCAATCagtcactacgattatttcggtaact | SEQ_DUP; | 382 |
| | .........................11112222......................... | DUP_NUM; | |
| | A...aHaB....a.aB.aCHaB........A....I..aC.......aA......... | Cas9_Wa; | |
| | A.........A.....................Ca..ca..Ia.a..A.......Ba.. | Cas9_Cr; | |
| | ....N..NL............................k.................... | Cpf1_Wa; | |
| | ...LN...N........................N...N...N...M.N..LLN...M. | Cpf1_Cr; | |
| MSH6 | aagcaagagaatttgagaagatgaaTCAGTCAGtcactacgattatttcggtaactaa | SEQ_DUP; | 383 |
| | .........................11112222......................... | DUP_NUM; | |
| | ..aHaB....a.aB.aCHaB....A...A....I..aC.......aA........... | Cas9_Wa; | |
| | ........A.....................Ca..Ia...a.a..A.......Ba.... | Cas9_Cr; | |
| | ..N..NL............................k...................... | Cpf1_Wa; | |
| | .LN............................N...N...N...M.N..LLN...M.N. | Cpf1_Cr; | |
| ALMS1 | tgacctgtcatgtatggcaacagatAGTAAGTAtatcaaggcaatagtagaacacaaa | SEQ_DUP; | 384 |
| | .........................11112222......................... | DUP_NUM; | |
| | ..A....A...aA.....aC..A...A........aA.....A.JaB..........J | Cas9_Wa; | |
| | Baa.....Aa...a........a..A..............Ca....A..........A | Cas9_Cr; | |
| | ..k.....kL....N..NLL..N.............M..................M.M | Cpf1_Wa; | |
| | ..M....N...M....N....N.......N....k........M....N......... | Cpf1_Cr; | |
| DGUOK | cagtgctggtgttggatgtcaatgaTGATTGATttttctgaggaagtaaccaaacaag | SEQ_DUP; | 385 |
| | .........................11112222......................... | DUP_NUM; | |
| | A..aa.A.HaaC.A.....aC.ac..aC.......a.aaB.A...........aB.a. | Cas9_Wa; | |
| | A..Baa....A..J.....J....a................Ba...........Aa.. | Cas9_Cr; | |
| | ..k.................NLL...........N................N...... | Cpf1_Wa; | |
| | .....................LN...N...k...N..N...................L | Cpf1_Cr; | |
| ORC4 | cggcagtcataaatgggtgcgatgcTGTTTGTTactcgatttaaagcaagcatctagg | SEQ_DUP; | 386 |
| | .........................11112222......................... | DUP_NUM; | |
| | .A.......Haaa.a.aC.A..A...A...I..aC......A...A......aaaB.. | Cas9_Wa; | |
| | .Ca..A..A...a...........A..J.A.........A.aH........A...A.C | Cas9_Cr; | |
| | N....N...NLL..N....................................k..N... | Cpf1_Wa; | |
| | .....................k....N.........LLk............k.....N | Cpf1_Cr; | |
| NEB | tcccttgcccatgttttctttgtatAACAAACAcctgtgcgataagaaagcatccaga | SEQ_DUP; | 387 |
| | .........................11112222......................... | DUP_NUM; | |
| | ..A.....A........A.....J........a.a.aC...aB..A......aB.... | Cas9_Wa; | |
| | .....Baaa...Aaa......Ba.........a...A.aa....A..J.......A.C | Cas9_Cr; | |
| | ......................NLL..N..........k...k...M........... | Cpf1_Wa; | |
| | ..M.N..k............M.N..k..........k.....N.........N..... | Cpf1_Cr; | |
| NEB | actctctgtatctctggggtgtccaAAACAAACagtctcataatacgacatggacttc | SEQ_DUP; | 388 |
| | .........................11112222......................... | DUP_NUM; | |
| | ...A.......aaaa.A.............A....J......a....aa........A | Cas9_Wa; | |
| | aaa...A.aHa....Ca.a........aaJ...A...A...a.a......A..A.... | Cas9_Cr; | |
| | .....N....k...N...L.............M............L............ | Cpf1_Wa; | |
| | ...k....k.........M.N.........L........................... | Cpf1_Cr; | |
| TTN | aatgctaatggcattcaaaacaatgGTATGTATcccctgctttaattgttagcccatc | SEQ_DUP; | 389 |
| | .........................11112222......................... | DUP_NUM; | |
| | .....aA.............aA...A........A........A...A.......... | Cas9_Wa; | |
| | Aa.......A......A..BaH...A..........JCaaaa..A............A | Cas9_Cr; | |
| | ....k..........k....................NL............M...M.L. | Cpf1_Wa; | |
| | N.....................N........................L....N..... | Cpf1_Cr; | |
| BARD1 | gaagctttactcacaacatatctgaCTTTCTTTcttacttcgagggctaaaccacatt | SEQ_DUP; | 390 |
| | .........................11112222......................... | DUP_NUM; | |
| | ...................a.................a.aaA................ | Cas9_Wa; | |
| | .A.a.....A....A.aHa..A...Ca...A..Ba..Ba...A.Ba.....A....Aa | Cas9_Cr; | |
| | k..N............k............k............M.......k...k..N | Cpf1_Wa; | |
| | ...........................k...........N...LN............. | Cpf1_Cr; | |
| UGT1A8 | tcattcagatcacatgaccttcctgCAGCCAGCgggtgaagaacatgctcattgcctt | SEQ_DUP; | 391 |
| | .........................11112222......................... | DUP_NUM; | |
| | ...aC......a........A..A...aHaaa.aB.aB....A......A........ | Cas9_Wa; | |
| | .aa.a.a..BaH..Ca.a....Aa.Baa..A..Aa..A..........A...A.a... | Cas9_Cr; | |
| | ..........................NL..............NL.............. | Cpf1_Wa; | |
| | ................LN..N.......................N............. | Cpf1_Cr; | |
| TREX1 | gatgtcctggccctgctcagcatctGTCAGTCAgtggagaccacaggccctgctgcgg | SEQ_DUP; | 392 |
| | .........................11112222......................... | DUP_NUM; | |
| | ....aA....A....A.....A...A...a.aa.a......aA....A..a.aaHaaa | Cas9_Wa; | |
| | ..........aa...Aaa..A.a..A.Ca...a...a........Aa.a...Aaa..A | Cas9_Cr; | |
| | .......................................................... | Cpf1_Wa; | |
| | ..............L...........................L............... | Cpf1_Cr; | |
| COL7A1 | cagcctcgacacacgacccacaggcTCAGTCAGgggctggggacagaggcaaggtaag | SEQ_DUP; | 393 |
| | .........................11112222......................... | DUP_NUM; | |
| | .J.a......a.......aA....A...aaaA..aaaa...a.aA...aA...aaaA. | Cas9_Wa; | |
| | .Ca..A..Aa.a..A.a.a..AaaHa...A.a...a.....A......A.....A... | Cas9_Cr; | |
| | ............N............................................. | Cpf1_Wa; | |
| | ...................L.........N....N................N...... | Cpf1_Cr; | |
| IQCB1 | gaaaacccttccaataggcttgaatCAAGCAAGcatgctgcttgatgtagtttctgaa | SEQ_DUP; | 394 |
| | .........................11112222......................... | DUP_NUM; | |
| | ............aA..HaB.....A...A...A..A...aC.A..A.....aB...A. | Cas9_Wa; | |
| | ...a......AaaHBaa......A.....Ca..IA...A...A..A...........B | Cas9_Cr; | |
| | .N...k........k..N.............NLL........N............... | Cpf1_Wa; | |
| | LN...N...N........................Lk......Lk....N........M | Cpf1_Cr; | |
| DOK7 | acagatgaactgggctcactgctcaGCCTGCCTgccagcagcgggggcccccgagccc | SEQ_DUP; | 395 |
| | .........................11112222......................... | DUP_NUM; | |
| | C.aB...aaA......A....A...A...A...A..a.aaaaA.....a.A....A.. | Cas9_Wa; | |
| | ...Aa.a.......A....A.a.a..A.a..Aa..Aa..Aa..A..A.....Aaaaa. | Cas9_Cr; | |
| | .......................................................... | Cpf1_Wa; | |
| | .......................................................... | Cpf1_Cr; | |
| WFS1 | gccatcatggagatcaaggagtaccTGATTGATtgacatggcctccagggcaggcatg | SEQ_DUP; | 396 |
| | .........................11112222......................... | DUP_NUM; | |
| | ....aa.aC....aa.A..I..ac..aC..a....aA......aaA..aAJ..A.... | Cas9_Wa; | |
| | ..A.a.aa.Ca.......Ca........Aa.J.........A....Aa.aa....A.. | Cas9_Cr; | |
| | ...................................................N...N.. | Cpf1_Wa; | |
| | .......................................................... | Cpf1_Cr; | |
| CC2D2A | ctctttattaccattgagccccagcTGGTTGGTtcctggagagtccattcgagaaaag | SEQ_DUP; | 397 |
| | .........................11112222......................... | DUP_NUM; | |
| | ...........a.A.....A..aA..aA.....aaHa.A.......a.aB...aA... | Cas9_Wa; | |
| | ...AaaHa.......Aa......Aaaa..A........Baa........aa..BaH.. | Cas9_Cr; | |
| | ..................N........k..N.....N..............N...NL. | Cpf1_Wa; | |
| | ................L...............k......N.......M......LLk. | Cpf1_Cr; | |
| SPINK5 | gatgggaaaacatatgacaacagatGTGCGTGCactgtgtgctgagaatgcgtgagta | SEQ_DUP; | 398 |
| | .........................11112222......................... | DUP_NUM; | |
| | aaB........a......aC.a.aJa.A....a.a.A..aHaB..a.aHa.A...... | Cas9_Wa; | |
| | .A.a...........A......A..A.......A..JA.aJ.....AJ.J.....A.. | Cas9_Cr; | |
| | .N.M.........k......................M..................... | Cpf1_Wa; | |
| | .........................N.................LN............. | Cpf1_Cr; | |
| SH3TC2 | cactagactcacggtcaggcaggcaGGCCGGCCagcagggcacctgccttttccaaca | SEQ_DUP; | 399 |
| | .........................11112222......................... | DUP_NUM; | |
| | .a......aA...aA..aA..aA..aA...A.JaaA.....A............aaa. | Cas9_Wa; | |
| | A....A.a....A.aHa...a...A...A...Aa..Aa..A....A.aa..Aa...Ba | Cas9_Cr; | |
| | ............NL....N....................................... | Cpf1_Wa; | |
| | .................................N.......L................ | Cpf1_Cr; | |
| DDX41 | cagacatacctggttttgatggggtCATCCATCatacgtaatgcccttagccatctcc | SEQ_DUP; | 400 |
| | .........................11112222......................... | DUP_NUM; | |
| | .......aA....aC.aaaA......J......A....A......A............ | Cas9_Wa; | |
| | A....A..JA...Aa...............a.CaaHCa...A......Aaa....Aa. | Cas9_Cr; | |
| | ......................................k............L...... | Cpf1_Wa; | |
| | ............M.....N..............................N.....LN. | Cpf1_Cr; | |
| CDSN | accgctggagtcacccttcccagtgAGGCAGGCaggggtcgttaggggaggtgatacg | SEQ_DUP; | 401 |
| | .........................11112222......................... | DUP_NUM; | |
| | ..aa.A............a.a.aA..aA..aaaA..A...aaaa.aa.aC...a.a.a | Cas9_Wa; | |
| | A.....Aa.a......a.aaaHBaaa.......A...A......a............. | Cas9_Cr; | |
| | .......................................NLL................ | Cpf1_Wa; | |
| | .........................L......M..................N...... | Cpf1_Cr | |
| LAMA2 | ggcataaagtcactgccaacaagatCAAACAAAcaccgcattgagctcacagtcgatg | SEQ_DUP; | 402 |
| | .........................11112222......................... | DUP_NUM; | |
| | ....A.....A.......aC.....J.......A....a.A......A..aC.aaaB. | Cas9_Wa; | |
| | .A.....A.......a.a..Aa..A....Ca...A...A.aa.a......A.a.a... | Cas9_Cr; | |
| | .......k.................................................. | Cpf1_Wa; | |
| | .....k...k...........................LLN.......LLN.....k.. | Cpf1_Cr; | |
| SERAC1 | tttgacttccaacgaggggaagagaAGATAGATagcgaatattaacagagtattcagc | SEQ_DUP; | 403 |
| | .........................11112222......................... | DUP_NUM; | |
| | .........a.aaaaB.a.aB.aC..aC..aHaB........Ha.A......A..... | Cas9_Wa; | |
| | ...Ba.....A.Baa..A......................A.........A....... | Cas9_Cr; | |
| | ......N.....N.......N...k....NLL.......................... | Cpf1_Wa; | |
| | ...N..M...M....LN.....N...............k..................L | Cpf1_Cr; | |
| SERAC1 | cgctgaggctggtgtcatactccacAGATAGATataattcggagagcaggacagtctt | SEQ_DUP; | 404 |
| | .........................11112222......................... | DUP_NUM; | |
| | a.aA..aa.a............aC..aC........aa.a.A..aa...A......A. | Cas9_Wa; | |
| | aa...a.a.....A......a.J.A.aa.a.............BaH.....A....A. | Cas9_Cr; | |
| | ...........N......L.........................L...........M. | Cpf1_Wa; | |
| | ......M...M.M.N....L......................N.........N...k. | Cpf1_Cr; | |
| SLC26A4 | acacagccttctctgtctctcttggCAGTCAGTcggtcttggcagctgttgtaattgc | SEQ_DUP; | 405 |
| | .........................11112222......................... | DUP_NUM; | |
| | .A........A........aA..A...A..aA....aA..A..A..A.....A..... | Cas9_Wa; | |
| | ......A.a..Aa.Ba.a...a.a.a....A...a...a...a....A..A....... | Cas9_Cr; | |
| | ............NLL................N............N............. | Cpf1_Wa; | |
| | ...............................N......N....Lk...L......... | Cpf1_Cr; | |
| CFTR | cacttcttggtactcctgtcctgaaAGATAGATattaatttcaagatagaaagaggac | SEQ_DUP; | 406 |
| | .........................11112222......................... | DUP_NUM; | |
| | ..J.aA.......A....aB..aC..aC............aC..aB..a.aa...A.. | Cas9_Wa; | |
| | ..Ba.a.a.Ba......A.aa...aa...................Ba........... | Cas9_Cr; | |
| | .......N..NL.......k......N..N...........................N | Cpf1_Wa; | |
| | .Lk...M...M....N.....N..M...k.........................L... | Cpf1_Cr; | |
| RP1 | aaatgattggacagttttcatatagTGAATGAAgaaagggaaagtggggaaaacaagt | SEQ_DUP;_. | 407 |
| | .........................11112222......................... | DUP_NUM; | |
| | aC..aa...A..........aHaB..aB.aB..aaaB..a.aaaaB......A..Ha. | Cas9_Wa; | |
| | ................A.....Ba.................................. | Cas9_Cr; | |
| | M.............NL.............N.........kL...M............. | Cpf1_Wa; | |
| | M....LN..LN..k...LLk......LLk....N........................ | Cpf1_Cr; | |
| GLDC | ataagcccaggaaatgggcaagatgGAACGAACtggagccccatggggccgcactgac | SEQ_DUP; | 408 |
| | .........................11112222......................... | DUP_NUM; | |
| | A....aaB...aaA...aC.aaB..aB...aa.A......aaaa..A....a..aA.. | Cas9_Wa; | |
| | ..........Aaa..........A.........A...A.....Aaaa......Aa.a. | Cas9_Cr; | |
| | .....L............N....M.................................. | Cpf1_Wa; | |
| | ...LLN..LN...L............................................ | Cpf1_Cr; | |
| GNE | ctgagatacgtacctagccacatgcGAATGAATgatgctcatgtagtctttgttcttg | SEQ_DUP; | 409 |
| | .........................11112222......................... | DUP_NUM; | |
| | aCJ..A......A......aHaB.HaB..aC.A.....A..A.....A.....aA..a | Cas9_Wa; | |
| | .....a.......A...Aa.J.Aa.a...A............A.a.......a..... | Cas9_Cr; | |
| | .......N.................................................. | Cpf1_Wa; | |
| | ....LN..LN...................................N........M... | Cpf1_Cr; | |
| CYP17A1 | gagtcgatcagaaagaccaccttggGGATGGATgccttccagggagggcagctgccca | SEQ_DUP; | 410 |
| | .........................11112222......................... | DUP_NUM; | |
| | .aC...aB..a........aaaaCHaaC.A.......aaa.aaA..A..A........ | Cas9_Wa; | |
| | .........a..Ca.......Aa.aa.............Aa.Baa........A..A. | Cas9_Cr; | |
| | .....N....NL.....k..........................N............. | Cpf1_Wa; | |
| | ....L...L............L...................................L | Cpf1_Cr; | |
| ATM | actacacaaagagaatctagtgattACAGACAGtgtcccttgcaaaaggaagaaaata | SEQ_DUP; | 411 |
| | .........................11112222......................... | DUP_NUM; | |
| | ......aHaB....Ia.aC.....a...a.A..A.........aaB.aB.....aB.. | Cas9_Wa; | |
| | IA.a..A..A.a........Ca..I......A...A.....aaa...A.......... | Cas9_Cr; | |
| | .......k........N.............................N........... | Cpf1_Wa; | |
| | ......................k....LN..k....N...........LLN...N..k | Cpf1_Cr; | |
| FOXRED1 | aagtttccctggataaacacagaggGAGTGAGTggctttggcgtcttatggtgaggct | SEQ_DUP; | 412 |
| | .........................11112222......................... | DUP_NUM; | |
| | .....HaaCJ.......a.aaa.aHa.a.aA....aa.A......aa.a.aA...A.. | Cas9_Wa; | |
| | ....A.....Baaa........A.a...............A.....A..a........ | Cas9_Cr; | |
| | ...............NL..........kLL............................ | Cpf1_Wa; | |
| | ...L.............................................L........ | Cpf1_Cr; | |
| C12orf65 | ggctttgggagaagctgacgttgttATCCATCCccaggaatagctgtcactccggtcc | SEQ_DUP; | 413 |
| | .........................11112222......................... | DUP_NUM; | |
| | ..aaa.aB.A..a..A..A.............aaB...A..A.......aA....aC. | Cas9_Wa; | |
| | .A.aa..A...........A...A.......CaaHCaaaa........A...a.a.aa | Cas9_Cr; | |
| | M....................L.....k...............N..N.M.L...L... | Cpf1_Wa; | |
| | ................LN..............................N..N...... | Cpf1_Cr; | |
| GJB2 | tccacagtgttgggacaaggccaggCGTTCGTTgcacttcaccagccgctgcatggag | SEQ_DUP; | 414 |
| | .........................11112222......................... | DUP_NUM; | |
| | ..a.A..aaa....aA...aa.A...a..A..........A..A..A...aa.aB.A. | Cas9_Wa; | |
| | ..A...aa.a.......J..A....Aa...A..Ba....A.a.Ba.aa..Aa.a..A. | Cas9_Cr; | |
| | L.......................L.......N.................NL..N... | Cpf1_Wa; | |
| | .................................L...N................M... | Cpf1_Cr; | |
| BRCA2 | ctgaaaatgaagataacaaatatacTGCTTGCTgccagtagaaattctcataacttag | SEQ_DUP; | 415 |
| | .........................11112222......................... | DUP_NUM; | |
| | ....aB.aC.....J.......A...A..A...A..aB..............HaB... | Cas9_Wa; | |
| | .....A...............A.......A..A...A..Aa.........BaHa.... | Cas9_Cr; | |
| | .........N..................................M......N...... | Cpf1_Wa; | |
| | M...................k.......M.N......N..................N. | Cpf1_Cr; | |
| BRCA2 | ttgtttctccggctgcacagaaggcATTTATTTcagccaccaaggagttgtggcacca | SEQ_DUP; | 416 |
| | .........................11112222......................... | DUP_NUM; | |
| | ......aa..A....aB.aA...........A.......aa.A..a.aA...J..... | Cas9_Wa; | |
| | ..A..J....Ba.aa..A..A.a......A.......Ba..Aa.aa............ | Cas9_Cr; | |
| | .....N....L....k.......k...k...L..................k...kL.. | Cpf1_Wa; | |
| | ....................N...............k.....Lk.......M.k...k | Cpf1_Cr; | |
| EIF2AK4 | tcctcagcagctcggtggagtggagCACTCACTtcgggcgagcgctcggccagtgccc | SEQ_DUP; | 417 |
| | .........................11112222......................... | DUP_NUM; | |
| | ..A..A...aa.aa.a.aa.A..........aaa.a.a.A...aA...a.A...A... | Cas9_Wa; | |
| | .aaaHCaaHa..A..A.a............A.a.aHa.Ba...A...A.a.a..Aa.. | Cas9_Cr; | |
| | .......................................................NL. | Cpf1_Wa; | |
| | L......................................................... | Cpf1_Cr; | |
| HEXA | gaaatccttccagtcagggccatagGATAGATAtacggttcaggtaccagggggcaga | SEQ_DUP; | 418 |
| | .........................11112222......................... | DUP_NUM; | |
| | ........A...aaA....HaaC..aC.....aA..J.aA.....aaaaA..a.a.aB | Cas9_Wa; | |
| | .........CaaHbaa...a....Aa..............A...Ba.....Aa.J... | Cas9_Cr; | |
| | ...........NLL....M...........NLL......................M.. | Cpf1_Wa; | |
| | M....M...M.M............................N.......LLN....... | Cpf1_Cr; | |
| PEX12 | acagaaaggccagtagacagggataAGGCAGGCaacacccccaacagctttcttcaga | SEQ_DUP; | 419 |
| | .........................11112222......................... | DUP_NUM; | |
| | B..aA...A..a...aaaC...aA..aa..............A.........a.A.Ha | Cas9_Wa; | |
| | A.aaaHa.......Aa......A..........A...A..A.aaaaa..A..A..Ba. | Cas9_Cr; | |
| | .......................................................... | Cpf1_Wa; | |
| | .M.N........N........N...................................N | Cpf1_Cr; | |
| MKS1 | agacagtgcaagcggaaggtgacagTGCCTGCCtgtggtctctgtgcggagtccaaag | SEQ_DUP; | 420 |
| | .........................11112222......................... | DUP_NUM; | |
| | .a.A...a.aaB.aa.a...a.A...A...a.aA.....a.a.aa.A......a.aA. | Cas9_Wa; | |
| | .A..A...A....A..JA.........A....Aa.JAa......a.a....A..J..a | Cas9_Cr; | |
| | .......................................................... | Cpf1_Wa; | |
| | ..........................L......k........................ | Cpf1_Cr; | |
| NPC1 | tactcacggagctgcccatgtgggcAAGTAAGTgcctcttccgcgcgctccacgcggc | SEQ_DUP; | 421 |
| | .........................11112222......................... | DUP_NUM; | |
| | ...aa.A..A.....a.aaA...A...a.A........a.a.A......a.aA..A.. | Cas9_Wa; | |
| | a......A.aha....A..Aaa.......A.........Aa.a.Baa.a.a.a.aa.a | Cas9_Cr; | |
| | .......................................................... | Cpf1_Wa; | |
| | ....N...N...............................................LN | Cpf1_Cr; | |
| PNPLA6 | agcgttgtacgcggaggagcgcagcGCCAGCCAgccgcacgaagcagcgggcccggga | SEQ_DUP; | 422 |
| | .........................11112222......................... | DUP_NUM; | |
| | ..A...a.aa.aa.a.A..a.A...A...a..A...aB.A..a.aaA...aaa.a.aa | Cas9_Wa; | |
| | .Ca....A......A.aJ......A.a..A.aa..Aa..Aa.a.a....A..A...Aa | Cas9_Cr; | |
| | ....L..N.......kL..........N.............................. | Cpf1_Wa; | |
| | ...................LN.............L.......N............... | Cpf1_Cr; | |
| LDLR | gcagccagctctgcgtgaacctggaGGGTGGGTggctacaagtgccagtgtgaggaag | SEQ_DUP; | 423 |
| | .........................11112222......................... | DUP_NUM; | |
| | ...A....a.a.aB....aaHaaaHaaa.aA......a.A...a.a.a.aaB.aA... | Cas9_Wa; | |
| | A.aa..A..Aa..A.a..A.....Aa..............A..A.....Aa.J..... | Cas9_Cr; | |
| | .............L............................................ | Cpf1_Wa; | |
| | .L................N..............LN............L.......... | Cpf1_Cr; | |
| FKRP | ggcccccgtgtcaccgtcctggtgcGGGAGGGAgttcgaggcatttgacaacgcggtg | SEQ_DUP; | 424 |
| | .........................11112222......................... | DUP_NUM; | |
| | ...a.A.....A....aa.a.aaa.aaa.A...a.aA.....a.....a.aa.A...a | Cas9_Wa; | |
| | A..Aa..Aaaaa....a.aa..aa.....A..J.......Ba....A......A..A. | Cas9_Cr; | |
| | .........................................................N | Cpf1_Wa; | |
| | .....L...L..................N............................. | Cpf1_Cr; | |
| CHEK2 | atttaccttccaagagtttttgacaTGATTGATgtattcatctcttaatgccttagga | SEQ_DUP; | 425 |
| | .........................11112222......................... | DUP_NUM; | |
| | ........Ha.A.....a.I..aC..aC.A...............A....HaaC.... | Cas9_Wa; | |
| | .........Aa.Baa.............A.............BahCa.a......Aa. | Cas9_Cr; | |
| | .......k.M.M.............k....NLL.........k........N...... | Cpf1_Wa; | |
| | .........................N..........M.k................... | Cpf1_Cr; | |
| NPHP4 | aactcggcgacccccagcgtggcgtGGAGCGGAGCgtgtgctccgtggtgatggccaggc | SEQ_DUP; | 426 |
| | .........................1111122222......................... | DUP_NUM; | |
| | .aa.a.......a.a.aa.a.aa.a.aa.a.a.a.A....a.aa.aC.aA...aA....A | Cas9_Wa; | |
| | aH.....A.aH.A..Aaaaa..A....A......A....A.....AJaa..........A | Cas9_Cr; | |
| | ..N..N...................................................... | Cpf1_Wa; | |
| | ....L....L.................................................. | Cpf1_Cr; | |
| IDUA | caggcttcctgaactactacgatgcCTGCTCTGCTcggagggtctgcgcgccgccagccc | SEQ_DUP; | 427 |
| | .........................1111122222......................... | DUP_NUM; | |
| | ......aB.....aC.A...A.......A...aaHaaA...a.a.A..A...A....A.. | Cas9_Wa; | |
| | a..Aaa...A.Baa....A..A..A....Aa..A.a..A.a.......a..A.a.aa.aa | Cas9_Cr; | |
| | ..............L.............NL.............................. | Cpf1_Wa; | |
| | ...............L....................................L....... | Cpf1_Cr; | |
| CCNO | aggtctgtagatctagctgcgccacGGGCTGGGCTgggccgggccgggcagggggctacc | SEQ_DUP; | 428 |
| | .........................1111122222......................... | DUP_NUM; | |
| | ..A..aC....A..a.A....aaA..aaA..aaA..aaA..aaA..aaaaA......... | Cas9_Wa; | |
| | .a.......a......Ca...A..A.aa.a...A....A....Aa...Aa...A...... | Cas9_Cr; | |
| | ............................................................ | Cpf1_Wa; | |
| | ............................................................ | Cpf1_Cr; | |
| CCNO | gctgcgccacgggctgggccgggccGGGCAGGGCAgggggctaccaccccgcgccgcaga | SEQ_DUP; | 429 |
| | .........................1111122222......................... | DUP_NUM; | |
| | .A....aaA..aaA..aaA..aaA..aaA..aaaaA..........a.A..A..a.aaaA | Cas9_Wa; | |
| | .Ca...A..A.aa.a...A....Aa...Aa...A....A......A..Aa.aaaa.a.aa | Cas9_Cr; | |
| | ............................................................ | Cpf1_Wa; | |
| | ............................................................ | Cpf1_Cr; | |
| MYBPC3 | ctcatgcccttgagcctctttagcaTGCCGTGCCGcgcaggtcagtgacgccgtactgga | SEQ_DUP; | 430 |
| | .........................1111122222......................... | DUP_NUM; | |
| | .A.....a.A........A...A..a.A..a.A..aA...a.a..a..A....aaB.aa. | Cas9_Wa; | |
| | a.a.aa.a...Aaa.....Aa.a.....A...Aa...Aa.a.a....a.....A.aa... | Cas9_Cr; | |
| | ........N..N....................N.........k................. | Cpf1_Wa; | |
| | ....................................LLN..................... | Cpf1_Cr; | |
| CHRNE | agatgagggtgggggtagcttaccaGTGAGGTGAGatgagattcgtcagggtgaccttga | SEQ_DUP; | 431 |
| | .........................1111122222......................... | DUP_NUM; | |
| | aHaaa.aaaaA..A.......a.a.aa.a.aCIa.aC...A..Haaa.a.....a.aA.. | Cas9_Wa; | |
| | .A.....................A...Aa..................BaH.a.......A | Cas9_Cr; | |
| | ...............k....N.....................N................. | Cpf1_Wa; | |
| | ............................................................ | Cpf1_Cr; | |
| BRCA1 | attgtgctcactgtacttggaatgtTCTCATCTCAtttcccatttctctttcaggtgaca | SEQ_DUP; | 432 |
| | .........................1111122222......................... | DUP_NUM; | |
| | .A...J..A.....aaB..A.............................aa.a....HaB | Cas9_Wa; | |
| | ..A........A.aJa....A..J......Ba.a.Ca.a...Baaa...Ba.a..Ba... | Cas9_Cr; | |
| | .k.........N..N.........N..............N.......N...........k | Cpf1_Wa; | |
| | .........................................LN.........k....... | Cpf1_Cr; | |
| SGCA | tacaatcgggacagctttgataccaCTCGGCTCGGcagaggctggtgctggagattgggg | SEQ_DUP; | 433 |
| | .........................1111122222......................... | DUP_NUM; | |
| | ...aaa...A..J.aC........aA...aA..a.aA..aa.A..aa.aC..aaaa.... | Cas9_Wa; | |
| | a..Aa..A..Ca..I.A..A.......Aa.a.aH.A.a..A.....A.....A..J.... | Cas9_Cr; | |
| | kLL....L...............................k.................... | Cpf1_Wa; | |
| | ............................L........L.......N.............. | Cpf1_Cr; | |
| PNKP | aggaagcagcggcaggggacgcccgCGGCTCGGCTcgggcacactggacgtacctgtggg | SEQ_DUP; | 434 |
| | .........................1111122222......................... | DUP_NUM; | |
| | .A..a.aA..aaaa..A...a.aA...aA..JaaA......aa..A.....a.aaaaaB. | Cas9_Wa; | |
| | ...........A..A..A......A.aaa.a..A.a..A.a...A.a.a....A...Aa. | Cas9_Cr; | |
| | ......L..................................................... | Cpf1_Wa; | |
| | ........................L..............LLN.......L.......... | Cpf1_Cr; | |
| DNAAF3 | gtcccagtcgctgacaccgcgccggGCGTCGCGTCgtagcgggagcccaggtagtggcgc | SEQ_DUP; | 435 |
| | .........................1111122222......................... | DUP_NUM; | |
| | ..A..AJ.a.....a.A..aaa.A..a.A..A..a.aaa.A....aA..a.aa.A..aa. | Cas9_Wa; | |
| | A.a....aaa...a.a...A.aa.a.aa...A..a.a..a....A.....Aaa....... | Cas9_Cr; | |
| | ............NL...........L.................................. | Cpf1_Wa; | |
| | ....................L....................................... | Cpf1_Cr; | |
| MUTYH | cccttcctcccctggagtcacctgcATCCATATCCATccggtatagtagttgatcacagtgg | SEQ_DUP; | 436 |
| | .........................111111222222......................... | DUP_NUM; | |
| | .........aa.A......A...............aA....A..A..aC.....a.aA.... | Cas9_Wa; | |
| | AaaHBaaa.Baa.aaaa......a.aa..A.CaaH..CaaHCaaH......J......Ca.a | Cas9_Cr; | |
| | .....................NLL..NL...L..................L...M.L...L. | Cpf1_Wa; | |
| | ........M............M....................N................... | Cpf1_Cr; | |
| SLC22A5 | aggatgaccatatcagtgggctattTTGGGCTTGGGCtttcgcttgatactcctaacttgca | SEQ_DUP; | 437 |
| | .........................111111222222......................... | DUP_NUM; | |
| | .a.........a.aaA.......aaA...aaA.....A.J.aC............A...aaa | Cas9_Wa; | |
| | ...Ba.......Aa...Ca......A.........A.....A..Ba.a......A.aa...A | Cas9_Cr; | |
| | ....k..N............kL............M.............k.....N......k | Cpf1_Wa; | |
| | .........................M.......N..........L...........LN.... | Cpf1_Cr; | |
| FOXC1 | agcagcagctcgtcgtccctgagtcACGGCGACGGCGgcggcggcggcggcggcgggggagg | SEQ_DUP; | 438 |
| | .........................111111222222......................... | DUP_NUM; | |
| | A..A...A..A....Ha.A....aa.a..aa.aa.aa.aa.aa.aa.aa.aaaaa.aA...a | Cas9_Wa; | |
| | .a.aa..A..A..A.a..a..aaa.....a.a..A..A..A..A..A..A..A..A..A... | Cas9_Cr; | |
| | ..............L........................L...................... | Cpf1_Wa; | |
| | ....................................L......................... | Cpf1_Cr; | |
| RMRP | ttcagcacgaaccacgtcctcagctTCACAGATCACAGAgtagtattttatagccctaaagaaa | SEQ_DUP; | 439 |
| | .........................11111112222222......................... | DUP_NUM; | |
| | A...aB.....A......A.......aC....Ha.A..A.........A.......aB....a. | Cas9_Wa; | |
| | ...Aa.Ba..A.a...Aa.a..aa.a..A.Ba.a...Ca.a............J....Aaa... | Cas9_Cr; | |
| | .......................NL......................NL............... | Cpf1_Wa; | |
| | ............................M.......k...k....................... | Cpf1_Cr; | |
| CHRNE | ggtcccctgccggtgcctctgccccTCAAACATCAAACAcgagctcgctccgtggctttttcag | SEQ_DUP; | 440 |
| | .........................11111112222222......................... | DUP_NUM; | |
| | ....A..aa.A.....A............J......a.A...A....a.aA.........A... | Cas9_Wa; | |
| | H.aa....aaaa..Aa....Aa.a..Aaaa.a...A.Ca...A.a...A.a.a.aa....A... | Cas9_Cr; | |
| | ....................L.....L..................................... | Cpf1_Wa; | |
| | ......k......k..............................................L... | Cpf1_Cr; | |
| RBCK1 | ccaggtccccgcctcataccagcccGACGAGGGACGAGGaggagcgagcgcgcctggcgggcga | SEQ_DUP; | 441 |
| | .........................11111112222222......................... | DUP_NUM; | |
| | A.....A.J........A...a..a.aaa..a.aa.aa.a.a.a.a.A...aa.aaa.a.aa.a | Cas9_Wa; | |
| | .Aa..Aa....aaaa.aa.a...Aa..Aaa..A......A.........A...A.a.aa...A. | Cas9_Cr; | |
| | .............................L.................................. | Cpf1_Wa; | |
| | ..........L..................................................... | Cpf1_Cr; | |
| ABHD12 | ctgtacttgccactgaaaatggatgGCTCTTAGCTCTTAgcttcttcgcggatattagtgaatg | SEQ_DUP; | 442 |
| | .........................11111112222222......................... | DUP_NUM; | |
| | ....A.....aB...HaaC.aA......A......A.......aHaaC.....aHaB..aa.aC | Cas9_Wa; | |
| | ....Ca..I.A..JAa.a.............A.a....A.a....A.Ba.Ba.a.......... | Cas9_Cr; | |
| | ......N......k...............N......................N......N.... | Cpf1_Wa; | |
| | ............................L.M......LN..L.........N..M.N....... | Cpf1_Cr; | |
| MSH6 | tggctttaatgcagcaaggcttgctAATCTCCCAATCTCCCagaggaagttattcaaaagggacat | SEQ_DUP; | |
| | .........................1111111122222222......................... | DUP_NUM; | |
| | ......A..A...aA...A...................a.aaB.A..........aaa.....aB. | Cas9_Wa; | |
| | ..A.....A.......A..A....A...A...Ca.aaa..Ca.aaa............BaH..... | Cas9_Cr; | |
| | ........N.............M.....k..............N.........L.......L.... | Cpf1_Wa; | |
| | ...N........N...........LN........k....L...M...k.....N....N....... | Cpf1_Cr; | |
| LAMA2 | ttgaagaagaggaagaagatacagaACGTGTTCACGTGTTCtccagcttatgattatcttagaggt | SEQ_DUP; | 444 |
| | .........................1111111122222222......................... | DUP_NUM; | |
| | .aB.a.aaB.ab.aC....aB..a.A.....a.A.......A..I..aC........a.aA.ha.A | Cas9_Wa; | |
| | ...Aa.....................A....A....BaJa....BaJaa..A.........Ca... | Cas9_Cr; | |
| | .....k......M....M.....N.............................NL......N...L | Cpf1_Wa; | |
| | ...N.............................................................. | Cpf1_Cr; | |
| TCTN2 | agacgtcaatcctccttttgatcagCTCTGCTCCTCTGCTCtgctgggacgacgacacgtggtgtc | SEQ_DUP; | 445 |
| | .........................1111111122222222......................... | DUP_NUM; | |
| | A..............aC...A....A.......A....A..aaa..aJ.a....a.aa.A..I..a | Cas9_Wa; | |
| | J.......A..a..CaaHaa......Ca..A.a..A.aa.a..A.a..A.....A..A..A.a... | Cas9_Cr; | |
| | ...NL...........k.......................k......................... | Cpf1_Wa; | |
| | .........................L........................................ | Cpf1_Cr; | |
| BRCA2 | gaatttgacaggataatagaaaatcAAGAAAAAAAGAAAAAtccttaaaggcttcaaaaagcactc | SEQ_DUP; | 446 |
| | .........................1111111122222222......................... | DUP_NUM; | |
| | ..a..HaaC.....aB.......aB......aB............aA......J..A.......aC | Cas9_Wa; | |
| | .............A..............Ca..I.............CaaHI.....A.Ba...... | Cas9_Cr; | |
| | ..............N..N.........k...................................... | Cpf1_Wa; | |
| | ....N..k.......k.........k........k..................k............ | Cpf1_Cr; | |
| TCAP | tcatggctacctcagagctgagctgCGAGGTGTCGAGGTGTcggaggagaactgtgagcgccggga | SEQ_DUP; | 447 |
| | .........................1111111122222222......................... | DUP_NUM; | |
| | aA........a.A..a.A..a.a.aa.A..a.aa.A..aa.aa.aB...a.a.a.A..aaa.aA.. | Cas9_Wa; | |
| | .....Ca....A..Aa.a....A....A..A.......a.J.....a.J.......A......A.a | Cas9_Cr; | |
| | .................................................................. | Cpf1_Wa; | |
| | .....................L......N.............L................N..LLN. | Cpf1_Cr; | |
| LDLR | gatggtggccccgactgcaaggacaAATCTGACAATCTGACgaggaaaactgcggtatgggcgggg | SEQ_DUP; | 448 |
| | .........................1111111122222222......................... | DUP_NUM; | |
| | a.aA....a...A...aa........a.......a..a.aaB.....a.aA...aaa.aaaA..Ha | Cas9_Wa; | |
| | .a...........Aaaa..A..A.....A...Ca..IA..Ca..IA........A..A.......J | Cas9_Cr; | |
| | ....L....L........................................................ | Cpf1_Wa; | |
| | ...k........N..........Lk......................................... | Cpf1_Cr; | |
| ITPA | agcctatgcgctctgcacgtttgcaCTCAGCACCTCAGCACcggggacccaagccagcccgtgcgc | SEQ_DUP; | 449 |
| | .........................1111111122222222......................... | DUP_NUM; | |
| | ...a.A.J..A...AJ..A...J..A....J..A....aaaa......A...A...a.a.A...A. | Cas9_Wa; | |
| | ....a..Aa....A.a.a..A.a.....A.a.aH.A.aa.a..A.aa.....AaaH..Aa..Aaa. | Cas9_Cr; | |
| | .........NL.................M..............k...................... | Cpf1_Wa; | |
| | .......................L.....N............................L....... | Cpf1_Cr; | |
| PEX1 | tccagcaggacaacagatggctgcaTCCACACTGTCCACACTGcctctgagaaagccacctctagggt | SEQ_DUP; | 450 |
| | .........................111111111222222222......................... | DUP_NUM; | |
| | A..aa......aC.aA..A..J.......AJ.......A.....a.aB..A........HaaA..... | Cas9_Wa; | |
| | ..a.CaaH.A.....A..A......A..A.CaaHa.a...aa.a.a..Aa.a........Aa.aa.a. | Cas9_Cr; | |
| | ..L.....................L........................L........L......... | Cpf1_Wa; | |
| | ..............................k...............................N..... | Cpf1_Cr; | |
| IGFALS | gcctgttgcccgccagcaccagctcGCGCAGGCTGCGCAGGCTgcccaggccgcggaacgccgcatcg | SEQ_DUP; | 451 |
| | .........................111111111222222222......................... | DUP_NUM; | |
| | A..A...AJ..A.....A...a.A..aA..a.A..aA..A....aA..a.aaB..A..A....aaaa. | Cas9_Wa; | |
| | ..Aa..Aa.....Aaa.aa..A.aa..A.a.a.a...A..A.a...A..Aaa...Aa.a....A.aa. | Cas9_Cr; | |
| | ............................N....................................... | Cpf1_Wa; | |
| | .................................LLN............L................... | Cpf1_Cr; | |
| SEPN1 | gcagccgccgccagccgcagccatgGGCCGGGCCCGGCCGGGCCCggccgggccaacgcgggccgcccag | SEQ_DUP; | 452 |
| | .........................11111111112222222222......................... | DUP_NUM; | |
| | ..A..A...A..A..A....aaA..aaA...aA..aaA...aA..aaA.....a.aaA..A....A.... | Cas9_Wa; | |
| | a.aa..A..Aa.aa.aa..Aa.a..Aa.....Aa...Aaa..Aa...Aaa..Aa...Aa..A.a...Aa. | Cas9_Cr; | |
| | ....kL...NLL.......................................................... | Cpf1_Wa; | |
| | .................................N.................................... | Cpf1_Cr; | |
| CYP1B1 | agcccaagacagaggtgttggcagtGGTGGCATGAGGTGGCATGAggaatagtgacaggcacaaagctgg | SEQ_DUP; | 453 |
| | .........................11111111112222222222......................... | DUP_NUM; | |
| | ...a...a.aa.A..aA..a.aa.aA...a.aa.aA...a.aaB...a.a.J.aA......A..aa.aB. | Cas9_Wa; | |
| | .......Aaa....A.........JA.........A.........A..............A...A.a... | Cas9_Cr; | |
| | ..........N.............................N............................. | Cpf1_Wa; | |
| | ........................LN...............k.....L...N.............M.k.. | Cpf1_Cr; | |
| CYP27A1 | gtgcaggcgcgcgagcacaacccatGGCTGCGCTGGGCTGCGCTGggctgcgcgaggctgaggtgggcgc | SEQ_DUP; | 454 |
| | .........................11111111112222222222......................... | DUP_NUM; | |
| | .aa.a.aJa.A..........aA..a.A..aaA..a.A..aaA..a.a.a.aA..a.aa.aaa.A..a.a | Cas9_Wa; | |
| | aH......A..JA.a.a...A.a..AaaH...A..A.a....A..A.a....A..A.a....A....... | Cas9_Cr; | |
| | ...................................................................... | Cpf1_Wa; | |
| | ...................................................................... | Cpf1_Cr; | |
| RMRP | ttcagcacgaaccacgtcctcagctTCACAGAGTATCACAGAGTAgtattttatagccctaaagaaattg | SEQ_DUP; | 455 |
| | .........................11111111112222222222......................... | DUP_NUM; | |
| | A...aB.....A......A......Ha.A.......Ha.A..A........A.......aB....a.A.. | Cas9_Wa; | |
| | ...Aa.Ba..A.a...Aa.a..aa.a..A.Ba.a......Ca.a............J....Aaa...... | Cas9_Cr; | |
| | ......................NL.......................NL........M............ | Cpf1_Wa; | |
| | ...............................M.......k...k.........................k | Cpf1_Cr; | |
| SPG11 | catggaggcatttgcttgtcagcacTTCCAGGTTATTCCAGGTTAgttaccacttcattactggagggca | SEQ_DUP; | 456 |
| | .........................11111111112222222222......................... | DUP_NUM; | |
| | a.aA.....A...AJ..A........aA........aA...A................aaJaaA....A. | Cas9_Wa; | |
| | a.Baaa.......A.....A....a..A.a.Baa.......Baa..........Aa.a.Ba....A.... | Cas9_Cr; | |
| | ................N..NLL............k...N.........NLL....N..NLL....N...N | Cpf1_Wa; | |
| | .........................................L..............k............. | Cpf1_Cr; | |
| SPG11 | gtaggagagcatggatctctgggtgCAGATCCTCCCAGATCCTCCatactagcttcccctgaggccagtg | SEQ_DUP; | 457 |
| | .........................11111111112222222222......................... | DUP_NUM; | |
| | a.a.A..HaaC....Haaa.A..aC........aC...J........A........a.aA...a.A.... | Cas9_Wa; | |
| | .a............A.....Ca.a......A..JCaaHaaa...CaaHaa...A...A.Baaaa.....A | Cas9_Cr; | |
| | NL...................k..................................L.........L... | Cpf1_Wa; | |
| | ........................M...................................N.....N... | Cpf1_Cr; | |
| BRCA1 | taatgagctggcatgagtatttgtgCCACATGGCTCCACATGGCTccacatgcaagtttgaaacagaact | SEQ_DUP; | 458 |
| | .........................11111111112222222222......................... | DUP_NUM; | |
| | a.A..aA..Ha.A.....a.A......aA........aA........A...A...aB....aB......J | Cas9_Wa; | |
| | ..A.........A...A..........J..Aa.a....A.aa.a....A.aa.a...A..........A. | Cas9_Cr; | |
| | ............N..............................k..............L.........L. | Cpf1_Wa; | |
| | ...............................N.....Lk.....N..........M.........L.... | Cpf1_Cr; | |
| NCF4 | gttttcgtcatcgaggtgaagacaaAAGGAGGATCAAGGAGGATCcaagtacctcatctaccgccgctac | SEQ_DUP; | 459 |
| | .........................11111111112222222222......................... | DUP_NUM; | |
| | ..A.....a.aa.aB.a......aaHaaC....aaHaaC..J..A.............A..A.....A.. | Cas9_Wa; | |
| | a........Ba..a.Ca..........A..........Ca........CaaH....Aa.a.Ca..Aa.aa | Cas9_Cr; | |
| | ...................N......kL.......................................L.. | Cpf1_Wa; | |
| | ..k...........N..........N............................................ | Cpf1_Cr; | |
| SLC22A5 | gaggtgccccacagctgccgccgctACCGGCTCGCCACCGGCTCGCCaccatcgccaacttctcggcgcttg | SEQ_DUP; | 460 |
| | .........................1111111111122222222222......................... | DUP_NUM; | |
| | .A.......A..A..A..A.....aA...A.....aA...A........A..........aa.A...aaA.. | Cas9_Wa; | |
| | .Aa.a......Aaaa.a..A..Aa.aa.a..Aa..A.a.aa.aa..A.a.aa.aa.Ca.aa..A.Ba.a..A | Cas9_Cr; | |
| | ........................................................................ | Cpf1_Wa; | |
| | ...................................N...................L................ | Cpf1_Cr; | |
| KCNQ1 | gtggtgttcttcgggacggagtacgTGGTCCGCCTCTGGTCCGCCTCtggtccgccggctgccgcagcaagt | SEQ_DUP; | 461 |
| | .........................1111111111122222222222......................... | DUP_NUM; | |
| | .A......aaa..aa.A...a.aA...A.....aA...A.....aA...A..aA..A..A..AJ..A...a. | Cas9_Wa; | |
| | ..A..J......BaJBa....A......A..J..aa.aa.a....aa.aa.a....aa.aa..A..Aa.a.. | Cas9_Cr; | |
| | ...L.........................N..NL..................L..........L........ | Cpf1_Wa; | |
| | ...............................................N........................ | Cpf1_Cr; | |
| MYO7A | gctgtgaacccctaccagctgctctCCATCTACTCGCCATCTACTCGccagagcacatccgccagtatacca | SEQ_DUP; | 462 |
| | .........................1111111111122222222222......................... | DUP_NUM; | |
| | .aB..........A..A..............A..........A..Ja.A.......A..JA........... | Cas9_Wa; | |
| | H.....A......Aaaa..Aa..A..A.a.aa.Ca..A.aHaa.Ca..A.aHaa....A.a.CaaHaa.... | Cas9_Cr; | |
| | ...M........L...................................L....................... | Cpf1_Wa; | |
| | .............................................M....N..N..N.......L....... | Cpf1Cr; | |
| CEP57 | ccacaagccctagccatgccgtggtAGCCAATGTTCAGCCAATGTTCagcttgtcttgcatctaatgaagca | SEQ_DUP; | 463 |
| | .........................1111111111122222222222......................... | DUP_NUM; | |
| | ..A.....A....A..a.aA..A.....A....A.....A....A...A....A........aB.a...... | Cas9_Wa; | |
| | .....aa.a...Aaa...Aa...Aa.......Aa.....Ba..Aa.....Ba..A....a...A.Ca..... | Cas9_Cr; | |
| | kL........k..NL...k....L................................NL.........NL... | Cpf1_Wa; | |
| | ........N..........N......................N..LN...N.......k...........N. | Cpf1_Cr; | |
| ACADM | gaaatggcaatgaaagttgaactagCTAGAATGAGTTACTAGAATGAGTTAccagagagcagcttgggaggt | SEQ_DUP; | 464 |
| | tgat | | |
| | .........................11111111111112222222222222..................... | DUP_NUM; | |
| | .... | | |
| | .aA....aB..A..aB....A..HaB.Ha.A.....HaB.Ha.A......a.a.A..A...aaa.aa..aC. | Cas9_Wa; | |
| | ...a | | |
| | ...A........A.............A...A............A............Aa......A..A.... | Cas9_Cr; | |
| | .... | | |
| | .......M....k.M........................N..................N............N | Cpf1_Wa; | |
| | .... | | |
| | ........N............N.......................L...................Lk..... | Cpf1_Cr; | |
| | .... | | |
| GAMT | gaggggcaccttgtgtgtctgccgtGGGGCCCAGTCCCGGGGCCCAGTCCCggagccgctggaagacgccgt | SEQ_DUP; | 465 |
| | catt | | |
| | .........................11111111111112222222222222..................... | DUP_NUM; | |
| | .... | | |
| | aA......a.a.A...A..a.aaaA....A....aaaA....A....aa.A..A..aaB.a..A..A..J.. | Cas9_Wa; | |
| | A... | | |
| | ...A.......A.aa........a.JAa......Aaa...aaa....Aaa...aaa....Aa.a.......A | Cas9_Cr; | |
| | .aa. | | |
| | .................................N........................L............L | Cpf1_Wa; | |
| | .... | | |
| | ..............................L........LLN...........................N.. | Cpf1_Cr; | |
| | .... | | |
| CHRNE | ccacctcttcggcattgtacgtctgAGAGCTGCGGAGCCAGAGCTGCGGAGCCagggccgggagcccacccc | SEQ_DUP; | 466 |
| | agaagc | | |
| | .........................1111111111111122222222222222................... | DUP_NUM; | |
| | ...... | | |
| | ......aA.J..A...A...a.a.A..a.aa.A...a.A..a.aa.A...aaA..aaa.A.........aB. | Cas9_Wa; | |
| | A.a... | | |
| | ...A.aa.aa.a.Ba..A......A..a......A..A...Aa.....A..A....Aa....Aa.....Aaa | Cas9_Cr; | |
| | .aaaa. | | |
| | .N.....................L......NL.....N.................................. | Cpf1_Wa; | |
| | ...... | | |
| | ............L.............L............L.............N.................. | Cpf1_Cr; | |
| | ...... | | |
| RMRP | gccttcagcacgaaccacgtcctcaGCTTCACAGAGTAGTGCTTCACAGAGTAGTattttatagccctaaag | SEQ_DUP; | 467 |
| | aaattgtg | | |
| | .........................111111111111111222222222222222................. | DUP_NUM; | |
| | ........ | | |
| | J..A...aB.....A......A......Ha.A..a.A.......Ha.A..A........A.......aB... | Cas9_Wa; | |
| | .a.A...I | | |
| | ..A...Aa.Ba..A.a...Aa.a..aa.a..A.Ba.a..........A.Ba.a...........J....Aaa | Cas9_Cr; | |
| | ........ | | |
| | ..........................NL.......................NL............NL..... | Cpf1_Wa; | |
| | ........ | | |
| | .......................................M........k...k................... | Cpf1_Cr; | |
| | .....k.. | | |
| ABCC8 | acagcggtgtgaccaagatatggaaGAGGGAGAGGGAGGCGAGGGAGAGGGAGGCaaaggccacggagggcg | SEQ_DUP; | 468 |
| | agaagtcg | | |
| | .........................111111111111111222222222222222................. | DUP_NUM; | |
| | ........ | | |
| | .aa.a.a.....aC...aaB.a.aaa.a.aaa.aa.a.aaa.a.aaa.aA....aA....aa.aaa.a.aB. | Cas9_Wa; | |
| | A..aA... | | |
| | A.....A..A........aa.........................A..............A.....Aa.a.. | Cas9_Cr; | |
| | ....A... | | |
| | ..........................................M............................. | Cpf1_Wa; | |
| | ........ | | |
| | LLN....L.....L........L....L......k........L.........N..............LN.. | Cpf1_Cr; | |
| | N....... | | |
| MMAB | tctctccagccctcttaccgtctctCGGCCCGGCGGCACACGGCCCGGCGGCACAcggcccggcagaaatgc | SEQ_DUP; | 469 |
| | agcgccga | | |
| | .........................111111111111111222222222222222................. | DUP_NUM; | |
| | ........ | | |
| | ....A..........A......aA...aa.aA.....aA...aa.aA.....aA...aA..aB...A..a.A | Cas9_Wa; | |
| | ..a.A..a | | |
| | a.aaa.a.a.aa..Aaa.a...Aa..a.a.a..Aaa..A..A.a.a..Aaa..A..A.a.a..Aaa..A... | Cas9_Cr; | |
| | ....A..A | | |
| | L.....L..N..........L.......L........N.................................. | Cpf1_Wa; | |
| | ........ | | |
| | .............................................k.......................... | LCpf1_Cr; | |
| | .......L | | |
| WFS1 | actggctggtcctcgccgcgaagcaGGGCCGTCGCGAGGCTGGGCCGTCGCGAGGCTgtgaagctgcttcgc | SEQ_DUP; | 470 |
| | cggtgcttgg | | |
| | .........................11111111111111112222222222222222............... | DUP_NUM; | |
| | .......... | | |
| | A..aA.....A..a.aB.A..aaA..A..a.a.aA..aaA..A..a.a.aA..a.aB.A..A....A..aa. | Cas9_Wa; | |
| | A...aa.aa. | | |
| | ......A...A....aa.a.aa.a....A....Aa..a.a....A....Aa..a.a....A.......A..A | Cas9_Cr; | |
| | .Ba.aa.... | | |
| | ........................................................................ | Cpf1_Wa; | |
| | .......... | | |
| | ......................................LN......................L.....N... | Cpf1_Cr; | |
| | .......... | | |
| CRB2 | cgctgggcggcctgcccctgcccttGGCGCGGCCCCGGCCCGGCGCGGCCCCGGCCCggcgcggcccctggc | SEQ_DUP; | 471 |
| | gcccgagagc | | |
| | .........................11111111111111112222222222222222............... | DUP_NUM; | |
| | .......... | | |
| | aaa.aA...A......A....aa.a.aA....aA...aa.a.aA....aA...aa.a.aA.....aa.A... | Cas9_Wa; | |
| | aJa.A..... | | |
| | a....A.a....A..Aa..Aaaa..Aaa....A.a..Aaaa..Aaa..A.a..Aaaa..Aaa..A.a..Aaa | Cas9_Cr; | |
| | a...A.aaa. | | |
| | ..........N...................................N......................... | Cpf1_Wa; | |
| | .......... | | |
| | ........................................................................ | Cpf1_Cr; | |
| | .......L.. | | |
| HPS1 | gtcctgcaggtgctggggcaggtgtGGGCCTCCCCTGCTGGGGGCCTCCCCTGCTGGgggctgtggtcagaa | SEQ_DUP; | 472 |
| | agttcagccg | | |
| | .........................11111111111111112222222222222222............... | DUP_NUM; | |
| | .......... | | |
| | .A..aa.A..aaaA..aa.a.aaA........A..aaaaA........A..aaaaA..a.aA...aB..A.. | Cas9_Wa; | |
| | ..A..aA..a | | |
| | .a.....aa..A.....A..J..A........JAa.aaaa..A......Aa.aaaa..A......A...... | BaCas9_Cr; | |
| | a.......Ba | | |
| | ....................N.................................L...............L. | Cpf1_Wa; | |
| | .......... | | |
| | .................................................k...................... | Cpf1_Cr; | |
| | .......... | | |
| DNAAF2 | ggtgaccccggagcccgcagccccaGCCACGCAGGTATCGTGCCACGCAGGTATCGTggcctccgtcctccg | SEQ_DUP; | 473 |
| | cgcgactcct | | |
| | .........................11111111111111112222222222222222............... | DUP_NUM; | |
| | .......... | | |
| | .....aa.A...A..A.....A....A..aA....a.A....A..aA....a.aA.....A......a.a.a | Cas9_Wa; | |
| | .......aHa | | |
| | ..........Aaaa....Aaa.a..Aaaa..Aa.a.a.....Ca.J.Aa.a.a.....Ca.J..Aa.aa..a | Cas9_Cr; | |
| | a.aa.a.a.. | | |
| | .......L...................................................M............ | Cpf1_Wa; | |
| | ...M...... | | |
| | ........................................................................ | Cpf1_Cr; | |
| | .......... | | |
| RMRP | cctaggatacaggccttcagcacgaACCACGTCCTCAGCTTCACCACGTCCTCAGCTTCacagagtagtatt | SEQ_DUP; | 474 |
| | ttatagccctaa | | |
| | .........................1111111111111111122222222222222222............. | DUP_NUM; | |
| | ............ | | |
| | aaC....aA...J..A...aB.....A......A.........A......A......Ha.A..A........ | Cas9_Wa; | |
| | .A.......aB. | | |
| | ...J.aa.......A...Aa.Ba..A.a...Aa.a..aa.a..A.Ba.aa.a..aa.a..A.Ba.a...... | Cas9_Cr; | |
| | ......J....A | | |
| | .....................................NL.......................NL........ | Cpf1_Wa; | |
| | .......NL... | | |
| | ..LN.................................................M.......k...k...... | Cpf1_Cr; | |
| | ............ | | |
| RMRP | gatacaggccttcagcacgaaccacGTCCTCAGCTTCACAGAGTCCTCAGCTTCACAGAgtagtattttata | SEQ_DUP; | 475 |
| | gccctaaagaaa | | |
| | .........................1111111111111111122222222222222222............. | DUP_NUM; | |
| | ............ | | |
| | ..aA...J..A...aB.....A......A......Ha.A......A......Ha.A..A.........A... | Cas9_Wa; | |
| | ....aB....a. | | |
| | aa.......A...Aa.Ba..A.a...Aa.a..aa.a..A.Ba.a.....aa.a..A.Ba.a........... | Cas9_Cr; | |
| | .J....Aaa... | | |
| | .................................NL......................NL............. | Cpf1_Wa; | |
| | ..NL........ | | |
| | ................................................M.......k...k........... | Cpf1_Cr; | |
| | ............ | | |
| PALB2 | caagacagactgagtctttcaaatgAGCAAGTTGGGGTGTGCAGCAAGTTGGGGTGTGCagcaagttcgtcc | SEQ_DUP; | 476 |
| | agcaacttctgt | | |
| | .........................1111111111111111122222222222222222............. | DUP_NUM; | |
| | ............ | | |
| | ...a..Ha.A..........a.A...A..aaaa.a.A..A...A..aaaa.a.A..A...A...A....A.. | Cas9_Wa; | |
| | ......A..aC. | | |
| | .a...A....A...A.....a..Ba.......A.............AJ.a.............AJ.a....B | Cas9_Cr; | |
| | a..aa..A..A. | | |
| | ......N...k....M........................kL............N................N | Cpf1_Wa; | |
| | ............ | | |
| | .......N................N................N............N................. | Cpf1_Cr; | |
| | ...M........ | | |
| PNKP | cgcggctcgcggcgtctgggtttgtGTTGTCGATGGCGACCCGTTGTCGATGGCGACCCgtttcccttgctt | SEQ_DUP; | 477 |
| | cagggctgtctc | | |
| | .........................1111111111111111122222222222222222............. | DUP_NUM; | |
| | ............ | | |
| | A...a.aa.A..HaaA...a.A..A..aC.aa.a....A..A..aC.aa.a....A........A.....aa | Cas9_Wa; | |
| | A..A.J...... | | |
| | Aa...A.a..A.a.a..A..a.............Ja.....A..AaaH....a.....A..AaaH..Baaa. | Cas9_Cr; | |
| | ..A.Ba....A. | | |
| | ............................................k....N................N..... | Cpf1_Wa; | |
| | ............ | | |
| | ........................................................................ | Cpf1_Cr; | |
| | ............ | | |
| F12 | atgaagcctaggggacaccggggtcGGAGGCGCCGCCTGGGTTGGAGGCGCCGCCTGGGTTggggtctggca | SEQ_DUP; | 478 |
| | ctgtgccaggtcgc | | |
| | .........................111111111111111111222222222222222222........... | DUP_NUM; | |
| | .............. | | |
| | .A....aaaa.....aaaA..aa.aa.A..A..HaaA..aa.aa.A..A..HaaA..aaaA.J.aA....a. | Cas9_Wa; | |
| | A...aA..a..A.. | | |
| | ....A......Aa.......A.aa.....a.....A.aa.aa...........A.aa.aa...........a | Cas9_Cr; | |
| | ...A.a....Aa.J | | |
| | ................................................................N....... | Cpf1_Wa; | |
| | ..........N... | | |
| | ....L.................L................................................. | Cpf1_Cr; | |
| | .............. | | |
| YARS2 | gagcttctttaaacaactcttttaaCTCCTGATCAGACATGACCTCCTGATCAGACATGACctccagtgcat | SEQ_DUP; | 479 |
| | ctatgctactgtga | | |
| | .........................111111111111111111222222222222222222........... | DUP_NUM; | |
| | .............. | | |
| | ..........................aC...a....a.......aC...a....a.......a.A....... | Cas9_Wa; | |
| | A.....a.aC.... | | |
| | .........A.Ba.....A..A.aH.....A.aa...Ca...A....Aa.aa...Ca...A....Aa.aa.. | Cas9_Cr; | |
| | ..A.Ca....A..A | | |
| | .............NL............N...k............k........................... | Cpf1_Wa; | |
| | .............. | | |
| | ..N.............................................................M.k..... | Cpf1_Cr; | |
| | .............L | | |
| LDLR | cagctggcgctgtgatggtggccccGACTGCAAGGACAAATCTGACTGCAAGGACAAATCTgacgaggaaaa | SEQ_DUP; | 480 |
| | ctgcggtatgggcg | | |
| | .........................111111111111111111222222222222222222........... | DUP_NUM; | |
| | .............. | | |
| | .aa.A..a.aC.aa.aA....a...A...aa........a...A...aa........a..a.aaB.....a. | Cas9_Wa; | |
| | aA...aaa.aaaA. | | |
| | aHa.aa..A...A.a...........Aaaa..A..A.....A...Ca..IA..A.....A...Ca..IA... | Cas9_Cr; | |
| | .....A..A..... | | |
| | .................L....L................................................. | Cpf1_Wa; | |
| | .............. | | |
| | ..........N.....k...........N.....k...........Lk........................ | Cpf1_Cr; | |
| | .............. | | |
| KPTN | tcgggagatgggaccgtcctgcaggACCGACCACATCTGCAGAACCGACCACATCTGCAGAacctctgcgtg | SEQ_DUP; | 481 |
| | gagagcgaggattc | | |
| | .........................111111111111111111222222222222222222........... | DUP_NUM; | |
| | .............. | | |
| | a.aC.aaa...A....A..aa...a.........A..aB...a.........A..aB......a.a.aa.a. | Cas9_Wa; | |
| | a.aHaaC....a.A | | |
| | .Ca.aIaH..........Aa..aa..A....Aa..Aa.a.Ca..A....Aa..Aa.a.Ca..A....Aa.a. | Cas9_Cr; | |
| | .A........A... | | |
| | ........................................................................ | Cpf1_Wa; | |
| | .............. | | |
| | .....................N.................N..........L..................... | Cpf1_Cr; | |
| | .N............ | | |
| MSH6 | ttgctaatctcccagaggaagttatTCAAAAGGGACATAGAAAATCAAAAGGGACATAGAAAAgcaagagaa | SEQ_DUP; | 482 |
| | tttgagaagatgaatc | | |
| | .........................11111111111111111112222222222222222222......... | DUP_NUM; | |
| | ................ | | |
| | ..........a.aaB.A..........aaa.....aB.........aaa.....aB...A...aHaB....a | Cas9_Wa; | |
| | .aB.aCHaB....A.. | | |
| | ....A...A...Ca.aaa............BaH.......A........Ca..I.....A.........A.. | Cas9_Cr; | . |
| | ................ | | |
| | ..M.....k..............N.........L..........N..NL....................... | Cpf1_Wa; | |
| | ................ | | |
| | ......k....L...M...k.....k....L...M...k.....N....N.......N....LN........ | Cpf1_Cr; | |
| | ................ | | |
| BARD1 | cttctgcgtggaccttcaggaatttCATACTTTTCTTCCTGTTCACATACTTTTCTTCCTGTTCAcatactt | SEQ_DUP; | 483 |
| | ttcttcgtagacatgctt | | |
| | .........................1111111111111111111122222222222222222222....... | DUP_NUM; | |
| | .................. | | |
| | .a.a.aa.......aaB..J...............A..J.................A..J............ | Cas9_Wa; | |
| | ..A..a....A....... | | |
| | .Aa..A.Ba..A.....Aa.Ba......Ba...A...Ba.Baa...Ba.a....A...Ba.Baa...Ba.a. | Cas9_Cr; | |
| | ..A...Ba.Ba.....A. | | |
| | .......N......N.........N...........NL.......kL........k..NL....NL...... | Cpf1_Wa; | |
| | ...k..NL....NL.... | | |
| | .....M...................M..................M........................... | Cpf1_Cr; | |
| | ........k.......N. | | |
| MRE11A | ctgcacctacctttgatctgtctttGAAGTGGTAGGAAAAATGTCGAAGTGGTAGGAAAAATGTCttcttcc | SEQ_DUP; | 484 |
| | acatctgattcatctacc | | |
| | .........................1111111111111111111122222222222222222222....... | DUP_NUM; | |
| | .................. | | |
| | ..........aC...A......aB.a.aA..aaB.....A..aB.a.aA..aaB.....A............ | Cas9_Wa; | |
| | I..aC............. | | |
| | ..Ca.aI.A.aa..Aa......Ca...a.....................a...................a.B | Cas9_Cr; | |
| | a.Baa.a.Ca....BaHC | | |
| | .....k....k.........................k..........k........................ | Cpf1_Wa; | |
| | ................N. | | |
| | ....LN........Lk.........LN........Lk..................................N | Cpf1_Cr; | |
| | ..........N..Lk... | | |
| PYGL | ctccacgcccacgatgccgcggatgCTGATCTGCCGCCGCTTCTCCTGATCTGCCGCCGCTTCTCctggtcc | SEQ_DUP; | 485 |
| | gtcaggggcttcgccatg | | |
| | .........................1111111111111111111122222222222222222222....... | DUP_NUM; | |
| | .................. | | |
| | ..A.....aC.A..aHaaC.A..aC...A..A..A........aC...A..A..A........aA...A... | Cas9_Wa; | |
| | aaaA....A....aA..a | | |
| | .a..Ba.aa.a.aaa.a....Aa.a.....A...Ca..Aa.aa.a.Ba.aa...Ca..Aa.aa.a.Ba.aa. | Cas9_Cr; | |
| | ...aa..a.....A.Ba. | | |
| | ...N..NL.............N...L.....................................N........ | Cpf1_Wa; | |
| | ...........N...... | | |
| | ........................................................................ | Cpf1_Cr; | |
| | .................. | | |
| SLC34A1 | acccggtggccgggctggtggtgggGATCCTGGTGACCGTGCTGGTGATCCTGGTGACCGTGCTGGTgcaga | SEQ_DUP; | 486 |
| | gctccagcacctccacatcc | | |
| | .........................111111111111111111111222222222222222222222..... | DUP_NUM; | |
| | .................... | | |
| | aa.aA..aaA..aa.aa.aaaaC....aa.a...a.A..aa.aC....aa.a...a.A..aa.A..a.A..J | Cas9_Wa; | |
| | ..A................. | | |
| | ..aa..AaaH....Aa...A............CaaH.....Aa...A..J...CaaH.....Aa...A..J. | Cas9_Cr; | |
| | .A..J.A.aa..A.aa.aa. | | |
| | ....................L................................................... | Cpf1_Wa; | |
| | .................... | | |
| | ...L.................................................................... | Cpf1_Cr; | |
| | .................... | | |
| SLC25A13 | actccgctgtaagtggtttggccagCCCGGGCAGCCACCTGTAATCTCCCCGGGCAGCCACCTGTAATCTCg | SEQ_DUP; | 487 |
| | tcttgataacatcagcaggggtca | | |
| | .........................1111111111111111111111122222222222222222222222. | DUP_NUM; | |
| | ........................ | | |
| | .A..A...a.aA...aA...A...aaA..A......A..........aaA..A......A.......A.... | Cas9_Wa; | |
| | aC........A..aaaA....... | | |
| | a..Ca.a.aa.a..............Aa..Aaa...A..Aa.aa.....Ca.aaaa...A..Aa.aa..... | Cas9_Cr; | |
| | Ca.aI.a.......A.Ca..A... | | |
| | ..M...k.............M.....L.............k.............................L. | Cpf1_Wa; | |
| | ........................ | | |
| | .....................N......................N..........M.N.............. | Cpf1_Cr; | |
| | .....k..............M... | | |
| PKP2 | cggccgcctggccgacagtcaagtgCGCTCTCCTCCCGCTGGAATCCACGCTCTCCTCCCGCTGGAATCCAc | SEQ_DUP; | 488 |
| | ggcgacactgggcccagcttccct | | |
| | .........................1111111111111111111111122222222222222222222222. | DUP_NUM; | |
| | ........................ | | |
| | .A...aA..a...A....a.a.A..........A..aaB......A..........A..aaB......aa.a | Cas9_Wa; | |
| | .....aaA....A.........a. | | |
| | aaa..A..Aa.aa...Aa..A...a.....A.aJa.aa.aaa.a.....CaaHa.a.a.aa.aaa.a..... | Cas9_Cr; | |
| | CaaHa..A..A.a....Aaa..A. | | |
| | .......NLL....NLL........................................L..........L... | Cpf1_Wa; | |
| | ........L..........L.... | | |
| | ...................LLN....................LLN........................... | Cpf1_Cr; | |
| | ....................LLN. | | |
| AMH | gccgacgggccgtgcgcgctgcgcgAGCTCAGCGTAGACCTCCGCGCCAGCTCAGCGTAGACCTCCGCGCCg | SEQ_DUP; | 489 |
| | agcgctccgtactcatccccgaga | | |
| | .........................1111111111111111111111122222222222222222222222. | DUP_NUM; | |
| | ........................ | | |
| | ..aaA..a.a.a.A..a.a.a.A....a.A..a......a.A...A....a.A..a......a.A..a.a.A | Cas9_Wa; | |
| | .J..A...........a.a..... | | |
| | aa.aa.aa..A...Aa...A.aJa..A.a...A.a..A.....Aa.aa.a.aa..A.a..A.....Aa.aa. | Cas9_Cr; | |
| | a.aa...A.a.aa...A.ahCaaa | | |
| | ................................................................L....... | Cpf1_Wa; | |
| | ...............L........ | | |
| | ........................................................................ | Cpf1_Cr; | |
| | ..............N..N...... | | |
| HPS4 | gatcatggccagacaagcatccgttCTCCTTCCTGCCATCTGGACAAGCCTCCTTCCTGCCATCTGGACAAG | SEQ_DUP; | 490 |
| | Cttcgtcaggggatgtgggatctggg | | |
| | .........................11111111111111111111111122222222222222222222222 | DUP_NUM; | |
| | 2......................... | | |
| | ..aA...a....A.....A...........A......aa....A..........A......aa....A.... | Cas9_Wa; | |
| | A...aaaaC.a.aaaC...aaaa.aA | | |
| | a.aa...Ca....Aa...A...A.CaaH.Ba.aa.Baa..Aa.Ca....A...Aa.aa.Baa..Aa.Ca... | Cas9_Cr; | |
| | .A...A.Ba..a.............C | | |
| | ....................L......................L..N.....NL.................. | Cpf1_Wa; | |
| | ....NL..................NL | | |
| | ....................L...N...................L...N............L......L... | Cpf1_Cr; | |
| | ...................L...... | | |
| ASL | tgcccctggcttcccacagccacgcCGTGGCACTGACCCGAGACTCTGAGCGTGGCACTGACCCGAGACTCT | SEQ_DUP; | 491 |
| | GAGcggctgctggaggtgcggaagcgga | | |
| | .........................11111111111111111111111112222222222222222222222 | DUP_NUM; | |
| | 222......................... | | |
| | ...aA.........A....A..a.aA....a....a.a.....a.a.a.aA....a....a.a.....a.a. | Cas9_Wa; | |
| | aA..A..aa.aa.a.aaB.aHaaC.... | | |
| | aaHaa..Aaaa...A.Baaa.a..Aa.a.aa....A.a...AaaH...A.aH...A....A.a...AaaH.. | Cas9_Cr; | |
| | .A.aH...A..A..A........A..J. | | |
| | .................................NLL.................................... | Cpf1_Wa; | |
| | ............................ | | |
| | ..............................................................L.......LL | Cpf1_Cr; | |
| | N...L....N...............L.. | | |
| EPG5 | tcagagcttggtcaggggtgaaagcAGAGTTTATCACCAATTCCCCTTCAATAAGAGTTTATCACCAATTCC | SEQ_DUP; | 492 |
| | CCTTCAATAactctccggagctgggagtcctctt | | |
| | .........................11111111111111111111111111112222222222222222222 | DUP_NUM; | |
| | 222222222......................... | | |
| | .A...aA...aaaa.aB..A.Ha.A........................Ha.A................... | Cas9_Wa; | |
| | ..........aa.A..aaa.A............. | | |
| | ..Aa.ba....A.....a...........A.......Ca.aa...Baaaa.Ba.............Ca.aa. | Cas9_Cr; | |
| | ..Baaaa.Ba....A.aHaa....A........a | | |
| | ...............k......NL......N......................k.M.......NLL...NL. | Cpf1_Wa; | |
| | .........k.M.......NLL...NL....... | | |
| | .................N..........N..N.............N..........N..N.......L.... | Cpf1_Cr; | |
| | ..L...........k.....L............. | | |
| ABCC8 | ttgggcacaagaagaaaaaccacatGAGCTGATTGGTGTCGATGGCAACCAGATTAGAGCTGATTGGTGTCG | SEQ_DUP; | 493 |
| | ATGGCAACCAGATTAcagatctgtccagcagtcatttctc | | |
| | .........................11111111111111111111111111111112222222222222222 | DUP_NUM; | |
| | 222222222222222......................... | | |
| | A.....aB.aB..........a.a..aC..aa.A..aC.aA...I..aC...a.a..aC..aa.A..aC.aA | Cas9_Wa; | |
| | ...I..aC.....aC...A....A..A............. | | |
| | ..........A.a...........Aa.a.....A..........a.J...A..Aa.........A....... | Cas9_Cr; | |
| | ...a.J...A..Aa......A...Ca...aa..A...a.. | | |
| | .......................k...............................N................ | Cpf1_Wa; | |
| | ....N.........N....................N.... | | |
| | .........................N..............................N............... | Cpf1_Cr; | |
| | .......................L........L.....L. | | |
| LAMB3 | cttgccttcggtgtggtcccggcaaTTGTCACACACACCTCCATATGCCCCCTGGCTGGCGGCAAACACAGC | SEQ_DUP; | 494 |
| | GGGGTCAAAGTGACATGTCTCTGAGTGCCCTTGTCACACACACCTCCATATGCCCCCTGGCTGGCGGCAAAC | | |
| | ACAGCGGGGTCAAAGTGACATGTCTCTGAGTGCCCattgcagtcgcaccctggaaaaaga | | |
| | .........................11111111111111111111111111111111111111111111111 | DUP_NUM; | |
| | 111111111111111111111111111111222222222222222222222222222222222222222222 | | |
| | 22222222222222222222222222222222222......................... | | |
| | .....aa.a.aA....aA.....a.J.J..............A......aA..aa.aAJ.......a.aaaA | Cas9_Wa; | |
| | ......a.a....A....Ha.a.A.....a.J.J.............A......aA..aa.aAJ.......a | | |
| | .aaaA.....a.a....A....Ha.a.A......A..a..A......aaB....aHa.A. | | |
| | a...Ba...Aa.Ba.......Jaaa..A......a.a.a.a.aa.aa.....Aaaaa...A...A..A...A | Cas9_Cr; | |
| | .a..A.....a.......A....a.a......AaaJ...a.a.a.a.aa.aa.....Aaaaa...A...A.. | | |
| | A...A.a..A.....a.......A....a.a......AaaJ...A...a.a.aaaH. | | |
| | .....................N..N....NL.........L.......N..............L...M.... | Cpf1_Wa; | |
| | .....................................................N..............L... | | |
| | M..........................................................N | | |
| | ..N..................M....................k..............k.............. | Cpf1_Cr; | |
| | ..........................M....................k..............k......... | | |
| | ..............................LLk........................... | | |

The +/−1 base differences in shifts between Watson and Crick tracks is so that cleavage positions are to the immediate left of the indicated base in both cases (which wouldn't be an issue if we were labelling the spaces between bases rather than the bases themselves).

The Cpf1 cleavage sites are staggered on the two strands, leaving an overhang in the double-stranded break, not indicated in these schematics

The cleavage sites are labeled according to the Legend column in the table of PAM sequences below, Table 9 with an upper-case letter is it's the only matching PAM sequence, and a lower-case letter if it's the first of more-then-one matching PAM sequence.

Motifs are scanned for in flanking regions of size 50 and the table includes flanking regions of size 25, so cleavage sites should be shown even if the PAM site itself does not fall within the displayed sequence (as the distance between the cleavage site and the furthest position in the PAM site is no more than 25 bases). The above tracks, from top to bottom are shown for specific genes: See, Table 6

The variants identified in Table 6 with insertion lengths between 2 and 40 were then prioritized for therapeutic applications where the following microduplications were identified. See, Table 7. The headings of Table 7 are as follows:

Sequence ID: Arbitrary number assigned to each sequence.
VARIANT: of the form CHR-POS-REF-ALT, where CHR is the chromosome and POS is the start position of the reference (REF) allele in GRCh37, and ALT is the alternate allele; variants have been left-normalized with vt. genome.sph.umich.edu/wiki/Vt.
INSERT_LENGTH: length in nucleotides of the inserted sequence in the variant. (This is one less than the number of characters in ALT, as the first character of ALT is the REF base within the genome.)
ALLELE_ID: allele ID from the ClinVar VCF (version clinvar_20180225.vcf.gz)
GENE_INFO: of the form SYMBOL: ENTREZID, from the ClinVar VCF. Note: some variants have more than one gene listed in GENEINFO, and this column just shows the first of them, in the interest of space.
CLNDN: the associated disease name from the Clin Var vcf; if there is more than one disease listed in the vcf just the first is shown here in the interest of space.
MAX_AF: allele frequency for the variant from the gnomAD genomes or exomes (version 2.0.2), whichever one is larger.
Microduplication Sequences: The information on potential CRISPR cut-sites that shows the duplicated sequence, the two copies are enclosed by square brackets and separated by a vertical bar, with 5 flanking bases on either side. A base is shown in lower case if there is a predicted CRISPR cleavage site immediately to the left of the base.

+0 TABLE 7
Preferred Microduplication Seq.uences For Clinical Application

| | | | | | | | NNNNN[Duplication 1\| |
|---|---|---|---|---|---|---|---|
| | | | | | | | Duplication 2]NNNNN |
| Seq. | VARIANT | INSERT_ | ALLELE_ | GENE_ | | MAX_ | lowercase = Cas9/Cpf1 |
| ID | (CHR-POS-REF-ALT) | LENGTH | ID | INFO | CLNDN | AF | cut site |
| Seq. | 4-3494833-A- | 4 | 16312 | DOK7: | Congenital | 0.0011653 | GcTcA[gcCt\|gCct]gCcaG |
| ID. | AGCCT | | | 285489 | myasthenic | | (SEQ ID: 495) |
| 1 | | | | | syndrome | | |
| Seq. | 9-126135887-T- | 16 | 178866 | CRB2: | Focal | 0.0009596 | cccTt[ggcGCGGcccCGgcc |
| ID. | TGGCGCGGCCCC | | | 286204 | segmental | | c\|GgcgcgGcccCGgccc]Gg |
| 2 | GGCCC | | | | glomeru- | | cgc (SEQ ID: 496) |
| | | | | | losclero- | | |
| | | | | | sis 9 | | |
| Seq. | 15-72638920-G- | 4 | 18928 | HEXA: | Tay- | 0.0008041 | catAg[gaTA\|Gata]tACgG |
| ID. | GGATA | | | 3073 | Sachs | | (SEQ ID: 497) |
| 3 | | | | | disease | | |
| Seq. | 2-1481219-A- | 4 | 421275 | TPO: | not | 0.0006493 | GGaga[CggC\|CgGc]cgcgc |
| ID. | ACGGC | | | 7173 | provided | | (SEQ ID: 498) |
| 4 | | | | | | | |
| Seq. | 19-47983175-G- | 18 | 106552 | KPTN: | Mental | 0.0005026 | gcagg[AcCGACcaCatctgc |
| ID. | GACCGACCACAT | | | 11133 | retarda- | | aga\|AcCGACcaCatctgCag |
| 5 | CTGCAGA | | | | tion, | | A]AccTc (SEQ ID: 499) |
| | | | | | autosomal | | |
| | | | | | recessive | | |
| | | | | | 41 | | |
| Seq. | 19-50365057-T- | 17 | 19886 | PNKP: | Early | 0.0002277 | ttTGt[GTtgTcgAtggCGaC |
| ID. | TGTTGTCGATGG | | | 11284 | infantile | | Cc\|GTtgtcgatGgCGaCCc] |
| 6 | CGACCC | | | | epileptic | | GTttc (SEQ ID: 500) |
| | | | | | encepha- | | |
| | | | | | lopathy | | |
| | | | | | 10 | | |
| Seq. | 11-126144895-G- | 4 | 101651 | FOXR | Mito- | 0.0002261 | agagg[gAgt\|gaGT]GGctT |
| ID. | GGAGT | | | ED1: | chondrial | | (SEQ ID: 501) |
| 7 | | | | 55572 | complex | | |
| | | | | | I | | |
| | | | | | deficien- | | |
| | | | | | cy | | |
| Seq. | 2-38298287-T- | 10 | 79358 | CYP1B1: | Glaucoma, | 0.0002156 | GcaGt[ggTGgCatGalgGTG |
| ID. | TGGTGGCATGA | | | 1545 | congenital | | gcatgA]gGaAt |
| 8 | | | | | | | (SEQ ID: 502) |
| Seq. | 1-158651385-G- | 3 | 27886 | SPTA1: | Elliptocy- | 0.0001939 | cGCag[cAa\|Caa]CTggt |
| ID. | GCAA | | | 6708 | tosis 2 | | (SEQ ID: 503) |
| 9 | | | | | | | |
| Seq. | 6-158535858-A- | 4 | 423256 | SERAC1: | 3-methyl | 0.0001666 | aGaga[AgaT\|agat]aGCGA |
| ID. | AAGAT | | | 84947 | gluta- | | (SEQ ID: 504) |
| 10 | | | | | conic | | |
| | | | | | aciduria | | |
| | | | | | with | | |
| | | | | | deafness | | |
| | | | | | encepha- | | |
| | | | | | lopathy, | | |
| | | | | | and | | |
| | | | | | Leigh- | | |
| | | | | | like | | |
| | | | | | syndrome | | |
| Seq. | 9-35658024-A- | 15 | 29250 | RMRP: | Metaphy | 0.0001567 | cctCA[gcttcAcAgaGtAG |
| ID. | AGCTTCACAGAG | | | 6023 | seal | | t\|GCTtcAcagagtAGT]ATT |
| 11 | TAGT | | | | chondro | | Tt (SEQ ID: 505) |
| | | | | | dysplasia, | | |
| | | | | | McKusick | | |
| | | | | | type | | |
| Seq. | 19-7620610-C- | 4 | 21646 | PNPLA6: | Laurence- | 0.0001307 | gcAGc[gccA\|gCca]Gccgc |
| ID. | CGCCA | | | 10908 | Moon | | (SEQ ID: 506) |
| 12 | | | | | syndrome | | |
| Seq. | 1-26126724-G- | 10 | 190596 | SELENON: | Eichsfeld | 0.0001207 | ccaTG[ggcCGGgccC\|ggcc |
| ID. | GGGCCGGGCCC | | | 57190 | type | | gGgccC]ggccg (SEQ |
| 13 | | | | | congenital | | ID: 507) |
| | | | | | muscular | | |
| | | | | | dystrophy | | |
| Seq. | 19-47984017-C- | 2 | 264742 | KPTN: | not | 0.0001056 | cTaCc[tA\|ta]ctggt |
| ID. | CTA | | | 11133 | provided | | (SEQ ID: 508) |
| 14 | | | | | | | |
| Seq. | 6-1612016-C- | 6 | 136584 | FOXC1: | not | 0.0001046 | Gagtc[AcGGcg\|acggcg]g |
| ID. | CACGGCG | | | 2296 | provided | | cggc (SEQ ID: 509) |
| 15 | | | | | | | |
| Seq. | 7-107412534-C- | 3 | 70627 | SLC26A3: | Congenital | 0.0000969 | gatcC[tgaltga]tAAAT |
| ID. | CTGA | | | 1811 | secretory | | (SEQ ID: 510) |
| 16 | | | | | diarrhea, | | |
| | | | | | chloride | | |
| | | | | | type | | |
| Seq. | 5-176942945-T- | 4 | 207209 | DDX41: | Acute | 0.0000853 | GGGGT[CaTC\|CaTc]atac |
| ID. | TCATC | | | 51428 | myeloid | | g (SEQ ID: 511) |
| 17 | | | | | leukemia | | |
| Seq. | 7-95751240-G- | 23 | 21042 | SLC2 | Citrulli- | 0.0000853 | gcCAg[cccGggcaGCCaCCt |
| ID. | GCCCGGGCAGCC | | | 5A13: | nemia | | gTaatCTc\|cccGggcagCca |
| 18 | ACCTGTAATCTC | | | 10165 | type II | | CCtgTaatCTc]GtcTt |
| | | | | | | | (SEQ ID: 512) |
| Seq. | 17-37821635-G- | 8 | 441954 | TCAP: | not | 0.0000813 | agcTg[cGagGtGt\|cGaGGt |
| ID. | GCGAGGTGT | | | 8557 | provided | | gt]cgGag |
| 19 | | | | | | | (SEQ ID: 513) |
| Seq. | 9-35658027-T- | 10 | 29249 | RMRP: | Metaphy | 0.0000752 | CAgct[tcAcAgaGtA\|tcAc |
| ID. | TTCACAGAGTA | | | 6023 | seal | | agaGtA]GTatT (SEQ |
| 20 | | | | | chondro- | | ID: 514) |
| | | | | | dysplasia, | | |
| | | | | | McKusick | | |
| | | | | | type | | |
| Seq. | 17-56283862-G- | 4 | 71256 | MKS1: | Joubert | 0.0000732 | gAcAG[TgcC\|TgCc]tGtgg |
| ID. | GTGCC | | | 54903 | syndrome | | (SEQ ID: 515) |
| 21 | | | | | | | |
| Seq. | 17-48245341-A- | 5 | 467925 | SGCA: | Limb- | 0.000069 | TACCa[cTcgg\|cTcgG]cag |
| ID. | ACTCGG | | | 6442 | girdle | | Ag (SEQ ID: 516) |
| 22 | | | | | muscular | | |
| | | | | | dystrophy, | | |
| | | | | | type | | |
| | | | | | 2D | | |
| Seq. | 14-50100653-A- | 16 | 205407 | DNAAF2: | Kartagener | 0.000065 | cccCA[gccacgcaGgtatCG |
| ID. | AGCCACGCAGGT | | | 55172 | syndrome | | T\|GccAcgcaGgtatcGT]Gg |
| 23 | ATCGT | | | | | | cct (SEQ ID: 517) |
| Seq. | 3-121514389-T- | 4 | 393234 | IQCB1: | Nephro- | 0.0000648 | TGAat[CAAg\|caag]catGC |
| ID. | TCAAG | | | 9657 | nophthi- | | (SEQ ID: 518) |
| 24 | | | | | sis | | |
| Seq. | 20-3199224-A- | 8 | 214745 | ITPA: | Epileptic | 0.0000647 | [TgcA[cTCagcAc\|cTcAgc |
| ID. | ACTCAGCAC | | | 3704 | encepha- | | ac]cGgGg (SEQ ID: |
| 25 | | | | | lopathy, | | 519) |
| | | | | | early | | |
| | | | | | infantile, | | |
| | | | | | 35 | | |
| Seq. | 10-100183554-T- | 16 | 20316 | HPS1: | Hermansky- | 0.0000647 | ggtgT[GGGCCTCccctgctg |
| ID. | TGGGCCTCCCCT | | | 3257 | Pudlak | | G\|GgGCCTCccctgctgG]Gg |
| 26 | GCTGG | | | | syndrome | | Gct (SEQ ID: 520) |
| | | | | | 1 | | |
| Seq. | 6-135754331-A- | 2 | 214240 | AHI1: | Joubert | 0.0000646 | TGtaa[AC\|AC]aaaag |
| ID. | AAC | | | 54806 | syndrome | | (SEQ ID: 521) |
| 27 | | | | | 3 | | |
| Seq. | 1-216498866-G- | 4 | 57777 | USH2A: | Retinitis | 0.0000646 | gcggg[tggC\|TgGC]tgcca |
| ID. | GTGGC | | | 7399 | pigmentosa | | (SEQ ID: 522) |
| 28 | | | | | | | |
| Seq. | 18-21114427-C- | 4 | 410327 | NPC1: | Niemann- | 0.0000646 | tgGgC[AAgT\|aAGT]GCCT |
| ID. | | | | 4864 | Pick | | C (SEQ ID: 523) |
| 29 | CAAGT | | | | disease | | |
| | | | | | type C1 | | |
| Seq. | 9-35658027-T- | 7 | 264540 | RMRP: | not | 0.0000646 | CAgct[tcacAga\|tcAcag |
| ID. | TTCACAGA | | | 6023 | provided | | a]GtAGT |
| 30 | | | | | | | (SEQ ID: 524) |
| Seq. | 6-129571327-A- | 8 | 46903 | LAMA2: | Merosin | 0.0000646 | aCAgA[acGTGTtC\|aCGtgt |
| ID. | AACGTGTTC | | | 3908 | deficient | | tC]tCCag |
| 31 | | | | | congenital | | (SEQ ID: 525) |
| | | | | | muscular | | |
| | | | | | dystrophy | | |
| Seq. | 11-94169012-T- | 20 | 150739 | MRE11: | Hereditary | 0.0000646 | TctTt[gaaGTggtAggAAaA |
| ID. | TGAAGTGGTAGG | | | 4361 | cancer- | | AtgTc\|GaaGtggtAGGAAaA |
| 32 | AAAAATGTC | | | | predispo- | | ATGTC]TTCTt |
| | | | | | sing | | (SEQ ID: 526) |
| | | | | | syndrome | | |
| Seq. | 2-215595181-T- | 20 | 133182 | BARD1: | Hereditary | 0.0000609 | aaTTt[cATActTTTcTtcct |
| ID. | TCATACTTTTCTT | | | 580 | cancer- | | GttcA\|cataCtTTTcttCct |
| 33 | CCTGTTCA | | | | predispo- | | Gttca]cAtaC |
| | | | | | sing | | (SEQ ID: 527) |
| | | | | | syndrome | | |
| Seq. | 17-33434458-T- | 2 | 242729 | RAD51D: | Hereditary | 0.0000569 | caGTt[tA\|TA]tCAag |
| ID. | TTA | | | 5892 | cancer- | | (SEQ ID: 528) |
| 34 | | | | | predispo- | | |
| | | | | | sing | | |
| | | | | | syndrome | | |
| Seq. | 19-1399807-T- | 13 | 23341 | GAMT: | Deficiency | 0.0000557 | Gccgt[gggGccCAgtccc\| |
| ID. | TGGGGCCCAGTC | | | 2593 | of | | GgggcCCAGtccc]GGagc |
| 35 | CC | | | | guanidino- | | (SEQ ID: 529) |
| | | | | | acetate | | |
| | | | | | methyl- | | |
| | | | | | trans- | | |
| | | | | | ferase | | |
| Seq. | 11-95560975-T- | 11 | 39648 | CEP57: | Mosaic | 0.0000528 | GTggt[AGCcAATgtTC\|AGC |
| ID. | TAGCCAATGTTC | | | 9702 | variegated | | caAtgttc]AgCtt |
| 36 | | | | | aneuploidy | | (SEQ ID: 530) |
| | | | | | syndrome | | |
| | | | | | 2 | | |
| Seq. | 6-112390619-T- | 2 | 21424 | WISP3: | Progres- | 0.0000488 | CAAgT[aC\|ac]Tcaga |
| ID. | TAC | | | 8838 | sive | | (SEQ ID: 531) |
| 37 | | | | | pseudor | | |
| | | | | | heumatoid | | |
| | | | | | dysplasia | | |
| Seq. | 2-241808397-G- | 2 | 200432 | AGXT: | Primary | 0.0000411 | cAtgg[cA\|Ca]gccgg |
| ID. | GCA | | | 189 | hyper- | | (SEQ ID: 532) |
| 38 | | | | | oxaluria, | | |
| | | | | | type I | | |
| Seq. | 7-107335062-G- | 4 | 52676 | SLC26A4: | Pendred's | 0.0000407 | ctTgG[cAgT\|CagT]CgGtc |
| ID. | GCAGT | | | 5172 | syndrome | | (SEQ ID: 533) |
| 39 | | | | | | | |
| Seq. | 5-131705914-T- | 11 | 47395 | SLC22A5: | Renal | 0.0000385 | CCgct[acCggCtcGCclacC |
| ID. | TACCGGCTCGCC | | | 6584 | carnitine | | ggCtcGCc]Accat |
| 40 | | | | | transport | | (SEQ ID: 534) |
| | | | | | defect | | |
| Seq. | 22-26860623-T- | 24 | 19168 | HPS4: | Hermansky- | 0.0000366 | cCgTt[ctcCttCctGccatC |
| ID. | TCTCCTTCCTGC | | | 89781 | Pudlak | | tgGacAaGc\|cTCcttCctGc |
| 41 | CATCTGGACAAG | | | | syndrome | | catCtgGacAAGc]tTCgt |
| | C | | | | 4 | | (SEQ ID: 535) |
| Seq. | 5-54529099-C- | 5 | 143224 | CCNO: | Primary | 0.0000351 | Gggcc[Gggca\|Gggca]gGG |
| ID. | CGGGCA | | | 10309 | ciliary | | gG (SEQ ID: 536) |
| 42 | | | | | dyskinesia | | |
| Seq. | 4-6290805-A- | 16 | 19558 | WFS1: | Diabetes | 0.000035 | aagcA[GgGccGtCgcGAggc |
| ID. | AGGGCCGTCGCG | | | 7466 | mellitus | | T\|GgGccGtCgcGAgGct]Gt |
| 43 | AGGCT | | | | AND | | Gaa (SEQ ID: 537) |
| | | | | | insipidus | | |
| | | | | | with | | |
| | | | | | optic | | |
| | | | | | atrophy | | |
| | | | | | AND | | |
| | | | | | deafness | | |
| Seq. | 9-35658017-C- | 17 | 29253 | RMRP: | Metaphy | 0.0000327 | accAC[GtcctCAgcttcAcA |
| ID. | CGTCCTCAGCTT | | | 6023 | seal | | ga\|GtCcTCagcTtcAcaga] |
| 44 | CACAGA | | | | chondro | | GtAGT (SEQ ID: 538) |
| | | | | | dysplasia, | | |
| | | | | | McKusick | | |
| | | | | | type | | |
| Seq. | 1-76226858-G- | 13 | 18626 | ACADM: | Medium- | 0.0000325 | acTag[ctagAaTGAGTta\|c |
| ID. | GCTAGAATGAGT | | | 34 | chain | | tagAaTgAGTTa]CcAgA |
| 45 | TA | | | | acyl- | | (SEQ ID: 539) |
| | | | | | coenzyme | | |
| | | | | | A | | |
| | | | | | dehydro | | |
| | | | | | genase | | |
| | | | | | deficieny | | |
| Seq. | 5-147466073-T- | 4 | 406655 | SPINK5: | not | 0.0000324 | Cagat[gTgC\|GTGc]acTgt |
| ID. | TGTGC | | | 11005 | provided | | (SEQ ID: 540) |
| 46 | | | | | | | |
| Seq. | 6-31085224-G- | 4 | 16742626 | CDSN: | Peeling | 0.0000324 | cagtg[aggC\|Aggc]aGGgg |
| ID. | GAGGC | | | 1041 | skin | | (SEQ ID: 541) |
| 47 | | | | | syndrome | | |
| Seq. | 5-54529084-C- | 5 | 14322828 | CCNO: | Primary | 0.0000324 | Gccac[Gggct\|GggcT]Ggg |
| ID. | CGGGCT | | | 10309 | ciliary | | cC (SEQID: 542) |
| 48 | | | | | dyskinesia | | |
| Seq. | 2-219646907-T- | 10 | 26407676 | CYP27A1: | not | 0.0000324 | cccAT[ggctGcgcTG\|gGcT |
| ID. | TGGCTGCGCTG | | | 1593 | provided | | GcgcTG]gGcTg |
| 49 | | | | | | | (SEQ ID: 543) |
| Seq. | 22-37260985-A- | 10 | 22472121 | NCF4: | Chronic | 0.0000324 | GACaa[aaggAGGAtc\|aagg |
| ID. | AAAGGAGGATC | | | 4689 | granulo | | aGgATc]CAAgt |
| 50 | | | | | matous | | (SEQ ID: 544) |
| | | | | | disease | | |
| Seq. | 1-154960775-G- | 2 | 22651515 | FLAD1: | Glutaric | 0.0000323 | ttgag[Gc\|Gc]aGtGg |
| ID. | GGC | | | 80308 | aciduria, | | (SEQ ID: 545) |
| 51 | | | | | type 2 | | |
| Seq. | 2-228566952-A- | 2 | 35388989 | SLC19A3: | Basal | 0.0000323 | TCtCA[Tc\|tc]AtgGa |
| ID. | ATC | | | 80704 | ganglia | | (SEQ ID: 546) |
| 52 | | | | | disease, | | |
| | | | | | biotin- | | |
| | | | | | responsive | | |
| Seq. | 8-74888632-C-CGT | 2 | 200167 | TMEM70: | not | 0.0000323 | CgGgc[gT\|GT]cCtCC |
| ID. | | | | 54968 | provided | | (SEQ ID: 547) |
| 53 | | | | | | | |
| Seq. | 2-152364571-A- | 4 | 29086 | NEB: | Nemaline | 0.0000323 | gTCCA[AAaclaaAc]aGtCt |
| ID. | AAAAC | | | 4703 | myopathy | | (SEQ ID: 548) |
| 54 | | | | | 2 | | |
| Seq. | 4-15575920-C- | 4 | 214183 | CC2D2A: | Joubert | 0.0000323 | CCagc[tggT\|tgGt]tectG |
| ID. | CTGGT | | | 57545 | syndrome | | (SEQ ID: 549) |
| 55 | | | | | 9 | | |
| Seq. | 6-129835627-T- | 4 | 98901 | LAMA2: | Merosin | 0.0000323 | aaGAt[cAAA\|caAA]caCCg |
| ID. | TCAAA | | | 3908 | deficient | | (SEQ ID: 550) |
| 56 | | | | | congenital | | |
| | | | | | muscular | | |
| | | | | | dystrophy | | |
| Seq. | 6-158538811-C- | 4 | 211210 | SERAC1: | not | 0.0000323 | tCcac[AgaT\|aGAT]ATAat |
| ID. | CAGAT | | | 84947 | provided | | (SEQ ID: 551) |
| 57 | | | | | | | |
| Seq. | 12-123738316-T- | 4 | 211578 | C12or | not | 0.0000323 | TtGtT[ATcC\|ATcc]ccagg |
| ID. | TATCC | | | f65: | provided | | (SEQ ID: 552) |
| 58 | | | | 91574 | | | |
| Seq. | 13-20763209-G- | 4 | 186855 | GJB2: | Deafness, | 0.0000323 | cCaGg[cgTT\|cgTt]gcACt |
| ID. | GCGTT | | | 2706 | autosomal | | (SEQ ID: 553) |
| 59 | | | | | recessive | | |
| | | | | | 1A | | |
| Seq. | 13-32972540-C- | 4 | 180697 | BRCA2: | Hereditary | 0.0000323 | aAggC[ATtT\|aTtT]CAGcc |
| ID. | CATTT | | | 675 | cancer- | | (SEQ ID: 554) |
| 60 | | | | | predispo- | | |
| | | | | | sing | | |
| | | | | | syndrome | | |
| Seq. | 2-48033707-T- | 8 | 94955 | MSH6: | Lynch | 0.0000323 | TTgCt[aATcTCCc\|aatctc |
| ID. | TAATCTCCC | | | 2956 | syndrome | | cc]agagg |
| 61 | | | | | | | (SEQ ID: 555) |
| Seq. | 12-124171469-G- | 8 | 462224 | TCTN2: | Meckel- | 0.0000323 | aTCAG[ctcTGcTc\|cTcTgc |
| ID. | GCTCTGCTC | | | 79867 | Gruber | | Tc]tgcTg |
| 62 | | | | | syndrome | | (SEQ ID: 556) |
| Seq. | 15-44867171-C- | 10 | 465073 | SPG11: | Spastic | 0.0000323 | agCAc[TtcCaGgtta\|Ttcc |
| ID. | CTTCCAGGTTA | | | 80208 | paraplegia | | AGgttA]GT |
| 63 | | | | | 11, | | Tac (SEQ ID: 557) |
| | | | | | autosomal | | |
| | | | | | recessive | | |
| Seq. | 17-41246723-G- | 10 | 703900 | BRCA1: | Hereditary | 0.0000323 | [TGTG[CCacAtggcT\|CCac |
| ID. | GCCACATGGCT | | | 672 | cancer- | | atgGcT]cCacA |
| 64 | | | | | predispo- | | (SEQ ID: 558) |
| | | | | | sing | | |
| | | | | | syndrome | | |
| Seq. | 11-76867001-T- | 11 | 408477 | MYO7A: | not | 0.0000323 | gCTCT[CcAtCtaCtcG\|CcA |
| ID. | TCCATCTACTCG | | | 4647 | provided | | tctaCtcg]CcAga |
| 65 | | | | | | | (SEQ ID: 559) |
| Seq. | 17-4804916-G- | 14 | 468369 | CHRNE: | Myasthenic | 0.0000323 | gTctg[AgaGctgcGgAgcc\| |
| ID. | GAGAGCTGCGG | | | 1145 | syndrome, | | aGagctGcGgAgccJaGggc |
| 66 | AGCC | | | | congenital, | | (SEQ ID: 560) |
| | | | | | 4a, | | |
| | | | | | slow- | | |
| | | | | | channel | | |
| Seq. | 7-117188810-A- | 4 | 186745 | CFTR: | Cystic | 0.0000294 | ctgaa[agat\|aGAT]ATTAA |
| ID. | AAGAT | | | 1080 | fibrosis | | (SEQ ID: 561) |
| 67 | | | | | | | |
| Seq. | 5-148407494-A- | 4 | 244469 | SH3TC2: | not | 0.0000244 | aggCa[ggCc\|GgCc]agcag |
| ID. | AGGCC | | | 79628 | provided | | (SEQ ID: 562) |
| 68 | | | | | | | |
| Seq. | 10-104590547-G- | 4 | 16816 | CYP17A1: | Congenital | 0.0000214 | cttgg[ggaT\|gGAT]GCCTt |
| ID. | GGGAT | | | 1586 | adrenal | | (SEQ ID: 563) |
| 69 | | | | | hyperplasia | | |
| Seq. | 20-400315-C- | 7 | 150333 | RBCK1: | Poly- | 0.0000213 | AgCcc[GacgaGg\|gacGag |
| ID. | CGACGAGG | | | 10616 | glucosan | | G]aGgAg (SEQ ID: 564) |
| 70 | | | | | body | | |
| | | | | | myopathy | | |
| | | | | | 1 with or | | |
| | | | | | without | | |
| | | | | | immuno | | |
| | | | | | deficiency | | |
| Seq. | 1-94508433-G-GAC | 2 | 359278 | ABCA4: | Stargardt | 0.0000203 | aACCg[Ac\|ac]aGcTt |
| ID. | | | | 24 | disease | | (SEQ ID: 565) |
| 71 | | | | | 1 | | |
| Seq. | 2-73635784-T- | 4 | 393242 | ALMS1:7 | Alstrom | 0.0000203 | CagAT[agTA\|aGTA]TatcA |
| ID. | TAGTA | | | 840 | syndrome | | (SEQ ID: 566) |
| 72 | | | | | | | |
| Seq. | 2-74185326-A- | 4 | 23194 | DGUOK: | Mitochond- | 0.0000203 | aatga[TgaT\|TgAT]TttTc |
| ID. | ATGAT | | | 1716 | rial | | (SEQ ID: 567) |
| 73 | | | | | DNA- | | |
| | | | | | depletion | | |
| | | | | | syndrome 3, | | |
| | | | | | hepatocere- | | |
| | | | | | bral | | |
| Seq. | 17-4805917-A- | 5 | 422179 | CHRNE: | not | 0.0000203 | taCcA[gtgaG\|gtgaG]atG |
| ID. | AGTGAG | | | 1145 | provided | | AG (SEQ ID: 568) |
| 74 | | | | | | | |
| Seq. | 5-176813493-G- | 21 | 27972 | SLC34A1: | Fanconi | 0.0000203 | gtgGG[GAtcCtGgtgacCGt |
| ID. | GGATCCTGGTGA | | | 6569 | renotubular | | gctgGT\|gAtcCtGgtgacCG |
| 75 | CCGTGCTGGT | | | | syndrome 2 | | tgctGGt]gcAga |
| | | | | | | | (SEQ ID: 569) |
| Seq. | 22-29115401-A- | 4 | 185622 | CHEK2: | Hereditary | 0.000018 | TGacA[tgat\|tgat]GTAtT |
| ID. | ATGAT | | | 11200 | cancer- | | (SEQ ID: 570) |
| 76 | | | | | predispo- | | |
| | | | | | sing | | |
| | | | | | syndrome | | |
| Seq. | 2-48033769-A- | 4 | 94970 | MSH6: | Hereditary | 0.0000166 | ATGAa[TCAg\|TcaG]tcact |
| ID. | ATCAG | | | 2956 | nonpoly- | | (SEQ ID: 571) |
| 77 | | | | | posis | | |
| | | | | | colon | | |
| | | | | | cancer | | |
| Seq. | 17-33903147-A- | 4 | 358425 | PEX12: | Infantile | 0.0000163 | GgatA[AggC\|AGGC]aACAc |
| ID. | AAGGC | | | 5193 | Refsum's | | (SEQ ID: 572) |
| 78 | | | | | disease | | |
| Seq. | 17-41244495-T- | 5 | 69430 | BRCA1: | Hereditary | 0.0000163 | aATgt[TCTCA\|tcTcA]ttT |
| ID. | TTCTCA | | | 672 | cancer- | | cc (SEQ ID: 573) |
| 79 | | | | | predispo- | | |
| | | | | | sing | | |
| | | | | | syndrome | | |
| Seq. | 9-35658012-A- | 17 | 29260 | RMRP: | Metaphy | 0.0000162 | cacGA[accACGtcctCAgct |
| ID. | AACCACGTCCTC | | | 6023 | seal | | tC\|AcCacGtcctCagcTtc] |
| 80 | AGCTTC | | | | chondro | | Acaga (SEQ ID: 574) |
| | | | | | dysplasia, | | |
| | | | | | McKusick | | |
| | | | | | type | | |
| Seq. | 12-32902980-A- | 18 | 414707 | YARS2: | Mitochon- | 0.0000162 | TtTaa[CtcCTgatcAGacaT |
| ID. | ACTCCTGATCAG | | | 51067 | drial | | GAc\|CtcCtgatcAGacaTGA |
| 81 | ACATGAC | | | | diseases | | c]CtCca (SEQ ID: 575) |
| Sec | 11-17470110-T- | 31 | 429214 | ABCC8: | Persistent | 0.0000162 | CaCat[gagCTgaTtGGtgTc |
| ID. | TGAGCTGATTGG | | | 6833 | hyperin- | | gATGgCaacCaGatta\|gagC |
| 82 | TGTCGATGGCAA | | | | sulinemic | | TgaTtGGtgTcgATGgcaacC |
| | CCAGATTA | | | | hypogly- | | aGAtta]CAGaT (SEQ |
| | | | | | cemia of | | ID: 576) |
| | | | | | infancy | | |
| Seq. | 16-1841827-C- | 9 | 23168 | IGFALS: | Acid- | 0.0000142 | AgCtc[Gcgcaggct\|gcgcA |
| ID. | CGCGCAGGCT | | | 3483 | labile | | ggCt]GcccA (SEQ ID: |
| 83 | | | | | subunit | | 577) |
| | | | | | deficiency | | |
| Seq. | 15-40268931-G- | 4 | 414416 | EIF2AK4: | Familial | 0.0000134 | tGGAG[CACT\|Cact]tcgg |
| ID. | GCACT | | | 440275 | pulmonary | | g (SEQ ID: 578) |
| 84 | | | | | capillary | | |
| | | | | | hemangioma- | | |
| | | | | | tosis | | |
| Seq. | 6-38850803-T-TAG | 2 | 456184 | DNA1H8: | Primary | 0.0000128 | (Tgat[Ag\|Ag]aCACc |
| ID. | | | | 769 | ciliary | | (SEQ ID: 579) |
| 85 | | | | | dyskinesia | | |
| Seq. | 9-21971020-A- | 3 | 182930 | CDKN2A: | Hereditary | 0.0000128 | GggCa[GAc\|gAC]GgCcc |
| ID. | AGAC | | | 1029 | cancer- | | (SEQ ID: 580) |
| 86 | | | | | predispo- | | |
| | | | | | sing | | |
| | | | | | syndrome | | |
| Seq. | 2-234669554-G- | 4 | 428001 | UGT1A: | Crigler- | 0.0000122 | tCctG[cagc\|cagC]ggGtg |
| ID. | GCAGC | | | 7361 | Najjar | | (SEQ ID: 581) |
| 87 | | | | | syndrome | | |
| Seq. | 1-5935033-T- | 5 | 101577 | NPHP4: | not | 0.0000122 | GgcGt[GgaGc\|GgAgc]gTG |
| ID. | TGGAGC | | | 261734 | provided | | Tg (SEQ ID: 582) |
| 88 | | | | | | | |
| Seq. | 7-92134156-A- | 9 | 99009 | PEX1: | not | 0.0000122 | CtGCa[tCCacactg\|tCcAc |
| ID. | ATCCACACTG | | | 5189 | provided | | ActG]cctct |
| 89 | | | | | | | (SEQ ID: 583) |
| Seq. | 11-17452431-A- | 15 | 214503 | ABCC8: | not | 0.0000122 | Tggaa[gAgggagAggGaGg |
| ID. | AGAGGGAGAGG | | | 6833 | provided | | c\|gAgggagAggGAGgc]aA |
| 90 | GAGGC | | | | | | AGg (SEQ ID: 584) |
| Seq. | 2-152354227-T- | 4 | 448783 | NEB: | Nemaline | 0.0000106 | tgtat[aAcA\|AACa]CcTgt |
| ID. | TAACA | | | 4703 | myopathy | | (SEQ ID: 585) |
| 91 | | | | | 2 | | |
| Seq. | 19-47258867-C- | 4 | 267103 | FKRP: | Congenital | 0.0000088 | GgtgC[gggalgGGa]gTtc |
| ID. | CGGGA | | | 79147 | muscular | | G (SEQ ID: 586) |
| 92 | | | | | dystrophy- | | |
| | | | | | dystrogly- | | |
| | | | | | canopathy | | |
| | | | | | (with or | | |
| | | | | | without | | |
| | | | | | mental re- | | |
| | | | | | tardation) | | |
| | | | | | type B5 | | |
| Seq. | 4-995488-C- | 5 | 26960 | IDUA: | Mucopo | 0.0000088 | GaTgc[CTGct\|cTgct]cgg |
| ID. | CCTGCT | | | 3425 | lysaccha | | aG (SEQ ID: 587) |
| 93 | | | | | ridosis | | |
| | | | | | type I | | |
| Seq. | 5-176831303-C- | 18 | 390679 | F12: | Hereditary | 0.0000087 | gggtc[gGaGgcGCcgcctgg |
| ID. | CGGAGGCGCCG | | | 2161 | angione | | gtt\|gGaGGcGCcgcctgggt |
| 94 | CCTGGGTT | | | | uroticedema | | t]GgGgt (SEQ ID: 588) |
| | | | | | with | | |
| | | | | | normal | | |
| | | | | | C1 | | |
| | | | | | esterase | | |
| | | | | | inhibitor | | |
| | | | | | activity | | |
| Seq. | 2-179442173-G- | 4 | 391746 | TTN: | Limb- | 0.0000082 | caaTG[gTAT\|GTAT]CcCc |
| ID. | GGTAT | | | 7273 | girdle | | t (SEQ ID: 589) |
| 95 | | | | | muscular | | |
| | | | | | dystrophy, | | |
| | | | | | type | | |
| | | | | | 2J | | |
| Seq. | 12-109998851-T- | 15 | 200227 | MMAB: | not | 0.0000082 | tCtcT[CggcCcgGcggCac |
| ID. | TCGGCCCGGCGG | | | 326625 | provided | | A\|CggcCcgGcggCacA]Cg |
| 96 | CACA | | | | | | gcC (SEQ ID: 590) |
| Seq. | 17-41244840-C- | 2 | 46039 | BRCA1: | Hereditary | 0.0000081 | TtcaC[at]aT]tCaAa |
| ID. | CAT | | | 672 | cancer- | | (SEQ ID: 591) |
| 97 | | | | | predispo | | |
| | | | | | sing | | |
| | | | | | syndrome | | |
| Seq. | 3-48508650-G- | 3 | 19220 | TREX1: | Aicardi | 0.0000081 | gAggG[tgA\|tGA]TGtcC |
| ID. | GTGA | | | 11277 | Goutieres | | (SEQ ID: 592) |
| 98 | | | | | syndrome | | |
| | | | | | 1 | | |
| Seq. | 2-26415259-C- | 4 | 199995 | HADHA: | not | 0.0000081 | cctcc[tgat\|tgaT]aGAtg |
| ID. | CTGAT | | | 3030 | provided | | (SEQ ID: 593) |
| 99 | | | | | | | |
| Seq. | 2-148696793-C- | 4 | 39252 | ORC4: | Meier- | 0.0000081 | GatGc[TgtT\|tgTT]acTcg |
| ID. | CTGTT | | | 5000 | Gorlin | | (SEQ ID: 594) |
| 100 | | | | | syndrome | | |
| | | | | | 2 | | |
| Seq. | 3-48508676-T- | 4 | 131925 | TREX1: | Aicardi | 0.0000081 | catCT[gTcalgTca]GtGgA |
| ID. | TGTCA | | | 11277 | Goutieres | | (SEQ ID: 595) |
| 101 | | | | | syndrome | | |
| | | | | | 1 | | |
| Seq. | 8-55537899-G- | 4 | 21010 | RP1: | Retinitis | 0.0000081 | tataG[Tgaa\|tgAA]gaaaG |
| ID. | GTGAA | | | 6101 | pigmentosa | | (SEQ ID: 596) |
| 102 | | | | | 1 | | |
| Seq. | 11-108119732-T- | 4 | 264571 | ATM: | Ataxia- | 0.0000081 | tgaTt[ACag\|AcaG]TGtCc |
| ID. | TACAG | | | 472 | telangiec- | | (SEQ ID: 597) |
| 103 | | | | | tasia | | |
| | | | | | syndrome | | |
| Seq. | 15-44876096-G- | 10 | 409235 | SPG11: | Spastic | 0.0000081 | ggGtg[CAGATcCTcc\|caga |
| ID. | GCAGATCCTCC | | | 80208 | paraplegia | | tcCTCc]atact (SEQ |
| 104 | | | | | 11, | | ID: 598) |
| | | | | | autosomal | | |
| | | | | | recessive | | |
| Seq. | 19-55673062-G- | 5 | 404089 | DNAAF3: | Primary | 0.0000076 | gccgg[gcgtC\|GcGTc]Gta |
| ID. | GGCGTC | | | 352909 | ciliary | | gc (SEQ ID: 599) |
| 105 | | | | | dyskines | | |
| | | | | | ia | | |
| Seq. | 19-50364929-G- | 5 | 203579 | PNKP: | Early | 0.0000071 | gCccg[CggcT\|cggct]cGG |
| ID. | GCGGCT | | | 11284 | infantile | | gc (SEQ ID: 600) |
| 106 | | | | | epileptic | | |
| | | | | | encephalo- | | |
| | | | | | pathy | | |
| | | | | | 10 | | |
| Seq. | 13-32911149-A- | 2 | 248942 | BRCA2: | Breast- | 0.0000044 | gacaa[tg\|tG]AGAAT |
| ID. | ATG | | | 675 | ovarian | | (SEQ ID: 601) |
| 107 | | | | | cancer, | | |
| | | | | | familial | | |
| | | | | | 2 | | |
| Seq. | 13-32912466-C- | 4 | 66246 | BRCA2: | Hereditary | 0.0000044 | tatAC[TgCt\|tgCt]gCCag |
| ID. | CTGCT | | | 675 | cancer- | | (SEQ ID: 602) |
| 108 | | | | | predispo- | | |
| | | | | | sing | | |
| | | | | | syndrome | | |
| Sec | 7-65551736-C- | 25 | 200151 | ASL: | not | 0.0000044 | CacGc[cGtGgcACtgaCcCG |
| ID. | CCGTGGCACTGA | | | 435 | provided | | AgactcTgag\|cgTGGcACTG |
| 109 | CCCGAGACTCTG | | | | | | aCcCGAgactctgaG]cgGC |
| | AG | | | | | | (SEQ ID: 603) |
| Seq. | 17-4802524-C- | 7 | 467870 | CHRNE: | Myasthenic | 0.0000043 | gcCcC[TcaaaCa\|TCAaac |
| ID. | CTCAAACA | | | 1145 | syndrome, | | a]CGAgC (SEQ ID: 604) |
| 110 | | | | | congenital, | | |
| | | | | | 4a, slow- | | |
| | | | | | channel | | |
| Seg | 19-2251669-G- | 23 | 23664 | AMH: | Persistent | 0.0000043 | gcgcG[AgctcAGcGtAGaCc |
| ID. | GAGCTCAGCGTA | | | 268 | mullerian | | TcCgcgcc\|AgctcAGcGtAG |
| 111 | GACCTCCGCGCC | | | | duct | | aCcTcCgcGcc]gagcg |
| | | | | | syndrome, | | (SEQ ID: 605) |
| | | | | | type I | | |
| Seq. | 14-77757715-G- | 2 | 266684 | POMT2: | Congenital | 0.0000042 | aaTag[gt\|gt]GGTga |
| ID. | GGT | | | 29954 | muscular | | (SEQ ID: 606) |
| 112 | | | | | dystrophy- | | |
| | | | | | dystrogly- | | |
| | | | | | canopathy | | |
| | | | | | with | | |
| | | | | | brain | | |
| | | | | | and eye | | |
| | | | | | anomalies, | | |
| | | | | | type | | |
| | | | | | A2 | | |
| Seq. | 17-41242962-T- | 2 | 69791 | BRCA1: | Hereditary | 0.0000042 | taccT[gA\|ga]GTGGt |
| ID. | TGA | | | 672 | cancer- | | (SEQ ID: 607) |
| 113 | | | | | predispo- | | |
| | | | | | sing | | |
| | | | | | syndrome | | |
| Seq. | 2-48026541-G-GGT | 2 | 94667 | MSH6: | Hereditary | 0.0000041 | ccCtg[gt\|gt]gCaga |
| ID. | | | | 2956 | nonpoly- | | (SEQ ID: 608) |
| 114 | | | | | posis | | |
| | | | | | colon | | |
| | | | | | cancer | | |
| Seq. | 5-13793822-C-CTA | 2 | 395008 | DNAH5: | Primary | 0.0000041 | CgcAC[tA\|tA]tctca |
| ID. | | | | 1767 | ciliary | | (SEQ ID: 609) |
| 115 | | | | | dyskinesia | | |
| Seq. | 7-117243689-G- | 2 | 68231 | CFTR: | Cystic | 0.0000041 | cgtgG[gA\|ga]gTagC |
| ID. | GGA | | | 1080 | fibrosis | | (SEQ ID: 610) |
| 116 | | | | | | | |
| Seq. | 16-23647131-T- | 2 | 478268 | PALB2: | Hereditary | 0.0000041 | GTagt[CG\|CG]ccCtg |
| ID. | TCG | | | 79728 | cancer- | | (SEQ ID: 611) |
| 117 | | | | | predispo- | | |
| | | | | | sing | | |
| | | | | | syndrome | | |
| Seq. | 17-41245704-G- | 2 | 249143 | BRCA1: | Breast- | 0.0000041 | aGAAG[AC\|AC]TTcCt |
| ID. | GAC | | | 672 | ovarian | | (SEQ ID: 612) |
| 118 | | | | | cancer, | | |
| | | | | | familial | | |
| | | | | | 1 | | |
| Seq. | 2-26502064-C- | 4 | 425487 | HADHB: | not | 0.0000041 | CtgGC[cgCT\|cgCt]gectT |
| ID. | CCGCT | | | 3032 | provided | | (SEQ ID: 613) |
| 119 | | | | | | | |
| Seq. | 2-48030716-T- | 4 | 214607 | MSH6: | Hereditary | 0.0000041 | TTCct[aatg\|AATg]aCATT |
| ID. | TAATG | | | 2956 | nonpoly- | | (SEQ ID: 614) |
| 120 | | | | | posis | | |
| | | | | | colon | | |
| | | | | | cancer | | |
| Seq. | 2-48032775-T- | 4 | 451638 | MSH6: | Hereditary | 0.0000041 | tttgt[tgaA\|TgAA]tTaaG |
| ID. | TTGAA | | | 2956 | nonpoly- | | (SEQ ID: 615) |
| 121 | | | | | posis | | |
| | | | | | colon | | |
| | | | | | cancer | | |
| Seq. | 2-48033355-C- | 4 | 419562 | MSH6: | Hereditary | 0.0000041 | ACTgC[AaCalaACa]tTtga |
| ID. | CAACA | | | 2956 | cancer- | | (SEQ ID: 616) |
| 122 | | | | | predispo- | | |
| | | | | | sing | | |
| | | | | | syndrome | | |
| Seq. | 2-48033448-C- | 4 | 231582 | MSH6: | Hereditary | 0.0000041 | caTTc[aTTalatta]gTagA |
| ID. | CATTA | | | 2956 | cancer- | | (SEQ ID: 617) |
| 123 | | | | | predispo- | | |
| | | | | | sing | | |
| | | | | | syndrome | | |
| Seq. | 2-48033767-G- | 4 | 182191 | MSH6: | Hereditary | 0.0000041 | agATG[AATC\|AaTc]aGtca |
| ID. | GAATC | | | 2956 | nonpoly- | | SEQ ID: 618) |
| 124 | | | | | posis | | |
| | | | | | colon | | |
| | | | | | cancer | | |
| Seq. | 2-215646137-A- | 4 | 232427 | BARD1: | Hereditary | 0.0000041 | TcTGA[cttT\|ctTT]ctTAc |
| ID. | ACTTT | | | 580 | cancer- | | (SEQ ID: 619) |
| 125 | | | | | predispo- | | |
| | | | | | sing | | |
| | | | | | syndrome | | |
| Seq. | 3-48626421-C- | 4 | 411522 | COL7A1: | Recessive | 0.0000041 | Caggc[tCAg\|tcaG]Gggct |
| ID. | CTCAG | | | 1294 | dystrophic | | (SEQ ID: 620) |
| 126 | | | | | epiderm | | |
| | | | | | olysis | | |
| | | | | | bullosa | | |
| Seq. | 4-6302429-C- | 4 | 211040 | WFS1: | not | 0.0000041 | GTacC[Tgat\|tgaT]TGacA |
| ID. | CTGAT | | | 7466 | provided | | (SEQ ID: 621) |
| 127 | | | | | | | |
| Seg | 9-6553398-G- | 4 | 266395 | GLDC: | Non- | 0.0000041 | agatG[gaAC\|GaaC]tGGAg |
| ID. | GGAAC | | | 2731 | ketotic | | (SEQ ID: 622) |
| 128 | | | | | hypergly | | |
| | | | | | cinemia | | |
| Seq. | 9-36246039-C- | 4 | 265527 | GNE: | Inclusion | 0.0000041 | catgc[gaAT\|gaAt]GATGC |
| ID. | CGAAT | | | 10020 | body | | (SEQ ID: 623) |
| 129 | | | | | myopathy | | |
| | | | | | 2 | | |
| Seq. | 19-11222244-A- | 4 | 228160 | LDLR: | Familial | 0.0000041 | ctgga[gggT\|ggGT]ggCTa |
| ID. | AGGGT | | | 3949 | hypercho- | | (SEQ ID: 624) |
| 130 | | | | | lesterol | | |
| | | | | | emia | | |
| Seq. | 11-47367805-A- | 5 | 178182 | MYBPC3: | Cardiomyo- | 0.0000041 | tAgCA[tGccG\|tGccG]cgc |
| ID. | ATGCCG | | | 4607 | pathy | | aG (SEQ ID: 625) |
| 131 | | | | | | | |
| Seq. | 1-45798772-C- | 6 | 232268 | MUTYH: | Hereditary | 0.0000041 | CctgC[atcCaT\|atccat]c |
| ID. | CATCCAT | | | 4595 | cancer- | | cggt (SEQ ID: 626) |
| 132 | | | | | predispo- | | |
| | | | | | sing | | |
| | | | | | syndrome | | |
| Seq. | 5-131726404-T- | 6 | 359651 | SLC22A5: | not | 0.0000041 | ctAtt[tTGGgc\|tTgggC]t |
| ID. | TTTGGGC | | | 6584 | provided | | TtCg (SEQ ID: 627) |
| 133 | | | | | | | |
| Seq. | 20-25288616-G- | 7 | 15065 | ABHD12: | Polyneu | 0.0000041 | ggATG[GCTctta\|GcTcTT |
| ID. | GGCTCTTA | | | 26090 | ropathy, | | a]gcTtc (SEQ ID: 628) |
| 134 | | | | | hearing | | |
| | | | | | loss, | | |
| | | | | | ataxia, | | |
| | | | | | retinitis | | |
| | | | | | pigmentosa, | | |
| | | | | | and | | |
| | | | | | cataract | | |
| Seq. | 13-32918751-C- | 8 | 262824 | BRCA2: | Breast- | 0.0000041 | AAAtc[aAgaaAaa\|AaGAAA |
| ID. | CAAGAAAAA | | | 675 | ovarian | | AA]TCCTt (SEQ ID: |
| 135 | | | | | cancer, | | 629) |
| | | | | | familial | | |
| | | | | | 2 | | |
| Seq. | 19-11216255-A- | 8 | 245718 | LDLR: | Familial | 0.0000041 | GGaca[AaTcTGAc\|aaTctG |
| ID. | AAATCTGAC | | | 3949 | hyperch | | ac]gAGga (SEQ ID: |
| 136 | | | | | olesterol | | 630) |
| | | | | | emia | | |
| Seq. | 11-2591894-G- | 11 | 247639 | KCNQ1: | Long | 0.0000041 | gtacG[TGgtcCgcctc\|Tgg |
| ID. | GTGGTCCGCCTC | | | 3784 | QT | | TcCGCctc]tggTc (SEQ |
| 137 | | | | | syndrome | | ID: 631) |
| | | | | | 1 | | |
| Seq. | 16-23641191-G- | 17 | 244957 | PALB2: | Hereditary | 0.0000041 | aAatg[AgCAagttGgGgTGt |
| ID. | GAGCAAGTTGG | | | 79728 | cancer- | | gc\|AgCAagttGgGgtGtGC] |
| 138 | GGTGTGC | | | | predis- | | AgCAa (SEQ ID: 632) |
| | | | | | posing | | |
| | | | | | syndrome | | |
| Seq. | 19-11216242-C- | 18 | 18772 | LDLR: | Familial | 0.0000041 | GccCC[gactgcAaGgaCAAa |
| ID. | CGACTGCAAGG | | | 3949 | hypercho- | | TcT\|gActgcaaGGaCAAaTc |
| 139 | ACAAATCT | | | | lestero- | | t]GacgA (SEQ ID: 633) |
| | | | | | lemia | | |
| Seq. | 2-48033727-T- | 19 | 94960 | MSH6: | Hereditary | 0.0000041 | GTTaT[tCaaaagggacaTaG |
| ID. | TTCAAAAGGGAC | | | 2956 | nonpoly- | | aAAA\|tCaaaagGGacaTaGa |
| 140 | ATAGAAAA | | | | posis | | AAa\|gcaaG (SEQ ID: |
| | | | | | colon | | 634) |
| | | | | | cancer | | |
| Seq. | 14-51411077-G- | 20 | 187131 | PYGL: | Glycogen | 0.0000041 | ggatg[cTGaTctGCcgCCgc |
| ID. | GCTGATCTGCCG | | | 5836 | storage | | Ttctc\|CtgatctGCcgCCgc |
| 141 | CCGCTTCTC | | | | disease, | | Ttctc]Ctggt (SEQ ID: |
| | | | | | type VI | | 635) |
| Seq. | 12-33030862-G- | 23 | 398966 | PKP2: | Arrhyth | 0.0000041 | aagTg[CGCTCtCctcCcgct |
| ID. | GCGCTCTCCTCC | | | 5318 | mogenic | | ggaatcCA\|CgctctCctcCc |
| 142 | CGCTGGAATCCA | | | | right | | gctggAaTccA]cggcg |
| | | | | | ventri- | | (SEQ ID: 636) |
| | | | | | cular | | |
| | | | | | cardiom- | | |
| | | | | | yopathy, | | |
| | | | | | type 9 | | |
| Seq. | 18-43493697-C- | 28 | 469361 | EPG5: | Absent | 0.0000041 | Aaagc[AGAgtttATCACCaa |
| ID. | CAGAGTTTATCA | | | 57724 | corpus | | TtcCCcttcaaTalaGagTTt |
| 143 | CCAATTCCCCTT | | | | callosum | | ATCaccaaTtcCCCttcaaT |
| | CAATA | | | | cataract | | a]aCtct (SEQ ID: 637) |
| | | | | | immuno | | |
| | | | | | deficiency | | |

As noted earlier there are over 2000 duplications annotated as pathogenic in Clin Var that do not appear in gnomAD at all, and hence are not listed in table 6 above, but may nonetheless be promising candidates for MMEJ. In particular the developers of gnomAD have “made every effort to exclude individuals with severe pediatric diseases from the gnomAD data set” (gnomad.broadinstitute.org/faq), and because of this, allele frequencies for dominant diseases in particular may be underestimated in gnomAD, or the variants may be entirely absent. To illustrate some of the potential MMEJ candidates of this sort, in Table 8 below we list those duplications of length 4-20 that satisfy all of the conditions from column dup2iP from Table 4 except they are absent from gnomAD, and for which the OMIM ID associated with the Clin Var entry in listed as having an autosomal dominant mode of inheritance. The columns are the same as for Table 7, although the MAX_AF column is excluded, as these variants do not appear in gnomAD.

TABLE 8
Additonal Microduplication Sequences Associated with Autosomal Dominant Diseases

| | | | | | | NNNNN |
|---|---|---|---|---|---|---|
| | | | | | | [Duplication 1\| |
| | | | | | | Duplication 2] |
| Sequence | | INS_ | | GENE | | NNNNN lowercase = |
| ID | VARIANT | LEN | ALLELEID | INFO | CLNDN | Cas9/Cpf1 cut site |
| B1- | 1-149898309-T- | 4 | 205400 | SF3B4: | Nager syndrome | Aggat[Tggg\|TgGG]agcag |
| 638 | TTGGG | | | 10262 | | (SEQ ID: 810) |
| B2- | 2-16082251-A- | 4 | 426707 | MYCN: | Feingold syndrome 1 | TtTGa[CTCg\|CtCg]ctAca |
| 639 | ACTCG | | | 4613 | | (SEQ ID: 811) |
| B3- | 2-48033635-T- | 4 | 94949 | MSH6: | Hereditary nonpolyposis | agAcT[ATTa\|AtTa]CGtT |
| 640 | TATTA | | | 2956 | colon cancer | C (SEQ ID: 812) |
| B4- | 2-145156539-T- | 4 | 442547 | ZEB2: | Mowat-Wilson syndrome | GttAt[ggAg\|GGaG]TCca |
| 641 | TGGAG | | | 9839 | | T (SEQ ID: 813) |
| B5- | 2-145156576-A- | 4 | 101526 | ZEB2: | Mowat-Wilson syndrome | atAaa[gAgT\|GAGT]Ctttt |
| 642 | AGAGT | | | 9839 | | (SEQ ID: 814) |
| B6- | 2-166848432-T- | 4 | 187710 | SCN1A: | Severe myoclonic | agGat[gACC\|gAcc]gcGat |
| 643 | TGACC | | | 6323 | epilepsy in infancy | (SEQ ID: 815) |
| B7- | 2-166848788-G- | 4 | 187727 | SCN1A: | Severe myoclonic | aaGGG[ACAT\|aCaT]CAt |
| 644 | GACAT | | | 6323 | epilepsy in infancy | ca (SEQ ID: 816) |
| B8- | 2-166897853-G- | 4 | 187812 | SCN1A: | Severe myoclonic | aaaTg[ggTC\|GgTC]cATC |
| 645 | GGGTC | | | 6323 | epilepsy in infancy | a (SEQ ID: 817) |
| B9- | 3-128200679-A- | 4 | 213545 | GATA2: | Lymphedema, primary, | TaGta[gAgg\|GAgG]CcaC |
| 646 | AGAGG | | | 2624 | with myelodysplasia | a (SEQ ID: 818) |
| B10- | 3-128200780-G- | 4 | 227183 | GATA2: | Dendritic cell, | tctgG[cggC\|CgGC]cgact |
| 647 | GCGGC | | | 2624 | monocyte, B lymphocyte, | (SEQ ID: 819) |
| | | | | | and natural killer | |
| | | | | | lymphocyte deficiency | |
| B11- | 5-36985675-C- | 4 | 207223 | NIPBL: | Cornelia de Lange | cgagC[tgaA\|tGaA]GCCTt |
| 648 | CTGAA | | | 25836 | syndrome 1 | (SEQ ID: 820) |
| B12- | 5-37019478-C- | 4 | 207238 | NIPBL: | Cornelia de Lange | AAAAc[ACTg\|actg]Agac |
| 649 | CACTG | | | 25836 | syndrome 1 | T (SEQ ID: 821) |
| B13- | 5-37045600-T- | 4 | 428429 | NIPBL: | Cornelia de Lange | cAcTt[cTaA\|ctAA]CaAA |
| 650 | TCTAA | | | 25836 | syndrome 1 | c (SEQ ID: 822) |
| B14- | 5-112173974-T- | 4 | 453932 | APC: | Familial adenomatous | AGTgt[CAgc\|cagc]caTtc |
| 651 | TCAGC | | | 324 | polyposis 1 | (SEQ ID: 823) |
| B15- | 5-176696704-A- | 4 | 207196 | NSD1: | Sotos syndrome 1 | TCtaa[tgAC\|TGAC]taTtt |
| 652 | ATGAC | | | 64324 | | (SEQ ID: 824) |
| B16- | 6-7578754-A- | 4 | 456063 | DSP: | Arrhythmogenic right | TtaCa[ggtt\|GGTT]CtTaa |
| 653 | AGGTT | | | 1832 | ventricular | (SEQ ID: 825) |
| | | | | | cardiomyopathy, type 8 | |
| B17- | 6-42689651-T- | 4 | 28213 | PRPH2: | Patterned dystrophy of | cCggt[agTA\|AGTA]CTtC |
| 654 | TAGTA | | | 5961 | retinal pigment | A (SEQ ID: 826) |
| | | | | | epithelium | |
| B18- | 7-155604807-G- | 4 | 76761 | SHH: | Holoprosencephaly 3 | gccag[Cagc\|CaGC]agCAt |
| 655 | GCAGC | | | 6469 | | (SEQ ID: 827) |
| B19- | 9-135779065-T- | 4 | 459513 | TSC1: | Tuberous sclerosis 1 | gagct[gCtg\|Gctg]ctttG |
| 656 | TGCTG | | | 7248 | | (SEQ ID: 828) |
| B20- | 10-76789950-G- | 4 | 39483 | KAT6B: | Young Simpson syndrome | caACG[cCAa\|ccaA]catT |
| 657 | GCCAA | | | 23522 | | G (SEQ ID: 829) |
| B21- | 10-88678975-C- | 4 | 397998 | BMPRIA: | Juvenile polyposis | AGcTc[tAtT\|tatt]tgaTt |
| 658 | CTATT | | | 657 | syndrome | (SEQ ID: 830) |
| B22- | 11-31823171-C- | 4 | 424532 | PAX6: | Aniridia 1 | CAagc[aAAg\|aAag]Atgg |
| 659 | CAAAG | | | 5080 | | a (SEQ ID: 831) |
| B23- | 11-31823224-G- | 4 | 485944 | PAX6: | Aniridia 1 | ttCtg[gagT\|GaGt]CGCtA |
| 660 | GGAGT | | | 5080 | | (SEQ ID: 832) |
| B24- | 11-44129659-C- | 4 | 264541 | EXT2: | Multiple exostoses | TGaAC[tgCT\|tgCt]cATg |
| 661 | CTGCT | | | 2132 | type 2 | G (SEQ ID: 833) |
| B25- | 11-47372123-C- | 4 | 248635 | MYBPC3: | Familial hypertrophic | TggCc[TcAg\|tcag]cagGg |
| 662 | CTCAG | | | 4607 | cardiomyopathy 4 | (SEQ ID: 834) |
| B26- | 12-32977056-T- | 4 | 399705 | PKP2: | Arrhythmogenic right | cTtCt[cAtc\|caTc]gcttt |
| 663 | TCATC | | | 5318 | ventricular | (SEQ ID: 835) |
| | | | | | cardiomyopathy, type 9 | |
| B27- | 12-114832576-A- | 4 | 462429 | TBX5: | Aortic valve disease 2 | ctaTa[aACg\|AAcg]CAGt |
| 664 | AAACG | | | 6910 | | c (SEQ ID: 836) |
| B28- | 13-32900728-C- | 4 | 66682 | BRCA2: | Breast-ovarian cancer, | CcaCc[ctta/ctta]GttCt |
| 665 | CCTTA | | | 675 | familial 2 | (SEQ ID: 837) |
| B29- | 13-32903617-T- | 4 | 180558 | BRCA2: | Familial cancer of | Ctcat[gatA\|GATA]CtACt |
| 666 | TGATA | | | 675 | breast | (SEQ ID: 838) |
| B30- | 13-32906470-G- | 4 | 234629 | BRCA2: | Hereditary cancer- | gAAAG[TCaA\|Tcaa]tgcC |
| 667 | GTCAA | | | 675 | predisposing syndrome | a (SEQ ID: 839) |
| B31- | 13-32906777-G- | 4 | 183659 | BRCA2: | Hereditary cancer- | aGTTG[taCC\|tACc]gTctt |
| 668 | GTACC | | | 675 | predisposing syndrome | (SEQ ID: 840) |
| B32- | 13-32907062-G- | 4 | 261077 | BRCA2: | Hereditary breast and | tcttG[cagt\|caGt]AaagC |
| 669 | GCAGT | | | 675 | ovarian cancer syndrome | (SEQ ID: 841) |
| B33- | 13-32910470-A- | 4 | 261113 | BRCA2: | Breast-ovarian cancer, | cTtAa[cTAg\|CtAG]ctCtt |
| 670 | ACTAG | | | 675 | familial 2 | (SEQ ID: 842) |
| B34- | 13-32913932-G- | 4 | 261296 | BRCA2: | Breast-ovarian cancer, | aCtTG[TgAc\|TGaC]taGct |
| 671 | GTGAC | | | 675 | familial 2 | (SEQ ID: 843) |
| B35- | 13-32914339-T- | 4 | 66619 | BRCA2: | Hereditary cancer- | GtgAT[gttA\|gTTA]gttTG |
| 672 | TGTTA | | | 675 | predisposing syndrome | (SEQ ID: 844) |
| B36- | 13-32914758-A- | 4 | 180628 | BRCA2: | Familial cancer of | acTGa[gcAT\|gcaT]AgtC |
| 673 | AGCAT | | | 675 | breast | T (SEQ ID: 845) |
| B37- | 13-32914858-G- | 4 | 183912 | BRCA2: | Hereditary cancer- | aaatG[gaaA\|GAAa]AAaC |
| 674 | GGAAA | | | 675 | predisposing syndrome | C (SEQ ID: 846) |
| B38- | 13-32929209-G- | 4 | 261402 | BRCA2: | Breast-ovarian cancer, | CTTTG[{TCC\|TTcc]aCctt |
| 675 | GTTCC | | | 675 | familial 2 | (SEQ ID: 847) |
| B39- | 13-32936669-T- | 4 | 131698 | BRCA2: | Hereditary breast and | Tgtgt[gaCa\|GaCa]ctcca |
| 676 | TGACA | | | 675 | ovarian cancer syndrome | (SEQ ID: 848) |
| B40- | 13-32936731-G- | 4 | 261438 | BRCA2: | Breast-ovarian cancer, | agaTg[gaTC\|GaTc]atatg |
| 677 | GGATC | | | 675 | familial 2 | (SEQ ID: 849) |
| B41- | 13-32937507-A- | 4 | 67186 | BRCA2: | Breast-ovarian cancer, | acaga[tggg\|Tggg]TGgTA |
| 678 | ATGGG | | | 675 | familial 2 | (SEQ ID: 850) |
| B42- | 13-32944599-C- | 4 | 261474 | BRCA2: | Breast-ovarian cancer, | tgacc[CTAg\|ctaG]AccTT |
| 679 | CCTAG | | | 675 | familial 2 | (SEQ ID: 851) |
| B43- | 13-32950917-G- | 4 | 131727 | BRCA2: | Hereditary cancer- | ccCAG[ctta\|ctTA]ccttg |
| 680 | GCTTA | | | 675 | predisposing syndrome | (SEQ ID: 852) |
| B44- | 14-95577661-G- | 4 | 463789 | DICER1: | DICER1-related | ttttg[ggTA\|ggta]GcACT |
| 681 | GGGTA | | | 23405 | pleuropulmonary | (SEQ ID: 853) |
| | | | | | blastoma cancer | |
| | | | | | predisposition | |
| | | | | | syndrome | |
| B45- | 16-2121893-C- | 4 | 27434 | TSC2: | Tuberous sclerosis | TGccC[TAct\|taCt]cCCtG |
| 682 | CTACT | | | 7249 | syndrome | (SEQ ID: 854) |
| B46- | 16-23637659-A- | 4 | 180720 | PALB2: | Hereditary cancer- | CTTta[cAAc\|CaaC]cgGC |
| 683 | ACAAC | | | 79728 | predisposing syndrome | t (SEQ ID: 855) |
| B47- | 17-29559831-A- | 4 | 425126 | NF1: | Neurofibromatosis, | aGgca[CTgt\|ctgt]AcGGT |
| 684 | ACTGT | | | 4763 | type 1 | (SEQ ID: 856) |
| B48- | 17-29657434-T- | 4 | 401825 | NF1: | Neurofibromatosis, | TCTCT[AtTa\|aTtA]gTA |
| 685 | TATTA | | | 4763 | type 1 | Ag (SEQ ID: 857) |
| B49- | 17-42426621-G- | 4 | 31049 | GRN: | Frontotemporal | gcCtG[CtgC\|Ctgc]ctGGa |
| 686 | GCTGC | | | 2896 | dementia, | (SEQ ID: 858) |
| | | | | | ubiquitin-positive | |
| B50- | 17-59763312-T- | 4 | 402202 | BRIP1: | Familial cancer of | Ctggt[gaTA\|gata]GATga |
| 687 | TGATA | | | 83990 | breast | (SEQ ID: 859) |
| B51- | 18-48586254-T- | 4 | 36159 | SMAD4: | Juvenile polyposis | GagCT[tgCa\|TgCa]TtccA |
| 688 | TTGCA | | | 4089 | syndrome | (SEQ ID: 860) |
| B52- | 19-11200255-A- | 4 | 245343 | LDLR: | Familial | CTGgA[cCgT\|CcgT]Cgc |
| 689 | ACCGT | | | 3949 | hypercholesterolemia | CT (SEQ ID: 861) |
| B53- | 19-11216012-C- | 4 | 245559 | LDLR | Familial | ctgcc[cggt\|cgGT]GcTCa |
| 690 | CCGGT | | | 3949 | hypercholesterolemia | (SEQ ID: 862) |
| B54- | 19-11218164-G- | 4 | 245855 | LDLR: | Familial | gacTg[gTcA\|gtCa]gATg |
| 691 | GGTCA | | | 3949 | hypercholesterolemia | A (SEQ ID: 863) |
| B55- | 19-11222196-A- | 4 | 245974 | LDLR: | Familial | atcGa[tgag\|TgAG]tgtca |
| 692 | ATGAG | | | 3949 | hypercholesterolemia | (SEQ ID: 864) |
| B56- | 19-11222247-G- | 4 | 245999 | LDLR: | Familial | gaggG[TggC\|TggC]taCaa |
| 693 | GTGGC | | | 3949 | hypercholesterolemia | (SEQ ID: 865) |
| B57- | 19-11224220-T- | 4 | 228168 | LDLR: | Familial | agctt[gAcA\|gAca]GAGcc |
| 694 | TGACA | | | 3949 | hypercholesterolemia | (SEQ ID: 866) |
| B58- | 19-11224266-G- | 4 | 246144 | LDLR: | Familial | cagaG[aCaT\|ACat]cCagg |
| 695 | GACAT | | | 3949 | hypercholesterolemia | (SEQ ID: 867) |
| B59- | 19-11226884-C- | 4 | 246281 | LDLR: | Familial | TCAcc[ctAg\|ctaG]gtATg |
| 696 | CCTAG | | | 3949 | hypercholesterolemia | (SEQ ID: 868) |
| B60- | 19-11231165-C- | 4 | 246517 | LDLR: | Familial | CatGC[tgCT\|tgCt]GgccA |
| 697 | CTGCT | | | 3949 | hypercholesterolemia | (SEQ ID: 869) |
| B61- | 19-11233960-C- | 4 | 390629 | LDLR: | Familial | CtcCc[ggct\|gGCt]gcctG |
| 698 | CGGCT | | | 3949 | hypercholesterolemia | (SEQ ID: 870) |
| B62- | 19-11240244-T- | 4 | 18744 | LDLR: | Familial | ggctt[aaga\|aAGA]ACaTC |
| 699 | TAAGA | | | 3949 | hypercholesterolemia | (SEQ ID: 871) |
| B63- | 22-24133984-G- | 4 | 469954 | SMARCB1: | Rhabdoid tumor | acaag[Agat\|aGAT]ACcC |
| 700 | GAGAT | | | 6598 | predisposition | C (SEQ ID: 872) |
| | | | | | syndrome 1 | |
| B64- | 22-29121073-C- | 4 | 222871 | CHEK2: | Familial cancer of | tGaTC[{TCt\|ttct]AtgtA |
| 701 | CTTCT | | | 11200 | breast | (SEQ ID: 873) |
| B65- | 22-29130554-G- | 4 | 471012 | CHEK2: | Familial cancer of | gaGAG[gACT\|gaCt]ggCt |
| 702 | GGACT | | | 11200 | breast | G (SEQ ID: 874) |
| B66- | 22-29886645-A- | 4 | 227465 | NEFH: | Charcot-Marie-Tooth | CAgCa[agCc\|aGCc]tccag |
| 703 | AAGCC | | | 4744 | disease, axonal, | (SEQ ID: 875) |
| | | | | | type 2CC | |
| B67- | 22-41572424-C- | 4 | 247756 | EP300: | Rubinstein-Taybi | ccacC[atgt\|aTGT]gCAtg |
| 704 | CATGT | | | 2033 | syndrome 2 | (SEQ ID: 876) |
| B68- | 1-153800743-G- | 5 | 423707 | GATAD2B: | Mental retardation, | cCAGG[acATC\|acAtc]At |
| 705 | GACATC | | | 57459 | autosomal dominant 18 | Ctc (SEQ ID: 877) |
| B69- | 1-155317481-T- | 5 | 440033 | ASH1L: | MENTAL RETARDATION, | tTCat[cctTg\|ccttg]tagAG |
| 706 | TCCTTG | | | 55870 | AUTOSOMAL DOMINANT 52 | (SEQ ID: 878) |
| B70- | 2-166848901-A- | 5 | 187732 | SCN1A: | Severe myoclonic | CGAaA[tACTt\|TAcTT]T |
| 707 | ATACTT | | | 6323 | epilepsy in infancy | tcTa (SEQ ID: 879) |
| B71- | 3-39453229-G- | 5 | 75607 | RPSA: | Asplenia, isolated | gggaG[GtcaT\|GtcAT]gcc |
| 708 | GGTCAT | | | 3921 | congenital | TG (SEQ ID: 880) |
| B72- | 3-136162210-A- | 5 | 431522 | STAG1: | STAG1-related disorder | TCcca[TaaaC\|tAaac]TGT |
| 709 | ATAAAC | | | 10274 | | Cc (SEQ ID: 881) |
| B73- | 3-181430665-A- | 5 | 272798 | SOX2: | Microphthalmia | CAgcA[tGatG\|tGAtg]CA |
| 710 | ATGATG | | | 6657 | syndromic 3 | GGa (SEQ ID: 882) |
| B74- | 5-86682703-T- | 5 | 239865 | RASA1: | Capillary malformation- | CATGt[{TTTA\|TttTa]gA |
| 711 | TTTTTA | | | 5921 | arteriovenous | TgA (SEQ ID: 883) |
| | | | | | malformation | |
| B75- | 8-116617181-A- | 5 | 432085 | TRPS1: | Trichorhinophalangeal | tggcA[atctG\|atctG]gtgtt |
| 712 | AATCTG | | | 7227 | dysplasia type I | (SEQ ID: 884) |
| B76- | 11-2797207-C- | 5 | 442554 | KCNQ1: | Long QT syndrome 1 | Cttac[gatGt\|GaTgt]gCGg |
| 713 | CGATGT | | | 3784 | | G (SEQ ID: 885) |
| B77- | 11-31811508-A- | 5 | 191325 | PAX6: | Aniridia 1 | TgAGA[CAtat\|cAtat]caG |
| 714 | ACATAT | | | 5080 | | Gt (SEQ ID: 886) |
| B78- | 11-31812376-G- | 5 | 461519 | PAX6: | Aniridia 1 | tgCaG[gagtA\|GagTa]tGa |
| 715 | GGAGTA | | | 5080 | | gG (SEQ ID: 887) |
| B79- | 11-64577297-G- | 5 | 398421 | MEN1: | Multiple endocrine | tcTgg[gcGgt\|GcgGt]Gaag |
| 716 | GGCGGT | | | 4221 | neoplasia, type 1 | c (SEQ ID: 888) |
| B80- | 13-32900712-T- | 5 | 261019 | BRCA2: | Breast-ovarian cancer, | tCtTT[agctA\|AGctA]CAc |
| 717 | TAGCTA | | | 675 | familial 2 | CA (SEQ ID: 889) |
| B81- | 13-32932021-T- | 5 | 261432 | BRCA2: | Breast-ovarian cancer, | TGGCt[cataC\|CATAc]cc |
| 718 | TCATAC | | | 675 | familial 2 | TCc (SEQ ID: 890) |
| B82- | 15-48718033-A- | 5 | 400146 | FBN1: | Marfan syndrome | aaAca[{CGtg\|tcGtG]Aata |
| 719 | ATCGTG | | | 2200 | | A (SEQ ID: 891) |
| B83- | 17-29653013-T- | 5 | 467484 | NF1: | Neurofibromatosis, | Tgctt[ACgaC\|acGac]AaC |
| 720 | TACGAC | | | 4763 | type 1 | gt (SEQ ID: 892) |
| B84- | 17-48264062-G- | 5 | 413977 | COL1A1: | Osteogenesis | tGcgG[cTgcC\|cTGcc]ctct |
| 721 | GCTGCC | | | 1277 | imperfecta type I | g (SEQ ID: 893) |
| B85- | 19-11216237-G- | 5 | 245693 | LDLR: | Familial | tggTG[GccCC\|Gcccc]gac |
| 722 | GGCCCC | | | 3949 | hypercholesterolemia | tg (SEQ ID: 894) |
| B86- | 19-11224008-T- | 5 | 246065 | LDLR: | Familial | AcgCT[GgacC\|Ggacc]gg |
| 723 | TGGACC | | | 3949 | hypercholesterolemia | agc (SEQ ID: 895) |
| B87- | 19-11230868-C- | 5 | 246422 | LDLR: | Familial | TCcCc[agagg\|agAGG]at |
| 724 | CAGAGG | | | 3949 | hypercholesterolemia | ATG (SEQ ID: 896) |
| B88- | 19-11230879-G- | 5 | 246429 | LDLR: | Familial | tATGG[TTCTC\|Ttctc]Ttc |
| 725 | GTTCTC | | | 3949 | hypercholesterolemia | Ca (SEQ ID: 897) |
| B89- | 19-11233887-C- | 5 | 390628 | LDLR: | Familial | CcaCc[gtcag\|gtcAG]GCta |
| 726 | CGTCAG | | | 3949 | hypercholesterolemia | A (SEQ ID: 898) |
| B90- | 19-13136144-A- | 5 | 205791 | NFIX: | Sotos syndrome 2 | gGgcA[AGatC\|AGatc]cg |
| 727 | AAGATC | | | 4784 | | gcg (SEQ ID: 899) |
| B91- | 20-62044881-T- | 5 | 361908 | KCNQ2: | Benign familial | Gtcgt[AGggc\|AGgGc]cg |
| 728 | TAGGGC | | | 3785 | neonatal seizures 1 | CAg (SEQ ID: 900) |
| B92- | 2-239757079-G- | 6 | 204358 | TWIST2: | Barber-Say syndrome | gCgCg[AgCgcc\|Agcgcc] |
| 729 | GAGCGCC | | | 117581 | | AgCgc (SEQ ID: 901) |
| B93- | 9-140056954-C- | 6 | 384406 | GRIN1: | Mental retardation, | aactc[Cggcat\|CGgcat]cgg |
| 730 | CCGGCAT | | | 2902 | autosomal dominant 8 | gg (SEQ ID: 902) |
| B94- | 10-102510456-A- | 6 | 28839 | PAX2: | Renal coloboma | TacTa[CgagAc\|cgAgac]C |
| 731 | ACGAGAC | | | 5076 | syndrome | gGCa (SEQ ID: 903) |
| B95- | 3-123383092-T- | 7 | 259621 | MYLK: | Visceral myopathy | gtgCT[CGCtttc\|cgCtttc]c |
| 732 | TCGCTTTC | | | 4638 | | TGga (SEQ ID: 904) |
| B96- | 6-7580155-G- | 7 | 197069 | DSP: | Arrhythmogenic right | caaGg[gaAaaTc\|gaAaatc] |
| 733 | GGAAAATC | | | 1832 | ventricular | gAgat (SEQ ID: 905) |
| | | | | | cardiomyopathy, | |
| | | | | | type 8 | |
| B97- | 13-32937340-C- | 7 | 67137 | BRCA2: | Breast-ovarian | GAagC[agAAgaT\|agaAg |
| 734 | CAGAAGAT | | | 675 | cancer, familial 2 | at]cGGCt (SEQ ID: 906) |
| B98- | 13-32968822-C- | 7 | 262861 | BRCA2: | Breast-ovarian cancer, | CATtc[taGgact\|TAGgaC |
| 735 | CTAGGACT | | | 675 | familial 2 | T]Tgccc (SEQ ID: 907) |
| B99- | 16-2142114-T- | 7 | 442559 | PKD1: | Polycystic kidney | aAcgt[CGtaatC\|CGTaatc] |
| 736 | TCGTAATC | | | 5310 | disease, adult | gCtgg (SEQ ID: 908) |
| | | | | | type | |
| B100- | 17-7578448-G- | 7 | 27419 | TP53: | Osteosarcoma | gATGG[ccATGgc\|ccAtg |
| 737 | GCCATGGC | | | 7157 | | gc]GCgga (SEQ ID: 909) |
| B101- | 17-48273539-C- | 7 | 414018 | COL1A1: | Osteogenesis | gTagC[ACCAtCa\|aCcaT |
| 738 | CACCATCA | | | 1277 | imperfecta type I | Ca]tTtcC (SEQ ID: 910) |
| B102- | 18-48575180-G- | 7 | 36142 | SMAD4: | Juvenile polyposis | gataG[TgTCtGT\|Tgtctgt] |
| 739 | GTGTCTGT | | | 4089 | syndrome | GIGAA (SEQ ID: 911) |
| B103- | 19-11213391-G- | 7 | 362671 | LDLR: | Familial | aAccg[CtgcAtT\|CtGCatt] |
| 740 | GCTGCATT | | | 3949 | hypercholesterolemia | CcTCa (SEQ ID: 912) |
| B104- | 19-11216256-A- | 7 | 245719 | LDLR: | Familial | GacAA[aTcTGAc\|aTCtga |
| 741 | AATCTGAC | | | 3949 | hypercholesterolemia | c]gAGGa (SEQ ID: 913) |
| B105- | 19-11231163-T- | 7 | 246518 | LDLR: | Familial | ggCat[GCtgCTg\|GCtgCT |
| 742 | TGCTGCTG | | | 3949 | hypercholesterolemia | g]gccAG (SEQ ID: 914) |
| B106- | 22-32200157-T- | 7 | 259349 | DEPDC5: | Epilepsy, familial | ggtgT[GgatttG\|gGatTtG]g |
| 743 | TGGATTTG | | | 9681 | focal, with | TgTg (SEQ ID: 915) |
| | | | | | variable foci 1 | |
| B107- | 2-166848493-G- | 8 | 187713 | SCN1A: | Severe myoclonic | aaaAg[AAAATTCC\|AAa |
| 744 | GAAAATTCC | | | 6323 | epilepsy in | atTCC]aacag (SEQ ID: |
| | | | | | infancy | 916) |
| B108- | 6-33405537-C- | 8 | 456125 | SYNGAP1: | Mental retardation, | TCtgc[ctgGatga\|cTgGAt |
| 745 | CCTGGATGA | | | 8831 | autosomal dominant 5 | Ga]CAtgc (SEQ ID: 917) |
| B109- | 8-61728946-C- | 8 | 207553 | CHD7: | CHARGE association | tcaGC[tcTTatcT\|TCttAtc |
| 746 | CTCTTATCT | | | 55636 | | T]TcatT (SEQ ID: 918) |
| B110- | 8-116427276-C- | 8 | 432065 | TRPS1: | Trichorhinophalangeal | tcAcc[GtTgtTTt\|GtTGtT |
| 747 | CGTTGTTTT | | | 7227 | dysplasia type I | TT]GTtta (SEQ ID: 919) |
| B111- | 11-64572225-C- | 8 | 419786 | MEN1: | Multiple endocrine | tCgcc[ccAcggct\|ccAcGgc |
| 748 | CCCACGGCT | | | 4221 | neoplasia, type 1 | t]ccTcG (SEQ ID: 920) |
| B112- | 13-32914325-A- | 8 | 261315 | BRCA2: | Breast-ovarian cancer, | tAAaa[TATCaCCt\|tatcAc |
| 749 | ATATCACCT | | | 675 | familial 2 | Ct]tGtga (SEQ ID: 921) |
| B113- | 13-32915019-A- | 8 | 261369 | BRCA2: | Hereditary breast and | GAGAA[cattCaTg\|cattCa |
| 750 | ACATTCATG | | | 675 | ovarian cancer syndrome | TG]ttttG (SEQ ID: 922) |
| B114- | 16-23619228-C- | 8 | 465434 | PALB2: | Familial cancer of | ccCac[gctgaGag\|gcTgAG |
| 751 | CGCTGAGAG | | | 79728 | breast | aG]TCGtc (SEQ ID: |
| | | | | | | 923) |
| B115- | 16-23634319-T- | 8 | 465443 | PALB2: | Familial cancer of | ctTCT[acttGTtG\|aCTtgTt |
| 752 | TACTTGTTG | | | 79728 | breast | G]atCag (SEQ ID: 924) |
| B116- | 17-17118378-C- | 8 | 467434 | FLCN: | Multiple | GCTtc[aaTctTat\|aATcTT |
| 753 | CAATCTTAT | | | 201163 | fibrofolliculomas | at]tcAgg (SEQ ID: 925) |
| B117- | 19-1220430-A- | 8 | 469353 | STK11: | Peutz-Jeghers syndrome | gCacA[AggacatC\|AgGac |
| 754 | AAGGACATC | | | 6794 | | AtC]Aagcc (SEQ ID: |
| | | | | | | 926) |
| B118- | 19-11216107-T- | 8 | 245617 | LDLR: | Familial | aagaT[GgcTcgGa\|ggctcg |
| 755 | TGGCTCGGA | | | 3949 | hypercholesterolemia | Ga]TgaGT (SEQ ID: |
| | | | | | | 927) |
| B119- | 19-11221433-A- | 8 | 245939 | LDLR: | Familial | Gccca[GcgaaGat\|gcgAaG |
| 756 | AGCGAAGAT | | | 3949 | hypercholesterolemia | at]GcgAa (SEQ ID: 928) |
| B120- | 19-11226844-C- | 8 | 246266 | LDLR: | Familial | tactc[gCtGgtga\|gCtGGTg |
| 757 | CGCTGGTGA | | | 3949 | hypercholesterolemia | a]CTGAA (SEQ ID: |
| | | | | | | 929) |
| B121- | 19-42794719-G- | 8 | 424692 | CIC: | MENTAL RETARDATION, | cggcg[caAgAgAc\|CaAGa |
| 758 | GCAAGAGAC | | | 23152 | AUTOSOMAL DOMINANT 45 | gac]ccgAa (SEQ ID: |
| | | | | | | 930) |
| B122- | 7-142458427-A- | 9 | 46926 | PRSS1: | Hereditary pancreatitis | gAtGa[TgacAAgAt\|Tgac |
| 759 | ATGACAAGAT | | | 5644 | | aAgAt]cgttg (SEQ ID: |
| | | | | | | 931) |
| B123- | 10-43607601-T- | 9 | 28980 | RET: | Familial medullary | CggCt[ggAgTgtGa\|GgAg |
| 760 | TGGAGTGTGA | | | 5979 | thyroid carcinoma | TgtGa]GgAgt (SEQ ID: |
| | | | | | | 932) |
| B124- | 10-43609946-T- | 9 | 36267 | RET: | Multiple endocrine | GaGct[GtGcCgcac\|gtgCc |
| 761 | TGTGCCGCAC | | | 5979 | neoplasia, type 2a | gcAc]gGtga (SEQ ID: |
| | | | | | | 933) |
| B125- | 17-42992481-A- | 9 | 188181 | GFAP: | Alexander's disease | ccGca[gccgCagct\|gCcgC |
| 762 | AGCCGCAGCT | | | 2670 | | Agct]cTcgC (SEQ ID: |
| | | | | | | 934) |
| B126- | 5-176673777-A- | 10 | 207192 | NSD1: | Sotos syndrome 1 | GTcaa[aagAGATtcC\|aag |
| 763 | AAAGAGATTC | | | 64324 | | agATtcC]AGgct (SEQ |
| | C | | | | | ID: 935) |
| B127- | 10-76789773-A- | 10 | 47605 | KAT6B: | Young Simpson syndrome | CggGa[gCtGCAgcat\|GCt |
| 764 | AGCTGCAGCA | | | 23522 | | GCAgCAt]GCtGC (SEQ |
| | T | | | | | ID: 936) |
| B128- | 13-48835344-C- | 10 | 21019 | ITM2B: | Dementia, familial | gaAaC[TTTaaTTTGT\|ttT |
| 765 | CTTTAATTTGT | | | 9445 | Danish | AaTTTGT]tcTTg (SEQ |
| | | | | | | ID: 937) |
| B129- | 17-42328803-A- | 10 | 32797 | SLC4A1: | Spherocytosis type 4 | GgGCa[catcTgGgtG\|catct |
| 766 | ACATCTGGGT | | | 6521 | | gggtG]atact (SEQ ID: |
| | G | | | | | 938) |
| B130- | 19-11218087-C- | 10 | 434258 | LDLR: | Familial | gACcC[AacaagTtCA\|Aac |
| 767 | CAACAAGTTC | | | 3949 | hypercholesterolemia | aaGTtCa]AGtgT (SEQ |
| | A | | | | | ID: 939) |
| B131- | 3-138664603-T- | 11 | 354105 | FOXL2: | Blepharophimosis, | GagcT[GgcccgGcggC\|Gg |
| 768 | TGGCCCGGCG | | | 668 | ptosis, and | cCcgGcggc]GgcGc (SEQ |
| | GC | | | | epicanthus inversus | ID: 940) |
| B132- | 8-61742962-G- | 11 | 481218 | CHD7: | CHARGE association | caaag[aAGAaaCTAtt\|aA |
| 769 | GAAGAAACTA | | | 55636 | | GAaACtATt]aTtGA |
| | TT | | | | | (SEQ ID: 941) |
| B133- | 8-61777949-T- | 11 | 194201 | CHD7: | CHARGE association | TgAAT[aACCctCtgtc\|aac |
| 770 | TAACCCTCTGT | | | 5563 | | CcTctgtc]aGCTg (SEQ |
| | C | | | 6 | | ID: 942) |
| B134- | 19-11216021-A- | 11 | 434823 | LDLR: | Familial | gcTCa[CctgtggTCCc\|Cct |
| 771 | ACCTGTGGTC | | | 3949 | hypercholesterolemia | gtggtCCC]gCcag (SEQ |
| | CC | | | | | ID: 943) |
| B135- | 19-11216250-A- | 11 | 245708 | LDLR: | Familial | tgcAa[GGaCAAatcTG\|G |
| 772 | AGGACAAATC | | | 3949 | hypercholesterolemia | gaCAaaTctG]acgAG |
| | TG | | | | | (SEQ ID: 944) |
| B136- | 10-43609939-G- | 12 | 36265 | RET: | Multiple endocrine | gtgcG[aCGaGctGTGcC\|a |
| 773 | GACGAGCTGT | | | 5979 | neoplasia, type 2a | cGagctGtGcC]gcAcg |
| | GCC | | | | | (SEQ ID: 945) |
| B137- | 19-11227654-T- | 12 | 424332 | LDLR: | Familial | CcccT[tctccttggcCG\|TCtc |
| 774 | TTCTCCTTGGC | | | 3949 | hypercholesterolemia | cTtggccG]Tcttt (SEQ ID: |
| | CG | | | | | 946) |
| B138- | 15-73617341-T- | 13 | 361763 | HCN4: | Sick sinus syndrome 2, | Ccctt[ggtgAgCacgctg\|ggt |
| 775 | TGGTGAGCAC | | | 10021 | autosomal dominant | GAgCAcgctG]accAC |
| | GCTG | | | | | (SEQ ID: 947) |
| B139- | 19-11216242-C- | 13 | 245695 | LDLR: | Familial | GcccC[gactgcAaGgaCA\| |
| 776 | CGACTGCAAG | | | 3949 | hypercholesterolemia | gACtgcAaGGaCA]AaTct |
| | GACA | | | | | (SEQ ID: 948) |
| B140- | 19-11233897-A- | 13 | 246545 | LDLR: | Familial | GCtaA[AggTCAGCtccac\| |
| 777 | AAGGTCAGCT | | | 3949 | hypercholesterolemia | AggTcAGCTccAc]agccg |
| | CCAC | | | | | (SEQ ID: 949) |
| B141- | 5-176637714-G- | 14 | 394847 | NSD1: | Beckwith-Wiedemann | tGggG[CAgCAaAtcAAG |
| 778 | GCAGCAAATC | | | 64324 | syndrome | ct\|CAgcaaAtcAAgct]CTa |
| | AAGCT | | | | | tT (SEQ ID: 950) |
| B142- | 6-117996952-G- | 14 | 480769 | NUS1: | MENTAL RETARDATION, | cGctG[ctgcCgcGccGcct\|c |
| 779 | GCTGCCGCGC | | | 116150 | AUTOSOMAL DOMINANT 55, | tgcCgcGccGcct]ctgcc |
| | CGCCT | | | | WITH SEIZURES | (SEQ ID: 951) |
| B143- | 13-32971081-T- | 14 | 261554 | BRCA2: | Breast-ovarian cancer, | CATAt[aCtgCAtGCaAAt |
| 780 | TACTGCATGC | | | 675 | familial 2 | g\|AcTgCAtgCaaAtg]ATc |
| | AAATG | | | | | cC (SEQ ID: 952) |
| B144- | 19-11216249-A- | 14 | 245706 | LDLR: | Familial | ctgcA[aGGaCAAaTctgac\| |
| 781 | AAGGACAAAT | | | 3949 | hypercholesterolemia | aGGacAAaTctGac]gAGg |
| | CTGAC | | | | | a (SEQ ID: 953) |
| B145- | 2-189853347-A- | 15 | 107099 | COL3A1: | Ehlers-Danlos syndrome, | ggtgA[accTGGgcAagCtG |
| 782 | AACCTGGGCA | | | 1281 | type 4 | G\|acctGGgcAagCtGg]Tc |
| | AGCTGG | | | | | CIT (SEQ ID: 954) |
| B146- | 18-59992633-G- | 15 | 204405 | TNFR | Paget disease of bone | tgCTg[cTcTgcGcgctGcTc |
| 783 | GCTCTGCGCG | | | SF11A: | 2, early-onset | \|cTcTgcGcgCtgcTc]gccC |
| | CTGCTC | | | 8792 | | g (SEQ ID: 955) |
| B147- | 3-41280628-C- | 16 | 227080 | CTNNB1: | Exudative | gatcC[TAgctaTCgTTctttt] |
| 784 | CTAGCTATCG | | | 1499 | vitreoretinopathy 1 | tAGCtAtCgTTctTTt]cAct |
| | TTCTTTT | | | | | c (SEQ ID: 956) |
| B148- | 3-71019923-G- | 16 | 102121 | FOXP1: | Mental retardation | cgTgg[cTGcTcTgcAtGtttt |
| 785 | GCTGCTCTGC | | | 27086 | with language impairment | \|CTgctcTgcAtGTtTT]TA |
| | ATGTTTT | | | | and with or without | ata (SEQ ID: 957) |
| | | | | | autistic features | |
| B149- | 11-31815221-T- | 16 | 190738 | PAX6: | Aniridia 1 | GGaaT[TggtTgGTAGAc |
| 786 | TTGGTTGGTA | | | 5080 | | ActG\|tggtTggTAGacactG |
| | GACACTG | | | | | ]gtgCT (SEQ ID: 958) |
| B150- | 16-23647409-C- | 16 | 466297 | PALB2: | Hereditary cancer- | TGTCC[TCttctgCtgCTtC |
| 787 | CTCTTCTGCTG | | | 79728 | predisposing syndrome | Tt\|TctTCtgCtgCTtCTt]Tc |
| | CTTCTT | | | | | (TC (SEQ ID: 959) |
| B151- | 19-49469932-T- | 16 | 31527 | FTL: | Neuroferritinopathy | tggGt[ggcCcgGaggcTgg |
| 788 | TGGCCCGGAG | | | 2512 | | Gc\|GgcCcgGaggcTgggc]t |
| | GCTGGGC | | | | | gGgc (SEQ ID: 960) |
| B152- | 3-138664693-T- | 17 | 19905 | FOXL2: | Blepharophimosis, | Cgggt[gGgGgtGcgGcgga |
| 789 | TGGGGGTGCG | | | 668 | ptosis, and | ggc\|gGgGgtGcgGcggagg |
| | GCGGAGGC | | | | epicanthus inversus | c]gGgGg (SEQ ID: 961) |
| B153- | 3-138664705-G- | 17 | 171758 | FOXL2: | Blepharophimosis, | cgGcg[gaggcgGgGgtGCg |
| 790 | GGAGGCGGGG | | | 668 | ptosis, and | Gcc\|gaggcgGgGgtGCggC |
| | GTGCGGCC | | | | epicanthus inversus | c]ggCgg (SEQ ID: 962) |
| B154- | 3-138664707-A- | 17 | 178773 | FOXL2: | Blepharophimosis, | Gcgga[ggcgGgGgtGCggc |
| 791 | AGGCGGGGGT | | | 668 | ptosis, and | cgg\|ggcgGgGgtGCggCcg |
| | GCGGCCGG | | | | epicanthus inversus | g]CggGC (SEQ ID: 963) |
| B155- | 9-140674107-A- | 17 | 431918 | EHMT1: | Chromosome 9q deletion | ttcCa[CccaAagcaGCTgta |
| 792 | ACCCAAAGCA | | | 79813 | syndrome | cT\|CccaAagcaGCTgTAc |
| | GCTGTACT | | | | | T]TcTcC (SEQ ID: 964) |
| B156- | 10-103990523-G- | 17 | 459553 | PITX3: | Cataract, posterior | CCCCg[cccaGgccCtgcag |
| 793 | GCCCAGGCCC | | | 5309 | polar, 4 | GGc\|ccCAggccCtgcagGG |
| | TGCAGGGC | | | | | c]ccCAg (SEQ ID: 965) |
| B157- | 16-2134369-G- | 17 | 75949 | TSC2: | Tuberous sclerosis | cccTg[agcaaGtcCAGCtcc |
| 794 | GAGCAAGTCC | | | 7249 | syndrome | Tc\|AgcAaGtcCAGCtcCT |
| | AGCTCCTC | | | | | c]tccCg (SEQ ID: 966) |
| B158- | 17-17118596-T- | 17 | 247655 | FLCN: | Hereditary cancer- | AaCgt[GCgGcTgcGtGGa |
| 795 | TGCGGCTGCG | | | 201163 | predisposing | cCtc\|GcgGcTgcgtGGaCC |
| | TGGACCTC | | | | syndrome | Tc]cacga (SEQ ID: 967) |
| B159- | 19-11216246-T- | 17 | 362682 | LDLR: | Familial | Cgact[gcAagGaCAAaTct |
| 796 | TGCAAGGACA | | | 3949 | hypercholesterolemia | GAc\|gcAagGaCAAaTctG |
| | AATCTGAC | | | | | ac]gAGga (SEQ ID: 968) |
| B160- | 11-47353677-T- | 18 | 23642 | MYBPC3: | Hypertrophic | GcCCt[GcagAcaTaGaTg |
| 797 | TGCAGACATA | | | 4607 | cardiomyopathy | CcCCc\|gcagacaTaGaTGC |
| | GATGCCCCC | | | | | cCCC]gtcaa (SEQ ID: |
| | | | | | | 969) |
| B161- | 18-59992620-T- | 18 | 204404 | TNFR | Familial expansile | Ctgtt[cGcGctGctgCTgcT |
| 798 | TCGCGCTGCT | | | SF11A: | osteolysis | cTg\|cGcgctGctgCTgcTcT |
| | GCTGCTCTG | | | 8792 | | g]cGcgC (SEQ ID: 970) |
| B162- | 18-59992630-G- | 18 | 21338 | TNFR | Familial expansile | tGctg[CTgcTcTgcGcgctG |
| 799 | GCTGCTCTGC | | | SF11A: | osteolysis | ctc\|CTgctcTgcGcgCtgcTc] |
| | GCGCTGCTC | | | 8792 | | gccCg (SEQ ID: 971) |
| B163- | 3-138664581-G- | 19 | 171757 | FOXL2: | Blepharophimosis, | cggTg[gcTGggcTggcaGg |
| 800 | GGCTGGGCTG | | | 668 | ptosis, and | GcTGa\|gcTGggcTggcaG |
| | GCAGGGCTGA | | | | epicanthus inversus | gGcTGa]gcTGg (SEQ |
| | | | | | | ID: 972) |
| B164- | 12-12870830-G- | 19 | 181495 | CDKN1B: | Multiple endocrine | cCAgg[caggcgGAGcAC |
| 801 | GCAGGCGGAG | | | 1027 | neoplasia, type 4 | CccAagc\|cagGcgGAGcA |
| | CACCCCAAGC | | | | | ccccAagc]ccTCg (SEQ |
| | | | | | | ID: 973) |
| B165- | 19-11216046-G- | 19 | 245580 | LDLR: | Familial | caGtg[caACAgcTcCacCt |
| 802 | GCAACAGCTC | | | 3949 | hypercholesterolemia | gcatC\|CaAcagcTcCacctg |
| | CACCTGCATC | | | | | CatC]ccCca (SEQ ID: |
| | | | | | | 974) |
| B166- | 19-11218155-G- | 19 | 228148 | LDLR: | Familial | gaCtg[ccgGgacTGgtcagat |
| 803 | GCCGGGACTG | | | 3949 | hypercholesterolemia | gaa\|cCGGgacTGgtcagAT |
| | GTCAGATGAA | | | | | gAA]CCCAt (SEQ ID: |
| | | | | | | 975) |
| B167- | 19-11224421-G- | 19 | 434302 | LDLR: | Familial | tcgtg[gtGgAtCCTGttcaTg |
| 804 | GGTGGATCCT | | | 3949 | hypercholesterolemia | ggt\|gTGGAtCCTgttcatgg |
| | GTTCATGGGT | | | | | gT]GCGTA (SEQ ID: |
| | | | | | | 976) |
| B168- | 2-131355422-C- | 20 | 20229 | CFC1: | Heterotaxy, visceral, | acccc[GcgcaCcCcTgtgccc |
| 805 | CGCGCACCCC | | | 55997 | 2, autosomal | aCct\|gcgcACcCcTgtgcCC |
| | TGTGCCCACC | | | | | aCct]gcgcc (SEQ ID: |
| | T | | | | | 977) |
| B169- | 3-138664638-T- | 20 | 354106 | FOXL2: | Blepharophimosis, | Gcggt[GgggcagGcgGcGg |
| 806 | TGGGGCAGGC | | | 668 | ptosis, and | tGcggc\|GgggcagGcgGcG |
| | GGCGGTGCGG | | | | epicanthus inversus | gtGcgGC]ggcCg (SEQ |
| | C | | | | | ID: 978) |
| B170- | 9-135778022-C- | 20 | 397173 | TSC1: | Tuberous sclerosis 1 | ATTcc[tctcgGtCatGctGC |
| 807 | CTCTCGGTCAT | | | 7248 | | agCTg\|tctCGGtCatGCtgC |
| | GCTGCAGCTG | | | | | |
| | | | | | | agCTg]tCtGa (SEQ ID: |
| | | | | | | 979) |
| B171- | 19-11216243-G- | 20 | 434241 | LDLR: | Familial | ccCCg[actgcAaGGacaAa |
| 808 | GACTGCAAGG | | | 3949 | hypercholesterolemia | TcTGAc\|aCtgcAaGGaCA |
| | ACAAATCTGA | | | | | AaTctGac]gAGga (SEQ |
| | C | | | | | ID: 980) |
| B172- | 20-10632292-C- | 20 | 270939 | JAG1: | Alagille syndrome 1 | GTCTC[CTtAcaGCTgCc |
| 809 | CCTTACAGCT | | | 182 | | tCtgtTgT\|CTtAcaGCTgC |
| | GCCTCTGTTGT | | | | | ctCtgtTgt]gacag (SEQ |
| | | | | | | ID: 981) |

### IV. Protospacer Adjacent Motif (PAM) Sequences

Below are exemplary PAM sequences. addgene.org/crispr/guide/#pam-table. blog.addgene.org/xcas9-engineering-a-crispr-variant-with-pam-flexibility Table 9.

TABLE 9
Exemplary PAM Sequences

| | Species_and_ | | | | | | SEQ ID |
|---|---|---|---|---|---|---|---|
| Legend | Variant_of_Cas9 | Side | Pattern | Expanded | CleavageSite | Width | NOS: |
| A/a | xCas9_NG | 3′ | NG | [ACGT]G | −3 from start | 2 | 982, 983 |
| B/b | xCas9_GAA | 3′ | GAA | GAA | −3 from start | 3 | 984, 985 |
| C/c | xCas9_GAT | 3′ | GAT | GAT | −3 from start | 3 | 986, 987 |
| D/d | SpCas9 | 3′ | NGG | [ACGT]GG | −3 from start | 3 | 988, 989 |
| E/e | SpCas9 VRER | 3′ | NGCG | [ACGT]GCG | −3 from start | 4 | 990, 991 |
| | variant | | | | | | |
| F/f | SpCas9 EQR | 3′ | NGAG | [ACGT]GAG | −3 from start | 4 | 992, 993 |
| | variant | | | | | | |
| G/g | SpCas9 VQR | 3′ | NGAN\|NGNG | [ACGT]GA[ACGT]\| | −3 from start | 4 | 994, 995 |
| | variant | | | [ACGT]G[ACGT]G | | | |
| H/h | SaCas9 | 3′ | NNGRRT | [ACGT][ACGT]G | −3 from start | 6 | 996, 997 |
| | | | | [AG][AG]T | | | |
| I/i | NMe1 | 3′ | NNNNGATT | [ACGT][ACGT] | −3 from start | 8 | 998, 999 |
| | | | | [ACGT][ACGT]GATT | | | |
| J/j | CjeCas9 | 3′ | NNNNRYAC | [ACGT][ACGT][ACGT] | −3 from start | 8 | 1000, |
| | | | | [ACGT][AG][CT]AC | | | 1001 |
| K/k | AsCpf1 and | 3′ | TTTV | TTT[ACG] | approx +18 | 4 | 1002, |
| | LbCpf1 | | | | from end | | 1003 |
| M/m | AsCpf1 and | 5′ | TYCV | T[CT]C[ACG] | approx +18 | 4 | 1004, |
| | LbCpf1 RR | | | | from end | | 1005 |
| | variant | | | | | | |
| N/n | AsCpf1 RVR | 5′ | TATV | TAT[ACG] | approx +18 | 4 | 1006, |
| | variant | | | | from end | | 1007 |
| O/o | FnCpf1 | 5′ | TTV | TT[ACG] | approx +18 | 3 | 1008, |
| | | | | | from end | | 1009 |

### V. Adeno-Associated Virus Nucleic Acid Delivery Platforms

Adeno-associated virus (AAV) is a small virus which infects humans and some other primate species. AAV is not currently known to cause disease. In many cases, AAV vectors integrate into the host cell genome, making it useful as gene therapy delivery platform. Gene therapy vectors using AAV can infect both dividing and quiescent cells and persist in an extrachromosomal state without integrating into the genome of the host cell, although in the native virus some integration of virally carried genes into the host genome does occur. Deyle et al., (August 2009). “Adeno-associated virus vector integration”. Current Opinion in Molecular Therapeutics 11(4):442-447. These features make AAV a very attractive candidate for creating viral vectors for gene therapy. Grieger et al., (2005). “Adeno-associated virus as a gene therapy vector: vector development, production and clinical applications” Advances in Biochemical Engineering/Biotechnology. Advances in Biochemical Engineering/Biotechnology 99:119-145 Recent human clinical trials using AAV for gene therapy in the retina have shown promise. Maguire et al., (May 2008) “Safety and efficacy of gene transfer for Leber's congenital amaurosis” The New England Journal of Medicine 358 (21): 2240-2248. AAV belongs to the genus Dependoparvovirus, which in turn belongs to the family Parvoviridae. The virus is a small (20 nm) replication-defective, nonenveloped virus.

Wild-type AAV has attracted considerable interest from gene therapy researchers due to a number of features. Chief amongst these is the virus's apparent lack of pathogenicity. It can also infect non-dividing cells and has the ability to stably integrate into the host cell genome at a specific site (designated AAVS1) in the human chromosome 19. Kotin et al., (March 1990). “Site-specific integration by adeno-associated virus”. PNAS USA 87(6):2211-2215; and Surosky et al., (October 1997) “Adeno-associated virus Rep proteins target DNA sequences to a unique locus in the human genome” Journal of Virology 71(10):7951-7959. This feature makes it somewhat more predictable than retroviruses, which present the threat of a random insertion and of mutagenesis, which is sometimes followed by development of a cancer.

The AAV genome integrates most frequently into the site mentioned, while random incorporations into the genome take place with a negligible frequency. Development of AAVs as gene therapy vectors, however, has eliminated this integrative capacity by removal of the rep and cap from the DNA of the vector. The desired gene together with a promoter to drive transcription of the gene is inserted between the inverted terminal repeats (ITR) that aid in concatemer formation in the nucleus after the single-stranded vector DNA is converted by host cell DNA polymerase complexes into double-stranded DNA. AAV-based gene therapy vectors form episomal concatemers in the host cell nucleus. In non-dividing cells, these concatemers remain intact for the life of the host cell. In dividing cells, AAV DNA is lost through cell division, since the episomal DNA is not replicated along with the host cell DNA. Random integration of AAV DNA into the host genome is detectable but occurs at very low frequency. AAVs also present very low immunogenicity, seemingly restricted to generation of neutralizing antibodies, while they induce no clearly defined cytotoxic response. This feature, along with the ability to infect quiescent cells present their dominance over adenoviruses as vectors for human gene therapy. Daya et al., (October 2008). “Gene therapy using adeno-associated virus vectors” Clinical Microbiology Reviews 21(4):583-593; Chirmule et al., (September 1999) “Immune responses to adenovirus and adeno-associated virus in humans” Gene Therapy 6(9):1574-1583; Hernandez et al., (October 1999) “Latent adeno-associated virus infection elicits humoral but not cell-mediated immune responses in a nonhuman primate model”. Journal of Virology 73(10):8549-8558; and Ponnazhagan et al., (April 1997) “Adeno-associated virus 2-mediated gene transfer in vivo: organ-tropism and expression of transduced sequences in mice” Gene 190 (1):203-210.

### VI. Pharmaceutical Formulations and Compositions

The present invention further provides pharmaceutical compositions (e.g., comprising the nucleases described above). The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary (e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product.

Agents that enhance uptake of oligonucleotides at the cellular level may also be added to the pharmaceutical and other compositions of the present invention. For example, cationic lipids, such as lipofectin (U.S. Pat. No. 5,705,188), cationic glycerol derivatives, and polycationic molecules, such as polylysine (WO 97/30731), also enhance the cellular uptake of oligonucleotides.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. The administering physician can easily determine optimum dosages, dosing methodologies and repetition rates. Optimum dosages may vary depending on the relative potency of individual oligonucleotides, and can generally be estimated based on EC₅₀s found to be effective in in vitro and in vivo animal models or based on the examples described herein. In general, dosage is from 0.01 μg to 100 g per kg of body weight, and may be given once or more daily, weekly, monthly or yearly. The treating physician can estimate repetition rates for dosing based on measured residence times and concentrations of the drug in bodily fluids or tissues. Following successful treatment, it may be desirable to have the subject undergo maintenance therapy to prevent the recurrence of the disease state, wherein the compound is administered in maintenance doses, ranging from 0.01 μg to 100 g per kg of body weight, once or more daily, to once every 20 years.

### EXPERIMENTAL

### Example I

### Human Subjects

Cells for reprogramming TCAP iPSC lines were recovered, with consent, from a skin biopsy from a patient with LGMD2G under a UMMS-IRB-approved protocol and assigned a de-identified ID number unlinked to the patient's medical record. The consent process included conditions for sharing de-identified samples and information with other investigators. No PHI will be shared at any time per HIPAA guidelines.

### Example II

### Cell Culture

LGMD2G primary dermal fibroblasts were isolated from a skin biopsy from a patient with LGMD2G as described³¹. Fibroblasts were reprogrammed using the CytoTune 2.0 iPS Sendai Virus Reprogramming Kit (Thermo-Fisher) according to the manufacturer's directions. Clonal lines were expanded for 6-10 passages before banking. Immunostaining was performed to confirm the absence of Sendai virus and expression of OCT4. Human iPSCs were cultured in iPS-Brew XF medium (Miltenyi Biotec) and passaged every 3-5 days with Passaging Solution (Miltenyi Biotec) according to the manufacturer's directions.

Myoblasts were induced from iPSCs using a modification of the Genea Biocells protocol¹¹. Following the generation of differentiated myotubes as described, cells were reseeded and cultured in human primary myoblast medium³². CD56+ cells were purified by FACS using an anti-CD56-APC antibody (BD Biosciences) or MACS (Miltenyi Biotec) according to the manufacturer's directions. Myogenicity was confirmed by immunostaining myoblast and myotube cultures using the mouse monoclonal antibodies MyoD clone 5.8 (Dako) and MF20 (DSHB) (data not shown).

A lymphoblastoid cell line from B lymphocytes (B-LCL) derived from a patient with HPS1 who was homozygous for the 16-bp microduplication was purchased from Coriell (Catalog GM14606). A lymphoblastoid cell line from B lymphocytes (B-LCL) derived from a patient with Tay-sachs who was homozygous for the GATA microduplication in HEXA was purchased from Coriell (Catalog GM11852) These cell lines was cultured following the recommended procedure using RPM1 1640 with 2 mM L-glutamine, 15% FBS and 1% penicillin/streptomycin.

HEK293T cells were cultured following the recommended procedure using Dulbecco's modified Eagle's medium (DMEM), 10% FBS and 1% penicillin/streptomycin.

All cultures were maintained in a humidified incubator with 5% CO₂ at 37° C.

### Example III

### Purification of SpyCas9 and LbaCas12a

Protein purification for 3×NLS-SpCas9 and LbaCas12a-2×NLS followed a common protocol. The generation and characterization of the 3×NLS-SpCas9 (Addgene #114365) and LbaCas12a-2×NLS (Addgene #114366) constructs have been described (Wu et al. Nature Medicine (in press) & Liu et al. Nucleic Acids Research (PMID 30892626). The pET21a plasmid backbone (Novagen) is used to drive the expression of a hexa-His-tagged version of each protein. The plasmid expressing 3×NLS-SpCas9 (or LbaCas12a-2×NLS) was transformed into Escherichia coli Rosetta (DE3) pLysS cells (EMD Millipore) for protein production. Cells were grown at 37° C. to an OD600 of ˜0.2, then shifted to 18° C. and induced at an OD600 of ˜0.4 for 16 h with isopropyl β-D-1-thiogalactopyranoside (IPTG, 1 mM final concentration).

Following induction, cells were pelleted by centrifugation and then resuspended with Ni-NTA buffer (20 mM TRIS pH 7.5, 1 M NaCl, 20 mM imidazole, 1 mM TCEP) supplemented with HALT Protease Inhibitor Cocktail, EDTA-Free (100×) (ThermoFisher) and lysed with M-110s Microfluidizer (Microfluidics) following the manufacturer's instructions. The protein was purified from the cell lysate using Ni-NTA resin, washed with five volumes of Ni-NTA buffer and then eluted with elution buffer (20 mM TRIS, 500 mM NaCl, 500 mM imidazole, 10% glycerol, pH 7.5). The 3×NLS-SpCas9 (or LbaCas12a protein) was dialysed overnight at 4° C. in 20 mM HEPES, 500 mM NaCl, 1 mM EDTA, 10% glycerol, pH 7.5.

Subsequently, the protein was step dialysed from 500 mM NaCl to 200 mM NaCl (final dialysis buffer: 20 mM HEPES, 200 mM NaCl, 1 mM EDTA, 10% glycerol, pH 7.5). Next, the protein was purified by cation exchange chromatography (5 ml HiTrap-S column, buffer A: 20 mM HEPES pH 7.5, 1 mM TCEP; buffer B: 20 mM HEPES pH 7.5, 1 M NaCl, 1 mM TCEP; flow rate 5 ml/min, column volume (CV) 5 ml) followed by size-exclusion chromatography (SEC) on a Superdex-200 (16/60) column (isocratic size-exclusion running buffer: 20 mM HEPES pH 7.5, 150 mM NaCl, 1 mM TCEP for 3×NLS-SpCas9 or 20 mM HEPES pH 7.5, 300 mM NaCl, 1 mM TCEP for LbCpf1-2×NLS).

The primary protein peak from the SEC was concentrated in an Ultra-15 Centrifugal Filters Ultracel-30K (Amicon) to a concentration around 100 μM based on absorbance at 280 nm. The purified protein quality was assessed by SDS-PAGE/Coomassie staining to be >95% pure and protein concentration was quantified with a Pierce BCA Protein Assay Kit (ThermoFisher Scientific). Protein was stored at −80° C. until further use.

### Example IV

### In Vitro Transcription of Guide RNAs

The DNA cassette containing the U6 promoter and the sgRNA framework for SpyCas9 was cloned from pLKO1-puro vector³³ into pBluescript SK II+backbone (Liu et al., Nucleic Acids Research, submitted). Plasmids expressing each guide RNA from the U6 promoter were constructed by annealing oligonuleotides encoding guide RNA and cloning it into BfuAI cleavage sites in this vector. Templates for in vitro transcription of SpyCas9 guides were amplified from the cognate plasmids using NEB Q5 High-Fidelity DNA Polymerase for 30 cycles (98° C., 15 s; 65° C. 25 s; 72° C. 20 s) using primer sets designed to include the T7 scaffold.

To generate CRISPR RNA (crRNA) for LbaCas12a, templates for in vitro transcription were generated by PCR amplification of oligonucleotides designed to include the T7 scaffold along with the guide RNA and a 15-mer overlap sequence to allow annealing between the oligos. The oligonucleotides encoded the full-length direct repeat crRNA sequence (Liu et al. Nucleic Acids Research, (PMID 30892626). Thirty cycles of amplification were conducted using NEB Q5 High-Fidelity DNA polymerase (98° C., 15 s; 60° C. 25 s; 72° C. 20 s). The PCR products were purified using Zymo DNA Clean & Concentrator Kit (Zymo Cat. #D4005).

In vitro transcription reactions were performed using the HiScribe T7 High Yield RNA Synthesis Kit using 300 ng of PCR product as template (NEB Cat. #E2040S). After incubation for 16 h at 37° C., samples were treated with DNase I for 40 min at 37° C. to remove any DNA contamination. Each guide RNA was purified using the Zymo RNA Clean and Concentrator Kit. Final RNA concentration was measured using Nanodrop and RNA was stored at −80° C. until further use.

### Example V

### Electroporation of Cell Lines

3×NLS-SpyCas9 protein was precomplexed with sgRNAs either purchased from Synthego or made in-house by T7 transcription and electroporated into cells using the Neon transfection system (Thermo Fisher).

### Electroporation of IPSCs

After washing with PBS, iPSCs were dissociated into single cells with 3:1 TrypLE:0.5 mM EDTA and neutralized with Ham's F10+20% FBS. To form RNP complexes, 20 pmol 3×NLS-SpyCas9 protein and 25 pmol gRNA were combined in 10 μl Neon Buffer R and incubated for 10 min at room temperature. iPSCs (1×10⁵) were resuspended in 10 μl RNP-Buffer R mix and then nucleofected as follows: pulse voltage 1,500 V, pulse width 20 ms, pulse number 1.

After transfection, the cells were plated onto Matrigel-coated 24-well plates with iPS Brew XF supplemented with 10 μM Y27632 for expansion and grown in a humidified incubator at 37° C., 5% CO₂, for 4 days before harvesting them for analysis. iPSC-derived myoblasts were electroporated using two pulses of 1,400 V and 20 ms width and plated onto a 24-well dish containing pre-warmed antibiotic-free human primary myoblast growth medium and cultured for four to six days before analysis.

### Electroporation of HPS1 Patient-Derived B-LCL Cells

Forty (40) pmol of 3×NLS-SpyCas9 protein was precomplexed with 50 pmol of sgRNA in buffer R for 10-20 min at room temperature in a final volume of 12 μl. Three hundred thousand cells per reaction were resuspended in 10 μl of RNP-buffer R mix and electroporated with 2 pulses at 1,700V for 20 ms using the 10-μl tip. Cells were then plated in 24-well plates with 500 μl of pre-equilibrated antibiotic-free culture medium and grown in a humidified incubator at 37° C. and 5% CO₂ for 7 days before indel analysis.

For the PARP-1 inhibition experiments, 300,000 HPS1 patient-derived B-LCL cells were treated with 10 μM or 20 μM rucaparib camsylate (Sigma-Aldrich PZ0036) in standard growth medium for 24 h. Treated cells were electroporated with SpyCas9 RNPs following previously described protocol. Following another 24 h incubation in rucaparib-containing medium, cells were resuspended in PARP-1 inhibitor-free medium and harvested for analysis after 7 days.

### Electroporation of HEK293T Cells

Twenty (20) pmol of 3×NLS-SpyCas9 protein and 25 pmol of in vitro transcribed sgRNA were pre-complexed in Neon Buffer R for 10-20 min at room temperature. One hundred thousand cells per reaction were resuspended in 10 μl of RNP-buffer R mix and nucleofected with SpyCas9 guide RNA complex using two pulses at 1,150 V for 20 ms using the 10-μl tip. Cells were then plated in 24-well plates with 500 μl of pre-equilibrated antibiotic-free culture medium and grown for 3 days before analysis. F or Cas 12a editing experiments at endogenous microduplications, 80 pmol of LbaCas12a protein was pre-complexed with 100 pmol of in vitro transcribed crRNA and 100,000 cells per reaction were nucleofected as described above.

### Example VI

### Indel Analysis By TIDE

Genomic DNA was extracted from HEK293T cells using GenElute Mammalian Genomic DNA Miniprep Kit (Sigma Aldrich) according to the manufacturer's instructions. The DNA region containing the 24-bp microduplication was amplified using genomic DNA as template and primers using NEB Q5 High-Fidelity DNA Polymerase (98° C., 15 s; 67° C. 25 s; 72° C. 20 s)×30 cycles. See, Table 10.

TABLE 10
List of primers used to amplify genomic regions for TIDE analysis

| Primer name | Primer Sequence |
|---|---|
| Endo_24bp_F | GAAGCGCTACCTGATTCCAATTC (SEQ ID NO: 1010) |
| Endo_24bp_R | TGGCAGTTAGGAAGGTTGTATCG (SEQ ID NO: 1011) |

Subsequently, the PCR product was purified using the DNA Clean & Concentrator-5 kit (Zymo research) and sequenced. Sanger sequencing trace data were analysed using the TIDE webtool at tide.nki.nl/to infer the compositions of indels created at the sites of DSBs34.

### Example VII

### Library Construction for Illumina Deep Sequencing

Library construction for deep sequencing was performed using a modified version of a previously described protocol²⁶. In brief, iPSCs and myoblasts were harvested following nuclease treatment and genomic DNA was extracted using the GenElute Mammalian Genomic DNA Miniprep Kit (Sigma GIN350). Genomic loci spanning the target sites were PCR amplified with locus-specific primers carrying tails complementary to the TruSeq adapters (Deepseq_TCAP_primer_fwd and Deepseq_TCAP_primer_rev). Fifty (50) nanograms of input genomic DNA was PCR amplified with Q5 High-Fidelity DNA Polymerase (New England Biolabs): (98° C., 15 s; 67° C., 25 s; 72° C., 20 s)×30 cycles. Next, 0.1 μl of each PCR reaction was amplified with barcoded primers to reconstitute the TruSeq adaptors using Q5 High-Fidelity DNA Polymerase (New England Biolabs): (98° C., 15 s; 67° C., 25 s; 72° C., 20 s)×10 cycles. Products were qualitatively analysed by gel electrophoresis. Equal amounts of the products were pooled and gel-purified using QIAquick Gel Extraction Kit (Qiagen Cat. #28704). The purified library was deep sequenced using a paired-end 150-bp Illumina MiSeq run.

### Example VIII

### Illumina Deep Sequencing Analysis

MiSeq data analysis was performed using Unix-based software tools. First, d FastQC (version 0.11.3; bioinformatics.babraham.ac.uk/projects/fastqc/) was used to determine the quality of paired-end sequencing reads (R1 and R2 fastq files). Next, a paired-end read merger (PEAR; version 0.9.8)³⁵ was used to pool raw paired-end reads and generate single merged high-quality full-length reads.

Reads were then filtered according to quality via FASTQ³⁶ for a mean PHRED quality score above 30 and a minimum per base score above 24. After that, BWA (version 0.7.5) and SAMtools (version 0.1.19) were used to align each group of filtered reads to a corresponding reference sequence.

To determine lesion type, frequency, size and distribution, all edited reads from each experimental replicate were combined and aligned, as described above. Lesion types and frequencies were then catalogued in a text output format at each base using bam-readcount. For each treatment group, the average background lesion frequencies (based on lesion type, position and frequency) of the triplicate negative control group were subtracted to obtain the nuclease-dependent lesion frequencies.

### Example IX

### Library Construction for UMI-Based Illumina Deep Sequencing

The construction of the UMI-based library used a linear amplification step to incorporate UMIs within the amplicons from the target locus¹⁵. HPS1 B-LCL cells and HEK293 Ts were harvested following nuclease treatment for genomic DNA extraction using the GenElute Mammalian Genomic DNA Miniprep Kit (Sigma G1N350).

Randomized unique molecular identifiers (UMIs) were incorporated within the 5′ locus-specific primers carrying tails complementary to TruSeq adaptors. In brief, 50 ng of input genomic DNA was linear amplified with NEB Q5 High-Fidelity DNA Polymerase (98° C., 15 s; 67° C., 25 s; 72° C., 20 s) for 10 cycles using the 5′ locus-specific primer with TruSeq adaptor conjugated with a UMI sequence.

Next a 5′ constant primer along with the 3′ locus-specific primer with TruSeq adaptor were added and further amplified for 30 cycles. Indexes were then incorporated using barcoded primers to diluted PCR products using NEB Q5 High-Fidelity DNA Polymerase (98° C., 15 s; 67° C., 25 s; 72° C., 20 s) for 10 cycles. Products were qualitatively analysed by gel electrophoresis. Equal amounts of the products were pooled and gel-purified using QIAquick Gel Extraction Kit (Qiagen Cat. #28704) for DNA recovery. The purified library was deep sequenced using a paired-end 150-bp Illumina MiSeq run.

### Example X

### UMI-Based Deep Sequencing Analysis

The analysis of the UMI-tagged deep sequencing reads was adapted from a previous protocol¹⁵. Initially, BWA (version 0.7.5) and SAMtools (version 0.1.19) were used to align each group of filtered merged-read pairs to a corresponding reference sequence, ignoring the unique molecular barcodes. Next, a custom Python and PySAM script was used to process mapped reads into counts of UMI-labelled reads for each target. The mapped reads were filtered by requiring a mapping value (MAPQ) larger than 30. Alignments were categorized into different categories of indels using VarScan 237.

Next, UMI duplicates were identified to create a minimal set of amplicons that can account for the full set of reads with unique UMIs. For each unique UMI, a minimum of five observations of the same sequence was required to consider the sequence to have a low likelihood of being an artefact (sequencing error in the UMI element). For sequences that met this threshold, all common sequences associated with the UMI were consolidated to one read for analysis of the distribution of sequence modifications that were present at a locus. The resulting UMI number tables, which describe the type of each sequence modification and its length, were concatenated and loaded into GraphPad Prism 7 for data visualization. Microsoft Excel version 16.21.1 was used for statistical analysis.

### Example XI

### PacBio Library Preparation

Single molecule, real-time (SMRT) sequencing is modified from Pacific Biosciences (PacBio). Nuclease-treated patient-derived iPSCs were harvested for genomic DNA extraction with GenElute Mammalian Genomic DNA Miniprep Kit (Sigma G1N350). In brief, regions that flanked the TCAP target site were PCR amplified using locus-specific primers. See, Table 10. The forward primer was designed to have the barcode sequence followed by the UMI and locus-specific primer sequence. The reverse primer contains the barcode followed by the locus-specific primer sequence. Input DNA (25-50 ng) was PCR amplified with Phusion High Fidelity DNA Polymerase (New England Biolabs): (98° C., 15 s; 65° C., 25 s; 72° C., 18 s)×30 cycles. The products were qualitatively analysed by gel electrophoresis and subsequently gel purified with. QIAquick Gel Extraction Kit (Qiagen Cat. #28704). The purified products sequenced at the UMASS Medical School Deep Sequencing Core for SMRTbell Library Preparation using a Pacific Biosciences Sequel Instrument.

### Example XII

### PacBio Sequencing Data Analysis

For PacBio sequencing data analysis, Minimap2 (version 2.1438,) was used to align the raw Consensus_ROI (reads_of_insert.fastq) data to the 2-kb reference sequence. Alignment quality control and filtering were performed using custom Perl script to remove errors and filter out alignments with poor quality. For variation calling, a custom Python script was used to extract deletions or insertions larger than 5 bp for each read from the SAM files. Subsequently, deletions or insertions were classified into different groups on the basis of their length. IGV (version 2.4.16) was used for alignment visualization of the aligned reads using Quick consensus mode³⁹.

### Example XIII

Clonal Analysis of iPSCs

Following confirmation of MMEJ-mediated correction in the population of LGMD2G iPSCs, clonal analysis was performed. Cells from the corrected population were seeded into 96-well plates in the presence of Y27632 at a frequency of 0.8 cells per well. iPSC clones were cultured for several weeks in iPS Brew XF (Miltenyi Biotec) before being collected for sequence analysis by deep-sequencing.

### Example IVX

### Myoblast Differentiation and Detection of Telethonin Expression

iPSC-derived myoblasts were plated into 0.1% gelatin-coated 6-well plates at a density of 100,000 cells per well in myoblast expansion medium containing Ham's F-10 (Cellgro) supplemented with 20% fetal bovine serum (Hyclone, SH30071.03), 1.2 mM CaCl₂) (EMD OmniPur 3000) and 1% chick embryo extract isolated from day 12 SPF Premium Fertilized White Leghorn Chicken Eggs (Charles River, North Franklin, CT). After 4 days of expansion, the cells were incubated with myotube differentiation medium including DMEM/F12 (Thermo-Fisher) supplemented with 1% N2 (Thermo-Fisher, 17502-048) and 1% insulin-transferrin-selenium (Thermo-Fisher, 41400045).

After 10 days of differentiation, the cells were dissociated into single cells using TrypLE. Subsequently the cells were fixed with 2% PFA for 15 min and blocked with PBS including 2% BSA, 2% horse serum, 2% goat serum and 2% Triton X-100 for 20 min. The cells were then incubated with anti-telethonin antibody (Santa Cruz, sc-25327, 1:50) at 4° C. for 2 days and IgG goat anti-mouse secondary antibody labelled with Alexa 488 fluorophore (Invitrogen, A11017, 1:800) at room temperature for 1 h. The cells were suspended in flow buffer (PBS including 0.2% FBS) and flow cytometry was performed using a BD FACSAria IIu (UMMS Flow Cytometry Core Laboratory). Roughly 20,000 cells were included for analysis. FlowJo software (version 7.6) was used for data analysis.

### Example XV

### Survey of Microduplications in ClinVar and in Human Reference Populations

Annotations of pathogenicity from Clin Var (ftp.ncbi.nlm.nih.gov/pub/clinvar/vcf_GRCh37/clinvar_20180225.vcf.gz)²⁰ were combined with annotations of allele-frequencies from gnomAD console.cloud.google.com/storage/browser/gnomad-public/release/2.0.2/vcf)²¹ and from the 1000 Genome Project ftp.1000genomes.ebi.ac.uk/vol1/ftp/release/20130502/)⁴⁰ using the annotate function in bcf tools⁴¹ (1.9), after decomposition of multi-allelic sites and normalization of variants with vt42 (v0.5772) against a reference genome (broadinstitute.org/ftp/pub/seq/references/Homo_sapiens_assemblyl9.fasta). Most analyses were restricted to the intervals in ftp.broadinstitute.org/pub/ExAC_release/release1/resources/exome_calling_regions. v1.interval_list.

Insertions were extracted using vt (view-h-f “VTYPE=INDEL&&DLEN>0”); then duplications were identified, repeat units counted, internal shift-symmetries determined, and flanking genomic regions extracted using a modified version of the vt function annotate_indels. Additional processing (filtering, finding maximal allele frequencies among different populations, scanning for PAM sites and so on) was performed using R (3.4.3), including the VariantAnnotation (1.24.5) package⁴³.

Exact tandem repeats in the reference genome were identified using the Tandem Repeats Finder program (4.09) 44 and checked for exact matches elsewhere in the genome with bwa fastmap (0.7.17) 45. Examples of different lengths were manually selected to use for the tests of collapse of endogenous microduplications.

### Example XVI

### Code Availability Statement

Data analysis used a combination of publicly available software and custom code, as detailed in the Methods. Custom python (CRESA-lpp.py) and R (indel_background_filtering.R) scripts used in the Illumina data analysis and the shell script (Tcap_pacbio_analysis.sh) used for the analysis of the PacBio data are hosted on GitHub (github.com/locusliu/PCR_Amplicon_target_deep_seq). Scripts for the bioinformatic analysis of pathogenic microduplications are hosted at rambutan.umassmed.edu/duplications/.

### REFERENCES

1. Moreira, E. S. et al. Limb-girdle muscular dystrophy type 2G is caused by mutations in the gene encoding the sarcomeric protein telethonin. Nat. Genet. 24, 163-166 (2000).
2. El-Chemaly, S. & Young, L. R. Hermansky-Pudlak syndrome. Clin. Chest Med. 37, 505-511 (2016).
3. Sfeir, A. & Symington, L. S. Microhomology-mediated end joining: a back-up survival mechanism or dedicated pathway? Trends Biochem. Sci. 40, 701-714 (2015).
4. Bae, S., Kweon, J., Kim, H. S. & Kim, J.-S. Microhomology-based choice of Cas9 nuclease target sites. Nat. Methods 11, 705-706 (2014).
5. Kim, S.-I. et al. Microhomology-assisted scarless genome editing in human iPSCs. Nat. Commun. 9, 939 (2018).
6. Hisano, Y. et al. Precise in-frame integration of exogenous DNA mediated by CRISPR/Cas9 system in zebrafish. Sci. Rep. 5, 8841 (2015).
7. Sakuma, T., Nakade, S., Sakane, Y., Suzuki, K. T. & Yamamoto, T. MMEJ-assisted gene knock-in using TALENs and CRISPR-Cas9 with the PITCh systems. Nat. Protocols 11, 118-133 (2016).
8. Bertz, M., Wilmanns, M. & Rief, M. The titin-telethonin complex is a directed, superstable molecular bond in the muscle Z-disk. Proc. Natl Acad. Sci. USA 106, 13307-13310 (2009).
9. Nigro, V. & Savarese, M. Genetic basis of limb-girdle muscular dystrophies: the 2014 update. Acta Myol. 33, 1-12 (2014).
10. Kosicki, M., Tomberg, K. & Bradley, A. Repair of double-strand breaks induced by CRISPR-Cas9 leads to large deletions and complex rearrangements. Nat. Biotechnol. 36, 765-771 (2018). 10.1038/nbt.4192
11. Caron, L. et al. A human pluripotent stem cell model of facioscapulohumeral muscular dystrophy-affected skeletal muscles. Stem Cells Transl. Med. 5, 1145-1161 (2016).
12. Oh, J. et al. Positional cloning of a gene for Hermansky-Pudlak syndrome, a disorder of cytoplasmic organelles. Nat. Genet. 14, 300-306 (1996).
13. Richmond, B. et al. Melanocytes derived from patients with Hermansky-Pudlak syndrome types 1, 2, and 3 have distinct defects in cargo trafficking. J. Invest. Dermatol. 124, 420-427 (2005).
14. Brantly, M. et al. Pulmonary function and high-resolution CT findings in patients with an inherited form of pulmonary fibrosis, Hermansky-Pudlak syndrome, due to mutations in HPS-1. Chest 117, 129-136 (2000).
15. Bolukbasi, M. F. et al. Orthogonal Cas9-Cas9 chimeras provide a versatile platform for genome editing. Nat. Commun. 9, 4856 (2018).
16. Sharma, S. et al. Homology and enzymatic requirements of microhomology-dependent alternative end joining. Cell Death Dis. 6, e1697 (2015).
17. Wang, M. et al. PARP-1 and Ku compete for repair of DNA double strand breaks by distinct NHEJ pathways. Nucleic Acids Res. 34, 6170-6182 (2006).
18. Dutta, A. et al. Microhomology-mediated end joining is activated in irradiated human cells due to phosphorylation-dependent formation of the XRCC1 repair complex. Nucleic Acids Res. 45, 2585-2599 (2017).
19. Zetsche, B. et al. Cpf1 is a single RNA-guided endonuclease of a class 2 CRISPR-Cas system. Cell 163, 759-771 (2015).
20. Landrum, M. J. et al. Clin Var: improving access to variant interpretations and supporting evidence. Nucleic Acids Res. 46, D1062-D1067 (2018).
21. Lek, M. et al. Analysis of protein-coding genetic variation in 60,706 humans. Nature 536, 285-291 (2016).
22. Komor, A. C., Badran, A. H. & Liu, D. R. CRISPR-based technologies for the manipulation of eukaryotic genomes. Cell 168, 20-36 (2017).
23. Kim, E. et al. In vivo genome editing with a small Cas9 orthologue derived from Campylobacter jejuni. Nat. Commun. 8, 14500 (2017).
24. Edraki, A. et al. A compact, high-accuracy Cas9 with a dinucleotide PAM for in vivo genome editing. Mol. Cell 73, 714-726.e4 (2019).
25. Kleinstiver, B. P. et al. Engineered CRISPR-Cas9 nucleases with altered PAM specificities. Nature 523, 481-485 (2015).
26. Bolukbasi, M. F. et al. DNA-binding-domain fusions enhance the targeting range and precision of Cas9. Nat. Methods 12, 1150-1156 (2015).
27. Hu, J. H. et al. Evolved Cas9 variants with broad PAM compatibility and high DNA specificity. Nature 556, 57-63 (2018).
28. van Overbeek, M. et al. DNA repair profiling reveals nonrandom outcomes at Cas9-mediated breaks. Mol. Cell 63, 633-646 (2016).
29. Suzuki, K. et al. In vivo genome editing via CRISPR/Cas9 mediated homology-independent targeted integration. Nature 540, 144-149 (2016).
30. Shen, M. W. et al. Predictable and precise template-free CRISPR editing of pathogenic variants. Nature 563, 646-651 (2018).
31. Rittie, L. & Fisher, G. J. Isolation and culture of skin fibroblasts. Methods Mol. Med. 117, 83-98 (2005).
32. Stadler, G. et al. Establishment of clonal myogenic cell lines from severely affected dystrophic muscles—CDK4 maintains the myogenic population. Skelet. Muscle 1, 12 (2011).
33 Kearns, N. A. et al. Cas9 effector-mediated regulation of transcription and differentiation in human pluripotent stem cells. Development 141, 219-223 (2014).
34. Brinkman, E. K., Chen, T., Amendola, M. & van Steensel, B. Easy quantitative assessment of genome editing by sequence trace decomposition. Nucleic Acids Res. 42, e168 (2014).
35. Zhang, J., Kobert, K., Flouri, T. & Stamatakis, A. PEAR: a fast and accurate Illumina Paired-End reAd mergeR. Bioinformatics 30, 614-620 (2014).
36. Blankenberg, D. et al. Manipulation of FASTQ data with Galaxy. Bioinformatics 26, 1783-1785 (2010).
37. Koboldt, D. C. et al. VarScan 2: somatic mutation and copy number alteration discovery in cancer by exome sequencing. Genome Res. 22, 568-576 (2012).
38. Li, H. Minimap2: pairwise alignment for nucleotide sequences. Bioinformatics 34, 3094-3100 (2018).
39. Robinson, J. T. et al. Integrative genomics viewer. Nat. Biotechnol. 29, 24-26 (2011).
40. 1000 Genomes Project Consortium A global reference for human genetic variation. Nature 526, 68-74 (2015).
41. Li, H. et al. The Sequence Alignment/Map format and SAMtools. Bioinformatics 25, 2078-2079 (2009).
42. Tan, A., Abecasis, G. R. & Kang, H. M. Unified representation of genetic variants. Bioinformatics 31, 2202-2204 (2015).
43. Obenchain, V. et al. VariantAnnotation: a Bioconductor package for exploration and annotation of genetic variants. Bioinformatics 30, 2076-2078 (2014).
44. Benson, G. Tandem repeats finder: a program to analyze DNA sequences. Nucleic Acids Res. 27, 573-580 (1999).
45. Li, H. Exploring single-sample SNP and INDEL calling with whole-genome de novo assembly. Bioinformatics 28, 1838-1844 (2012).

1-6. (canceled)

7. A method, comprising;
a) providing;
i) a subject comprising a genomic locus having a microduplication mutation; and
ii) a pharmaceutical formulation comprising a programmable nuclease, said nuclease having sequence-specific DNA-binding affinity for an intervening region joining a first microhomologous sequence and a second microhomologous sequence within said microduplication mutation; and
b) administering said pharmaceutical formulation to said subject under conditions such that said microduplication mutation is replaced with a wild type sequence of said genomic locus.

8. The method of claim 6, wherein said wild type sequence replaces said microduplication mutation through DNA repair.

9. The method of claim 7, wherein said DNA repair is performed without assistance of an exogenously supplied donor DNA.

10. The method of claim 7, wherein said nuclease further comprises a protospacer adjacent motif binding domain having said sequence-specific DNA-binding affinity for said genomic locus protospacer adjacent motif sequence.

11. The method of claim 7, wherein said genomic locus is selected from the group consisting of TCAP, HPS1, HEXA, DOK7 and RAX2.

12. The method of claim 7, wherein said subject further exhibits at least one symptom of a disease caused by said target gene microduplication mutation.

13. The method of claim 7, wherein said disease is selected from the group consisting of limb-girdle muscular dystrophy 2G, Hermanksy-Pudlak syndrome, Tay-Sachs disease, familial limb-girdle myasthenia and cone-rod dystrophy 11.

14. The method of claim 7, wherein said administering further reduces said at least one symptom of said disease.

15. The method of claim 7, wherein said nuclease is selected from the group consisting of a Class II CRISPR single effector nuclease, a Cas9 nuclease, a Cas12 nuclease, a zinc finger nuclease and a transcription activator-like effector nuclease.

16. The method of claim 7, wherein said pharmaceutical formulation comprises an adeno-associated virus encoding said programmable nuclease.


SEQUENCE LISTING (1107 sequences)

SEQ ID NO: 1 - skipped

SEQ ID NO: 2 - DNA, 17 bp, synthetic construct
  misc_feature 1..17: note = Synthetic
  source 1..17: mol_type = other DNA
tggcgcggccccggccc

SEQ ID NO: 3-4 - skipped (2 entries)

SEQ ID NO: 5 - DNA, 19 bp, synthetic construct
  misc_feature 1..19: note = Synthetic
  source 1..19: mol_type = other DNA
gaccgaccacatctgcaga

SEQ ID NO: 6 - DNA, 18 bp, synthetic construct
  misc_feature 1..18: note = Synthetic
  source 1..18: mol_type = other DNA
tgttgtcgatggcgaccc

SEQ ID NO: 7 - skipped

SEQ ID NO: 8 - DNA, 11 bp, synthetic construct
  misc_feature 1..11: note = Synthetic
  source 1..11: mol_type = other DNA
tggtggcatga

SEQ ID NO: 9-10 - skipped (2 entries)

SEQ ID NO: 11 - DNA, 16 bp, synthetic construct
  misc_feature 1..16: note = Synthetic
  source 1..16: mol_type = other DNA
agcttcacagagtagt

SEQ ID NO: 12 - skipped

SEQ ID NO: 13 - DNA, 11 bp, synthetic construct
  misc_feature 1..11: note = Synthetic
  source 1..11: mol_type = other DNA
gggccgggccc

SEQ ID NO: 14-17 - skipped (4 entries)

SEQ ID NO: 18 - DNA, 24 bp, synthetic construct
  misc_feature 1..24: note = Synthetic
  source 1..24: mol_type = other DNA
gcccgggcagccacctgtaatctc

SEQ ID NO: 19 - skipped

SEQ ID NO: 20 - DNA, 11 bp, synthetic construct
  misc_feature 1..11: note = Synthetic
  source 1..11: mol_type = other DNA
ttcacagagta

SEQ ID NO: 21-22 - skipped (2 entries)

SEQ ID NO: 23 - DNA, 17 bp, synthetic construct
  misc_feature 1..17: note = Synthetic
  source 1..17: mol_type = other DNA
agccacgcaggtatcgt

SEQ ID NO: 24-25 - skipped (2 entries)

SEQ ID NO: 26 - DNA, 17 bp, synthetic construct
  misc_feature 1..17: note = Synthetic
  source 1..17: mol_type = other DNA
tgggcctcccctgctgg

SEQ ID NO: 27-31 - skipped (5 entries)

SEQ ID NO: 32 - DNA, 21 bp, synthetic construct
  misc_feature 1..21: note = Synthetic
  source 1..21: mol_type = other DNA
tgaagtggtaggaaaaatgtc

SEQ ID NO: 33 - DNA, 21 bp, synthetic construct
  misc_feature 1..21: note = Synthetic
  source 1..21: mol_type = other DNA
tcatacttttcttcctgttca

SEQ ID NO: 34 - skipped

SEQ ID NO: 35 - DNA, 14 bp, synthetic construct
  misc_feature 1..14: note = Synthetic
  source 1..14: mol_type = other DNA
tggggcccagtccc

SEQ ID NO: 36 - DNA, 12 bp, synthetic construct
  misc_feature 1..12: note = Synthetic
  source 1..12: mol_type = other DNA
tagccaatgttc

SEQ ID NO: 37-39 - skipped (3 entries)

SEQ ID NO: 40 - DNA, 12 bp, synthetic construct
  misc_feature 1..12: note = Synthetic
  source 1..12: mol_type = other DNA
taccggctcgcc

SEQ ID NO: 41 - DNA, 25 bp, synthetic construct
  misc_feature 1..25: note = Synthetic
  source 1..25: mol_type = other DNA
tctccttcctgccatctggacaagc

SEQ ID NO: 42 - skipped

SEQ ID NO: 43 - DNA, 17 bp, synthetic construct
  misc_feature 1..17: note = Synthetic
  source 1..17: mol_type = other DNA
agggccgtcgcgaggct

SEQ ID NO: 44 - DNA, 18 bp, synthetic construct
  misc_feature 1..18: note = Synthetic
  source 1..18: mol_type = other DNA
cgtcctcagcttcacaga

SEQ ID NO: 45 - DNA, 14 bp, synthetic construct
  misc_feature 1..14: note = Synthetic
  source 1..14: mol_type = other DNA
gctagaatgagtta

SEQ ID NO: 46-48 - skipped (3 entries)

SEQ ID NO: 49 - DNA, 11 bp, synthetic construct
  misc_feature 1..11: note = Synthetic
  source 1..11: mol_type = other DNA
tggctgcgctg

SEQ ID NO: 50 - DNA, 11 bp, synthetic construct
  misc_feature 1..11: note = Synthetic
  source 1..11: mol_type = other DNA
aaaggaggatc

SEQ ID NO: 51-62 - skipped (12 entries)

SEQ ID NO: 63 - DNA, 11 bp, synthetic construct
  misc_feature 1..11: note = Synthetic
  source 1..11: mol_type = other DNA
cttccaggtta

SEQ ID NO: 64 - DNA, 11 bp, synthetic construct
  misc_feature 1..11: note = Synthetic
  source 1..11: mol_type = other DNA
gccacatggct

SEQ ID NO: 65 - DNA, 12 bp, synthetic construct
  misc_feature 1..12: note = Synthetic
  source 1..12: mol_type = other DNA
tccatctactcg

SEQ ID NO: 66 - DNA, 15 bp, synthetic construct
  misc_feature 1..15: note = Synthetic
  source 1..15: mol_type = other DNA
gagagctgcggagcc

SEQ ID NO: 67-74 - skipped (8 entries)

SEQ ID NO: 75 - DNA, 22 bp, synthetic construct
  misc_feature 1..22: note = Synthetic
  source 1..22: mol_type = other DNA
ggatcctggtgaccgtgctggt

SEQ ID NO: 76-79 - skipped (4 entries)

SEQ ID NO: 80 - DNA, 18 bp, synthetic construct
  misc_feature 1..18: note = Synthetic
  source 1..18: mol_type = other DNA
aaccacgtcctcagcttc

SEQ ID NO: 81 - DNA, 19 bp, synthetic construct
  misc_feature 1..19: note = Synthetic
  source 1..19: mol_type = other DNA
actcctgatcagacatgac

SEQ ID NO: 82 - DNA, 32 bp, synthetic construct
  misc_feature 1..32: note = Synthetic
  source 1..32: mol_type = other DNA
tgagctgattggtgtcgatggcaaccagatta

SEQ ID NO: 83 - DNA, 10 bp, synthetic construct
  misc_feature 1..10: note = Synthetic
  source 1..10: mol_type = other DNA
cgcgcaggct

SEQ ID NO: 84-88 - skipped (5 entries)

SEQ ID NO: 89 - DNA, 10 bp, synthetic construct
  misc_feature 1..10: note = Synthetic
  source 1..10: mol_type = other DNA
atccacactg

SEQ ID NO: 90 - DNA, 16 bp, synthetic construct
  misc_feature 1..16: note = Synthetic
  source 1..16: mol_type = other DNA
agagggagagggaggc

SEQ ID NO: 91-93 - skipped (3 entries)

SEQ ID NO: 94 - DNA, 19 bp, synthetic construct
  misc_feature 1..19: note = Synthetic
  source 1..19: mol_type = other DNA
cggaggcgccgcctgggtt

SEQ ID NO: 95 - skipped

SEQ ID NO: 96 - DNA, 16 bp, synthetic construct
  misc_feature 1..16: note = Synthetic
  source 1..16: mol_type = other DNA
tcggcccggcggcaca

SEQ ID NO: 97-103 - skipped (7 entries)

SEQ ID NO: 104 - DNA, 11 bp, synthetic construct
  misc_feature 1..11: note = Synthetic
  source 1..11: mol_type = other DNA
gcagatcctcc

SEQ ID NO: 105-108 - skipped (4 entries)

SEQ ID NO: 109 - DNA, 26 bp, synthetic construct
  misc_feature 1..26: note = Synthetic
  source 1..26: mol_type = other DNA
ccgtggcactgacccgagactctgag

SEQ ID NO: 110 - skipped

SEQ ID NO: 111 - DNA, 24 bp, synthetic construct
  misc_feature 1..24: note = Synthetic
  source 1..24: mol_type = other DNA
gagctcagcgtagacctccgcgcc

SEQ ID NO: 112-136 - skipped (25 entries)

SEQ ID NO: 137 - DNA, 12 bp, synthetic construct
  misc_feature 1..12: note = Synthetic
  source 1..12: mol_type = other DNA
gtggtccgcctc

SEQ ID NO: 138 - DNA, 18 bp, synthetic construct
  misc_feature 1..18: note = Synthetic
  source 1..18: mol_type = other DNA
gagcaagttggggtgtgc

SEQ ID NO: 139 - DNA, 19 bp, synthetic construct
  misc_feature 1..19: note = Synthetic
  source 1..19: mol_type = other DNA
cgactgcaaggacaaatct

SEQ ID NO: 140 - DNA, 20 bp, synthetic construct
  misc_feature 1..20: note = Synthetic
  source 1..20: mol_type = other DNA
ttcaaaagggacatagaaaa

SEQ ID NO: 141 - DNA, 21 bp, synthetic construct
  misc_feature 1..21: note = Synthetic
  source 1..21: mol_type = other DNA
gctgatctgccgccgcttctc

SEQ ID NO: 142 - DNA, 24 bp, synthetic construct
  misc_feature 1..24: note = Synthetic
  source 1..24: mol_type = other DNA
gcgctctcctcccgctggaatcca

SEQ ID NO: 143 - DNA, 29 bp, synthetic construct
  misc_feature 1..29: note = Synthetic
  source 1..29: mol_type = other DNA
cagagtttatcaccaattccccttcaata

SEQ ID NO: 144 - skipped

SEQ ID NO: 145 - DNA, 16 bp, synthetic construct
  misc_feature 1..16: note = Synthetic
  source 1..16: mol_type = other DNA
ccagcaggggaggccc

SEQ ID NO: 146-148 - skipped (3 entries)

SEQ ID NO: 149 - DNA, 17 bp, synthetic construct
  misc_feature 1..17: note = Synthetic
  source 1..17: mol_type = other DNA
tgggcctcccctgctgg

SEQ ID NO: 150 - DNA, 20 bp, synthetic construct
  misc_feature 1..20: note = Synthetic
  source 1..20: mol_type = other DNA
agctgagctgcgaggtgtcg

SEQ ID NO: 151 - DNA, 20 bp, synthetic construct
  misc_feature 1..20: note = Synthetic
  source 1..20: mol_type = other DNA
cagcaggggaggcccccagc

SEQ ID NO: 152 - RNA, 44 nt, synthetic construct
  source 1..44: mol_type = other RNA
taatttctactaagtgtagatcagtcagggccataggatagata

SEQ ID NO: 153 - RNA, 44 nt, synthetic construct
  source 1..44: mol_type = other RNA
taatttctactaagtgtagatagtcagggccataggatagatat

SEQ ID NO: 154 - RNA, 43 nt, synthetic construct
  source 1..43: mol_type = other RNA
taatttctactgttgtagatcagtcagggccataggatagata

SEQ ID NO: 155-156 - skipped (2 entries)

SEQ ID NO: 157 - DNA, 10 bp, synthetic construct
  misc_feature 1..10: note = Synthetic
  source 1..10: mol_type = other DNA
aaggaggatc

SEQ ID NO: 158 - DNA, 455 bp, synthetic construct
  misc_feature 1..455: note = Synthetic
  source 1..455: mol_type = other DNA
gtgagccactgcgcccagcagattcaagctttttaaatggaattttgagctgatttagttgagacttacgtgcttagttgataaattttaattttatactaaaatattttacattaattcaagttaatttatttcagattgaatttagtggaagcttttgtagaagatgcagaattgaggcagactttacaagaagatttacttcgtcgattcccagatcttaaccgacttgccaagaagtttcaaagacaagcagcaaacttacaagattgttaccgactctatcagggtataaatcaactacctaatgttatacaggctctggaaaaacatgaaggtaacaagtgattttgtttttttgttttccttcaactcatacaatatatacttggcaatgtgctgtcctcataaagttggtggtggtgactcactcttaggacacattcagatttctt

SEQ ID NO: 159 - DNA, 338 bp, synthetic construct
  misc_feature 1..338: note = Synthetic
  source 1..338: mol_type = other DNA
gagcttatcaggttctccattggcaggcagggctctaagtgcagtaacttgatttgctgttgtatttgcttaggaagagcagcacttcagaaaagagtgatggcactgctgaggcgcattgagcatcccactgcaggaaacactgaggtatgcccttagcaacagaaacacccctcccaggcgcccaccctcaatttggaagcctcttgttacatatgtgtgatcaggaatagcttttgaagtaaatccaagatacgtgcatattacaagtataatatctgagtatttaatatacatcaagtttgaaacttggctgtagctgattgatgtttagctct

SEQ ID NO: 160 - DNA, 413 bp, synthetic construct
  misc_feature 1..413: note = Synthetic
  source 1..413: mol_type = other DNA
tgggtacgagtgtctgcgtatatctgtatgcttatttggctctatgcctgtgggtgcacttactctgtgtgtttagatcagtcagtttcatctctctagggggtctgtcttctgggcattgatggcaaatcattaatgtatttgttctttctttaggttttattgactgataccaatactcaatttgtagaacaaaccatagctataatgaagaacttgctagataatcatactgaaggcagctctgaacatctagggcaagctagcattgaaacaatgatgttaaatctggtcaggtaagcattctactgaaatgtagcagaaacattttaagagataagaaaaacctcttacacactgatactggtagtaattgataaaataactggccattctttactgcacacaaacta

SEQ ID NO: 161 - DNA, 23 bp, synthetic construct
  misc_feature 1..23: note = Synthetic
  source 1..23: mol_type = other DNA
accggttccggcggccggggctg

SEQ ID NO: 162 - DNA, 455 bp, synthetic construct; identical to SEQ ID NO: 158
  misc_feature 1..455: note = Synthetic
  source 1..455: mol_type = other DNA

SEQ ID NO: 163 - DNA, 116 bp, synthetic construct
  misc_feature 1..116: note = Synthetic
  source 1..116: mol_type = other DNA
ttgggagctaacggcttggagcttctttccagggatggggacctggaatttgagtactggtagacttttcgttgttcaaaccattccttcacaaattcctgaggaaggcccacagc

SEQ ID NO: 164 - DNA, 178 bp, synthetic construct
  misc_feature 1..178: note = Synthetic
  source 1..178: mol_type = other DNA
taccttgggcctgggccgcagagctgtgagaataccccagggccaggagcgcagtctccaccagctggctaaaaagcacatctttccgcaccaggacaaactcggcgtgttcttctctgttgtcatattcaagagagccgtccaactgctccacgacacaaaagacaggaatcatcaa

SEQ ID NO: 165 - DNA, 125 bp, synthetic construct
  misc_feature 1..125: note = Synthetic
  source 1..125: mol_type = other DNA
gtgcgcgggcggcggccggaagggcctcttcatgcggcggcggcgccggtagttgcccttctcgaacatgtcttcgcaggccgggtccagcgtccagtagttgcccttgcgctcgccgccgccct

SEQ ID NO: 166 - DNA, 276 bp, synthetic construct
  misc_feature 1..276: note = Synthetic
  source 1..276: mol_type = other DNA
ccgggctggagagggggatgttgaggaggctgggggtgggggcggggcatcgagggagctcctggtactggcggccccgactgtccccccagaagctgaaaatgttggacactcctgagaaggcgcctgcagccagagagcagagctgggtgagcggggtagacgcaccaccgctgccacgcccggtcctccctcgcccgcccgtcgcccgggatacctgacagggggttgcaagtgtcgctgctcttctcgcagtcctccatcaggggctcccca

SEQ ID NO: 167 - DNA, 338 bp, synthetic construct; identical to SEQ ID NO: 159
  misc_feature 1..338: note = Synthetic
  source 1..338: mol_type = other DNA

SEQ ID NO: 168 - DNA, 413 bp, synthetic construct; identical to SEQ ID NO: 160
  misc_feature 1..413: note = Synthetic
  source 1..413: mol_type = other DNA

SEQ ID NO: 169 - DNA, 1059 bp, synthetic construct
  misc_feature 1..1059: note = Synthetic
  source 1..1059: mol_type = other DNA
cccaattcaatgtagacagacgtcttttgaggttgtatccgctgctttgtcctcagagttctcacagttccaaggttagagagttggacactgagactggtttcctgctaaacagtatggtaaagaacagtcaagcaattgttggccagttctgtgcttttcctcctgaagagaaacttgacaccatggacaaaataaattgaccatcatcagtcagctaacatgtatgatgcctggaaaaaatgcccaggaatttacacactaaaatgtctggggctgggagcggtagctcatgcctataatcccagcactttgggaggctggagcaggactgcttgaggccaggagttcaagaccagcataagcaacagagtgagacccagtctctacaaaataatagtagtagtaataataaaatgtgtgggatatgtgtgatttgaatttttttttctgttgtcttaaatttttcaaacctgattatgtattatttgtgtaatttttgaagtattaatatagcatattttgaagctgatacttgatatacattccaatcacatctgataacttttttttttgttttggggggtgtacagagtcctgctctgtcacccaggctggagtgcagtggcgcaatctcagctcactgcaacctccgcctcctaagttcaagagattctcctgcctcagcctcctgagtagctgggtctacaagcgtgtgcaactatgcctggctaatttgtgtgtgtgtgtgtatatatatatacatatatatgtgtgtgtgtgtgtatatatatatataacatatatataacatatatatattatatatatataacatatatataacatatatatatgttatatatatataacatatatataacatatatatatatatatatataatatatatatatatatatatatatgtaatcccagcactttgggatatatgtgtatatatgttttttttttttgagacagaatcttgctctgttgccaggctagagtgcagtggcgtgatctcggcacactgcaacctccacctccctggttcag

SEQ ID NO: 170 - DNA, 104 bp, synthetic construct
  misc_feature 1..104: note = Synthetic
  source 1..104: mol_type = other DNA
gggggccattgtggaaaagagcctgcagggagagcaaacagcgcggtcatggcctcgggagctgtgcgcggcgcctcgggcagcgtctcccgccgcttgtcgcc

SEQ ID NO: 171 - DNA, 52 bp, synthetic construct
  misc_feature 1..52: note = Synthetic
  source 1..52: mol_type = other DNA
ctgccgctgcctgaaaccgtacagcttgcagccccatggacagcaccaggtg

SEQ ID NO: 172 - DNA, 52 bp, synthetic construct
  misc_feature 1..52: note = Synthetic
  source 1..52: mol_type = other DNA
cgtggctggagggatcaccaatcccaatcatgccaggggggtatgctctggc

SEQ ID NO: 173 - DNA, 52 bp, synthetic construct
  misc_feature 1..52: note = Synthetic
  source 1..52: mol_type = other DNA
tgtgggagatgccggccgagaagcgttatcttcggggccgtgctgctctttt

SEQ ID NO: 174 - DNA, 52 bp, synthetic construct
  misc_feature 1..52: note = Synthetic
  source 1..52: mol_type = other DNA
tgtggcctaccatctgggccgtgttaagagaggtgaggaaggctcagttttc

SEQ ID NO: 175 - DNA, 52 bp, synthetic construct
  misc_feature 1..52: note = Synthetic
  source 1..52: mol_type = other DNA
tccagtccccctaattagaagagccttgggaacttggtttaatgaacacaca

SEQ ID NO: 176 - DNA, 52 bp, synthetic construct
  misc_feature 1..52: note = Synthetic
  source 1..52: mol_type = other DNA
aaaactaatgagggatgccaaaatcttatcaggtaaggttaaagatgatttt

SEQ ID NO: 177 - DNA, 52 bp, synthetic construct
  misc_feature 1..52: note = Synthetic
  source 1..52: mol_type = other DNA
ttacgatgtcccagaggaagttggtccacccagtaggtggtggggctcactc

SEQ ID NO: 178 - DNA, 52 bp, synthetic construct
  misc_feature 1..52: note = Synthetic
  source 1..52: mol_type = other DNA
gaactgaccaatgagaatgcccagtaactgtcctttcagctgtgaaacacaa

SEQ ID NO: 179 - DNA, 52 bp, synthetic construct
  misc_feature 1..52: note = Synthetic
  source 1..52: mol_type = other DNA
tgataatgataagaactagaactgtggaggactgccacgtgactgtattcct

SEQ ID NO: 180 - DNA, 52 bp, synthetic construct
  misc_feature 1..52: note = Synthetic
  source 1..52: mol_type = other DNA
cccacgacactgcctgaagtagaagccaatcctgtgaggctgccagccatga

SEQ ID NO: 181 - DNA, 52 bp, synthetic construct
  misc_feature 1..52: note = Synthetic
  source 1..52: mol_type = other DNA
ggtgcatattttgaatatcctttcgttactttaaagcctctgtaataagact

SEQ ID NO: 182 - DNA, 52 bp, synthetic construct
  misc_feature 1..52: note = Synthetic
  source 1..52: mol_type = other DNA
gatgagccttttgattttggcactcaagagcaccaccgagaagatccacaac

SEQ ID NO: 183 - DNA, 52 bp, synthetic construct
  misc_feature 1..52: note = Synthetic
  source 1..52: mol_type = other DNA
aagggcagttcccagcggcctcaccaagggatgggctttcctgttctgtact

SEQ ID NO: 184 - DNA, 52 bp, synthetic construct
  misc_feature 1..52: note = Synthetic
  source 1..52: mol_type = other DNA
aggtaggcacaacttacgtaacagattaagagtgaagaagataatgaaattg

SEQ ID NO: 185 - DNA, 52 bp, synthetic construct
  misc_feature 1..52: note = Synthetic
  source 1..52: mol_type = other DNA
tgaaaaggctcgaaagactggacttaattactcccaaagcaggctttgactc

SEQ ID NO: 186 - DNA, 52 bp, synthetic construct
  misc_feature 1..52: note = Synthetic
  source 1..52: mol_type = other DNA
agtgtgcaggctcacaccaattgataagagtgtttactagacttggtgcctc

SEQ ID NO: 187 - DNA, 52 bp, synthetic construct
  misc_feature 1..52: note = Synthetic
  source 1..52: mol_type = other DNA
gcgaaaaaatgaggattcgtatcataagactggtgagttctgagtcttggag

SEQ ID NO: 188 - DNA, 52 bp, synthetic construct
  misc_feature 1..52: note = Synthetic
  source 1..52: mol_type = other DNA
ctcccagaagacccagccatccccattgcccccaagacagttccaccagctg

SEQ ID NO: 189 - DNA, 52 bp, synthetic construct
  misc_feature 1..52: note = Synthetic
  source 1..52: mol_type = other DNA
tggaaatgaccttggagattgtagcaagagagtgagcatgaggagcggcctg

SEQ ID NO: 190 - DNA, 52 bp, synthetic construct
  misc_feature 1..52: note = Synthetic
  source 1..52: mol_type = other DNA
agtaccttcaggttccttctcacataagagagaagcccagtattttctatca

SEQ ID NO: 191 - DNA, 52 bp, synthetic construct
  misc_feature 1..52: note = Synthetic
  source 1..52: mol_type = other DNA
aacctctacttcttactcacaacataacagagaagccgagtattttctacca

SEQ ID NO: 192 - DNA, 52 bp, synthetic construct
  misc_feature 1..52: note = Synthetic
  source 1..52: mol_type = other DNA
aaaatttaaatccacaattttgtcattcttcgcggacgagggtcgcagtcaa

SEQ ID NO: 193 - DNA, 52 bp, synthetic construct
  misc_feature 1..52: note = Synthetic
  source 1..52: mol_type = other DNA
acactctactctggcactttcaaagttaagtttctgcactgttaatttcttg

SEQ ID NO: 194 - DNA, 52 bp, synthetic construct
  misc_feature 1..52: note = Synthetic
  source 1..52: mol_type = other DNA
ctgcatctcaggagtgacaggggttggcggtggctttccccacattttcttt

SEQ ID NO: 195 - DNA, 52 bp, synthetic construct
  misc_feature 1..52: note = Synthetic
  source 1..52: mol_type = other DNA
taaaaatgtgagcgtttcagctgttggcgactgttgctcctagcaaccgagc

SEQ ID NO: 196 - DNA, 52 bp, synthetic construct
  misc_feature 1..52: note = Synthetic
  source 1..52: mol_type = other DNA
ccatgacagacacggccttggtcccggctggcatttttcactgttaaagtgt

SEQ ID NO: 197 - DNA, 52 bp, synthetic construct
  misc_feature 1..52: note = Synthetic
  source 1..52: mol_type = other DNA
gatcagtgactgtatatcttgttttccacacatgcctctgcattcaccttgt

SEQ ID NO: 198 - DNA, 52 bp, synthetic construct
  misc_feature 1..52: note = Synthetic
  source 1..52: mol_type = other DNA
ccacgggccagctgacctttgactgttgacgttggccacggtgcgcacccgg

SEQ ID NO: 199 - DNA, 52 bp, synthetic construct
  misc_feature 1..52: note = Synthetic
  source 1..52: mol_type = other DNA
tcttccgttccgcctgctctatctcaagtcacctacttccccttcgactggc

SEQ ID NO: 200 - DNA, 52 bp, synthetic construct
  misc_feature 1..52: note = Synthetic
  source 1..52: mol_type = other DNA
ccccgtacccgggtcccgcagacttaacgcgcaagccgcgcgtagggcccag

SEQ ID NO: 201 - DNA, 52 bp, synthetic construct
  misc_feature 1..52: note = Synthetic
  source 1..52: mol_type = other DNA
tcccacagctccagtaggtccagtcaaggccctggaggaagagaaagttcag

SEQ ID NO: 202 - DNA, 52 bp, synthetic construct
  misc_feature 1..52: note = Synthetic
  source 1..52: mol_type = other DNA
acccacagcaaatagcttgacccccttgccccttcagcctttgggcagctgt

SEQ ID NO: 203 - DNA, 52 bp, synthetic construct
  misc_feature 1..52: note = Synthetic
  source 1..52: mol_type = other DNA
ggaacaccgtcaccccgcggctgatggcgtcacttcaactacctgtctttcg

SEQ ID NO: 204 - DNA, 52 bp, synthetic construct
  misc_feature 1..52: note = Synthetic
  source 1..52: mol_type = other DNA
gtcatgattcctactttatacagtattcatctttgtggtgggaatatttgga

SEQ ID NO: 205 - DNA, 52 bp, synthetic construct
  misc_feature 1..52: note = Synthetic
  source 1..52: mol_type = other DNA
ggagggggagcgctcgcgagtagggcctgctgctggggctctgcttcgggct

SEQ ID NO: 206 - DNA, 52 bp, synthetic construct
  misc_feature 1..52: note = Synthetic
  source 1..52: mol_type = other DNA
acattttgtggtcatttctactcataactggagagcttggtcaagagatatt

SEQ ID NO: 207 - DNA, 52 bp, synthetic construct
  misc_feature 1..52: note = Synthetic
  source 1..52: mol_type = other DNA
ccctccatagaaagaagcaaaacttaacacaaagtcatctaattttttgaca

SEQ ID NO: 208 - DNA, 52 bp, synthetic construct
  misc_feature 1..52: note = Synthetic
  source 1..52: mol_type = other DNA
actactaccagtatctgctttagatccgtttgtcttgtgtatctaacaaccg

SEQ ID NO: 209 - DNA, 52 bp, synthetic construct
  misc_feature 1..52: note = Synthetic
  source 1..52: mol_type = other DNA
gcaataacccagcccaaaaaacttgttatcagctgtcggatcaatctgcaga

SEQ ID NO: 210 - DNA, 52 bp, synthetic construct
  misc_feature 1..52: note = Synthetic
  source 1..52: mol_type = other DNA
tttgtgcagcatatcagtgctttcattgccttaaaaattaagaaaaattatc

SEQ ID NO: 211 - DNA, 52 bp, synthetic construct
  misc_feature 1..52: note = Synthetic
  source 1..52: mol_type = other DNA
acaaactaagaagtcataccttggcaaggaagcgcatcaatatatcaccatt

SEQ ID NO: 212 - DNA, 52 bp, synthetic construct
  misc_feature 1..52: note = Synthetic
  source 1..52: mol_type = other DNA
agttcttggtttgcacgggaatgggttaaatttacagatgctatctccaagg

SEQ ID NO: 213 - DNA, 52 bp, synthetic construct
  misc_feature 1..52: note = Synthetic
  source 1..52: mol_type = other DNA
agatatccccggcagccgcaacctgccactggggcgagaagagcccgcccta

SEQ ID NO: 214 - DNA, 52 bp, synthetic construct
  misc_feature 1..52: note = Synthetic
  source 1..52: mol_type = other DNA
ctgctgcaatatttgccccggcgctaattccatggccactgccctcttcctg

SEQ ID NO: 215 - DNA, 52 bp, synthetic construct
  misc_feature 1..52: note = Synthetic
  source 1..52: mol_type = other DNA
aattatcactttcttcagcccccttaacaattttggttggacccaatggggc

SEQ ID NO: 216 - DNA, 52 bp, synthetic construct
  misc_feature 1..52: note = Synthetic
  source 1..52: mol_type = other DNA
agaaacaagagaaacagcacaagttaagacacaggtaatacagtctgtgtcc

SEQ ID NO: 217 - DNA, 52 bp, synthetic construct
  misc_feature 1..52: note = Synthetic
  source 1..52: mol_type = other DNA
gagactcatgagacaagatattgataacacagaaggtaggtctgttttgctt

SEQ ID NO: 218 - DNA, 52 bp, synthetic construct
  misc_feature 1..52: note = Synthetic
  source 1..52: mol_type = other DNA
tgcaaatctggcaaggctgtaggccaattctcattgcaggcagggcacctca

SEQ ID NO: 219 - DNA, 52 bp, synthetic construct
  misc_feature 1..52: note = Synthetic
  source 1..52: mol_type = other DNA
agagaattgaagagaggtgcaggcgttaagctggaggattctaccagggaga

SEQ ID NO: 220 - DNA, 52 bp, synthetic construct
  misc_feature 1..52: note = Synthetic
  source 1..52: mol_type = other DNA
cacaaattgtgttctcacctgacctaacctgaaaaaagatcgcgttgccatc

SEQ ID NO: 221 - DNA, 52 bp, synthetic construct
  misc_feature 1..52: note = Synthetic
  source 1..52: mol_type = other DNA
ctgacatcccagggaagatgccatcaagagatgcagacactgagtgtgcgga

SEQ ID NO: 222 - DNA, 52 bp, synthetic construct
  misc_feature 1..52: note = Synthetic
  source 1..52: mol_type = other DNA
catggatggttctggaggaaaaatgttatgtatttcgaaccataaattcctt

SEQ ID NO: 223 - DNA, 52 bp, synthetic construct
  misc_feature 1..52: note = Synthetic
  source 1..52: mol_type = other DNA
aacttcacacacctttaatgtgcagttaagttgaggatgcttgtgttagtgt

SEQ ID NO: 224 - DNA, 52 bp, synthetic construct
  misc_feature 1..52: note = Synthetic
  source 1..52: mol_type = other DNA
agtgaatgctgaccccattgatagaggacggaaattctgtggagaccagctg

SEQ ID NO: 225 - DNA, 52 bp, synthetic construct
  misc_feature 1..52: note = Synthetic
  source 1..52: mol_type = other DNA
cttctgcttgctggcctggcacaggttaagtcctctcccccgactctttccc

SEQ ID NO: 226 - DNA, 52 bp, synthetic construct
  misc_feature 1..52: note = Synthetic
  source 1..52: mol_type = other DNA
ctggctccccaaagagccctggtagttaactccccctgctgggaagcaaaaa

SEQ ID NO: 227 - DNA, 52 bp, synthetic construct
  misc_feature 1..52: note = Synthetic
  source 1..52: mol_type = other DNA
gtggtccgcgccgcgctgaccactcaaggcaaggacccggctccgccggcct

SEQ ID NO: 228 - DNA, 52 bp, synthetic construct
  misc_feature 1..52: note = Synthetic
  source 1..52: mol_type = other DNA
actttgtggctcctggataactttattagagagatgaaactaaagaactctt

SEQ ID NO: 229 - DNA, 52 bp, synthetic construct
  misc_feature 1..52: note = Synthetic
  source 1..52: mol_type = other DNA
gtagaaccatcgctgtagcaattcgttaagtcagcaaagttacaccaaactg

SEQ ID NO: 230 - DNA, 52 bp, synthetic construct
  misc_feature 1..52: note = Synthetic
  source 1..52: mol_type = other DNA
attcctgatttcagtggctgcagacttgtgtacttctgtgccacacttagga

SEQ ID NO: 231 - DNA, 52 bp, synthetic construct
  misc_feature 1..52: note = Synthetic
  source 1..52: mol_type = other DNA
gaactttcacatcaatttctaactcttggttttgtgtccttgaataactgtt

SEQ ID NO: 232 - DNA, 52 bp, synthetic construct
  misc_feature 1..52: note = Synthetic
  source 1..52: mol_type = other DNA
cctgtggccattgaactatggaaggttaaaaaaaaaaaaataccacttttaa

SEQ ID NO: 233 - DNA, 52 bp, synthetic construct
  misc_feature 1..52: note = Synthetic
  source 1..52: mol_type = other DNA
ctctagagcgccagagaagattgttaacatttaaaatgttcatcactcagtt

SEQ ID NO: 234 - DNA, 52 bp, synthetic construct
  misc_feature 1..52: note = Synthetic
  source 1..52: mol_type = other DNA
aaaagaaggaaactgcagtggctgcaattcaagattgtaacaggtaaactgc

SEQ ID NO: 235 - DNA, 52 bp, synthetic construct
  misc_feature 1..52: note = Synthetic
  source 1..52: mol_type = other DNA
cctgcaggttggagcctcccctcttaacgtctctgtgcttgtggctctgggg

SEQ ID NO: 236 - DNA, 52 bp, synthetic construct
  misc_feature 1..52: note = Synthetic
  source 1..52: mol_type = other DNA
acccacctggatcggcctccgaccgttaactattcggtgcgttgggcagcgc

SEQ ID NO: 237 - DNA, 52 bp, synthetic construct
  misc_feature 1..52: note = Synthetic
  source 1..52: mol_type = other DNA
aaaatctacaatcaccttttcccccaacagtgtgcatatgcttaaggagttc

SEQ ID NO: 238 - DNA, 52 bp, synthetic construct
  misc_feature 1..52: note = Synthetic
  source 1..52: mol_type = other DNA
ccccataaacagcctcataagcaggttaacgtacgcacaccttccttctgat

SEQ ID NO: 239 - DNA, 52 bp, synthetic construct
  misc_feature 1..52: note = Synthetic
  source 1..52: mol_type = other DNA
tagtctgtgctctctgctgcctcccaatcacgggggccgtagtagaaggcca

SEQ ID NO: 240 - DNA, 52 bp, synthetic construct
  misc_feature 1..52: note = Synthetic
  source 1..52: mol_type = other DNA
caacattccactggtacatattcttaactctggttgccttcagcggaactga

SEQ ID NO: 241 - DNA, 52 bp, synthetic construct
  misc_feature 1..52: note = Synthetic
  source 1..52: mol_type = other DNA
tgctgtgcgcgtctgctccctgctgttatcagtctgtccagcacttccattg

SEQ ID NO: 242 - DNA, 52 bp, synthetic construct
  misc_feature 1..52: note = Synthetic
  source 1..52: mol_type = other DNA
ggcagcaggtgctctactgggagtcaagagagcagccaggctgagatggagt

SEQ ID NO: 243 - DNA, 52 bp, synthetic construct
  misc_feature 1..52: note = Synthetic
  source 1..52: mol_type = other DNA
gtacatgatgaggcccacgggccccaaggaagagcagctcccgcttggcgat

SEQ ID NO: 244 - DNA, 52 bp, synthetic construct
  misc_feature 1..52: note = Synthetic
  source 1..52: mol_type = other DNA
attgcaggtcactggaaggactgagccgcattcaggagcctggaggaactca

SEQ ID NO: 245 - DNA, 52 bp, synthetic construct
  misc_feature 1..52: note = Synthetic
  source 1..52: mol_type = other DNA
gaaagggacgaactggtgtaatgataatgtgcatatttattacatcggggca

SEQ ID NO: 246 - DNA, 52 bp, synthetic construct
  misc_feature 1..52: note = Synthetic
  source 1..52: mol_type = other DNA
tgtactttgagttccctcagccgttaacctgtgtgtggtgatatcaaagtag

SEQ ID NO: 247 - DNA, 52 bp, synthetic construct
  misc_feature 1..52: note = Synthetic
  source 1..52: mol_type = other DNA
ggacagctccaccaacatccacgagcctgcgggtcaccaacaccagcatcca

SEQ ID NO: 248 - DNA, 52 bp, synthetic construct
  misc_feature 1..52: note = Synthetic
  source 1..52: mol_type = other DNA
cctcgagcgtcccaccggctggaaattgcttcgtttaccacttcgccgtgtg

SEQ ID NO: 249 - DNA, 52 bp, synthetic construct
  misc_feature 1..52: note = Synthetic
  source 1..52: mol_type = other DNA
ttgtccaggtgagccaggccatcacttaaaggcaccgagcactttcttgcca

SEQ ID NO: 250 - DNA, 52 bp, synthetic construct
  misc_feature 1..52: note = Synthetic
  source 1..52: mol_type = other DNA
cttcatccacgttcaccttgccccaccagggcagtaacggcagacttctcct

SEQ ID NO: 251 - DNA, 52 bp, synthetic construct
  misc_feature 1..52: note = Synthetic
  source 1..52: mol_type = other DNA
tgccccacagggcagtaacggcagaccttctcctcaggagtcagatgcacca

SEQ ID NO: 252 - DNA, 52 bp, synthetic construct
  misc_feature 1..52: note = Synthetic
  source 1..52: mol_type = other DNA
gagaactgtagcttctaaagctcataagcataggtgtttggcaagacattct

SEQ ID NO: 253 - DNA, 52 bp, synthetic construct
  misc_feature 1..52: note = Synthetic
  source 1..52: mol_type = other DNA
cctcgatcatgcgccgcgcttcatgaactcagctcctgaatcaggtcgaagt

SEQ ID NO: 254 - DNA, 52 bp, synthetic construct
  misc_feature 1..52: note = Synthetic
  source 1..52: mol_type = other DNA
cgccatcgtaggcaggcggctcccacctgtactgtgcaggagtcctctccca

SEQ ID NO: 255 - DNA, 52 bp, synthetic construct
  misc_feature 1..52: note = Synthetic
  source 1..52: mol_type = other DNA
aacacccactcatcgctgtcacctgttgtcctctggggcatctggggctggc

SEQ ID NO: 256 - DNA, 52 bp, synthetic construct
  misc_feature 1..52: note = Synthetic
  source 1..52: mol_type = other DNA
atggggtcatcgggggctccaggggttaggaccattgagagctgctgagctt

SEQ ID NO: 257 - DNA, 52 bp, synthetic construct
  misc_feature 1..52: note = Synthetic
  source 1..52: mol_type = other DNA
gactctggcagctcaagctctaaggttaacacgatacggctgtccatacatc

SEQ ID NO: 258 - DNA, 52 bp, synthetic construct
  misc_feature 1..52: note = Synthetic
  source 1..52: mol_type = other DNA
cctgcgaagatggccgctgcgtcctccatcggattccgacgcctgcggagct

SEQ ID NO: 259 - DNA, 51 bp, synthetic construct
  misc_feature 1..51: note = Synthetic
  source 1..51: mol_type = other DNA
tgactgtatgcacaacaacgggcagttgtgggcagctgtgccttgccatcc

SEQ ID NO: 260 - DNA, 52 bp, synthetic construct
  misc_feature 1..52: note = Synthetic
  source 1..52: mol_type = other DNA
cggcgatggtaggggccgtcctcacttgccctgctggcagggcttgtgagct

SEQ ID NO: 261 - DNA, 52 bp, synthetic construct
  misc_feature 1..52: note = Synthetic
  source 1..52: mol_type = other DNA
ggcagggcttgtgagcttgctgtgtccgtcacaagagaaagcagcttcctga

SEQ ID NO: 262 - DNA, 52 bp, synthetic construct
  misc_feature 1..52: note = Synthetic
  source 1..52: mol_type = other DNA
tccacaaatttctggctaaagcgaaggaacactgaaaggttcaaaacctcca

SEQ ID NO: 263 - DNA, 52 bp, synthetic construct
  misc_feature 1..52: note = Synthetic
  source 1..52: mol_type = other DNA
acagaacaatcccagcctaaaacttaacatacacagaatgtctgagggtttg

SEQ ID NO: 264 - DNA, 52 bp, synthetic construct
  misc_feature 1..52: note = Synthetic
  source 1..52: mol_type = other DNA
aggagtggaagaaggcactgtgctcaagtgttggtggacaagtgaatttgct

SEQ ID NO: 265 - DNA, 52 bp, synthetic construct
  misc_feature 1..52: note = Synthetic
  source 1..52: mol_type = other DNA
tgtggtagagcaatcccaaagtggtggaaaaaaaagggtatcatgaagtaga

SEQ ID NO: 266 - DNA, 52 bp, synthetic construct
  misc_feature 1..52: note = Synthetic
  source 1..52: mol_type = other DNA
aggagaggttatgaagaatgcacacaacaattctccgtggcctgagaaaaca

SEQ ID NO: 267 - DNA, 52 bp, synthetic construct
  misc_feature 1..52: note = Synthetic
  source 1..52: mol_type = other DNA
ggcccgcctgctttcttggtggtgcaagggtgtgcccagcctggcttctctg

SEQ ID NO: 268 - DNA, 52 bp, synthetic construct
  misc_feature 1..52: note = Synthetic
  source 1..52: mol_type = other DNA
ggtcccagaccatggagtgagcctcttcccccaagcctgtgggtaaggacag

SEQ ID NO: 269 - DNA, 52 bp, synthetic construct
  misc_feature 1..52: note = Synthetic
  source 1..52: mol_type = other DNA
ttcatggagaacatgccgtggtacattggcaaggtacaaagccgttagagcc

SEQ ID NO: 270 - DNA, 52 bp, synthetic construct
  misc_feature 1..52: note = Synthetic
  source 1..52: mol_type = other DNA
tccggccctgcggtcagcgcgtcccttggtcggagacaccgagggtgtgacc

SEQ ID NO: 271 - DNA, 52 bp, synthetic construct
  misc_feature 1..52: note = Synthetic
  source 1..52: mol_type = other DNA
agtcgggcacggcctacgagtgcctccagcgccctgggcgagaaaggcctgg

SEQ ID NO: 272 - DNA, 52 bp, synthetic construct
  misc_feature 1..52: note = Synthetic
  source 1..52: mol_type = other DNA
cttcctcttcttctcatgtctccggttaggccacgtgcatggccactaggag

SEQ ID NO: 273 - DNA, 52 bp, synthetic construct
  misc_feature 1..52: note = Synthetic
  source 1..52: mol_type = other DNA
ccggtaggccacgtgcatggccactaaggagcgctggcgtggacacgaagat

SEQ ID NO: 274 - DNA, 52 bp, synthetic construct
  misc_feature 1..52: note = Synthetic
  source 1..52: mol_type = other DNA
ccctttttaatgcaaggaaaggctgttatgggtttcagatttatctgactgt

SEQ ID NO: 275 - DNA, 52 bp, synthetic construct
  misc_feature 1..52: note = Synthetic
  source 1..52: mol_type = other DNA
cttattttaactcctacttccaaggaatgttctgtcaaacctagtcatgatt

SEQ ID NO: 276 - DNA, 52 bp, synthetic construct
  misc_feature 1..52: note = Synthetic
  source 1..52: mol_type = other DNA
attgtaaaaatagtcatataaccccttcagatgttattttccaagcaggatt

SEQ ID NO: 277 - DNA, 52 bp, synthetic construct
  misc_feature 1..52: note = Synthetic
  source 1..52: mol_type = other DNA
atcaccagttttagccatcaatgggccaaagaccctaaagtacagagaggcc

SEQ ID NO: 278 - DNA, 52 bp, synthetic construct
  misc_feature 1..52: note = Synthetic
  source 1..52: mol_type = other DNA
caaacgaaaattatggcaggttgttaacgaggcattggatgattcagaggat

SEQ ID NO: 279 - DNA, 52 bp, synthetic construct
  misc_feature 1..52: note = Synthetic
  source 1..52: mol_type = other DNA
tttctgaaatagaagatagtaccaaggcaagtcttttccaaagtattgttta

SEQ ID NO: 280 - DNA, 52 bp, synthetic construct
  misc_feature 1..52: note = Synthetic
  source 1..52: mol_type = other DNA
gtttctttagagccgattacctgtgttaccctttcggtaagacatgtttaaa

SEQ ID NO: 281 - DNA, 52 bp, synthetic construct
  misc_feature 1..52: note = Synthetic
  source 1..52: mol_type = other DNA
tgcccctttcgtctatttgtcagacggaatgttacaatttactggcaataaa

SEQ ID NO: 282 - DNA, 52 bp, synthetic construct
  misc_feature 1..52: note = Synthetic
  source 1..52: mol_type = other DNA
agatgtcttctcctaattgtgagataatattatcaaagtcctttatcacttt

SEQ ID NO: 283 - DNA, 52 bp, synthetic construct
  misc_feature 1..52: note = Synthetic
  source 1..52: mol_type = other DNA
caggagcacccgcatgaccctggggaacgccatgggtaatggtgccagtctt

SEQ ID NO: 284 - DNA, 52 bp, synthetic construct
  misc_feature 1..52: note = Synthetic
  source 1..52: mol_type = other DNA
gtccatgttggctgacctgtgtctcaagagatttgtaggcctgaacgtagaa

SEQ ID NO: 285 - DNA, 52 bp, synthetic construct
  misc_feature 1..52: note = Synthetic
  source 1..52: mol_type = other DNA
cccagctgctgctgcagggtagccaggccagtacagctcctccaggcctggc

SEQ ID NO: 286 - DNA, 52 bp, synthetic construct
  misc_feature 1..52: note = Synthetic
  source 1..52: mol_type = other DNA
caggagcccgagtctatattgcctgccagagatgtactgaagggggagtctg

SEQ ID NO: 287 - DNA, 52 bp, synthetic construct
  misc_feature 1..52: note = Synthetic
  source 1..52: mol_type = other DNA
atgccaaagaaaaaatagctcctttaacctttagaagggcatgactcaacat

SEQ ID NO: 288 - DNA, 52 bp, synthetic construct
  misc_feature 1..52: note = Synthetic
  source 1..52: mol_type = other DNA
cttctcggaggaggcagatgcagacaagaccagacacctccctgcttagcag

SEQ ID NO: 289 - DNA, 52 bp, synthetic construct
  misc_feature 1..52: note = Synthetic
  source 1..52: mol_type = other DNA
ataagctcttgtgacctcctggttgttaagtgttggccagatgtctatgtcc

SEQ ID NO: 290 - DNA, 52 bp, synthetic construct
  misc_feature 1..52: note = Synthetic
  source 1..52: mol_type = other DNA
ctgagttcccaccggatgagacctcttctcttttatagccagaagttcccca

SEQ ID NO: 291 - DNA, 52 bp, synthetic construct
  misc_feature 1..52: note = Synthetic
  source 1..52: mol_type = other DNA
aacagctctcagcctggatgctccattggactcctcttggaccagcatgaag

SEQ ID NO: 292 - DNA, 52 bp, synthetic construct
  misc_feature 1..52: note = Synthetic
  source 1..52: mol_type = other DNA
cataccttggcaagatcatacagacaagaggacctgtcgacagtagttcttc

SEQ ID NO: 293 - DNA, 52 bp, synthetic construct
  misc_feature 1..52: note = Synthetic
  source 1..52: mol_type = other DNA
tgggtctccaaatcccagagggtaattggtatagcccatcctttccacttcc

SEQ ID NO: 294 - DNA, 52 bp, synthetic construct
  misc_feature 1..52: note = Synthetic
  source 1..52: mol_type = other DNA
ttcacctggcgtctgaaagagaattaacgccgcggagtacgcgaacgcactg

SEQ ID NO: 295 - DNA, 52 bp, synthetic construct
  misc_feature 1..52: note = Synthetic
  source 1..52: mol_type = other DNA
aagcccaaagccttcaaagacacttaaccttggcaccttcttccactggagg

SEQ ID NO: 296 - DNA, 52 bp, synthetic construct
  misc_feature 1..52: note = Synthetic
  source 1..52: mol_type = other DNA
gtgccgcgccgcgttgcccatctccaaggaggctcagcacctcatccaggcc

SEQ ID NO: 297 - DNA, 52 bp, synthetic construct
  misc_feature 1..52: note = Synthetic
  source 1..52: mol_type = other DNA
aagcagtatttttttttccaactagttggggacatgattttcgtcaagatta

SEQ ID NO: 298 - DNA, 52 bp, synthetic construct
  misc_feature 1..52: note = Synthetic
  source 1..52: mol_type = other DNA
tccttctgttccggtgatggctcttaacggccacagctaatcccagggtaca

SEQ ID NO: 299 - DNA, 52 bp, synthetic construct
  misc_feature 1..52: note = Synthetic
  source 1..52: mol_type = other DNA
tgtccattacttcaatatttttaataaccgtcactctcaagaagcttgcagg

SEQ ID NO: 300 - DNA, 52 bp, synthetic construct
  misc_feature 1..52: note = Synthetic
  source 1..52: mol_type = other DNA
gtactacatgaagtggagccaagataacgtggttctttatgacgcagagcta

SEQ ID NO: 301 - DNA, 52 bp, synthetic construct
  misc_feature 1..52: note = Synthetic
  source 1..52: mol_type = other DNA
cagaaagggtcccactgctactaacttagcctcctctttgtcaggccaagca

SEQ ID NO: 302 - DNA, 52 bp, synthetic construct
  misc_feature 1..52: note = Synthetic
  source 1..52: mol_type = other DNA
gtgctgactactaccgctatctgataagagtctgtaaaggaactgtagtcgc

SEQ ID NO: 303 - DNA, 52 bp, synthetic construct
  misc_feature 1..52: note = Synthetic
  source 1..52: mol_type = other DNA
ctcttaccagcggtattgctgagcgttgactcagggaagtgttccagaaaaa

SEQ ID NO: 304 - DNA, 52 bp, synthetic construct
  misc_feature 1..52: note = Synthetic
  source 1..52: mol_type = other DNA
atggacagcagggtctgctgaggggaagaggccggcctgggtgaggcccagg

SEQ ID NO: 305 - DNA, 52 bp, synthetic construct
  misc_feature 1..52: note = Synthetic
  source 1..52: mol_type = other DNA
ggcggcccagccccgcagcggccacaagcagcatagccgggcgagcggatcc

SEQ ID NO: 306 - DNA, 52 bp, synthetic construct
  misc_feature 1..52: note = Synthetic
  source 1..52: mol_type = other DNA
agcagttggctgcggggagacccttaaccaccagtggccagcagatcatcca

SEQ ID NO: 307 - DNA, 52 bp, synthetic construct
  misc_feature 1..52: note = Synthetic
  source 1..52: mol_type = other DNA
ttcagccaaggcctgcagcagatgattggaccatgtgagtcggctctggcca

SEQ ID NO: 308 - DNA, 52 bp, synthetic construct
  misc_feature 1..52: note = Synthetic
  source 1..52: mol_type = other DNA
cttcagcctgcacgcggttgtcctcaacgctgatcatcatcgtgcagtgctg

SEQ ID NO: 309 - DNA, 52 bp, synthetic construct
  misc_feature 1..52: note = Synthetic
  source 1..52: mol_type = other DNA
ctcgcccccagcgcggtggccagcgccgtgtcctggcctgccatcggcttcc

SEQ ID NO: 310 - DNA, 52 bp, synthetic construct
  misc_feature 1..52: note = Synthetic
  source 1..52: mol_type = other DNA
cgtactggctgctgtatgggatgatggcggacgcagcccacgggaagcatgg

SEQ ID NO: 311 - DNA, 52 bp, synthetic construct
  misc_feature 1..52: note = Synthetic
  source 1..52: mol_type = other DNA
ggcctggcggacaatgctgaagagcttgggggtggctggggtgctggtggct

SEQ ID NO: 312 - DNA, 52 bp, synthetic construct
  misc_feature 1..52: note = Synthetic
  source 1..52: mol_type = other DNA
gcacagtccgaccctgagcacgcccaatgttccccgcaggccggaacagccc

SEQ ID NO: 313 - DNA, 52 bp, synthetic construct
  misc_feature 1..52: note = Synthetic
  source 1..52: mol_type = other DNA
cactctcgggtcaccacaggtgcctccacacatctgcccaattgctggagac

SEQ ID NO: 314 - DNA, 52 bp, synthetic construct
  misc_feature 1..52: note = Synthetic
  source 1..52: mol_type = other DNA
agggaagctcttcatcctcactagattaagttctcttctgaggactctaatt

SEQ ID NO: 315 - DNA, 52 bp, synthetic construct
  misc_feature 1..52: note = Synthetic
  source 1..52: mol_type = other DNA
tagataagttctcttctgaggactcttaatttcttggcccctcttcggtaac

SEQ ID NO: 316 - DNA, 52 bp, synthetic construct
  misc_feature 1..52: note = Synthetic
  source 1..52: mol_type = other DNA
cttttgttttattctcatgaccactaattagtaatattcatcacttgaccat

SEQ ID NO: 317 - DNA, 52 bp, synthetic construct
  misc_feature 1..52: note = Synthetic
  source 1..52: mol_type = other DNA
gaatgtctcacaaataaaccaagataatgctggtacatctgagtcacacaag

SEQ ID NO: 318 - DNA, 52 bp, synthetic construct
  misc_feature 1..52: note = Synthetic
  source 1..52: mol_type = other DNA
tgaggtactgtactttaaagaggtcaacttcaagtgtagactcattgtcctg

SEQ ID NO: 319 - DNA, 52 bp, synthetic construct
  misc_feature 1..52: note = Synthetic
  source 1..52: mol_type = other DNA
actcactttaccacgacaaactgctaaccaggagagctccatcttaaacaac

SEQ ID NO: 320 - DNA, 52 bp, synthetic construct
  misc_feature 1..52: note = Synthetic
  source 1..52: mol_type = other DNA
ttagcctccagctggatagtaaatgttaacaccaagttctgacgaaaaggat

SEQ ID NO: 321 - DNA, 52 bp, synthetic construct
  misc_feature 1..52: note = Synthetic
  source 1..52: mol_type = other DNA
tggcgggcgcctggccgagcctccaggttgtgtgcattgtgggtggggggcc

SEQ ID NO: 322 - DNA, 52 bp, synthetic construct
  misc_feature 1..52: note = Synthetic
  source 1..52: mol_type = other DNA
cccacctgggggcctacctgctgctggtgaagcccttggccagggcaaaagc

SEQ ID NO: 323 - DNA, 52 bp, synthetic construct
  misc_feature 1..52: note = Synthetic
  source 1..52: mol_type = other DNA
aaaaggatgacaggaacatactgggaagccacttctcctaggaccctgccca

SEQ ID NO: 324 - DNA, 52 bp, synthetic construct
  misc_feature 1..52: note = Synthetic
  source 1..52: mol_type = other DNA
tcaaagtcaactgcaagcgagttatggtggagtttagacccagccaggtaac

SEQ ID NO: 325 - DNA, 52 bp, synthetic construct
  misc_feature 1..52: note = Synthetic
  source 1..52: mol_type = other DNA
ctgctgtaatggtgctggtgaatgcttgtgtacttcaaaggcaagtggcaat

SEQ ID NO: 326 - DNA, 52 bp, synthetic construct
  misc_feature 1..52: note = Synthetic
  source 1..52: mol_type = other DNA
acggctcagacgagcaaggctgtcgttaagtgtggccctgcctttgctattg

SEQ ID NO: 327 - DNA, 52 bp, synthetic construct
  misc_feature 1..52: note = Synthetic
  source 1..52: mol_type = other DNA
agcccccaagacgtgctcccaggacggagtttcgctgccacgatgggaagtg

SEQ ID NO: 328 - DNA, 52 bp, synthetic construct
  misc_feature 1..52: note = Synthetic
  source 1..52: mol_type = other DNA
gagtgcctgtgccccgacggcttccaagctggtggcccagcgaagatgcgaa

SEQ ID NO: 329 - DNA, 52 bp, synthetic construct
  misc_feature 1..52: note = Synthetic
  source 1..52: mol_type = other DNA
atctgtgcctccctgccccgcagatccaacccccactcgcccaagtttacct

SEQ ID NO: 330 - DNA, 52 bp, synthetic construct
  misc_feature 1..52: note = Synthetic
  source 1..52: mol_type = other DNA
aggctaaaggtcagctccacagccgttaaggacacagcacacaaccacccga

SEQ ID NO: 331 - DNA, 52 bp, synthetic construct
  misc_feature 1..52: note = Synthetic
  source 1..52: mol_type = other DNA
agcgcagatagccgttggggaccttggcgctggtgccgctttgaggggtcca

SEQ ID NO: 332 - DNA, 52 bp, synthetic construct
  misc_feature 1..52: note = Synthetic
  source 1..52: mol_type = other DNA
ctggggctggagaagacggcgttccggcatatacgcggtgtccacccttctc

SEQ ID NO: 333 - DNA, 52 bp, synthetic construct
  misc_feature 1..52: note = Synthetic
  source 1..52: mol_type = other DNA
ggaggaaggggctcacctttgggtcttggggatacagcaggaagtgagggtc

SEQ ID NO: 334 - DNA, 52 bp, synthetic construct
  misc_feature 1..52: note = Synthetic
  source 1..52: mol_type = other DNA
ctgggggagactcacctcgatgtaattggtctgtgaactctgtcccaaactc

SEQ ID NO: 335 - DNA, 52 bp, synthetic construct
  misc_feature 1..52: note = Synthetic
  source 1..52: mol_type = other DNA
agtttggtccccggactgaccagcattagagcagccgcagccccagctcctt

SEQ ID NO: 336 - DNA, 52 bp, synthetic construct
  misc_feature 1..52: note = Synthetic
  source 1..52: mol_type = other DNA
cctcggcttccagctctcggagcttaacggggaatctctgggtacaagatcc

SEQ ID NO: 337 - DNA, 52 bp, synthetic construct
  misc_feature 1..52: note = Synthetic
  source 1..52: mol_type = other DNA
cccaggggataccccagggatacttaacagctgcaccaacagcgtgtgcgct

SEQ ID NO: 338 - DNA, 52 bp, synthetic construct
  misc_feature 1..52: note = Synthetic
  source 1..52: mol_type = other DNA
tcatgcgcaggtcccagtcgctgacaaccgcgccgggcgtcgtagcgggagc

SEQ ID NO: 339 - DNA, 52 bp, synthetic construct
  misc_feature 1..52: note = Synthetic
  source 1..52: mol_type = other DNA
caggtgccgtccatccacagagcccaagaagcagcacatctagctcggggtt

SEQ ID NO: 340 - DNA, 52 bp, synthetic construct
  misc_feature 1..52: note = Synthetic
  source 1..52: mol_type = other DNA
gaaagggcgtcaccgctgtggtgttaagacaggatcatcctgggcattaagg

SEQ ID NO: 341 - DNA, 52 bp, synthetic construct
  misc_feature 1..52: note = Synthetic
  source 1..52: mol_type = other DNA
tcgggaatggactccctggtagcaattgaccaacagcagacagttgtccgtc

SEQ ID NO: 342 - DNA, 52 bp, synthetic construct
  misc_feature 1..52: note = Synthetic
  source 1..52: mol_type = other DNA
aggtaaccacgtggccagaggcacattccctccctaaagcggagaaaaagta

SEQ ID NO: 343 - DNA, 52 bp, synthetic construct
  misc_feature 1..52: note = Synthetic
  source 1..52: mol_type = other DNA
ctggcagccgcctccacctaggttcttgcccgcagggctgccggctcggatg

SEQ ID NO: 344 - DNA, 52 bp, synthetic construct
  misc_feature 1..52: note = Synthetic
  source 1..52: mol_type = other DNA
agctcattggaggtgaagtgctcccttgtgtgttgtggggatccaggccctg

SEQ ID NO: 345 - DNA, 52 bp, synthetic construct
  misc_feature 1..52: note = Synthetic
  source 1..52: mol_type = other DNA
agaacaggaagcaaagtaccaggccaagccacccaaaccttagaaagctgga

SEQ ID NO: 346 - DNA, 52 bp, synthetic construct
  misc_feature 1..52: note = Synthetic
  source 1..52: mol_type = other DNA
accctttcatattcatacctttctcttgagacttctgcccagacttcaggaa

SEQ ID NO: 347 - DNA, 52 bp, synthetic construct
  misc_feature 1..52: note = Synthetic
  source 1..52: mol_type = other DNA
gaacaggcactgctgccatgagactggctgagcctcaacatccgactcccga

SEQ ID NO: 348 - DNA, 52 bp, synthetic construct
  misc_feature 1..52: note = Synthetic
  source 1..52: mol_type = other DNA
tgagtaaacaaagcatctccaactcttgagtctggccctcggggagtgcatt

SEQ ID NO: 349 - DNA, 52 bp, synthetic construct
  misc_feature 1..52: note = Synthetic
  source 1..52: mol_type = other DNA
tcatgagttatcaattatttttcttaacctatctccatttcagtatatggaa

SEQ ID NO: 350 - DNA, 52 bp, synthetic construct
  misc_feature 1..52: note = Synthetic
  source 1..52: mol_type = other DNA
gaacagcaaaagttcctggttcctcttgctggcactgtcaaaatggaaatct

SEQ ID NO: 351 - DNA, 54 bp, synthetic construct
  misc_feature 1..54: note = Synthetic
  source 1..54: mol_type = other DNA
tctcccacaaaggcaatggcaaccgacacagctttctctgcatgccacctggag

SEQ ID NO: 352 - DNA, 54 bp, synthetic construct
  misc_feature 1..54: note = Synthetic
  source 1..54: mol_type = other DNA
cactcatgatgatgtgacctttgaggcgcagtggcacaggcctttggagatgag

SEQ ID NO: 353 - DNA, 54 bp, synthetic construct
  misc_feature 1..54: note = Synthetic
  source 1..54: mol_type = other DNA
atttggccgttattcagattccctggtgtgcagaagggctataaagtagcacga

SEQ ID NO: 354 - DNA, 54 bp, synthetic construct
  misc_feature 1..54: note = Synthetic
  source 1..54: mol_type = other DNA
ataaggaatggttctgagggtctcatctcatggagaaaaaaccaaataagcaga

SEQ ID NO: 355 - DNA, 54 bp, synthetic construct
  misc_feature 1..54: note = Synthetic
  source 1..54: mol_type = other DNA
tccaacctgcctcctcgcatcatggcacagccggggggctgcagatgatcgggt

SEQ ID NO: 356 - DNA, 54 bp, synthetic construct
  misc_feature 1..54: note = Synthetic
  source 1..54: mol_type = other DNA
cattttgttccatcagctgtcgcactatatctcattcgtaacctacaaaagaca

SEQ ID NO: 357 - DNA, 54 bp, synthetic construct
  misc_feature 1..54: note = Synthetic
  source 1..54: mol_type = other DNA
acaatattagaacaaattttttgatagagacaccattgcaaaacaacataaagt

SEQ ID NO: 358 - DNA, 54 bp, synthetic construct
  misc_feature 1..54: note = Synthetic
  source 1..54: mol_type = other DNA
atttgtcttttctggatgctcaagtacactcagagttacaaacccactttttgt

SEQ ID NO: 359 - DNA, 54 bp, synthetic construct
  misc_feature 1..54: note = Synthetic
  source 1..54: mol_type = other DNA
agctggatggaatttagccgtgtaaacacaaaagaaggatgaggtaaaactctg

SEQ ID NO: 360 - DNA, 54 bp, synthetic construct
  misc_feature 1..54: note = Synthetic
  source 1..54: mol_type = other DNA
tattatgtgttttacatttacgtgggagagtagccgacactttgcttgctatgg

SEQ ID NO: 361 - DNA, 54 bp, synthetic construct
  misc_feature 1..54: note = Synthetic
  source 1..54: mol_type = other DNA
gtccccgggcctctgtctcccgggcgtgtcctccagcagcgggccttcggggcc

SEQ ID NO: 362 - DNA, 54 bp, synthetic construct
  misc_feature 1..54: note = Synthetic
  source 1..54: mol_type = other DNA
actctgaagaacttttctcagacaatgtgagaataattttgtcttccaagtagc

SEQ ID NO: 363 - DNA, 54 bp, synthetic construct
  misc_feature 1..54: note = Synthetic
  source 1..54: mol_type = other DNA
gttgttgtagtccttgtgcaaataggtgtggtgacctgggtggggggtgggggc

SEQ ID NO: 364 - DNA, 54 bp, synthetic construct
  misc_feature 1..54: note = Synthetic
  source 1..54: mol_type = other DNA
atagagtctgtaaaggaactgtagtcgcgccctggtgaaattaggtcttcttag

SEQ ID NO: 365 - DNA, 54 bp, synthetic construct
  misc_feature 1..54: note = Synthetic
  source 1..54: mol_type = other DNA
gtatagagaccagcatcaagcagtttatatcaagactgatggcagaagagaaga

SEQ ID NO: 366 - DNA, 54 bp, synthetic construct
  misc_feature 1..54: note = Synthetic
  source 1..54: mol_type = other DNA
gcacacacacacacgctttttacctgagagtggttaaaatgtcactctgagagg

SEQ ID NO: 367 - DNA, 54 bp, synthetic construct
  misc_feature 1..54: note = Synthetic
  source 1..54: mol_type = other DNA
ccttgattttcttccttttgttcacatattcaaaagtgacttttggactttgtt

SEQ ID NO: 368 - DNA, 54 bp, synthetic construct
  misc_feature 1..54: note = Synthetic
  source 1..54: mol_type = other DNA
agcgcatgaatatgcctggtagaagacacttcctcctcagcctattctttttag

SEQ ID NO: 369 - DNA, 54 bp, synthetic construct
  misc_feature 1..54: note = Synthetic
  source 1..54: mol_type = other DNA
ggcctctttcggggcctctcctacctatactggtcaggttcgtcagctctggga

SEQ ID NO: 370 - DNA, 56 bp, synthetic construct
  misc_feature 1..56: note = Synthetic
  source 1..56: mol_type = other DNA
actgctggaacttcagggcccgcagcaacaactggtcacccttctccagggtcagc

SEQ ID NO: 371 - DNA, 56 bp, synthetic construct
  misc_feature 1..56: note = Synthetic
  source 1..56: mol_type = other DNA
ctccagactcgcacacggctgagggtgatgatgtcctggccctgctcagcatctgt

SEQ ID NO: 372 - DNA, 56 bp, synthetic construct
  misc_feature 1..56: note = Synthetic
  source 1..56: mol_type = other DNA
acgatatacacatctaccttgatcctgatgataaattcttgcaaaatctgttaaaa

SEQ ID NO: 373 - DNA, 56 bp, synthetic construct
  misc_feature 1..56: note = Synthetic
  source 1..56: mol_type = other DNA
agctcctcagccaggtccacgggcagacgacggccccaggcatcgcgcacgtccag

SEQ ID NO: 374 - DNA, 58 bp, synthetic construct
  misc_feature 1..58: note = Synthetic
  source 1..58: mol_type = other DNA
ctgtgccaaagggtggacccgcgggtggctggctgccagggcaacggcaatgtgattg

SEQ ID NO: 375 - DNA, 58 bp, synthetic construct
  misc_feature 1..58: note = Synthetic
  source 1..58: mol_type = other DNA
gcgggccctgcttcctggccggagacggccggccgcgccagcgaggtcccctccctga

SEQ ID NO: 376 - DNA, 58 bp, synthetic construct
  misc_feature 1..58: note = Synthetic
  source 1..58: mol_type = other DNA
caaatccttcctcttcacaccctcctgattgatagatgtaaaagcccttcccagattt

SEQ ID NO: 377 - DNA, 58 bp, synthetic construct
  misc_feature 1..58: note = Synthetic
  source 1..58: mol_type = other DNA
tgggccactctgcagaccgactggccgctcgctgcctttgctgtttctcggctggaac

SEQ ID NO: 378 - DNA, 58 bp, synthetic construct
  misc_feature 1..58: note = Synthetic
  source 1..58: mol_type = other DNA
tttttttggagatgattttattcctaatgaatgacattctaataggctgtgaggaaga

SEQ ID NO: 379 - DNA, 58 bp, synthetic construct
  misc_feature 1..58: note = Synthetic
  source 1..58: mol_type = other DNA
tttaaggtgaaagtacattttttgttgaatgaattaagtgaaactgccagcatactca

SEQ ID NO: 380 - DNA, 58 bp, synthetic construct
  misc_feature 1..58: note = Synthetic
  source 1..58: mol_type = other DNA
ttactttaacaggaagaggtactgcaacaaacatttgatgggacggcaatagcaaatg

SEQ ID NO: 381 - DNA, 58 bp, synthetic construct
  misc_feature 1..58: note = Synthetic
  source 1..58: mol_type = other DNA
cattattttcaactcactaccattcattaattagtagaagattattctcaaaatgttg

SEQ ID NO: 382 - DNA, 58 bp, synthetic construct
  misc_feature 1..58: note = Synthetic
  source 1..58: mol_type = other DNA
aaaagcaagagaatttgagaagatgaatcaatcagtcactacgattatttcggtaact

SEQ ID NO: 383 - DNA, 58 bp, synthetic construct
  misc_feature 1..58: note = Synthetic
  source 1..58: mol_type = other DNA
aagcaagagaatttgagaagatgaatcagtcagtcactacgattatttcggtaactaa

SEQ ID NO: 384 - DNA, 58 bp, synthetic construct
  misc_feature 1..58: note = Synthetic
  source 1..58: mol_type = other DNA
tgacctgtcatgtatggcaacagatagtaagtatatcaaggcaatagtagaacacaaa

SEQ ID NO: 385 - DNA, 58 bp, synthetic construct
  misc_feature 1..58: note = Synthetic
  source 1..58: mol_type = other DNA
cagtgctggtgttggatgtcaatgatgattgatttttctgaggaagtaaccaaacaag

SEQ ID NO: 386 - DNA, 58 bp, synthetic construct
  misc_feature 1..58: note = Synthetic
  source 1..58: mol_type = other DNA
cggcagtcataaatgggtgcgatgctgtttgttactcgatttaaagcaagcatctagg

SEQ ID NO: 387 - DNA, 58 bp, synthetic construct
  misc_feature 1..58: note = Synthetic
  source 1..58: mol_type = other DNA
tcccttgcccatgttttctttgtataacaaacacctgtgcgataagaaagcatccaga

SEQ ID NO: 388 - DNA, 58 bp, synthetic construct
  misc_feature 1..58: note = Synthetic
  source 1..58: mol_type = other DNA
actctctgtatctctggggtgtccaaaacaaacagtctcataatacgacatggacttc

SEQ ID NO: 389 - DNA, 58 bp, synthetic construct
  misc_feature 1..58: note = Synthetic
  source 1..58: mol_type = other DNA
aatgctaatggcattcaaaacaatggtatgtatcccctgctttaattgttagcccatc

SEQ ID NO: 390 - DNA, 58 bp, synthetic construct
  misc_feature 1..58: note = Synthetic
  source 1..58: mol_type = other DNA
gaagctttactcacaacatatctgactttctttcttacttcgagggctaaaccacatt

SEQ ID NO: 391 - DNA, 58 bp, synthetic construct
  misc_feature 1..58: note = Synthetic
  source 1..58: mol_type = other DNA
tcattcagatcacatgaccttcctgcagccagcgggtgaagaacatgctcattgcctt

SEQ ID NO: 392 - DNA, 58 bp, synthetic construct
  misc_feature 1..58: note = Synthetic
  source 1..58: mol_type = other DNA
gatgtcctggccctgctcagcatctgtcagtcagtggagaccacaggccctgctgcgg

SEQ ID NO: 393 - DNA, 58 bp, synthetic construct
  misc_feature 1..58: note = Synthetic
  source 1..58: mol_type = other DNA
cagcctcgacacacgacccacaggctcagtcaggggctggggacagaggcaaggtaag

SEQ ID NO: 394 - DNA, 58 bp, synthetic construct
  misc_feature 1..58: note = Synthetic
  source 1..58: mol_type = other DNA
gaaaacccttccaataggcttgaatcaagcaagcatgctgcttgatgtagtttctgaa

SEQ ID NO: 395 - DNA, 58 bp, synthetic construct
  misc_feature 1..58: note = Synthetic
  source 1..58: mol_type = other DNA
acagatgaactgggctcactgctcagcctgcctgccagcagcgggggcccccgagccc

SEQ ID NO: 396 - DNA, 58 bp, synthetic construct
  misc_feature 1..58: note = Synthetic
  source 1..58: mol_type = other DNA
gccatcatggagatcaaggagtacctgattgattgacatggcctccagggcaggcatg

SEQ ID NO: 397 - DNA, 58 bp, synthetic construct
  misc_feature 1..58: note = Synthetic
  source 1..58: mol_type = other DNA
ctctttattaccattgagccccagctggttggttcctggagagtccattcgagaaaag

SEQ ID NO: 398 - DNA, 58 bp, synthetic construct
  misc_feature 1..58: note = Synthetic
  source 1..58: mol_type = other DNA
gatgggaaaacatatgacaacagatgtgcgtgcactgtgtgctgagaatgcgtgagta

SEQ ID NO: 399 - DNA, 58 bp, synthetic construct
  misc_feature 1..58: note = Synthetic
  source 1..58: mol_type = other DNA
cactagactcacggtcaggcaggcaggccggccagcagggcacctgccttttccaaca

SEQ ID NO: 400 - DNA, 58 bp, synthetic construct
  misc_feature 1..58: note = Synthetic
  source 1..58: mol_type = other DNA
cagacatacctggttttgatggggtcatccatcatacgtaatgcccttagccatctcc

SEQ ID NO: 401 - DNA, 58 bp, synthetic construct
  misc_feature 1..58: note = Synthetic
  source 1..58: mol_type = other DNA
accgctggagtcacccttcccagtgaggcaggcaggggtcgttaggggaggtgatacg

SEQ ID NO: 402 - DNA, 58 bp, synthetic construct
  misc_feature 1..58: note = Synthetic
  source 1..58: mol_type = other DNA
ggcataaagtcactgccaacaagatcaaacaaacaccgcattgagctcacagtcgatg

SEQ ID NO: 403 - DNA, 58 bp, synthetic construct
  misc_feature 1..58: note = Synthetic
  source 1..58: mol_type = other DNA
tttgacttccaacgaggggaagagaagatagatagcgaatattaacagagtattcagc

SEQ ID NO: 404 - DNA, 58 bp, synthetic construct
  misc_feature 1..58: note = Synthetic
  source 1..58: mol_type = other DNA
cgctgaggctggtgtcatactccacagatagatataattcggagagcaggacagtctt

SEQ ID NO: 405 - DNA, 58 bp, synthetic construct
  misc_feature 1..58: note = Synthetic
  source 1..58: mol_type = other DNA
acacagccttctctgtctctcttggcagtcagtcggtcttggcagctgttgtaattgc

SEQ ID NO: 406 - DNA, 58 bp, synthetic construct
  misc_feature 1..58: note = Synthetic
  source 1..58: mol_type = other DNA
cacttcttggtactcctgtcctgaaagatagatattaatttcaagatagaaagaggac

SEQ ID NO: 407 - DNA, 58 bp, synthetic construct
  misc_feature 1..58: note = Synthetic
  source 1..58: mol_type = other DNA
aaatgattggacagttttcatatagtgaatgaagaaagggaaagtggggaaaacaagt

SEQ ID NO: 408 - DNA, 58 bp, synthetic construct
  misc_feature 1..58: note = Synthetic
  source 1..58: mol_type = other DNA
ataagcccaggaaatgggcaagatggaacgaactggagccccatggggccgcactgac

SEQ ID NO: 409 - DNA, 58 bp, synthetic construct
  misc_feature 1..58: note = Synthetic
  source 1..58: mol_type = other DNA
ctgagatacgtacctagccacatgcgaatgaatgatgctcatgtagtctttgttcttg

SEQ ID NO: 410 - DNA, 58 bp, synthetic construct
  misc_feature 1..58: note = Synthetic
  source 1..58: mol_type = other DNA
gagtcgatcagaaagaccaccttggggatggatgccttccagggagggcagctgccca

SEQ ID NO: 411 - DNA, 58 bp, synthetic construct
  misc_feature 1..58: note = Synthetic
  source 1..58: mol_type = other DNA
actacacaaagagaatctagtgattacagacagtgtcccttgcaaaaggaagaaaata

SEQ ID NO: 412 - DNA, 58 bp, synthetic construct
  misc_feature 1..58: note = Synthetic
  source 1..58: mol_type = other DNA
aagtttccctggataaacacagagggagtgagtggctttggcgtcttatggtgaggct

SEQ ID NO: 413 - DNA, 58 bp, synthetic construct
  misc_feature 1..58: note = Synthetic
  source 1..58: mol_type = other DNA
ggctttgggagaagctgacgttgttatccatccccaggaatagctgtcactccggtcc

SEQ ID NO: 414 - DNA, 58 bp, synthetic construct
  misc_feature 1..58: note = Synthetic
  source 1..58: mol_type = other DNA
tccacagtgttgggacaaggccaggcgttcgttgcacttcaccagccgctgcatggag

SEQ ID NO: 415 - DNA, 58 bp, synthetic construct
  misc_feature 1..58: note = Synthetic
  source 1..58: mol_type = other DNA
ctgaaaatgaagataacaaatatactgcttgctgccagtagaaattctcataacttag

SEQ ID NO: 416 - DNA, 58 bp, synthetic construct
  misc_feature 1..58: note = Synthetic
  source 1..58: mol_type = other DNA
ttgtttctccggctgcacagaaggcatttatttcagccaccaaggagttgtggcacca

SEQ ID NO: 417 - DNA, 58 bp, synthetic construct
  misc_feature 1..58: note = Synthetic
  source 1..58: mol_type = other DNA
tcctcagcagctcggtggagtggagcactcacttcgggcgagcgctcggccagtgccc

SEQ ID NO: 418 - DNA, 58 bp, synthetic construct
  misc_feature 1..58: note = Synthetic
  source 1..58: mol_type = other DNA
gaaatccttccagtcagggccataggatagatatacggttcaggtaccagggggcaga

SEQ ID NO: 419 - DNA, 58 bp, synthetic construct
  misc_feature 1..58: note = Synthetic
  source 1..58: mol_type = other DNA
acagaaaggccagtagacagggataaggcaggcaacacccccaacagctttcttcaga

SEQ ID NO: 420 - DNA, 58 bp, synthetic construct
  misc_feature 1..58: note = Synthetic
  source 1..58: mol_type = other DNA
agacagtgcaagcggaaggtgacagtgcctgcctgtggtctctgtgcggagtccaaag

SEQ ID NO: 421 - DNA, 58 bp, synthetic construct
  misc_feature 1..58: note = Synthetic
  source 1..58: mol_type = other DNA
tactcacggagctgcccatgtgggcaagtaagtgcctcttccgcgcgctccacgcggc

SEQ ID NO: 422 - DNA, 58 bp, synthetic construct
  misc_feature 1..58: note = Synthetic
  source 1..58: mol_type = other DNA
agcgttgtacgcggaggagcgcagcgccagccagccgcacgaagcagcgggcccggga

SEQ ID NO: 423 - DNA, 58 bp, synthetic construct
  misc_feature 1..58: note = Synthetic
  source 1..58: mol_type = other DNA
gcagccagctctgcgtgaacctggagggtgggtggctacaagtgccagtgtgaggaag

SEQ ID NO: 424 - DNA, 58 bp, synthetic construct
  misc_feature 1..58: note = Synthetic
  source 1..58: mol_type = other DNA
ggcccccgtgtcaccgtcctggtgcgggagggagttcgaggcatttgacaacgcggtg

SEQ ID NO: 425 - DNA, 58 bp, synthetic construct
  misc_feature 1..58: note = Synthetic
  source 1..58: mol_type = other DNA
atttaccttccaagagtttttgacatgattgatgtattcatctcttaatgccttagga

SEQ ID NO: 426 - DNA, 60 bp, synthetic construct
  misc_feature 1..60: note = Synthetic
  source 1..60: mol_type = other DNA
aactcggcgacccccagcgtggcgtggagcggagcgtgtgctccgtggtgatggccaggc

SEQ ID NO: 427 - DNA, 60 bp, synthetic construct
  misc_feature 1..60: note = Synthetic
  source 1..60: mol_type = other DNA
caggcttcctgaactactacgatgcctgctctgctcggagggtctgcgcgccgccagccc

SEQ ID NO: 428 - DNA, 60 bp, synthetic construct
  misc_feature 1..60: note = Synthetic
  source 1..60: mol_type = other DNA
aggtctgtagatctagctgcgccacgggctgggctgggccgggccgggcagggggctacc

SEQ ID NO: 429 - DNA, 60 bp, synthetic construct
  misc_feature 1..60: note = Synthetic
  source 1..60: mol_type = other DNA
gctgcgccacgggctgggccgggccgggcagggcagggggctaccaccccgcgccgcaga

SEQ ID NO: 430 - DNA, 60 bp, synthetic construct
  misc_feature 1..60: note = Synthetic
  source 1..60: mol_type = other DNA
ctcatgcccttgagcctctttagcatgccgtgccgcgcaggtcagtgacgccgtactgga

SEQ ID NO: 431 - DNA, 60 bp, synthetic construct
  misc_feature 1..60: note = Synthetic
  source 1..60: mol_type = other DNA
agatgagggtgggggtagcttaccagtgaggtgagatgagattcgtcagggtgaccttga

SEQ ID NO: 432 - DNA, 60 bp, synthetic construct
  misc_feature 1..60: note = Synthetic
  source 1..60: mol_type = other DNA
attgtgctcactgtacttggaatgttctcatctcatttcccatttctctttcaggtgaca

SEQ ID NO: 433 - DNA, 60 bp, synthetic construct
  misc_feature 1..60: note = Synthetic
  source 1..60: mol_type = other DNA
tacaatcgggacagctttgataccactcggctcggcagaggctggtgctggagattgggg

SEQ ID NO: 434 - DNA, 60 bp, synthetic construct
  misc_feature 1..60: note = Synthetic
  source 1..60: mol_type = other DNA
aggaagcagcggcaggggacgcccgcggctcggctcgggcacactggacgtacctgtggg

SEQ ID NO: 435 - DNA, 60 bp, synthetic construct
  misc_feature 1..60: note = Synthetic
  source 1..60: mol_type = other DNA
gtcccagtcgctgacaccgcgccgggcgtcgcgtcgtagcgggagcccaggtagtggcgc

SEQ ID NO: 436 - DNA, 62 bp, synthetic construct
  misc_feature 1..62: note = Synthetic
  source 1..62: mol_type = other DNA
cccttcctcccctggagtcacctgcatccatatccatccggtatagtagttgatcacagtgg

SEQ ID NO: 437 - DNA, 62 bp, synthetic construct
  misc_feature 1..62: note = Synthetic
  source 1..62: mol_type = other DNA
aggatgaccatatcagtgggctattttgggcttgggctttcgcttgatactcctaacttgca

SEQ ID NO: 438 - DNA, 62 bp, synthetic construct
  misc_feature 1..62: note = Synthetic
  source 1..62: mol_type = other DNA
agcagcagctcgtcgtccctgagtcacggcgacggcggcggcggcggcggcggcgggggagg

SEQ ID NO: 439 - DNA, 64 bp, synthetic construct
  misc_feature 1..64: note = Synthetic
  source 1..64: mol_type = other DNA
ttcagcacgaaccacgtcctcagcttcacagatcacagagtagtattttatagccctaaagaaa

SEQ ID NO: 440 - DNA, 64 bp, synthetic construct
  misc_feature 1..64: note = Synthetic
  source 1..64: mol_type = other DNA
ggtcccctgccggtgcctctgcccctcaaacatcaaacacgagctcgctccgtggctttttcag

SEQ ID NO: 441 - DNA, 64 bp, synthetic construct
  misc_feature 1..64: note = Synthetic
  source 1..64: mol_type = other DNA
ccaggtccccgcctcataccagcccgacgagggacgaggaggagcgagcgcgcctggcgggcga

SEQ ID NO: 442 - DNA, 64 bp, synthetic construct
  misc_feature 1..64: note = Synthetic
  source 1..64: mol_type = other DNA
ctgtacttgccactgaaaatggatggctcttagctcttagcttcttcgcggatattagtgaatg

SEQ ID NO: 443 - DNA, 66 bp, synthetic construct
  misc_feature 1..66: note = Synthetic
  source 1..66: mol_type = other DNA
tggctttaatgcagcaaggcttgctaatctcccaatctcccagaggaagttattcaaaagggacat

SEQ ID NO: 444 - DNA, 66 bp, synthetic construct
  misc_feature 1..66: note = Synthetic
  source 1..66: mol_type = other DNA
ttgaagaagaggaagaagatacagaacgtgttcacgtgttctccagcttatgattatcttagaggt

SEQ ID NO: 445 - DNA, 66 bp, synthetic construct
  misc_feature 1..66: note = Synthetic
  source 1..66: mol_type = other DNA
agacgtcaatcctccttttgatcagctctgctcctctgctctgctgggacgacgacacgtggtgtc

SEQ ID NO: 446 - DNA, 66 bp, synthetic construct
  misc_feature 1..66: note = Synthetic
  source 1..66: mol_type = other DNA
gaatttgacaggataatagaaaatcaagaaaaaaagaaaaatccttaaaggcttcaaaaagcactc

SEQ ID NO: 447 - DNA, 66 bp, synthetic construct
  misc_feature 1..66: note = Synthetic
  source 1..66: mol_type = other DNA
tcatggctacctcagagctgagctgcgaggtgtcgaggtgtcggaggagaactgtgagcgccggga

SEQ ID NO: 448 - DNA, 66 bp, synthetic construct
  misc_feature 1..66: note = Synthetic
  source 1..66: mol_type = other DNA
gatggtggccccgactgcaaggacaaatctgacaatctgacgaggaaaactgcggtatgggcgggg

SEQ ID NO: 449 - DNA, 66 bp, synthetic construct
  misc_feature 1..66: note = Synthetic
  source 1..66: mol_type = other DNA
agcctatgcgctctgcacgtttgcactcagcacctcagcaccggggacccaagccagcccgtgcgc

SEQ ID NO: 450 - DNA, 68 bp, synthetic construct
  misc_feature 1..68: note = Synthetic
  source 1..68: mol_type = other DNA
tccagcaggacaacagatggctgcatccacactgtccacactgcctctgagaaagccacctctagggt

SEQ ID NO: 451 - DNA, 68 bp, synthetic construct
  misc_feature 1..68: note = Synthetic
  source 1..68: mol_type = other DNA
gcctgttgcccgccagcaccagctcgcgcaggctgcgcaggctgcccaggccgcggaacgccgcatcg

SEQ ID NO: 452 - DNA, 70 bp, synthetic construct
  misc_feature 1..70: note = Synthetic
  source 1..70: mol_type = other DNA
gcagccgccgccagccgcagccatgggccgggcccggccgggcccggccgggccaacgcgggccgcccag

SEQ ID NO: 453 - DNA, 70 bp, synthetic construct
  misc_feature 1..70: note = Synthetic
  source 1..70: mol_type = other DNA
agcccaagacagaggtgttggcagtggtggcatgaggtggcatgaggaatagtgacaggcacaaagctgg

SEQ ID NO: 454 - DNA, 70 bp, synthetic construct
  misc_feature 1..70: note = Synthetic
  source 1..70: mol_type = other DNA
gtgcaggcgcgcgagcacaacccatggctgcgctgggctgcgctgggctgcgcgaggctgaggtgggcgc

SEQ ID NO: 455 - DNA, 70 bp, synthetic construct
  misc_feature 1..70: note = Synthetic
  source 1..70: mol_type = other DNA
ttcagcacgaaccacgtcctcagcttcacagagtatcacagagtagtattttatagccctaaagaaattg

SEQ ID NO: 456 - DNA, 70 bp, synthetic construct
  misc_feature 1..70: note = Synthetic
  source 1..70: mol_type = other DNA
catggaggcatttgcttgtcagcacttccaggttattccaggttagttaccacttcattactggagggca

SEQ ID NO: 457 - DNA, 70 bp, synthetic construct
  misc_feature 1..70: note = Synthetic
  source 1..70: mol_type = other DNA
gtaggagagcatggatctctgggtgcagatcctcccagatcctccatactagcttcccctgaggccagtg

SEQ ID NO: 458 - DNA, 70 bp, synthetic construct
  misc_feature 1..70: note = Synthetic
  source 1..70: mol_type = other DNA
taatgagctggcatgagtatttgtgccacatggctccacatggctccacatgcaagtttgaaacagaact

SEQ ID NO: 459 - DNA, 70 bp, synthetic construct
  misc_feature 1..70: note = Synthetic
  source 1..70: mol_type = other DNA
gttttcgtcatcgaggtgaagacaaaaggaggatcaaggaggatccaagtacctcatctaccgccgctac

SEQ ID NO: 460 - DNA, 72 bp, synthetic construct
  misc_feature 1..72: note = Synthetic
  source 1..72: mol_type = other DNA
gaggtgccccacagctgccgccgctaccggctcgccaccggctcgccaccatcgccaacttctcggcgcttg

SEQ ID NO: 461 - DNA, 72 bp, synthetic construct
  misc_feature 1..72: note = Synthetic
  source 1..72: mol_type = other DNA
gtggtgttcttcgggacggagtacgtggtccgcctctggtccgcctctggtccgccggctgccgcagcaagt

SEQ ID NO: 462 - DNA, 72 bp, synthetic construct
  misc_feature 1..72: note = Synthetic
  source 1..72: mol_type = other DNA
gctgtgaacccctaccagctgctctccatctactcgccatctactcgccagagcacatccgccagtatacca

SEQ ID NO: 463 - DNA, 72 bp, synthetic construct
  misc_feature 1..72: note = Synthetic
  source 1..72: mol_type = other DNA
ccacaagccctagccatgccgtggtagccaatgttcagccaatgttcagcttgtcttgcatctaatgaagca

SEQ ID NO: 464 - DNA, 76 bp, synthetic construct
  misc_feature 1..76: note = Synthetic
  source 1..76: mol_type = other DNA
gaaatggcaatgaaagttgaactagctagaatgagttactagaatgagttaccagagagcagcttgggaggttgat

SEQ ID NO: 465 - DNA, 76 bp, synthetic construct; identical to SEQ ID NO: 464
  misc_feature 1..76: note = Synthetic
  source 1..76: mol_type = other DNA

SEQ ID NO: 466 - DNA, 78 bp, synthetic construct
  misc_feature 1..78: note = Synthetic
  source 1..78: mol_type = other DNA
ccacctcttcggcattgtacgtctgagagctgcggagccagagctgcggagccagggccgggagcccaccccagaagc

SEQ ID NO: 467 - DNA, 80 bp, synthetic construct
  misc_feature 1..80: note = Synthetic
  source 1..80: mol_type = other DNA
gccttcagcacgaaccacgtcctcagcttcacagagtagtgcttcacagagtagtattttatagccctaaagaaattgtg

SEQ ID NO: 468 - DNA, 80 bp, synthetic construct
  misc_feature 1..80: note = Synthetic
  source 1..80: mol_type = other DNA
acagcggtgtgaccaagatatggaagagggagagggaggcgagggagagggaggcaaaggccacggagggcgagaagtcg

SEQ ID NO: 469 - DNA, 80 bp, synthetic construct
  misc_feature 1..80: note = Synthetic
  source 1..80: mol_type = other DNA
tctctccagccctcttaccgtctctcggcccggcggcacacggcccggcggcacacggcccggcagaaatgcagcgccga

SEQ ID NO: 470 - DNA, 82 bp, synthetic construct
  misc_feature 1..82: note = Synthetic
  source 1..82: mol_type = other DNA
actggctggtcctcgccgcgaagcagggccgtcgcgaggctgggccgtcgcgaggctgtgaagctgcttcgccggtgcttgg

SEQ ID NO: 471 - DNA, 82 bp, synthetic construct
  misc_feature 1..82: note = Synthetic
  source 1..82: mol_type = other DNA
cgctgggcggcctgcccctgcccttggcgcggccccggcccggcgcggccccggcccggcgcggcccctggcgcccgagagc

SEQ ID NO: 472 - DNA, 82 bp, synthetic construct
  misc_feature 1..82: note = Synthetic
  source 1..82: mol_type = other DNA
gtcctgcaggtgctggggcaggtgtgggcctcccctgctgggggcctcccctgctgggggctgtggtcagaaagttcagccg

SEQ ID NO: 473 - DNA, 82 bp, synthetic construct
  misc_feature 1..82: note = Synthetic
  source 1..82: mol_type = other DNA
ggtgaccccggagcccgcagccccagccacgcaggtatcgtgccacgcaggtatcgtggcctccgtcctccgcgcgactcct

SEQ ID NO: 474 - DNA, 84 bp, synthetic construct
  misc_feature 1..84: note = Synthetic
  source 1..84: mol_type = other DNA
cctaggatacaggccttcagcacgaaccacgtcctcagcttcaccacgtcctcagcttcacagagtagtattttatagccctaa

SEQ ID NO: 475 - DNA, 84 bp, synthetic construct
  misc_feature 1..84: note = Synthetic
  source 1..84: mol_type = other DNA
gatacaggccttcagcacgaaccacgtcctcagcttcacagagtcctcagcttcacagagtagtattttatagccctaaagaaa

SEQ ID NO: 476 - DNA, 84 bp, synthetic construct
  misc_feature 1..84: note = Synthetic
  source 1..84: mol_type = other DNA
caagacagactgagtctttcaaatgagcaagttggggtgtgcagcaagttggggtgtgcagcaagttcgtccagcaacttctgt

SEQ ID NO: 477 - DNA, 84 bp, synthetic construct
  misc_feature 1..84: note = Synthetic
  source 1..84: mol_type = other DNA
cgcggctcgcggcgtctgggtttgtgttgtcgatggcgacccgttgtcgatggcgacccgtttcccttgcttcagggctgtctc

SEQ ID NO: 478 - DNA, 86 bp, synthetic construct
  misc_feature 1..86: note = Synthetic
  source 1..86: mol_type = other DNA
atgaagcctaggggacaccggggtcggaggcgccgcctgggttggaggcgccgcctgggttggggtctggcactgtgccaggtcgc

SEQ ID NO: 479 - DNA, 86 bp, synthetic construct
  misc_feature 1..86: note = Synthetic
  source 1..86: mol_type = other DNA
gagcttctttaaacaactcttttaactcctgatcagacatgacctcctgatcagacatgacctccagtgcatctatgctactgtga

SEQ ID NO: 480 - DNA, 86 bp, synthetic construct
  misc_feature 1..86: note = Synthetic
  source 1..86: mol_type = other DNA
cagctggcgctgtgatggtggccccgactgcaaggacaaatctgactgcaaggacaaatctgacgaggaaaactgcggtatgggcg

SEQ ID NO: 481 - DNA, 86 bp, synthetic construct
  misc_feature 1..86: note = Synthetic
  source 1..86: mol_type = other DNA
tcgggagatgggaccgtcctgcaggaccgaccacatctgcagaaccgaccacatctgcagaacctctgcgtggagagcgaggattc

SEQ ID NO: 482 - DNA, 88 bp, synthetic construct
  misc_feature 1..88: note = Synthetic
  source 1..88: mol_type = other DNA
ttgctaatctcccagaggaagttattcaaaagggacatagaaaatcaaaagggacatagaaaagcaagagaatttgagaagatgaatc

SEQ ID NO: 483 - DNA, 90 bp, synthetic construct
  misc_feature 1..90: note = Synthetic
  source 1..90: mol_type = other DNA
cttctgcgtggaccttcaggaatttcatacttttcttcctgttcacatacttttcttcctgttcacatacttttcttcgtagacatgctt

SEQ ID NO: 484 - DNA, 90 bp, synthetic construct
  misc_feature 1..90: note = Synthetic
  source 1..90: mol_type = other DNA
ctgcacctacctttgatctgtctttgaagtggtaggaaaaatgtcgaagtggtaggaaaaatgtcttcttccacatctgattcatctacc

SEQ ID NO: 485 - DNA, 90 bp, synthetic construct
  misc_feature 1..90: note = Synthetic
  source 1..90: mol_type = other DNA
ctccacgcccacgatgccgcggatgctgatctgccgccgcttctcctgatctgccgccgcttctcctggtccgtcaggggcttcgccatg

SEQ ID NO: 486 - DNA, 92 bp, synthetic construct
  misc_feature 1..92: note = Synthetic
  source 1..92: mol_type = other DNA
acccggtggccgggctggtggtggggatcctggtgaccgtgctggtgatcctggtgaccgtgctggtgcagagctccagcacctccacatcc

SEQ ID NO: 487 - DNA, 96 bp, synthetic construct
  misc_feature 1..96: note = Synthetic
  source 1..96: mol_type = other DNA
actccgctgtaagtggtttggccagcccgggcagccacctgtaatctccccgggcagccacctgtaatctcgtcttgataacatcagcaggggtca

SEQ ID NO: 488 - DNA, 96 bp, synthetic construct
  misc_feature 1..96: note = Synthetic
  source 1..96: mol_type = other DNA
cggccgcctggccgacagtcaagtgcgctctcctcccgctggaatccacgctctcctcccgctggaatccacggcgacactgggcccagcttccct

SEQ ID NO: 489 - DNA, 96 bp, synthetic construct
  misc_feature 1..96: note = Synthetic
  source 1..96: mol_type = other DNA
gccgacgggccgtgcgcgctgcgcgagctcagcgtagacctccgcgccagctcagcgtagacctccgcgccgagcgctccgtactcatccccgaga

SEQ ID NO: 490 - DNA, 98 bp, synthetic construct
  misc_feature 1..98: note = Synthetic
  source 1..98: mol_type = other DNA
gatcatggccagacaagcatccgttctccttcctgccatctggacaagcctccttcctgccatctggacaagcttcgtcaggggatgtgggatctggg

SEQ ID NO: 491 - DNA, 100 bp, synthetic construct
  misc_feature 1..100: note = Synthetic
  source 1..100: mol_type = other DNA
tgcccctggcttcccacagccacgccgtggcactgacccgagactctgagcgtggcactgacccgagactctgagcggctgctggaggtgcggaagcgga

SEQ ID NO: 492 - DNA, 106 bp, synthetic construct
  misc_feature 1..106: note = Synthetic
  source 1..106: mol_type = other DNA
tcagagcttggtcaggggtgaaagcagagtttatcaccaattccccttcaataagagtttatcaccaattccccttcaataactctccggagctgggagtcctctt

SEQ ID NO: 493 - DNA, 112 bp, synthetic construct
  misc_feature 1..112: note = Synthetic
  source 1..112: mol_type = other DNA
ttgggcacaagaagaaaaaccacatgagctgattggtgtcgatggcaaccagattagagctgattggtgtcgatggcaaccagattacagatctgtccagcagtcatttctc

SEQ ID NO: 494 - DNA, 204 bp, synthetic construct
  misc_feature 1..204: note = Synthetic
  source 1..204: mol_type = other DNA
cttgccttcggtgtggtcccggcaattgtcacacacacctccatatgccccctggctggcggcaaacacagcggggtcaaagtgacatgtctctgagtgcccttgtcacacacacctccatatgccccctggctggcggcaaacacagcggggtcaaagtgacatgtctctgagtgcccattgcagtcgcaccctggaaaaaga

SEQ ID NO: 495 - DNA, 18 bp, synthetic construct
  misc_feature 1..18: note = Synthetic
  source 1..18: mol_type = other DNA
gctcagcctgcctgccag

SEQ ID NO: 496 - DNA, 42 bp, synthetic construct
  misc_feature 1..42: note = Synthetic
  source 1..42: mol_type = other DNA
cccttggcgcggccccggcccggcgcggccccggcccggcgc

SEQ ID NO: 497 - DNA, 18 bp, synthetic construct
  misc_feature 1..18: note = Synthetic
  source 1..18: mol_type = other DNA
cataggatagatatacgg

SEQ ID NO: 498 - DNA, 18 bp, synthetic construct
  misc_feature 1..18: note = Synthetic
  source 1..18: mol_type = other DNA
cataggatagatatacgg

SEQ ID NO: 499 - DNA, 46 bp, synthetic construct
  misc_feature 1..46: note = Synthetic
  source 1..46: mol_type = other DNA
gcaggaccgaccacatctgcagaaccgaccacatctgcagaacctc

SEQ ID NO: 500 - DNA, 44 bp, synthetic construct
  misc_feature 1..44: note = Synthetic
  source 1..44: mol_type = other DNA
tttgtgttgtcgatggcgacccgttgtcgatggcgacccgtttc

SEQ ID NO: 501 - DNA, 18 bp, synthetic construct
  misc_feature 1..18: note = Synthetic
  source 1..18: mol_type = other DNA
agagggagtgagtggctt

SEQ ID NO: 502 - DNA, 30 bp, synthetic construct
  misc_feature 1..30: note = Synthetic
  source 1..30: mol_type = other DNA
gcagtggtggcatgaggtggcatgaggaat

SEQ ID NO: 503 - DNA, 16 bp, synthetic construct
  misc_feature 1..16: note = Synthetic
  source 1..16: mol_type = other DNA
cgcagcaacaactggt

SEQ ID NO: 504 - DNA, 18 bp, synthetic construct
  misc_feature 1..18: note = Synthetic
  source 1..18: mol_type = other DNA
agagaagatagatagcga

SEQ ID NO: 505 - DNA, 40 bp, synthetic construct
  misc_feature 1..40: note = Synthetic
  source 1..40: mol_type = other DNA
cctcagcttcacagagtagtgcttcacagagtagtatttt

SEQ ID NO: 506 - DNA, 18 bp, synthetic construct
  misc_feature 1..18: note = Synthetic
  source 1..18: mol_type = other DNA
gcagcgccagccagccgc

SEQ ID NO: 507 - DNA, 30 bp, synthetic construct
  misc_feature 1..30: note = Synthetic
  source 1..30: mol_type = other DNA
ccatgggccgggcccggccgggcccggccg

SEQ ID NO: 508 - DNA, 14 bp, synthetic construct
  misc_feature 1..14: note = Synthetic
  source 1..14: mol_type = other DNA
ctacctatactggt

SEQ ID NO: 509 - DNA, 22 bp, synthetic construct
  misc_feature 1..22: note = Synthetic
  source 1..22: mol_type = other DNA
gagtcacggcgacggcggcggc

SEQ ID NO: 510 - DNA, 16 bp, synthetic construct
  misc_feature 1..16: note = Synthetic
  source 1..16: mol_type = other DNA
gatcctgatgataaat

SEQ ID NO: 511 - DNA, 18 bp, synthetic construct
  misc_feature 1..18: note = Synthetic
  source 1..18: mol_type = other DNA
ggggtcatccatcatacg

SEQ ID NO: 512 - DNA, 56 bp, synthetic construct
  misc_feature 1..56: note = Synthetic
  source 1..56: mol_type = other DNA
gccagcccgggcagccacctgtaatctccccgggcagccacctgtaatctcgtctt

SEQ ID NO: 513 - DNA, 26 bp, synthetic construct
  misc_feature 1..26: note = Synthetic
  source 1..26: mol_type = other DNA
agctgcgaggtgtcgaggtgtcggag

SEQ ID NO: 514 - DNA, 30 bp, synthetic construct
  misc_feature 1..30: note = Synthetic
  source 1..30: mol_type = other DNA
cagcttcacagagtatcacagagtagtatt

SEQ ID NO: 515 - DNA, 18 bp, synthetic construct
  misc_feature 1..18: note = Synthetic
  source 1..18: mol_type = other DNA
gacagtgcctgcctgtgg

SEQ ID NO: 516 - DNA, 20 bp, synthetic construct
  misc_feature 1..20: note = Synthetic
  source 1..20: mol_type = other DNA
taccactcggctcggcagag

SEQ ID NO: 517 - DNA, 42 bp, synthetic construct
  misc_feature 1..42: note = Synthetic
  source 1..42: mol_type = other DNA
ccccagccacgcaggtatcgtgccacgcaggtatcgtggcct

SEQ ID NO: 518 - DNA, 18 bp, synthetic construct
  misc_feature 1..18: note = Synthetic
  source 1..18: mol_type = other DNA
tgaatcaagcaagcatgc

SEQ ID NO: 519 - DNA, 26 bp, synthetic construct
  misc_feature 1..26: note = Synthetic
  source 1..26: mol_type = other DNA
ttgcactcagcacctcagcaccgggg

SEQ ID NO: 520 - DNA, 42 bp, synthetic construct
  misc_feature 1..42: note = Synthetic
  source 1..42: mol_type = other DNA
ggtgtgggcctcccctgctgggggcctcccctgctgggggct

SEQ ID NO: 521 - DNA, 14 bp, synthetic construct
  misc_feature 1..14: note = Synthetic
  source 1..14: mol_type = other DNA
tgtaaacacaaaag

SEQ ID NO: 522 - DNA, 18 bp, synthetic construct
  misc_feature 1..18: note = Synthetic
  source 1..18: mol_type = other DNA
gcgggtggctggctgcca

SEQ ID NO: 523 - DNA, 18 bp, synthetic construct
  misc_feature 1..18: note = Synthetic
  source 1..18: mol_type = other DNA
tgggcaagtaagtgcctc

SEQ ID NO: 524 - DNA, 24 bp, synthetic construct
  misc_feature 1..24: note = Synthetic
  source 1..24: mol_type = other DNA
cagcttcacagatcacagagtagt

SEQ ID NO: 525 - DNA, 26 bp, synthetic construct
  misc_feature 1..26: note = Synthetic
  source 1..26: mol_type = other DNA
acagaacgtgttcacgtgttctccag

SEQ ID NO: 526 - DNA, 50 bp, synthetic construct
  misc_feature 1..50: note = Synthetic
  source 1..50: mol_type = other DNA
tctttgaagtggtaggaaaaatgtcgaagtggtaggaaaaatgtcttctt

SEQ ID NO: 527 - DNA, 50 bp, synthetic construct
  misc_feature 1..50: note = Synthetic
  source 1..50: mol_type = other DNA
aatttcatacttttcttcctgttcacatacttttcttcctgttcacatac

SEQ ID NO: 528 - DNA, 14 bp, synthetic construct
  misc_feature 1..14: note = Synthetic
  source 1..14: mol_type = other DNA
cagtttatatcaag

SEQ ID NO: 529 - DNA, 36 bp, synthetic construct
  misc_feature 1..36: note = Synthetic
  source 1..36: mol_type = other DNA
gccgtggggcccagtcccggggcccagtcccggagc

SEQ ID NO: 530 - DNA, 32 bp, synthetic construct
  misc_feature 1..32: note = Synthetic
  source 1..32: mol_type = other DNA
gtggtagccaatgttcagccaatgttcagctt

SEQ ID NO: 531 - DNA, 14 bp, synthetic construct
  misc_feature 1..14: note = Synthetic
  source 1..14: mol_type = other DNA
caagtacactcaga

SEQ ID NO: 532 - DNA, 14 bp, synthetic construct
  misc_feature 1..14: note = Synthetic
  source 1..14: mol_type = other DNA
catggcacagccgg

SEQ ID NO: 533 - DNA, 18 bp, synthetic construct
  misc_feature 1..18: note = Synthetic
  source 1..18: mol_type = other DNA
cttggcagtcagtcggtc

SEQ ID NO: 534 - DNA, 32 bp, synthetic construct
  misc_feature 1..32: note = Synthetic
  source 1..32: mol_type = other DNA
ccgctaccggctcgccaccggctcgccaccat

SEQ ID NO: 535 - DNA, 58 bp, synthetic construct
  misc_feature 1..58: note = Synthetic
  source 1..58: mol_type = other DNA
ccgttctccttcctgccatctggacaagcctccttcctgccatctggacaagcttcgt

SEQ ID NO: 536 - DNA, 20 bp, synthetic construct
  misc_feature 1..20: note = Synthetic
  source 1..20: mol_type = other DNA
gggccgggcagggcaggggg

SEQ ID NO: 537 - DNA, 42 bp, synthetic construct
  misc_feature 1..42: note = Synthetic
  source 1..42: mol_type = other DNA
aagcagggccgtcgcgaggctgggccgtcgcgaggctgtgaa

SEQ ID NO: 538 - DNA, 44 bp, synthetic construct
  misc_feature 1..44: note = Synthetic
  source 1..44: mol_type = other DNA
accacgtcctcagcttcacagagtcctcagcttcacagagtagt

SEQ ID NO: 539 - DNA, 36 bp, synthetic construct
  misc_feature 1..36: note = Synthetic
  source 1..36: mol_type = other DNA
actagctagaatgagttactagaatgagttaccaga

SEQ ID NO: 540 - DNA, 18 bp, synthetic construct
  misc_feature 1..18: note = Synthetic
  source 1..18: mol_type = other DNA
cagatgtgcgtgcactgt

SEQ ID NO: 541 - DNA, 18 bp, synthetic construct
  misc_feature 1..18: note = Synthetic
  source 1..18: mol_type = other DNA
cagtgaggcaggcagggg

SEQ ID NO: 542 - DNA, 20 bp, synthetic construct
  misc_feature 1..20: note = Synthetic
  source 1..20: mol_type = other DNA
gccacgggctgggctgggcc

SEQ ID NO: 543 - DNA, 30 bp, synthetic construct
  misc_feature 1..30: note = Synthetic
  source 1..30: mol_type = other DNA
cccatggctgcgctgggctgcgctgggctg

SEQ ID NO: 544 - DNA, 30 bp, synthetic construct
  misc_feature 1..30: note = Synthetic
  source 1..30: mol_type = other DNA
gacaaaaggaggatcaaggaggatccaagt

SEQ ID NO: 545 - DNA, 14 bp, synthetic construct
  misc_feature 1..14: note = Synthetic
  source 1..14: mol_type = other DNA
ttgaggcgcagtgg

SEQ ID NO: 546 - DNA, 14 bp, synthetic construct
  misc_feature 1..14: note = Synthetic
  source 1..14: mol_type = other DNA
tctcatctcatgga

SEQ ID NO: 547 - DNA, 14 bp, synthetic construct
  misc_feature 1..14: note = Synthetic
  source 1..14: mol_type = other DNA
cgggcgtgtcctcc

SEQ ID NO: 548 - DNA, 18 bp, synthetic construct
  misc_feature 1..18: note = Synthetic
  source 1..18: mol_type = other DNA
gtccaaaacaaacagtct

SEQ ID NO: 549 - DNA, 18 bp, synthetic construct
  misc_feature 1..18: note = Synthetic
  source 1..18: mol_type = other DNA
ccagctggttggttcctg

SEQ ID NO: 550 - DNA, 18 bp, synthetic construct
  misc_feature 1..18: note = Synthetic
  source 1..18: mol_type = other DNA
aagatcaaacaaacaccg

SEQ ID NO: 551 - DNA, 18 bp, synthetic construct
  misc_feature 1..18: note = Synthetic
  source 1..18: mol_type = other DNA
tccacagatagatataat

SEQ ID NO: 552 - DNA, 18 bp, synthetic construct
  misc_feature 1..18: note = Synthetic
  source 1..18: mol_type = other DNA
ttgttatccatccccagg

SEQ ID NO: 553 - DNA, 18 bp, synthetic construct
  misc_feature 1..18: note = Synthetic
  source 1..18: mol_type = other DNA
ccaggcgttcgttgcact

SEQ ID NO: 554 - DNA, 18 bp, synthetic construct
  misc_feature 1..18: note = Synthetic
  source 1..18: mol_type = other DNA
aaggcatttatttcagcc

SEQ ID NO: 555 - DNA, 26 bp, synthetic construct
  misc_feature 1..26: note = Synthetic
  source 1..26: mol_type = other DNA
ttgctaatctcccaatctcccagagg

SEQ ID NO: 556 - DNA, 26 bp, synthetic construct
  misc_feature 1..26: note = Synthetic
  source 1..26: mol_type = other DNA
atcagctctgctcctctgctctgctg

SEQ ID NO: 557 - DNA, 30 bp, synthetic construct
  misc_feature 1..30: note = Synthetic
  source 1..30: mol_type = other DNA
agcacttccaggttattccaggttagttac

SEQ ID NO: 558 - DNA, 30 bp, synthetic construct
  misc_feature 1..30: note = Synthetic
  source 1..30: mol_type = other DNA
ttgtgccacatggctccacatggctccaca

SEQ ID NO: 559 - DNA, 32 bp, synthetic construct
  misc_feature 1..32: note = Synthetic
  source 1..32: mol_type = other DNA
gctctccatctactcgccatctactcgccaga

SEQ ID NO: 560 - DNA, 38 bp, synthetic construct
  misc_feature 1..38: note = Synthetic
  source 1..38: mol_type = other DNA
gtctgagagctgcggagccagagctgcggagccagggc

SEQ ID NO: 561 - DNA, 18 bp, synthetic construct
  misc_feature 1..18: note = Synthetic
  source 1..18: mol_type = other DNA
ctgaaagatagatattaa

SEQ ID NO: 562 - DNA, 18 bp, synthetic construct
  misc_feature 1..18: note = Synthetic
  source 1..18: mol_type = other DNA
aggcaggccggccagcag

SEQ ID NO: 563 - DNA, 18 bp, synthetic construct
  misc_feature 1..18: note = Synthetic
  source 1..18: mol_type = other DNA
cttggggatggatgcctt

SEQ ID NO: 564 - DNA, 24 bp, synthetic construct
  misc_feature 1..24: note = Synthetic
  source 1..24: mol_type = other DNA
agcccgacgagggacgaggaggag

SEQ ID NO: 565 - DNA, 14 bp, synthetic construct
  misc_feature 1..14: note = Synthetic
  source 1..14: mol_type = other DNA
aaccgacacagctt

SEQ ID NO: 566 - DNA, 18 bp, synthetic construct
  misc_feature 1..18: note = Synthetic
  source 1..18: mol_type = other DNA
cagatagtaagtatatca

SEQ ID NO: 567 - DNA, 18 bp, synthetic construct
  misc_feature 1..18: note = Synthetic
  source 1..18: mol_type = other DNA
aatgatgattgatttttc

SEQ ID NO: 568 - DNA, 20 bp, synthetic construct
  misc_feature 1..20: note = Synthetic
  source 1..20: mol_type = other DNA
taccagtgaggtgagatgag

SEQ ID NO: 569 - DNA, 52 bp, synthetic construct
  misc_feature 1..52: note = Synthetic
  source 1..52: mol_type = other DNA
gtggggatcctggtgaccgtgctggtgatcctggtgaccgtgctggtgcaga

SEQ ID NO: 570 - DNA, 18 bp, synthetic construct
  misc_feature 1..18: note = Synthetic
  source 1..18: mol_type = other DNA
tgacatgattgatgtatt

SEQ ID NO: 571 - DNA, 18 bp, synthetic construct
  misc_feature 1..18: note = Synthetic
  source 1..18: mol_type = other DNA
atgaatcagtcagtcact

SEQ ID NO: 572 - DNA, 18 bp, synthetic construct
  misc_feature 1..18: note = Synthetic
  source 1..18: mol_type = other DNA
ggataaggcaggcaacac

SEQ ID NO: 573 - DNA, 20 bp, synthetic construct
  misc_feature 1..20: note = Synthetic
  source 1..20: mol_type = other DNA
aatgttctcatctcatttcc

SEQ ID NO: 574 - DNA, 44 bp, synthetic construct
  misc_feature 1..44: note = Synthetic
  source 1..44: mol_type = other DNA
cacgaaccacgtcctcagcttcaccacgtcctcagcttcacaga

SEQ ID NO: 575 - DNA, 46 bp, synthetic construct
  misc_feature 1..46: note = Synthetic
  source 1..46: mol_type = other DNA
tttaactcctgatcagacatgacctcctgatcagacatgacctcca

SEQ ID NO: 576 - DNA, 72 bp, synthetic construct
  misc_feature 1..72: note = Synthetic
  source 1..72: mol_type = other DNA
cacatgagctgattggtgtcgatggcaaccagattagagctgattggtgtcgatggcaaccagattacagat

SEQ ID NO: 577 - DNA, 28 bp, synthetic construct
  misc_feature 1..28: note = Synthetic
  source 1..28: mol_type = other DNA
agctcgcgcaggctgcgcaggctgccca

SEQ ID NO: 578 - DNA, 18 bp, synthetic construct
  misc_feature 1..18: note = Synthetic
  source 1..18: mol_type = other DNA
tggagcactcacttcggg

SEQ ID NO: 579 - DNA, 14 bp, synthetic construct
  misc_feature 1..14: note = Synthetic
  source 1..14: mol_type = other DNA
ttgatagagacacc

SEQ ID NO: 580 - DNA, 16 bp, synthetic construct
  misc_feature 1..16: note = Synthetic
  source 1..16: mol_type = other DNA
gggcagacgacggccc

SEQ ID NO: 581 - DNA, 18 bp, synthetic construct
  misc_feature 1..18: note = Synthetic
  source 1..18: mol_type = other DNA
tcctgcagccagcgggtg

SEQ ID NO: 582 - DNA, 20 bp, synthetic construct
  misc_feature 1..20: note = Synthetic
  source 1..20: mol_type = other DNA
ggcgtggagcggagcgtgtg

SEQ ID NO: 583 - DNA, 28 bp, synthetic construct
  misc_feature 1..28: note = Synthetic
  source 1..28: mol_type = other DNA
ctgcatccacactgtccacactgcctct

SEQ ID NO: 584 - DNA, 40 bp, synthetic construct
  misc_feature 1..40: note = Synthetic
  source 1..40: mol_type = other DNA
tggaagagggagagggaggcgagggagagggaggcaaagg

SEQ ID NO: 585 - DNA, 18 bp, synthetic construct
  misc_feature 1..18: note = Synthetic
  source 1..18: mol_type = other DNA
tgtataacaaacacctgt

SEQ ID NO: 586 - DNA, 18 bp, synthetic construct
  misc_feature 1..18: note = Synthetic
  source 1..18: mol_type = other DNA
ggtgcgggagggagttcg

SEQ ID NO: 587 - DNA, 20 bp, synthetic construct
  misc_feature 1..20: note = Synthetic
  source 1..20: mol_type = other DNA
gatgcctgctctgctcggag

SEQ ID NO: 588 - DNA, 46 bp, synthetic construct
  misc_feature 1..46: note = Synthetic
  source 1..46: mol_type = other DNA
gggtcggaggcgccgcctgggttggaggcgccgcctgggttggggt

SEQ ID NO: 589 - DNA, 18 bp, synthetic construct
  misc_feature 1..18: note = Synthetic
  source 1..18: mol_type = other DNA
caatggtatgtatcccct

SEQ ID NO: 590 - DNA, 40 bp, synthetic construct
  misc_feature 1..40: note = Synthetic
  source 1..40: mol_type = other DNA
tctctcggcccggcggcacacggcccggcggcacacggcc

SEQ ID NO: 591 - DNA, 14 bp, synthetic construct
  misc_feature 1..14: note = Synthetic
  source 1..14: mol_type = other DNA
ttcacatattcaaa

SEQ ID NO: 592 - DNA, 16 bp, synthetic construct
  misc_feature 1..16: note = Synthetic
  source 1..16: mol_type = other DNA
gagggtgatgatgtcc

SEQ ID NO: 593 - DNA, 18 bp, synthetic construct
  misc_feature 1..18: note = Synthetic
  source 1..18: mol_type = other DNA
cctcctgattgatagatg

SEQ ID NO: 594 - DNA, 18 bp, synthetic construct
  misc_feature 1..18: note = Synthetic
  source 1..18: mol_type = other DNA
gatgctgtttgttactcg

SEQ ID NO: 595 - DNA, 18 bp, synthetic construct
  misc_feature 1..18: note = Synthetic
  source 1..18: mol_type = other DNA
catctgtcagtcagtgga

SEQ ID NO: 596 - DNA, 18 bp, synthetic construct
  misc_feature 1..18: note = Synthetic
  source 1..18: mol_type = other DNA
tatagtgaatgaagaaag

SEQ ID NO: 597 - DNA, 18 bp, synthetic construct
  misc_feature 1..18: note = Synthetic
  source 1..18: mol_type = other DNA
tgattacagacagtgtcc

SEQ ID NO: 598 - DNA, 30 bp, synthetic construct
  misc_feature 1..30: note = Synthetic
  source 1..30: mol_type = other DNA
gggtgcagatcctcccagatcctccatact

SEQ ID NO: 599 - DNA, 20 bp, synthetic construct
  misc_feature 1..20: note = Synthetic
  source 1..20: mol_type = other DNA
gccgggcgtcgcgtcgtagc

SEQ ID NO: 600 - DNA, 20 bp, synthetic construct
  misc_feature 1..20: note = Synthetic
  source 1..20: mol_type = other DNA
gcccgcggctcggctcgggc

SEQ ID NO: 601 - DNA, 14 bp, synthetic construct
  misc_feature 1..14: note = Synthetic
  source 1..14: mol_type = other DNA
gacaatgtgagaat

SEQ ID NO: 602 - DNA, 18 bp, synthetic construct
  misc_feature 1..18: note = Synthetic
  source 1..18: mol_type = other DNA
tatactgcttgctgccag

SEQ ID NO: 603 - DNA, 60 bp, synthetic construct
  misc_feature 1..60: note = Synthetic
  source 1..60: mol_type = other DNA
cacgccgtggcactgacccgagactctgagcgtggcactgacccgagactctgagcggct

SEQ ID NO: 604 - DNA, 24 bp, synthetic construct
  misc_feature 1..24: note = Synthetic
  source 1..24: mol_type = other DNA
gcccctcaaacatcaaacacgagc

SEQ ID NO: 605 - DNA, 56 bp, synthetic construct
  misc_feature 1..56: note = Synthetic
  source 1..56: mol_type = other DNA
gcgcgagctcagcgtagacctccgcgccagctcagcgtagacctccgcgccgagcg

SEQ ID NO: 606 - DNA, 14 bp, synthetic construct
  misc_feature 1..14: note = Synthetic
  source 1..14: mol_type = other DNA
aataggtgtggtga

SEQ ID NO: 607 - DNA, 14 bp, synthetic construct
  misc_feature 1..14: note = Synthetic
  source 1..14: mol_type = other DNA
tacctgagagtggt

SEQ ID NO: 608 - DNA, 14 bp, synthetic construct
  misc_feature 1..14: note = Synthetic
  source 1..14: mol_type = other DNA
ccctggtgtgcaga

SEQ ID NO: 609 - DNA, 14 bp, synthetic construct
  misc_feature 1..14: note = Synthetic
  source 1..14: mol_type = other DNA
cgcactatatctca

SEQ ID NO: 610 - DNA, 14 bp, synthetic construct
  misc_feature 1..14: note = Synthetic
  source 1..14: mol_type = other DNA
cgtgggagagtagc

SEQ ID NO: 611 - DNA, 14 bp, synthetic construct
  misc_feature 1..14: note = Synthetic
  source 1..14: mol_type = other DNA
gtagtcgcgccctg

SEQ ID NO: 612 - DNA, 14 bp, synthetic construct
  misc_feature 1..14: note = Synthetic
  source 1..14: mol_type = other DNA
agaagacacttcct

SEQ ID NO: 613 - DNA, 18 bp, synthetic construct
  misc_feature 1..18: note = Synthetic
  source 1..18: mol_type = other DNA
ctggccgctcgctgcctt

SEQ ID NO: 614 - DNA, 18 bp, synthetic construct
  misc_feature 1..18: note = Synthetic
  source 1..18: mol_type = other DNA
ttcctaatgaatgacatt

SEQ ID NO: 615 - DNA, 18 bp, synthetic construct
  misc_feature 1..18: note = Synthetic
  source 1..18: mol_type = other DNA
tttgttgaatgaattaag

SEQ ID NO: 616 - DNA, 18 bp, synthetic construct
  misc_feature 1..18: note = Synthetic
  source 1..18: mol_type = other DNA
actgcaacaaacatttga

SEQ ID NO: 617 - DNA, 18 bp, synthetic construct
  misc_feature 1..18: note = Synthetic
  source 1..18: mol_type = other DNA
cattcattaattagtaga

SEQ ID NO: 618 - DNA, 18 bp, synthetic construct
  misc_feature 1..18: note = Synthetic
  source 1..18: mol_type = other DNA
agatgaatcaatcagtca

SEQ ID NO: 619 - DNA, 18 bp, synthetic construct
  misc_feature 1..18: note = Synthetic
  source 1..18: mol_type = other DNA
tctgactttctttcttac

SEQ ID NO: 620 - DNA, 18 bp, synthetic construct
  misc_feature 1..18: note = Synthetic
  source 1..18: mol_type = other DNA
caggctcagtcaggggct

SEQ ID NO: 621 - DNA, 18 bp, synthetic construct
  misc_feature 1..18: note = Synthetic
  source 1..18: mol_type = other DNA
gtacctgattgattgaca

SEQ ID NO: 622 - DNA, 18 bp, synthetic construct
  misc_feature 1..18: note = Synthetic
  source 1..18: mol_type = other DNA
agatggaacgaactggag

SEQ ID NO: 623 - DNA, 18 bp, synthetic construct
  misc_feature 1..18: note = Synthetic
  source 1..18: mol_type = other DNA
catgcgaatgaatgatgc

SEQ ID NO: 624 - DNA, 18 bp, synthetic construct
  misc_feature 1..18: note = Synthetic
  source 1..18: mol_type = other DNA
ctggagggtgggtggcta

SEQ ID NO: 625 - DNA, 20 bp, synthetic construct
  misc_feature 1..20: note = Synthetic
  source 1..20: mol_type = other DNA
tagcatgccgtgccgcgcag

SEQ ID NO: 626 - DNA, 22 bp, synthetic construct
  misc_feature 1..22: note = Synthetic
  source 1..22: mol_type = other DNA
cctgcatccatatccatccggt

SEQ ID NO: 627 - DNA, 22 bp, synthetic construct
  misc_feature 1..22: note = Synthetic
  source 1..22: mol_type = other DNA
ctattttgggcttgggctttcg

SEQ ID NO: 628 - DNA, 24 bp, synthetic construct
  misc_feature 1..24: note = Synthetic
  source 1..24: mol_type = other DNA
ggatggctcttagctcttagcttc

SEQ ID NO: 629 - DNA, 26 bp, synthetic construct
  misc_feature 1..26: note = Synthetic
  source 1..26: mol_type = other DNA
aaatcaagaaaaaaagaaaaatcctt

SEQ ID NO: 630 - DNA, 26 bp, synthetic construct
  misc_feature 1..26: note = Synthetic
  source 1..26: mol_type = other DNA
ggacaaatctgacaatctgacgagga

SEQ ID NO: 631 - DNA, 32 bp, synthetic construct
  misc_feature 1..32: note = Synthetic
  source 1..32: mol_type = other DNA
gtacgtggtccgcctctggtccgcctctggtc

SEQ ID NO: 632 - DNA, 44 bp, synthetic construct
  misc_feature 1..44: note = Synthetic
  source 1..44: mol_type = other DNA
aaatgagcaagttggggtgtgcagcaagttggggtgtgcagcaa

SEQ ID NO: 633 - DNA, 46 bp, synthetic construct
  misc_feature 1..46: note = Synthetic
  source 1..46: mol_type = other DNA
gccccgactgcaaggacaaatctgactgcaaggacaaatctgacga

SEQ ID NO: 634 - DNA, 48 bp, synthetic construct
  misc_feature 1..48: note = Synthetic
  source 1..48: mol_type = other DNA
gttattcaaaagggacatagaaaatcaaaagggacatagaaaagcaag

SEQ ID NO: 635 - DNA, 50 bp, synthetic construct
  misc_feature 1..50: note = Synthetic
  source 1..50: mol_type = other DNA
ggatgctgatctgccgccgcttctcctgatctgccgccgcttctcctggt

SEQ ID NO: 636 - DNA, 56 bp, synthetic construct
  misc_feature 1..56: note = Synthetic
  source 1..56: mol_type = other DNA
aagtgcgctctcctcccgctggaatccacgctctcctcccgctggaatccacggcg

SEQ ID NO: 637 - DNA, 66 bp, synthetic construct
  misc_feature 1..66: note = Synthetic
  source 1..66: mol_type = other DNA
aaagcagagtttatcaccaattccccttcaataagagtttatcaccaattccccttcaataactct

SEQ ID NO: 638-758 - skipped (121 entries)

SEQ ID NO: 759 - DNA, 10 bp, synthetic construct
  misc_feature 1..10: note = Synthetic
  source 1..10: mol_type = other DNA
atgacaagat

SEQ ID NO: 760 - DNA, 10 bp, synthetic construct
  misc_feature 1..10: note = Synthetic
  source 1..10: mol_type = other DNA
tggagtgtga

SEQ ID NO: 761 - DNA, 10 bp, synthetic construct
  misc_feature 1..10: note = Synthetic
  source 1..10: mol_type = other DNA
tgtgccgcac

SEQ ID NO: 762 - DNA, 10 bp, synthetic construct
  misc_feature 1..10: note = Synthetic
  source 1..10: mol_type = other DNA
agccgcagct

SEQ ID NO: 763 - DNA, 11 bp, synthetic construct
  misc_feature 1..11: note = Synthetic
  source 1..11: mol_type = other DNA
aaagagattcc

SEQ ID NO: 764 - DNA, 11 bp, synthetic construct
  misc_feature 1..11: note = Synthetic
  source 1..11: mol_type = other DNA
agctgcagcat

SEQ ID NO: 765 - DNA, 11 bp, synthetic construct
  misc_feature 1..11: note = Synthetic
  source 1..11: mol_type = other DNA
ctttaatttgt

SEQ ID NO: 766 - DNA, 11 bp, synthetic construct
  misc_feature 1..11: note = Synthetic
  source 1..11: mol_type = other DNA
acatctgggtg

SEQ ID NO: 767 - DNA, 11 bp, synthetic construct
  misc_feature 1..11: note = Synthetic
  source 1..11: mol_type = other DNA
caacaagttca

SEQ ID NO: 768 - DNA, 12 bp, synthetic construct
  misc_feature 1..12: note = Synthetic
  source 1..12: mol_type = other DNA
tggcccggcggc

SEQ ID NO: 769 - DNA, 12 bp, synthetic construct
  misc_feature 1..12: note = Synthetic
  source 1..12: mol_type = other DNA
gaagaaactatt

SEQ ID NO: 770 - DNA, 12 bp, synthetic construct
  misc_feature 1..12: note = Synthetic
  source 1..12: mol_type = other DNA
taaccctctgtc

SEQ ID NO: 771 - DNA, 12 bp, synthetic construct
  misc_feature 1..12: note = Synthetic
  source 1..12: mol_type = other DNA
acctgtggtccc

SEQ ID NO: 772 - DNA, 12 bp, synthetic construct
  misc_feature 1..12: note = Synthetic
  source 1..12: mol_type = other DNA
aggacaaatctg

SEQ ID NO: 773 - DNA, 13 bp, synthetic construct
  misc_feature 1..13: note = Synthetic
  source 1..13: mol_type = other DNA
gacgagctgtgcc

SEQ ID NO: 774 - DNA, 13 bp, synthetic construct
  misc_feature 1..13: note = Synthetic
  source 1..13: mol_type = other DNA
ttctccttggccg

SEQ ID NO: 775 - DNA, 14 bp, synthetic construct
  misc_feature 1..14: note = Synthetic
  source 1..14: mol_type = other DNA
tggtgagcacgctg

SEQ ID NO: 776 - DNA, 14 bp, synthetic construct
  misc_feature 1..14: note = Synthetic
  source 1..14: mol_type = other DNA
cgactgcaaggaca

SEQ ID NO: 777 - DNA, 14 bp, synthetic construct
  misc_feature 1..14: note = Synthetic
  source 1..14: mol_type = other DNA
aaggtcagctccac

SEQ ID NO: 778 - DNA, 15 bp, synthetic construct
  misc_feature 1..15: note = Synthetic
  source 1..15: mol_type = other DNA
gcagcaaatcaagct

SEQ ID NO: 779 - DNA, 15 bp, synthetic construct
  misc_feature 1..15: note = Synthetic
  source 1..15: mol_type = other DNA
gctgccgcgccgcct

SEQ ID NO: 780 - DNA, 15 bp, synthetic construct
  misc_feature 1..15: note = Synthetic
  source 1..15: mol_type = other DNA
tactgcatgcaaatg

SEQ ID NO: 781 - DNA, 15 bp, synthetic construct
  misc_feature 1..15: note = Synthetic
  source 1..15: mol_type = other DNA
aaggacaaatctgac

SEQ ID NO: 782 - DNA, 16 bp, synthetic construct
  misc_feature 1..16: note = Synthetic
  source 1..16: mol_type = other DNA
aacctgggcaagctgg

SEQ ID NO: 783 - DNA, 16 bp, synthetic construct
  misc_feature 1..16: note = Synthetic
  source 1..16: mol_type = other DNA
gctctgcgcgctgctc

SEQ ID NO: 784 - DNA, 17 bp, synthetic construct
  misc_feature 1..17: note = Synthetic
  source 1..17: mol_type = other DNA
ctagctatcgttctttt

SEQ ID NO: 785 - DNA, 17 bp, synthetic construct
  misc_feature 1..17: note = Synthetic
  source 1..17: mol_type = other DNA
gctgctctgcatgtttt

SEQ ID NO: 786 - DNA, 17 bp, synthetic construct
  misc_feature 1..17: note = Synthetic
  source 1..17: mol_type = other DNA
ttggttggtagacactg

SEQ ID NO: 787 - DNA, 17 bp, synthetic construct
  misc_feature 1..17: note = Synthetic
  source 1..17: mol_type = other DNA
ctcttctgctgcttctt

SEQ ID NO: 788 - DNA, 17 bp, synthetic construct
  misc_feature 1..17: note = Synthetic
  source 1..17: mol_type = other DNA
tggcccggaggctgggc

SEQ ID NO: 789 - DNA, 18 bp, synthetic construct
  misc_feature 1..18: note = Synthetic
  source 1..18: mol_type = other DNA
tgggggtgcggcggaggc

SEQ ID NO: 790 - DNA, 18 bp, synthetic construct
  misc_feature 1..18: note = Synthetic
  source 1..18: mol_type = other DNA
ggaggcgggggtgcggcc

SEQ ID NO: 791 - DNA, 18 bp, synthetic construct
  misc_feature 1..18: note = Synthetic
  source 1..18: mol_type = other DNA
aggcgggggtgcggccgg

SEQ ID NO: 792 - DNA, 18 bp, synthetic construct
  misc_feature 1..18: note = Synthetic
  source 1..18: mol_type = other DNA
acccaaagcagctgtact

SEQ ID NO: 793 - DNA, 18 bp, synthetic construct
  misc_feature 1..18: note = Synthetic
  source 1..18: mol_type = other DNA
gcccaggccctgcagggc

SEQ ID NO: 794 - DNA, 18 bp, synthetic construct
  misc_feature 1..18: note = Synthetic
  source 1..18: mol_type = other DNA
gagcaagtccagctcctc

SEQ ID NO: 795 - DNA, 18 bp, synthetic construct
  misc_feature 1..18: note = Synthetic
  source 1..18: mol_type = other DNA
tgcggctgcgtggacctc

SEQ ID NO: 796 - DNA, 18 bp, synthetic construct
  misc_feature 1..18: note = Synthetic
  source 1..18: mol_type = other DNA
tgcaaggacaaatctgac

SEQ ID NO: 797 - DNA, 19 bp, synthetic construct
  misc_feature 1..19: note = Synthetic
  source 1..19: mol_type = other DNA
tgcagacatagatgccccc

SEQ ID NO: 798 - DNA, 19 bp, synthetic construct
  misc_feature 1..19: note = Synthetic
  source 1..19: mol_type = other DNA
tcgcgctgctgctgctctg

SEQ ID NO: 799 - DNA, 19 bp, synthetic construct
  misc_feature 1..19: note = Synthetic
  source 1..19: mol_type = other DNA
gctgctctgcgcgctgctc

SEQ ID NO: 800 - DNA, 20 bp, synthetic construct
  misc_feature 1..20: note = Synthetic
  source 1..20: mol_type = other DNA
ggctgggctggcagggctga

SEQ ID NO: 801 - DNA, 20 bp, synthetic construct
  misc_feature 1..20: note = Synthetic
  source 1..20: mol_type = other DNA
gcaggcggagcaccccaagc

SEQ ID NO: 802 - DNA, 20 bp, synthetic construct
  misc_feature 1..20: note = Synthetic
  source 1..20: mol_type = other DNA
gcaacagctccacctgcatc

SEQ ID NO: 803 - DNA, 20 bp, synthetic construct
  misc_feature 1..20: note = Synthetic
  source 1..20: mol_type = other DNA
gccgggactggtcagatgaa

SEQ ID NO: 804 - DNA, 20 bp, synthetic construct
  misc_feature 1..20: note = Synthetic
  source 1..20: mol_type = other DNA
ggtggatcctgttcatgggt

SEQ ID NO: 805 - DNA, 21 bp, synthetic construct
  misc_feature 1..21: note = Synthetic
  source 1..21: mol_type = other DNA
cgcgcacccctgtgcccacct

SEQ ID NO: 806 - DNA, 21 bp, synthetic construct
  misc_feature 1..21: note = Synthetic
  source 1..21: mol_type = other DNA
tggggcaggcggcggtgcggc

SEQ ID NO: 807 - DNA, 21 bp, synthetic construct
  misc_feature 1..21: note = Synthetic
  source 1..21: mol_type = other DNA
ctctcggtcatgctgcagctg

SEQ ID NO: 808 - DNA, 21 bp, synthetic construct
  misc_feature 1..21: note = Synthetic
  source 1..21: mol_type = other DNA
gactgcaaggacaaatctgac

SEQ ID NO: 809 - DNA, 21 bp, synthetic construct
  misc_feature 1..21: note = Synthetic
  source 1..21: mol_type = other DNA
ccttacagctgcctctgttgt

SEQ ID NO: 810 - DNA, 18 bp, synthetic construct
  misc_feature 1..18: note = Synthetic
  source 1..18: mol_type = other DNA
aggattgggtgggagcag

SEQ ID NO: 811 - DNA, 18 bp, synthetic construct
  misc_feature 1..18: note = Synthetic
  source 1..18: mol_type = other DNA
tttgactcgctcgctaca

SEQ ID NO: 812 - DNA, 18 bp, synthetic construct
  misc_feature 1..18: note = Synthetic
  source 1..18: mol_type = other DNA
agactattaattacgttc

SEQ ID NO: 813 - DNA, 18 bp, synthetic construct
  misc_feature 1..18: note = Synthetic
  source 1..18: mol_type = other DNA
gttatggagggagtccat

SEQ ID NO: 814 - DNA, 18 bp, synthetic construct
  misc_feature 1..18: note = Synthetic
  source 1..18: mol_type = other DNA
ataaagagtgagtctttt

SEQ ID NO: 815 - DNA, 18 bp, synthetic construct
  misc_feature 1..18: note = Synthetic
  source 1..18: mol_type = other DNA
aggatgaccgaccgcgat

SEQ ID NO: 816 - DNA, 18 bp, synthetic construct
  misc_feature 1..18: note = Synthetic
  source 1..18: mol_type = other DNA
aagggacatacatcatca

SEQ ID NO: 817 - DNA, 18 bp, synthetic construct
  misc_feature 1..18: note = Synthetic
  source 1..18: mol_type = other DNA
aaatgggtcggtccatca

SEQ ID NO: 818 - DNA, 18 bp, synthetic construct
  misc_feature 1..18: note = Synthetic
  source 1..18: mol_type = other DNA
tagtagagggaggccaca

SEQ ID NO: 819 - DNA, 18 bp, synthetic construct
  misc_feature 1..18: note = Synthetic
  source 1..18: mol_type = other DNA
tctggcggccggccgact

SEQ ID NO: 820 - DNA, 18 bp, synthetic construct
  misc_feature 1..18: note = Synthetic
  source 1..18: mol_type = other DNA
cgagctgaatgaagcctt

SEQ ID NO: 821 - DNA, 18 bp, synthetic construct
  misc_feature 1..18: note = Synthetic
  source 1..18: mol_type = other DNA
aaaacactgactgagact

SEQ ID NO: 822 - DNA, 18 bp, synthetic construct
  misc_feature 1..18: note = Synthetic
  source 1..18: mol_type = other DNA
cacttctaactaacaaac

SEQ ID NO: 823 - DNA, 18 bp, synthetic construct
  misc_feature 1..18: note = Synthetic
  source 1..18: mol_type = other DNA
agtgtcagccagccattc

SEQ ID NO: 824 - DNA, 18 bp, synthetic construct
  misc_feature 1..18: note = Synthetic
  source 1..18: mol_type = other DNA
tctaatgactgactattt

SEQ ID NO: 825 - DNA, 18 bp, synthetic construct
  misc_feature 1..18: note = Synthetic
  source 1..18: mol_type = other DNA
ttacaggttggttcttaa

SEQ ID NO: 826 - DNA, 18 bp, synthetic construct
  misc_feature 1..18: note = Synthetic
  source 1..18: mol_type = other DNA
ccggtagtaagtacttca

SEQ ID NO: 827 - DNA, 18 bp, synthetic construct
  misc_feature 1..18: note = Synthetic
  source 1..18: mol_type = other DNA
gccagcagccagcagcat

SEQ ID NO: 828 - DNA, 18 bp, synthetic construct
  misc_feature 1..18: note = Synthetic
  source 1..18: mol_type = other DNA
gagctgctggctgctttg

SEQ ID NO: 829 - DNA, 18 bp, synthetic construct
  misc_feature 1..18: note = Synthetic
  source 1..18: mol_type = other DNA
caacgccaaccaacattg

SEQ ID NO: 830 - DNA, 18 bp, synthetic construct
  misc_feature 1..18: note = Synthetic
  source 1..18: mol_type = other DNA
agctctatttatttgatt

SEQ ID NO: 831 - DNA, 18 bp, synthetic construct
  misc_feature 1..18: note = Synthetic
  source 1..18: mol_type = other DNA
caagcaaagaaagatgga

SEQ ID NO: 832 - DNA, 18 bp, synthetic construct
  misc_feature 1..18: note = Synthetic
  source 1..18: mol_type = other DNA
ttctggagtgagtcgcta

SEQ ID NO: 833 - DNA, 18 bp, synthetic construct
  misc_feature 1..18: note = Synthetic
  source 1..18: mol_type = other DNA
tgaactgcttgctcatgg

SEQ ID NO: 834 - DNA, 18 bp, synthetic construct
  misc_feature 1..18: note = Synthetic
  source 1..18: mol_type = other DNA
tggcctcagtcagcaggg

SEQ ID NO: 835 - DNA, 18 bp, synthetic construct
  misc_feature 1..18: note = Synthetic
  source 1..18: mol_type = other DNA
cttctcatccatcgcttt

SEQ ID NO: 836 - DNA, 18 bp, synthetic construct
  misc_feature 1..18: note = Synthetic
  source 1..18: mol_type = other DNA
ctataaacgaacgcagtc

SEQ ID NO: 837 - DNA, 18 bp, synthetic construct
  misc_feature 1..18: note = Synthetic
  source 1..18: mol_type = other DNA
ccacccttacttagttct

SEQ ID NO: 838 - DNA, 18 bp, synthetic construct
  misc_feature 1..18: note = Synthetic
  source 1..18: mol_type = other DNA
ctcatgatagatactact

SEQ ID NO: 839 - DNA, 18 bp, synthetic construct
  misc_feature 1..18: note = Synthetic
  source 1..18: mol_type = other DNA
gaaagtcaatcaatgcca

SEQ ID NO: 840 - DNA, 18 bp, synthetic construct
  misc_feature 1..18: note = Synthetic
  source 1..18: mol_type = other DNA
agttgtacctaccgtctt

SEQ ID NO: 841 - DNA, 18 bp, synthetic construct
  misc_feature 1..18: note = Synthetic
  source 1..18: mol_type = other DNA
tcttgcagtcagtaaagc

SEQ ID NO: 842 - DNA, 18 bp, synthetic construct
  misc_feature 1..18: note = Synthetic
  source 1..18: mol_type = other DNA
cttaactagctagctctt

SEQ ID NO: 843 - DNA, 18 bp, synthetic construct
  misc_feature 1..18: note = Synthetic
  source 1..18: mol_type = other DNA
acttgtgactgactagct

SEQ ID NO: 844 - DNA, 18 bp, synthetic construct
  misc_feature 1..18: note = Synthetic
  source 1..18: mol_type = other DNA
gtgatgttagttagtttg

SEQ ID NO: 845 - DNA, 18 bp, synthetic construct
  misc_feature 1..18: note = Synthetic
  source 1..18: mol_type = other DNA
actgagcatgcatagtct

SEQ ID NO: 846 - DNA, 18 bp, synthetic construct
  misc_feature 1..18: note = Synthetic
  source 1..18: mol_type = other DNA
aaatggaaagaaaaaacc

SEQ ID NO: 847 - DNA, 18 bp, synthetic construct
  misc_feature 1..18: note = Synthetic
  source 1..18: mol_type = other DNA
ctttgttccttccacctt

SEQ ID NO: 848 - DNA, 18 bp, synthetic construct
  misc_feature 1..18: note = Synthetic
  source 1..18: mol_type = other DNA
tgtgtgacagacactcca

SEQ ID NO: 849 - DNA, 18 bp, synthetic construct
  misc_feature 1..18: note = Synthetic
  source 1..18: mol_type = other DNA
agatggatcgatcatatg

SEQ ID NO: 850 - DNA, 18 bp, synthetic construct
  misc_feature 1..18: note = Synthetic
  source 1..18: mol_type = other DNA
acagatgggtgggtggta

SEQ ID NO: 851 - DNA, 18 bp, synthetic construct
  misc_feature 1..18: note = Synthetic
  source 1..18: mol_type = other DNA
tgaccctagctagacctt

SEQ ID NO: 852 - DNA, 18 bp, synthetic construct
  misc_feature 1..18: note = Synthetic
  source 1..18: mol_type = other DNA
cccagcttacttaccttg

SEQ ID NO: 853 - DNA, 18 bp, synthetic construct
  misc_feature 1..18: note = Synthetic
  source 1..18: mol_type = other DNA
ttttgggtaggtagcact

SEQ ID NO: 854 - DNA, 18 bp, synthetic construct
  misc_feature 1..18: note = Synthetic
  source 1..18: mol_type = other DNA
tgccctacttactccctg

SEQ ID NO: 855 - DNA, 18 bp, synthetic construct
  misc_feature 1..18: note = Synthetic
  source 1..18: mol_type = other DNA
ctttacaaccaaccggct

SEQ ID NO: 856 - DNA, 18 bp, synthetic construct
  misc_feature 1..18: note = Synthetic
  source 1..18: mol_type = other DNA
aggcactgtctgtacggt

SEQ ID NO: 857 - DNA, 18 bp, synthetic construct
  misc_feature 1..18: note = Synthetic
  source 1..18: mol_type = other DNA
tctctattaattagtaag

SEQ ID NO: 858 - DNA, 18 bp, synthetic construct
  misc_feature 1..18: note = Synthetic
  source 1..18: mol_type = other DNA
gcctgctgcctgcctgga

SEQ ID NO: 859 - DNA, 18 bp, synthetic construct
  misc_feature 1..18: note = Synthetic
  source 1..18: mol_type = other DNA
ctggtgatagatagatga

SEQ ID NO: 860 - DNA, 18 bp, synthetic construct
  misc_feature 1..18: note = Synthetic
  source 1..18: mol_type = other DNA
gagcttgcatgcattcca

SEQ ID NO: 861 - DNA, 18 bp, synthetic construct
  misc_feature 1..18: note = Synthetic
  source 1..18: mol_type = other DNA
ctggaccgtccgtcgcct

SEQ ID NO: 862 - DNA, 18 bp, synthetic construct
  misc_feature 1..18: note = Synthetic
  source 1..18: mol_type = other DNA
ctgcccggtcggtgctca

SEQ ID NO: 863 - DNA, 18 bp, synthetic construct
  misc_feature 1..18: note = Synthetic
  source 1..18: mol_type = other DNA
gactggtcagtcagatga

SEQ ID NO: 864 - DNA, 18 bp, synthetic construct
  misc_feature 1..18: note = Synthetic
  source 1..18: mol_type = other DNA
atcgatgagtgagtgtca

SEQ ID NO: 865 - DNA, 18 bp, synthetic construct
  misc_feature 1..18: note = Synthetic
  source 1..18: mol_type = other DNA
gagggtggctggctacaa

SEQ ID NO: 866 - DNA, 18 bp, synthetic construct
  misc_feature 1..18: note = Synthetic
  source 1..18: mol_type = other DNA
agcttgacagacagagcc

SEQ ID NO: 867 - DNA, 18 bp, synthetic construct
  misc_feature 1..18: note = Synthetic
  source 1..18: mol_type = other DNA
cagagacatacatccagg

SEQ ID NO: 868 - DNA, 18 bp, synthetic construct
  misc_feature 1..18: note = Synthetic
  source 1..18: mol_type = other DNA
tcaccctagctaggtatg

SEQ ID NO: 869 - DNA, 18 bp, synthetic construct
  misc_feature 1..18: note = Synthetic
  source 1..18: mol_type = other DNA
catgctgcttgctggcca

SEQ ID NO: 870 - DNA, 18 bp, synthetic construct
  misc_feature 1..18: note = Synthetic
  source 1..18: mol_type = other DNA
ctcccggctggctgcctg

SEQ ID NO: 871 - DNA, 18 bp, synthetic construct
  misc_feature 1..18: note = Synthetic
  source 1..18: mol_type = other DNA
ggcttaagaaagaacatc

SEQ ID NO: 872 - DNA, 18 bp, synthetic construct
  misc_feature 1..18: note = Synthetic
  source 1..18: mol_type = other DNA
acaagagatagatacccc

SEQ ID NO: 873 - DNA, 18 bp, synthetic construct
  misc_feature 1..18: note = Synthetic
  source 1..18: mol_type = other DNA
tgatcttctttctatgta

SEQ ID NO: 874 - DNA, 18 bp, synthetic construct
  misc_feature 1..18: note = Synthetic
  source 1..18: mol_type = other DNA
gagaggactgactggctg

SEQ ID NO: 875 - DNA, 18 bp, synthetic construct
  misc_feature 1..18: note = Synthetic
  source 1..18: mol_type = other DNA
cagcaagccagcctccag

SEQ ID NO: 876 - DNA, 18 bp, synthetic construct
  misc_feature 1..18: note = Synthetic
  source 1..18: mol_type = other DNA
ccaccatgtatgtgcatg

SEQ ID NO: 877 - DNA, 20 bp, synthetic construct
  misc_feature 1..20: note = Synthetic
  source 1..20: mol_type = other DNA
ccaggacatcacatcatctc

SEQ ID NO: 878 - DNA, 20 bp, synthetic construct
  misc_feature 1..20: note = Synthetic
  source 1..20: mol_type = other DNA
ttcatccttgccttgtagag

SEQ ID NO: 879 - DNA, 20 bp, synthetic construct
  misc_feature 1..20: note = Synthetic
  source 1..20: mol_type = other DNA
cgaaatactttacttttcta

SEQ ID NO: 880 - DNA, 20 bp, synthetic construct
  misc_feature 1..20: note = Synthetic
  source 1..20: mol_type = other DNA
gggaggtcatgtcatgcctg

SEQ ID NO: 881 - DNA, 20 bp, synthetic construct
  misc_feature 1..20: note = Synthetic
  source 1..20: mol_type = other DNA
tcccataaactaaactgtcc

SEQ ID NO: 882 - DNA, 20 bp, synthetic construct
  misc_feature 1..20: note = Synthetic
  source 1..20: mol_type = other DNA
cagcatgatgtgatgcagga

SEQ ID NO: 883 - DNA, 20 bp, synthetic construct
  misc_feature 1..20: note = Synthetic
  source 1..20: mol_type = other DNA
catgtttttattttagatga

SEQ ID NO: 884 - DNA, 20 bp, synthetic construct
  misc_feature 1..20: note = Synthetic
  source 1..20: mol_type = other DNA
tggcaatctgatctggtgtt

SEQ ID NO: 885 - DNA, 20 bp, synthetic construct
  misc_feature 1..20: note = Synthetic
  source 1..20: mol_type = other DNA
cttacgatgtgatgtgcggg

SEQ ID NO: 886 - DNA, 20 bp, synthetic construct
  misc_feature 1..20: note = Synthetic
  source 1..20: mol_type = other DNA
tgagacatatcatatcaggt

SEQ ID NO: 887 - DNA, 20 bp, synthetic construct
  misc_feature 1..20: note = Synthetic
  source 1..20: mol_type = other DNA
tgcaggagtagagtatgagg

SEQ ID NO: 888 - DNA, 20 bp, synthetic construct
  misc_feature 1..20: note = Synthetic
  source 1..20: mol_type = other DNA
tctgggcggtgcggtgaagc

SEQ ID NO: 889 - DNA, 20 bp, synthetic construct
  misc_feature 1..20: note = Synthetic
  source 1..20: mol_type = other DNA
tctttagctaagctacacca

SEQ ID NO: 890 - DNA, 20 bp, synthetic construct
  misc_feature 1..20: note = Synthetic
  source 1..20: mol_type = other DNA
tggctcataccataccctcc

SEQ ID NO: 891 - DNA, 20 bp, synthetic construct
  misc_feature 1..20: note = Synthetic
  source 1..20: mol_type = other DNA
aaacatcgtgtcgtgaataa

SEQ ID NO: 892 - DNA, 20 bp, synthetic construct
  misc_feature 1..20: note = Synthetic
  source 1..20: mol_type = other DNA
tgcttacgacacgacaacgt

SEQ ID NO: 893 - DNA, 20 bp, synthetic construct
  misc_feature 1..20: note = Synthetic
  source 1..20: mol_type = other DNA
tgcggctgccctgccctctg

SEQ ID NO: 894 - DNA, 20 bp, synthetic construct
  misc_feature 1..20: note = Synthetic
  source 1..20: mol_type = other DNA
tggtggccccgccccgactg

SEQ ID NO: 895 - DNA, 20 bp, synthetic construct
  misc_feature 1..20: note = Synthetic
  source 1..20: mol_type = other DNA
acgctggaccggaccggagc

SEQ ID NO: 896 - DNA, 20 bp, synthetic construct
  misc_feature 1..20: note = Synthetic
  source 1..20: mol_type = other DNA
tccccagaggagaggatatg

SEQ ID NO: 897 - DNA, 20 bp, synthetic construct
  misc_feature 1..20: note = Synthetic
  source 1..20: mol_type = other DNA
tatggttctcttctcttcca

SEQ ID NO: 898 - DNA, 20 bp, synthetic construct
  misc_feature 1..20: note = Synthetic
  source 1..20: mol_type = other DNA
ccaccgtcaggtcaggctaa

SEQ ID NO: 899 - DNA, 20 bp, synthetic construct
  misc_feature 1..20: note = Synthetic
  source 1..20: mol_type = other DNA
gggcaagatcagatccggcg

SEQ ID NO: 900 - DNA, 20 bp, synthetic construct
  misc_feature 1..20: note = Synthetic
  source 1..20: mol_type = other DNA
gtcgtagggcagggccgcag

SEQ ID NO: 901 - DNA, 22 bp, synthetic construct
  misc_feature 1..22: note = Synthetic
  source 1..22: mol_type = other DNA
gcgcgagcgccagcgccagcgc

SEQ ID NO: 902 - DNA, 22 bp, synthetic construct
  misc_feature 1..22: note = Synthetic
  source 1..22: mol_type = other DNA
aactccggcatcggcatcgggg

SEQ ID NO: 903 - DNA, 22 bp, synthetic construct
  misc_feature 1..22: note = Synthetic
  source 1..22: mol_type = other DNA
tactacgagaccgagaccggca

SEQ ID NO: 904 - DNA, 24 bp, synthetic construct
  misc_feature 1..24: note = Synthetic
  source 1..24: mol_type = other DNA
gtgctcgctttccgctttcctgga

SEQ ID NO: 905 - DNA, 24 bp, synthetic construct
  misc_feature 1..24: note = Synthetic
  source 1..24: mol_type = other DNA
caagggaaaatcgaaaatcgagat

SEQ ID NO: 906 - DNA, 24 bp, synthetic construct
  misc_feature 1..24: note = Synthetic
  source 1..24: mol_type = other DNA
gaagcagaagatagaagatcggct

SEQ ID NO: 907 - DNA, 24 bp, synthetic construct
  misc_feature 1..24: note = Synthetic
  source 1..24: mol_type = other DNA
cattctaggacttaggacttgccc

SEQ ID NO: 908 - DNA, 24 bp, synthetic construct
  misc_feature 1..24: note = Synthetic
  source 1..24: mol_type = other DNA
aacgtcgtaatccgtaatcgctgg

SEQ ID NO: 909 - DNA, 24 bp, synthetic construct
  misc_feature 1..24: note = Synthetic
  source 1..24: mol_type = other DNA
gatggccatggcccatggcgcgga

SEQ ID NO: 910 - DNA, 24 bp, synthetic construct
  misc_feature 1..24: note = Synthetic
  source 1..24: mol_type = other DNA
gtagcaccatcaaccatcatttcc

SEQ ID NO: 911 - DNA, 24 bp, synthetic construct
  misc_feature 1..24: note = Synthetic
  source 1..24: mol_type = other DNA
gatagtgtctgttgtctgtgtgaa

SEQ ID NO: 912 - DNA, 24 bp, synthetic construct
  misc_feature 1..24: note = Synthetic
  source 1..24: mol_type = other DNA
aaccgctgcattctgcattcctca

SEQ ID NO: 913 - DNA, 24 bp, synthetic construct
  misc_feature 1..24: note = Synthetic
  source 1..24: mol_type = other DNA
gacaaatctgacatctgacgagga

SEQ ID NO: 914 - DNA, 24 bp, synthetic construct
  misc_feature 1..24: note = Synthetic
  source 1..24: mol_type = other DNA
ggcatgctgctggctgctggccag

SEQ ID NO: 915 - DNA, 24 bp, synthetic construct
  misc_feature 1..24: note = Synthetic
  source 1..24: mol_type = other DNA
ggtgtggatttgggatttggtgtg

SEQ ID NO: 916 - DNA, 26 bp, synthetic construct
  misc_feature 1..26: note = Synthetic
  source 1..26: mol_type = other DNA
aaaagaaaattccaaaattccaacag

SEQ ID NO: 917 - DNA, 26 bp, synthetic construct
  misc_feature 1..26: note = Synthetic
  source 1..26: mol_type = other DNA
tctgcctggatgactggatgacatgc

SEQ ID NO: 918 - DNA, 26 bp, synthetic construct
  misc_feature 1..26: note = Synthetic
  source 1..26: mol_type = other DNA
tcagctcttatcttcttatcttcatt

SEQ ID NO: 919 - DNA, 26 bp, synthetic construct
  misc_feature 1..26: note = Synthetic
  source 1..26: mol_type = other DNA
tcaccgttgttttgttgttttgttta

SEQ ID NO: 920 - DNA, 26 bp, synthetic construct
  misc_feature 1..26: note = Synthetic
  source 1..26: mol_type = other DNA
tcgccccacggctccacggctcctcg

SEQ ID NO: 921 - DNA, 26 bp, synthetic construct
  misc_feature 1..26: note = Synthetic
  source 1..26: mol_type = other DNA
taaaatatcaccttatcaccttgtga

SEQ ID NO: 922 - DNA, 26 bp, synthetic construct
  misc_feature 1..26: note = Synthetic
  source 1..26: mol_type = other DNA
gagaacattcatgcattcatgttttg

SEQ ID NO: 923 - DNA, 26 bp, synthetic construct
  misc_feature 1..26: note = Synthetic
  source 1..26: mol_type = other DNA
cccacgctgagaggctgagagtcgtc

SEQ ID NO: 924 - DNA, 26 bp, synthetic construct
  misc_feature 1..26: note = Synthetic
  source 1..26: mol_type = other DNA
cttctacttgttgacttgttgatcag

SEQ ID NO: 925 - DNA, 26 bp, synthetic construct
  misc_feature 1..26: note = Synthetic
  source 1..26: mol_type = other DNA
gcttcaatcttataatcttattcagg

SEQ ID NO: 926 - DNA, 26 bp, synthetic construct
  misc_feature 1..26: note = Synthetic
  source 1..26: mol_type = other DNA
gcacaaggacatcaggacatcaagcc

SEQ ID NO: 927 - DNA, 26 bp, synthetic construct
  misc_feature 1..26: note = Synthetic
  source 1..26: mol_type = other DNA
aagatggctcggaggctcggatgagt

SEQ ID NO: 928 - DNA, 26 bp, synthetic construct
  misc_feature 1..26: note = Synthetic
  source 1..26: mol_type = other DNA
gcccagcgaagatgcgaagatgcgaa

SEQ ID NO: 929 - DNA, 26 bp, synthetic construct
  misc_feature 1..26: note = Synthetic
  source 1..26: mol_type = other DNA
tactcgctggtgagctggtgactgaa

SEQ ID NO: 930 - DNA, 26 bp, synthetic construct
  misc_feature 1..26: note = Synthetic
  source 1..26: mol_type = other DNA
cggcgcaagagaccaagagacccgaa

SEQ ID NO: 931 - DNA, 28 bp, synthetic construct
  misc_feature 1..28: note = Synthetic
  source 1..28: mol_type = other DNA
gatgatgacaagattgacaagatcgttg

SEQ ID NO: 932 - DNA, 28 bp, synthetic construct
  misc_feature 1..28: note = Synthetic
  source 1..28: mol_type = other DNA
cggctggagtgtgaggagtgtgaggagt

SEQ ID NO: 933 - DNA, 28 bp, synthetic construct
  misc_feature 1..28: note = Synthetic
  source 1..28: mol_type = other DNA
gagctgtgccgcacgtgccgcacggtga

SEQ ID NO: 934 - DNA, 28 bp, synthetic construct
  misc_feature 1..28: note = Synthetic
  source 1..28: mol_type = other DNA
ccgcagccgcagctgccgcagctctcgc

SEQ ID NO: 935 - DNA, 30 bp, synthetic construct
  misc_feature 1..30: note = Synthetic
  source 1..30: mol_type = other DNA
gtcaaaagagattccaagagattccaggct

SEQ ID NO: 936 - DNA, 30 bp, synthetic construct
  misc_feature 1..30: note = Synthetic
  source 1..30: mol_type = other DNA
cgggagctgcagcatgctgcagcatgctgc

SEQ ID NO: 937 - DNA, 30 bp, synthetic construct
  misc_feature 1..30: note = Synthetic
  source 1..30: mol_type = other DNA
gaaactttaatttgttttaatttgttcttg

SEQ ID NO: 938 - DNA, 30 bp, synthetic construct
  misc_feature 1..30: note = Synthetic
  source 1..30: mol_type = other DNA
gggcacatctgggtgcatctgggtgatact

SEQ ID NO: 939 - DNA, 30 bp, synthetic construct
  misc_feature 1..30: note = Synthetic
  source 1..30: mol_type = other DNA
gacccaacaagttcaaacaagttcaagtgt

SEQ ID NO: 940 - DNA, 32 bp, synthetic construct
  misc_feature 1..32: note = Synthetic
  source 1..32: mol_type = other DNA
gagctggcccggcggcggcccggcggcggcgc

SEQ ID NO: 941 - DNA, 32 bp, synthetic construct
  misc_feature 1..32: note = Synthetic
  source 1..32: mol_type = other DNA
caaagaagaaactattaagaaactattattga

SEQ ID NO: 942 - DNA, 32 bp, synthetic construct
  misc_feature 1..32: note = Synthetic
  source 1..32: mol_type = other DNA
tgaataaccctctgtcaaccctctgtcagctg

SEQ ID NO: 943 - DNA, 32 bp, synthetic construct
  misc_feature 1..32: note = Synthetic
  source 1..32: mol_type = other DNA
gctcacctgtggtccccctgtggtcccgccag

SEQ ID NO: 944 - DNA, 32 bp, synthetic construct
  misc_feature 1..32: note = Synthetic
  source 1..32: mol_type = other DNA
tgcaaggacaaatctgggacaaatctgacgag

SEQ ID NO: 945 - DNA, 34 bp, synthetic construct
  misc_feature 1..34: note = Synthetic
  source 1..34: mol_type = other DNA
gtgcgacgagctgtgccacgagctgtgccgcacg

SEQ ID NO: 946 - DNA, 34 bp, synthetic construct
  misc_feature 1..34: note = Synthetic
  source 1..34: mol_type = other DNA
ccccttctccttggccgtctccttggccgtcttt

SEQ ID NO: 947 - DNA, 36 bp, synthetic construct
  misc_feature 1..36: note = Synthetic
  source 1..36: mol_type = other DNA
cccttggtgagcacgctgggtgagcacgctgaccac

SEQ ID NO: 948 - DNA, 36 bp, synthetic construct
  misc_feature 1..36: note = Synthetic
  source 1..36: mol_type = other DNA
gccccgactgcaaggacagactgcaaggacaaatct

SEQ ID NO: 949 - DNA, 36 bp, synthetic construct
  misc_feature 1..36: note = Synthetic
  source 1..36: mol_type = other DNA
gctaaaggtcagctccacaggtcagctccacagccg

SEQ ID NO: 950 - DNA, 38 bp, synthetic construct
  misc_feature 1..38: note = Synthetic
  source 1..38: mol_type = other DNA
tggggcagcaaatcaagctcagcaaatcaagctctatt

SEQ ID NO: 951 - DNA, 38 bp, synthetic construct
  misc_feature 1..38: note = Synthetic
  source 1..38: mol_type = other DNA
cgctgctgccgcgccgcctctgccgcgccgcctctgcc

SEQ ID NO: 952 - DNA, 38 bp, synthetic construct
  misc_feature 1..38: note = Synthetic
  source 1..38: mol_type = other DNA
catatactgcatgcaaatgactgcatgcaaatgatccc

SEQ ID NO: 953 - DNA, 38 bp, synthetic construct
  misc_feature 1..38: note = Synthetic
  source 1..38: mol_type = other DNA
ctgcaaggacaaatctgacaggacaaatctgacgagga

SEQ ID NO: 954 - DNA, 40 bp, synthetic construct
  misc_feature 1..40: note = Synthetic
  source 1..40: mol_type = other DNA
ggtgaacctgggcaagctggacctgggcaagctggtcctt

SEQ ID NO: 955 - DNA, 40 bp, synthetic construct
  misc_feature 1..40: note = Synthetic
  source 1..40: mol_type = other DNA
tgctgctctgcgcgctgctcctctgcgcgctgctcgcccg

SEQ ID NO: 956 - DNA, 42 bp, synthetic construct
  misc_feature 1..42: note = Synthetic
  source 1..42: mol_type = other DNA
gatcctagctatcgttctttttagctatcgttcttttcactc

SEQ ID NO: 957 - DNA, 42 bp, synthetic construct
  misc_feature 1..42: note = Synthetic
  source 1..42: mol_type = other DNA
cgtggctgctctgcatgttttctgctctgcatgtttttaata

SEQ ID NO: 958 - DNA, 42 bp, synthetic construct
  misc_feature 1..42: note = Synthetic
  source 1..42: mol_type = other DNA
ggaattggttggtagacactgtggttggtagacactggtgct

SEQ ID NO: 959 - DNA, 42 bp, synthetic construct
  misc_feature 1..42: note = Synthetic
  source 1..42: mol_type = other DNA
tgtcctcttctgctgcttctttcttctgctgcttctttcttc

SEQ ID NO: 960 - DNA, 42 bp, synthetic construct
  misc_feature 1..42: note = Synthetic
  source 1..42: mol_type = other DNA
tgggtggcccggaggctgggcggcccggaggctgggctgggc

SEQ ID NO: 961 - DNA, 44 bp, synthetic construct
  misc_feature 1..44: note = Synthetic
  source 1..44: mol_type = other DNA
cgggtgggggtgcggcggaggcgggggtgcggcggaggcggggg

SEQ ID NO: 962 - DNA, 44 bp, synthetic construct
  misc_feature 1..44: note = Synthetic
  source 1..44: mol_type = other DNA
cggcggaggcgggggtgcggccgaggcgggggtgcggccggcgg

SEQ ID NO: 963 - DNA, 44 bp, synthetic construct
  misc_feature 1..44: note = Synthetic
  source 1..44: mol_type = other DNA
gcggaggcgggggtgcggccggggcgggggtgcggccggcgggc

SEQ ID NO: 964 - DNA, 44 bp, synthetic construct
  misc_feature 1..44: note = Synthetic
  source 1..44: mol_type = other DNA
ttccacccaaagcagctgtactcccaaagcagctgtacttctcc

SEQ ID NO: 965 - DNA, 44 bp, synthetic construct
  misc_feature 1..44: note = Synthetic
  source 1..44: mol_type = other DNA
ccccgcccaggccctgcagggccccaggccctgcagggccccag

SEQ ID NO: 966 - DNA, 44 bp, synthetic construct
  misc_feature 1..44: note = Synthetic
  source 1..44: mol_type = other DNA
ccctgagcaagtccagctcctcagcaagtccagctcctctcccg

SEQ ID NO: 967 - DNA, 44 bp, synthetic construct
  misc_feature 1..44: note = Synthetic
  source 1..44: mol_type = other DNA
aacgtgcggctgcgtggacctcgcggctgcgtggacctccacga

SEQ ID NO: 968 - DNA, 44 bp, synthetic construct
  misc_feature 1..44: note = Synthetic
  source 1..44: mol_type = other DNA
cgactgcaaggacaaatctgacgcaaggacaaatctgacgagga

SEQ ID NO: 969 - DNA, 46 bp, synthetic construct
  misc_feature 1..46: note = Synthetic
  source 1..46: mol_type = other DNA
gccctgcagacatagatgcccccgcagacatagatgcccccgtcaa

SEQ ID NO: 970 - DNA, 46 bp, synthetic construct
  misc_feature 1..46: note = Synthetic
  source 1..46: mol_type = other DNA
ctgttcgcgctgctgctgctctgcgcgctgctgctgctctgcgcgc

SEQ ID NO: 971 - DNA, 46 bp, synthetic construct
  misc_feature 1..46: note = Synthetic
  source 1..46: mol_type = other DNA
tgctgctgctctgcgcgctgctcctgctctgcgcgctgctcgcccg

SEQ ID NO: 972 - DNA, 48 bp, synthetic construct
  misc_feature 1..48: note = Synthetic
  source 1..48: mol_type = other DNA
cggtggctgggctggcagggctgagctgggctggcagggctgagctgg

SEQ ID NO: 973 - DNA, 48 bp, synthetic construct
  misc_feature 1..48: note = Synthetic
  source 1..48: mol_type = other DNA
ccaggcaggcggagcaccccaagccaggcggagcaccccaagccctcg

SEQ ID NO: 974 - DNA, 48 bp, synthetic construct
  misc_feature 1..48: note = Synthetic
  source 1..48: mol_type = other DNA
cagtgcaacagctccacctgcatccaacagctccacctgcatccccca

SEQ ID NO: 975 - DNA, 48 bp, synthetic construct
  misc_feature 1..48: note = Synthetic
  source 1..48: mol_type = other DNA
gactgccgggactggtcagatgaaccgggactggtcagatgaacccat

SEQ ID NO: 976 - DNA, 48 bp, synthetic construct
  misc_feature 1..48: note = Synthetic
  source 1..48: mol_type = other DNA
tcgtggtggatcctgttcatgggtgtggatcctgttcatgggtgcgta

SEQ ID NO: 977 - DNA, 50 bp, synthetic construct
  misc_feature 1..50: note = Synthetic
  source 1..50: mol_type = other DNA
accccgcgcacccctgtgcccacctgcgcacccctgtgcccacctgcgcc

SEQ ID NO: 978 - DNA, 50 bp, synthetic construct
  misc_feature 1..50: note = Synthetic
  source 1..50: mol_type = other DNA
gcggtggggcaggcggcggtgcggcggggcaggcggcggtgcggcggccg

SEQ ID NO: 979 - DNA, 50 bp, synthetic construct
  misc_feature 1..50: note = Synthetic
  source 1..50: mol_type = other DNA
attcctctcggtcatgctgcagctgtctcggtcatgctgcagctgtctga

SEQ ID NO: 980 - DNA, 50 bp, synthetic construct
  misc_feature 1..50: note = Synthetic
  source 1..50: mol_type = other DNA
ccccgactgcaaggacaaatctgacactgcaaggacaaatctgacgagga

SEQ ID NO: 981 - DNA, 50 bp, synthetic construct
  misc_feature 1..50: note = Synthetic
  source 1..50: mol_type = other DNA
gtctccttacagctgcctctgttgtcttacagctgcctctgttgtgacag

SEQ ID NO: 982-994 - skipped (13 entries)

SEQ ID NO: 995 - DNA, 20 bp, synthetic construct
  misc_feature 1..20: note = Synthetic
  source 1..20: mol_type = other DNA
acgtgaacgtacgtgacgtg

SEQ ID NO: 996 - skipped

SEQ ID NO: 997 - DNA, 14 bp, synthetic construct
  misc_feature 1..14: note = Synthetic
  source 1..14: mol_type = other DNA
acgtacgtgagagt

SEQ ID NO: 998 - skipped

SEQ ID NO: 999 - DNA, 20 bp, synthetic construct
  misc_feature 1..20: note = Synthetic
  source 1..20: mol_type = other DNA
acgtacgtacgtacgtgatt

SEQ ID NO: 1000 - skipped

SEQ ID NO: 1001 - DNA, 22 bp, synthetic construct
  misc_feature 1..22: note = Synthetic
  source 1..22: mol_type = other DNA
acgtacgtacgtacgtagctac

SEQ ID NO: 1002-1009 - skipped (8 entries)

SEQ ID NO: 1010 - DNA, 23 bp, synthetic construct
  misc_feature 1..23: note = Synthetic
  source 1..23: mol_type = other DNA
gaagcgctacctgattccaattc

SEQ ID NO: 1011 - DNA, 23 bp, synthetic construct
  misc_feature 1..23: note = Synthetic
  source 1..23: mol_type = other DNA
tggcagttaggaaggttgtatcg

SEQ ID NO: 1012 - DNA, 118 bp, synthetic construct
  misc_feature 1..118: note = Synthetic
  source 1..118: mol_type = other DNA
atggctacctcagagctgagctgcgaggtgtcgaggtgtcggaggagaactgtgagcgccgggaggccttctgggcagaatggaaggatctgacactgtccacacggcccgaggaggg

SEQ ID NO: 1013 - DNA, 27 bp, synthetic construct
  misc_feature 1..27: note = Synthetic
  misc_feature 18: note = n is a, c, g, or t
  source 1..27: mol_type = other DNA
agctgagctgcgaggtgntcgaggtgt

SEQ ID NO: 1014 - DNA, 151 bp, synthetic construct
  misc_feature 1..151: note = Synthetic
  source 1..151: mol_type = other DNA
gctgctccaggcatgtgggaagctgaagcggcagctctgcgccatgtaccggctgaactttctgaccacagcccccagcaggggaggcccccagcaggggaggcccacacctgccccagcacctgcaggaccaagtgcagaggctcatgcg

SEQ ID NO: 1015 - DNA, 33 bp, synthetic construct
  misc_feature 1..33: note = Synthetic
  misc_feature 19: note = n is a, c, g, or t
  source 1..33: mol_type = other DNA
ccagcaggggaggcccccnagcaggggaggccc

SEQ ID NO: 1016 - DNA, 49 bp, synthetic construct
  misc_feature 1..49: note = Synthetic
  source 1..49: mol_type = other DNA
cacagttctcctccgacacctcgagacctcgcagctcagctctgaggta

SEQ ID NO: 1017 - DNA, 40 bp, synthetic construct
  misc_feature 1..40: note = Synthetic
  source 1..40: mol_type = other DNA
ttctcctccgacacctcgcagctcagctctgaggtagcca

SEQ ID NO: 1018 - DNA, 47 bp, synthetic construct
  misc_feature 1..47: note = Synthetic
  source 1..47: mol_type = other DNA
gttctcctccgacacctcgacctcgaggtccgaggcagaggtgagca

SEQ ID NO: 1019 - DNA, 43 bp, synthetic construct
  misc_feature 1..43: note = Synthetic
  source 1..43: mol_type = other DNA
ttctcctccgacacctcgaacctcggatctgaggtatgagtga

SEQ ID NO: 1020 - DNA, 33 bp, synthetic construct; identical to SEQ ID NO: 1015
  misc_feature 1..33: note = Synthetic
  misc_feature 19: note = n is a, c, g, or t
  source 1..33: mol_type = other DNA

SEQ ID NO: 1021 - DNA, 26 bp, synthetic construct
  misc_feature 1..26: note = Synthetic
  source 1..26: mol_type = other DNA
agctgagctgcgaggtgtcgaggtgt

SEQ ID NO: 1022 - DNA, 18 bp, synthetic construct
  misc_feature 1..18: note = Synthetic
  source 1..18: mol_type = other DNA
agctgagctgcgaggtgt

SEQ ID NO: 1023 - DNA, 43 bp, synthetic construct
  misc_feature 1..43: note = Synthetic
  source 1..43: mol_type = other DNA
cagcccccagcaggggaggcccccagcaggggaggcccacacc

SEQ ID NO: 1024 - DNA, 43 bp, synthetic construct; identical to SEQ ID NO: 1023
  misc_feature 1..43: note = Synthetic
  source 1..43: mol_type = other DNA

SEQ ID NO: 1025 - DNA, 43 bp, synthetic construct; identical to SEQ ID NO: 1023
  misc_feature 1..43: note = Synthetic
  source 1..43: mol_type = other DNA

SEQ ID NO: 1026 - DNA, 43 bp, synthetic construct; identical to SEQ ID NO: 1023
  misc_feature 1..43: note = Synthetic
  source 1..43: mol_type = other DNA

SEQ ID NO: 1027 - DNA, 43 bp, synthetic construct; identical to SEQ ID NO: 1023
  misc_feature 1..43: note = Synthetic
  source 1..43: mol_type = other DNA

SEQ ID NO: 1028 - DNA, 43 bp, synthetic construct; identical to SEQ ID NO: 1023
  misc_feature 1..43: note = Synthetic
  source 1..43: mol_type = other DNA

SEQ ID NO: 1029 - DNA, 27 bp, synthetic construct
  misc_feature 1..27: note = Synthetic
  source 1..27: mol_type = other DNA
cagcccccagcaggggaggcccacacc

SEQ ID NO: 1030 - DNA, 38 bp, synthetic construct
  misc_feature 1..38: note = Synthetic
  source 1..38: mol_type = other DNA
cccccagcaggggaggcccccagcaggggaggcccaca

SEQ ID NO: 1031 - DNA, 22 bp, synthetic construct
  misc_feature 1..22: note = Synthetic
  source 1..22: mol_type = other DNA
cccccagcaggggaggcccaca

SEQ ID NO: 1032 - DNA, 38 bp, synthetic construct; identical to SEQ ID NO: 1030
  misc_feature 1..38: note = Synthetic
  source 1..38: mol_type = other DNA

SEQ ID NO: 1033 - DNA, 37 bp, synthetic construct
  misc_feature 1..37: note = Synthetic
  source 1..37: mol_type = other DNA
cccccagcaggggaggccccagcaggggagccccaca

SEQ ID NO: 1034 - DNA, 27 bp, synthetic construct
  misc_feature 1..27: note = Synthetic
  source 1..27: mol_type = other DNA
cccccagcaggggaggggaggcccaca

SEQ ID NO: 1035 - DNA, 32 bp, synthetic construct
  misc_feature 1..32: note = Synthetic
  source 1..32: mol_type = other DNA
cccccagcaggggagagcaggggaggcccaca

SEQ ID NO: 1036 - DNA, 38 bp, synthetic construct; identical to SEQ ID NO: 1030
  misc_feature 1..38: note = Synthetic
  source 1..38: mol_type = other DNA

SEQ ID NO: 1037 - DNA, 35 bp, synthetic construct
  misc_feature 1..35: note = Synthetic
  source 1..35: mol_type = other DNA
cccccagcaggggaggccagcaggggaggcccaca

SEQ ID NO: 1038 - DNA, 34 bp, synthetic construct
  misc_feature 1..34: note = Synthetic
  source 1..34: mol_type = other DNA
cccccagcaggggaggcagcaggggaggcccaca

SEQ ID NO: 1039 - DNA, 30 bp, synthetic construct
  misc_feature 1..30: note = Synthetic
  source 1..30: mol_type = other DNA
cccccagcaggggagcaggggaggcccaca

SEQ ID NO: 1040 - DNA, 36 bp, synthetic construct
  misc_feature 1..36: note = Synthetic
  source 1..36: mol_type = other DNA
cccccagcaggggaggcccagcaggggaggcccaca

SEQ ID NO: 1041 - DNA, 35 bp, synthetic construct
  misc_feature 1..35: note = Synthetic
  source 1..35: mol_type = other DNA
cccccagcaggggaggccccccaggggaggccaca

SEQ ID NO: 1042 - DNA, 37 bp, synthetic construct
  misc_feature 1..37: note = Synthetic
  source 1..37: mol_type = other DNA
cccccagcaggggaggcccccgcaggggaggcccaca

SEQ ID NO: 1043 - DNA, 31 bp, synthetic construct
  misc_feature 1..31: note = Synthetic
  source 1..31: mol_type = other DNA
cccccagcaggggaagcaggggaggcccaca

SEQ ID NO: 1044 - DNA, 28 bp, synthetic construct
  misc_feature 1..28: note = Synthetic
  source 1..28: mol_type = other DNA
ataggtgcctatatgtgatggatgggtg

SEQ ID NO: 1045 - DNA, 28 bp, synthetic construct
  misc_feature 1..28: note = Synthetic
  source 1..28: mol_type = other DNA
ctagccccagcaaagccaagccaaggtg

SEQ ID NO: 1046 - DNA, 28 bp, synthetic construct
  misc_feature 1..28: note = Synthetic
  source 1..28: mol_type = other DNA
catggtgctggccggcgtgggcgtggag

SEQ ID NO: 1047 - DNA, 28 bp, synthetic construct
  misc_feature 1..28: note = Synthetic
  source 1..28: mol_type = other DNA
tttgggtcagaagaaaagggaaaaggga

SEQ ID NO: 1048 - DNA, 51 bp, synthetic construct
  misc_feature 1..51: note = Synthetic
  source 1..51: mol_type = other DNA
tttgggacataggtacaaacatagtggaccataggtacaaacatagtggac

SEQ ID NO: 1049 - DNA, 54 bp, synthetic construct
  misc_feature 1..54: note = Synthetic
  source 1..54: mol_type = other DNA
cattggcctttggggtgcacttctgagcattggcctttggggtgcacttctgag

SEQ ID NO: 1050 - DNA, 58 bp, synthetic construct
  misc_feature 1..58: note = Synthetic
  source 1..58: mol_type = other DNA
aagtggatgttatgtgctcacactacaataagtggatgttatgtgctcacactacaat

SEQ ID NO: 1051 - DNA, 64 bp, synthetic construct
  misc_feature 1..64: note = Synthetic
  source 1..64: mol_type = other DNA
tctagggagtaaacatgcaaagattttggaactctagggagtaaacatgcaaagattttggaac

SEQ ID NO: 1052 - DNA, 72 bp, synthetic construct
  misc_feature 1..72: note = Synthetic
  source 1..72: mol_type = other DNA
aaggggcttataattcaatatccatacatgacatagaaggggcttataattcaatatccatacatgacatag

SEQ ID NO: 1053 - DNA, 75 bp, synthetic construct
  misc_feature 1..75: note = Synthetic
  source 1..75: mol_type = other DNA
ggttccactatgtagaaatccttccagtcagggccataggatagatatacggttcaggtaccagggggcagagag

SEQ ID NO: 1054 - DNA, 75 bp, synthetic construct
  misc_feature 1..75: note = Synthetic
  source 1..75: mol_type = other DNA
ccaaggtgatacatctttaggaaggtcagtcccggtatcctatctatatgccaagtccatggtcccccgtctctc

SEQ ID NO: 1055 - DNA, 70 bp, synthetic construct
  misc_feature 1..70: note = Synthetic
  source 1..70: mol_type = other DNA
cccttctctctgccccctggtacctgaaccgtatatctatcctatggccctgactggaaggatttctacg

SEQ ID NO: 1056 - DNA, 60 bp, synthetic construct
  misc_feature 1..60: note = Synthetic
  source 1..60: mol_type = other DNA
ccccctggtacctgaaccgtatatctatcctatggccctgactggaaggatttctacgta

SEQ ID NO: 1057 - DNA, 61 bp, synthetic construct
  misc_feature 1..61: note = Synthetic
  source 1..61: mol_type = other DNA
cccctggtacctgaaccgtatatctatcctatggccctgactggaaggatttctacgtagt

SEQ ID NO: 1058 - DNA, 66 bp, synthetic construct
  misc_feature 1..66: note = Synthetic
  source 1..66: mol_type = other DNA
cccttctctctgccccctggtacctgaaccgtatatctatcctatggccctgactggaaggatttc

SEQ ID NO: 1059 - DNA, 38 bp, synthetic construct
  misc_feature 1..38: note = Synthetic
  source 1..38: mol_type = other DNA
tttatctgtcccctccaccccacagtggggccactagg

SEQ ID NO: 1060 - DNA, 39 bp, synthetic construct
  misc_feature 1..39: note = Synthetic
  source 1..39: mol_type = other DNA
tttatctgtcccctccaccccacaagtggggccactagg

SEQ ID NO: 1061 - DNA, 36 bp, synthetic construct
  misc_feature 1..36: note = Synthetic
  source 1..36: mol_type = other DNA
tttatctgtcccctccaccccagtggggccactagg

SEQ ID NO: 1062 - DNA, 22 bp, synthetic construct
  misc_feature 1..22: note = Synthetic
  source 1..22: mol_type = other DNA
tttatctgtcccctccactagg

SEQ ID NO: 1063 - DNA, 33 bp, synthetic construct
  misc_feature 1..33: note = Synthetic
  source 1..33: mol_type = other DNA
tttatctgtcccctccacagtggggccactagg

SEQ ID NO: 1064 - DNA, 27 bp, synthetic construct
  misc_feature 1..27: note = Synthetic
  source 1..27: mol_type = other DNA
tttatctgtcccctccaccccactagg

SEQ ID NO: 1065 - DNA, 34 bp, synthetic construct
  misc_feature 1..34: note = Synthetic
  source 1..34: mol_type = other DNA
tttatctgtcccctccaccccatgggccactagg

SEQ ID NO: 1066 - DNA, 37 bp, synthetic construct
  misc_feature 1..37: note = Synthetic
  source 1..37: mol_type = other DNA
tttatctgtcccctccaccccaagtggggccactagg

SEQ ID NO: 1067 - DNA, 34 bp, synthetic construct
  misc_feature 1..34: note = Synthetic
  source 1..34: mol_type = other DNA
tttatctgtcccctccaccagtggggccactagg

SEQ ID NO: 1068 - DNA, 30 bp, synthetic construct
  misc_feature 1..30: note = Synthetic
  source 1..30: mol_type = other DNA
tttatctgtcccctcagtggggccactagg

SEQ ID NO: 1069 - DNA, 37 bp, synthetic construct
  misc_feature 1..37: note = Synthetic
  source 1..37: mol_type = other DNA
tttatctgtcccctccaccccacatggggccactagg

SEQ ID NO: 1070 - DNA, 32 bp, synthetic construct
  misc_feature 1..32: note = Synthetic
  source 1..32: mol_type = other DNA
tttatctgtcccctccaagtggggccactagg

SEQ ID NO: 1071 - DNA, 37 bp, synthetic construct
  misc_feature 1..37: note = Synthetic
  source 1..37: mol_type = other DNA
tttatctgtcccctccacccccagtggggccactagg

SEQ ID NO: 1072 - DNA, 29 bp, synthetic construct
  misc_feature 1..29: note = Synthetic
  source 1..29: mol_type = other DNA
tttatctgtcccctagtggggccactagg

SEQ ID NO: 1073 - DNA, 48 bp, synthetic construct
  misc_feature 1..48: note = Synthetic
  source 1..48: mol_type = other DNA
ggcggcggggagcagcatggagccggcggcggggagcagcatggagcc

SEQ ID NO: 1074 - DNA, 48 bp, synthetic construct; identical to SEQ ID NO: 1073
  misc_feature 1..48: note = Synthetic
  source 1..48: mol_type = other DNA

SEQ ID NO: 1075 - DNA, 48 bp, synthetic construct; identical to SEQ ID NO: 1073
  misc_feature 1..48: note = Synthetic
  source 1..48: mol_type = other DNA

SEQ ID NO: 1076 - DNA, 54 bp, synthetic construct; identical to SEQ ID NO: 1049
  misc_feature 1..54: note = Synthetic
  source 1..54: mol_type = other DNA

SEQ ID NO: 1077 - DNA, 54 bp, synthetic construct; identical to SEQ ID NO: 1049
  misc_feature 1..54: note = Synthetic
  source 1..54: mol_type = other DNA

SEQ ID NO: 1078 - DNA, 34 bp, synthetic construct
  misc_feature 1..34: note = Synthetic
  source 1..34: mol_type = other DNA
agctgagctgcgaggtgtcggaggagaactgtga

SEQ ID NO: 1079 - DNA, 40 bp, synthetic construct
  misc_feature 1..40: note = Synthetic
  source 1..40: mol_type = other DNA
agctgagctgcgaggtcgaggtgtcggaggagaactgtga

SEQ ID NO: 1080 - DNA, 25 bp, synthetic construct
  misc_feature 1..25: note = Synthetic
  source 1..25: mol_type = other DNA
agctgagctgcgaggtgaactgtga

SEQ ID NO: 1081 - DNA, 42 bp, synthetic construct
  misc_feature 1..42: note = Synthetic
  source 1..42: mol_type = other DNA
agctgagctgcgaggtgtcgaggtgtcggaggagaactgtga

SEQ ID NO: 1082 - DNA, 43 bp, synthetic construct
  misc_feature 1..43: note = Synthetic
  source 1..43: mol_type = other DNA
agctgagctgcgaggtgttcgaggtgtcggaggagaactgtga

SEQ ID NO: 1083 - DNA, 44 bp, synthetic construct
  misc_feature 1..44: note = Synthetic
  source 1..44: mol_type = other DNA
agctgagctgcgaggtgtgtcgaggtgtcggaggagaactgtga

SEQ ID NO: 1084 - DNA, 41 bp, synthetic construct
  misc_feature 1..41: note = Synthetic
  source 1..41: mol_type = other DNA
agctgagctgcgaggttcgaggtgtcggaggagaactgtga

SEQ ID NO: 1085 - DNA, 39 bp, synthetic construct
  misc_feature 1..39: note = Synthetic
  source 1..39: mol_type = other DNA
agctgagctgcgagtcgaggtgtcggaggagaactgtga

SEQ ID NO: 1086 - DNA, 35 bp, synthetic construct
  misc_feature 1..35: note = Synthetic
  source 1..35: mol_type = other DNA
agctgagctgcgaggtgttcggaggagaactgtga

SEQ ID NO: 1087 - DNA, 36 bp, synthetic construct
  misc_feature 1..36: note = Synthetic
  source 1..36: mol_type = other DNA
agctgagctgctcgaggtgtcggaggagaactgtga

SEQ ID NO: 1088 - DNA, 28 bp, synthetic construct
  misc_feature 1..28: note = Synthetic
  source 1..28: mol_type = other DNA
agctgagctgcgaggaggagaactgtga

SEQ ID NO: 1089 - DNA, 34 bp, synthetic construct; identical to SEQ ID NO: 1078
  misc_feature 1..34: note = Synthetic
  source 1..34: mol_type = other DNA

SEQ ID NO: 1090 - DNA, 42 bp, synthetic construct; identical to SEQ ID NO: 1081
  misc_feature 1..42: note = Synthetic
  source 1..42: mol_type = other DNA

SEQ ID NO: 1091 - DNA, 40 bp, synthetic construct; identical to SEQ ID NO: 1079
  misc_feature 1..40: note = Synthetic
  source 1..40: mol_type = other DNA

SEQ ID NO: 1092 - DNA, 43 bp, synthetic construct; identical to SEQ ID NO: 1082
  misc_feature 1..43: note = Synthetic
  source 1..43: mol_type = other DNA

SEQ ID NO: 1093 - DNA, 25 bp, synthetic construct
  misc_feature 1..25: note = Synthetic
  source 1..25: mol_type = other DNA
agctgagctgcgaggtgaactgtga

SEQ ID NO: 1094 - DNA, 35 bp, synthetic construct; identical to SEQ ID NO: 1086
  misc_feature 1..35: note = Synthetic
  source 1..35: mol_type = other DNA

SEQ ID NO: 1095 - DNA, 44 bp, synthetic construct; identical to SEQ ID NO: 1083
  misc_feature 1..44: note = Synthetic
  source 1..44: mol_type = other DNA

SEQ ID NO: 1096 - DNA, 41 bp, synthetic construct; identical to SEQ ID NO: 1084
  misc_feature 1..41: note = Synthetic
  source 1..41: mol_type = other DNA

SEQ ID NO: 1097 - DNA, 36 bp, synthetic construct; identical to SEQ ID NO: 1087
  misc_feature 1..36: note = Synthetic
  source 1..36: mol_type = other DNA

SEQ ID NO: 1098 - DNA, 39 bp, synthetic construct; identical to SEQ ID NO: 1085
  misc_feature 1..39: note = Synthetic
  source 1..39: mol_type = other DNA

SEQ ID NO: 1099 - DNA, 28 bp, synthetic construct
  misc_feature 1..28: note = Synthetic
  source 1..28: mol_type = other DNA
agctgagctgcgaggaggagaactgtga

SEQ ID NO: 1100 - DNA, 56 bp, synthetic construct
  misc_feature 1..56: note = Synthetic
  source 1..56: mol_type = other DNA
gtgatcatggctacctcagctgagctgcgaggtgtcgaggtgtcggaggagaactg

SEQ ID NO: 1101 - DNA, 56 bp, synthetic construct; identical to SEQ ID NO: 1100
  misc_feature 1..56: note = Synthetic
  source 1..56: mol_type = other DNA

SEQ ID NO: 1102 - DNA, 35 bp, synthetic construct
  misc_feature 1..35: note = Synthetic
  source 1..35: mol_type = other DNA
gtgatcatggctacctcagctgagctgcgaggtgt

SEQ ID NO: 1103 - DNA, 36 bp, synthetic construct
  misc_feature 1..36: note = Synthetic
  source 1..36: mol_type = other DNA
gtgatcatggctaccggaggtgtcggaggagaactg

SEQ ID NO: 1104 - DNA, 35 bp, synthetic construct; identical to SEQ ID NO: 1102
  misc_feature 1..35: note = Synthetic
  source 1..35: mol_type = other DNA

SEQ ID NO: 1105 - DNA, 35 bp, synthetic construct; identical to SEQ ID NO: 1102
  misc_feature 1..35: note = Synthetic
  source 1..35: mol_type = other DNA

SEQ ID NO: 1106 - DNA, 48 bp, synthetic construct
  misc_feature 1..48: note = Synthetic
  source 1..48: mol_type = other DNA
gtgatcatggctacctcagctgagctgcgaggtgtcggaggagaactg

SEQ ID NO: 1107 - DNA, 37 bp, synthetic construct
  misc_feature 1..37: note = Synthetic
  source 1..37: mol_type = other DNA
gtgatcatggctacctcgaggtgtcggaggagaactg